(19) United States
(12) Patent Application Publication
(10) Pub. No.: US 2024/0343755 A1
(43) Pub. Date: Oct. 17, 2024

(54) STABLE ISOQUINOLINE-CORTICOSTEROID CONJUGATES AND USES THEREOF

(71) Applicant: ALCON INC., Fribourg (CH)

(72) Inventors: Mitchell A. deLong, Chapel Hill, NC (US); Jill M. Sturdivant, Chapel Hill, NC (US); Cynthia L. Lichorowic, Durham, NC (US)

(21) Appl. No.: 18/557,899

(22) PCT Filed: Apr. 29, 2022
(86) PCT No.: PCT/US2022/027072

§ 371 Date: Oct. 27, 2023

Related U.S. Application Data
(60) Provisional application No. 63/181,862, filed on Apr. 29, 2021.

(51) Int. Cl. C07J 43/00; A61K 31/573; A61K 31/58; A61K 47/55; A61P 27/02; C07J 5/00; C07J 71/00

(52) U.S. Cl. CPC C07J 43/003; A61K 31/573; A61K 31/58; A61K 47/55; A61P 27/02; C07J 5/0053; C07J 5/0076; C07J 71/0031

(57) ABSTRACT

Provided herein are compounds and pharmaceutical compositions useful in modulating kinase activity, and related diseases. These compounds are conjugates of glucocorticoids with rho-kinase inhibitors. Also provided herein are methods of treating an eye disease or disorder in a subject. Also provided herein are methods of reducing intraocular pressure in a subject. Also provided herein are methods of modulating kinase activity in a cell. Also provided herein are methods of making the compounds provided herein, and compounds useful for the preparation of the compounds provided herein.

### RELATED APPLICATIONS

This application claims priority of U.S. Provisional Patent Application No. 63/181,862, filed Apr. 29, 2021, the entire contents of which are incorporated herein by reference.

### BACKGROUND

A variety of hormones, neurotransmitters and biologically active substances control, regulate or adjust the functions of living bodies by way of specific receptors located in cell membranes. Many of these receptors mediate the transmission of intracellular signals by activating guanine nucleotide-binding proteins (G proteins) to which the receptor is coupled. Such receptors are generically referred to as G-protein coupled receptors (GPCRs). The biological effects of activating or inhibiting these receptors is not direct, but is mediated by a host of intracellular proteins.

The importance of these secondary proteins has been recognized, and modulation of this class is now being investigated as intervention points in disease states. One of the most important classes of these downstream effectors is the “kinase” class. The various kinases have roles in the regulation of various physiological functions. For example, kinases have been implicated in a number of disease states.

Since the various kinases have roles in the regulation of various physiological functions and thereby have roles in many disease states, there is an urgent and continuing need for small molecule ligands which inhibit or modulate the activity of kinases. Without wishing to be bound by theory, it is thought that modulation of the activity of kinases, in particular ROCK and JAK kinases, by the compounds of the present disclosure is, at least in part, responsible for their beneficial effects.

Ocular inflammatory diseases or disorders, such as uveitis, an infectious corneal ulcer, endophthalmitis, an autoimmune disease of the cornea or ocular surface, or an ophthalmic manifestation of HIV disease, can slightly reduce vision or lead to severe vision loss or blindness. “Uveitis” is a general term describing a group of inflammatory diseases that produces swelling and destroys eye tissues. The term “uveitis” is used because the diseases often affect a part of the eye called the uvea. Nevertheless, uveitis is not limited to the uvea. These diseases also affect the lens, retina, optic nerve, and vitreous, producing reduced vision or blindness. Uveitis may be caused by diseases or disorders occurring in the eye or it can be part of an inflammatory disease affecting other parts of the body. Eye care professionals may describe such a disease or disorder more specifically as anterior uveitis, intermediate uveitis, posterior uveitis, or panuveitis uveitis. A less severe form of inflammation or irritation of the eye is known collectively as ‘dry eye’, which may also be treated with the compounds described herein.

To treat an ocular inflammatory disease or disorder, an eye care professional may prescribe corticosteroidal anti-inflammatory medication. Examples of such steroidal anti-inflammatory medication that can be used to treat an ocular inflammatory disease include, but are not limited to prednisone (sold under many brand names, such as DELTASONE and STERAPRED), methylprednisolone (MEDROL), prednisolone (PRELONE, PEDIAPRED), dexamethasone (DECADRON, HEXADROL), and hydrocortisone (ACTICORT, CORTEF).

Unfortunately, use of steroidal anti-inflammatory medication may cause deleterious side effects, such as increasing intraocular pressure. This side effect may result in glaucoma, or if the patient already suffers from glaucoma, it may further aggravate the patient's condition. In addition, at higher doses cataracts are possible with steroids. Further, many steroids have limited water solubility, which further limits their usefulness for delivery in an ophthalmic eyedrop.

Consequently, what is needed is a treatment for disease or disorder associated with kinase activity. What is also needed is a treatment for an ocular inflammatory disease or disorder that does not increase intraocular pressure. What is also needed is a treatment that reduces intraocular pressure. Such a treatment could have applications in treating an eye disease or disorder.

Isoquinoline-corticosteroid conjugates with improved stability are provided herein.

### SUMMARY

In one aspect, provided herein are compounds of Formula (I):

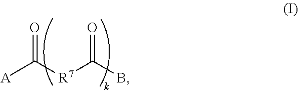

or a pharmaceutically acceptable salt thereof.

In one aspect, provided herein are compounds of Formula (Ia):

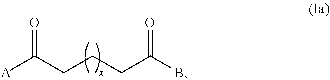

or a pharmaceutically acceptable salt thereof;
wherein
A is a corticosteroid moiety, linked through its primary alcohol to form an ester linkage.
B is a primary alcohol-containing rho kinase inhibitor, linked through its primary alcohol to form an ester linkage; and
x is 1 or 2 or 3.

In another aspect, provided herein are compounds of Formula (II):

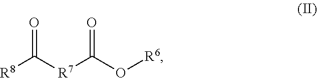

or a pharmaceutically acceptable salt thereof;
wherein
R⁶ is H, C₁₋₆ haloalkyl or C₁₋₆ alkyl;
R⁷ is C₁₋₆ alkyl, C₁₋₆ heteroalkyl, C₁₋₆ haloalkyl, C₃₋₁₀ cycloalkyl, C₃₋₁₀-cyclohaloalkyl aryl, heteroaryl, C₃₋₈ alkylene or a polyglycol of 5 to 10 units; and
R⁸ is a corticosteroidal moiety containing a primary alcohol, wherein R⁸ and the carbonyl to which it is attached form an ester linkage via that primary alcohol.

In another aspect, provided herein are compounds of Formula (III):

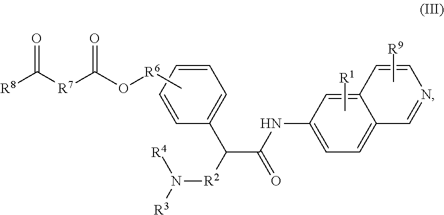

or a pharmaceutically acceptable salt thereof;
wherein
R¹ is H, —C₁₋₃ alkyl, —C₁₋₃ haloalkyl, or halogen;
R² is C₁₋₃ alkylene or C₁₋₃ haloalkylene;
R³ is an acid-labile protecting group;
R⁴ is H, ˜C₁₋₆ alkyl, or —C₁₋₆ haloalkyl;
R⁶ is C₁₋₆ alkylene or C₁₋₆ haloalkylene;
R⁷ is C₁₋₆ alkyl, C₁₋₆ heteroalkyl, C₁₋₆ haloalkyl, C₃₋₁₀ cycloalkyl, C₃₋₁₀-cyclohaloalkyl aryl, heteroaryl, C₃₋₈ alkylene or a polyglycol of 5 to 10 units; and
R⁸ is a corticosteroidal moiety containing a primary alcohol, wherein R⁸ and the carbonyl to which it is attached form an ester linkage via that primary alcohol; and
R⁹ is H, —C₁₋₃ alkyl, —C₁₋₃ haloalkyl, or halogen.

In another aspect, provided herein are methods of preparing a compound of Formula (IV):

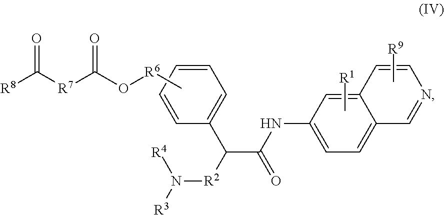

or a pharmaceutically acceptable salt thereof,
wherein
R¹ is H, —C₁₋₃ alkyl, —C₁₋₃ haloalkyl, or halogen;
R² is C₁₋₃ alkylene or C₁₋₃ haloalkylene;
R³ is H, —C₁₋₆ alkyl, or —C₁₋₆ haloalkyl;
R⁴ is H;
R⁶ is C₁₋₆ alkylene or C₁₋₃ haloalkylene;
R⁷ is C₁₋₆ alkyl, C₁₋₆ heteroalkyl, C₁₋₆ haloalkyl, C₃₋₁₀ cycloalkyl, C₃₋₁₀-Cyclohaloalkyl aryl, heteroaryl, C₃₋₈ alkylene, unsubstituted or a polyglycol of 5 to 10 units; and

R⁸ is a corticosteroidal moiety containing a primary alcohol, wherein R⁸ and the carbonyl to which it is attached form an ester linkage via that primary alcohol; and
R⁹ is H, —C₁₋₃ alkyl, —C₁₋₃ haloalkyl, or halogen.

In some embodiments of the formulae herein, the corticosteroidal moiety R⁸ is one of

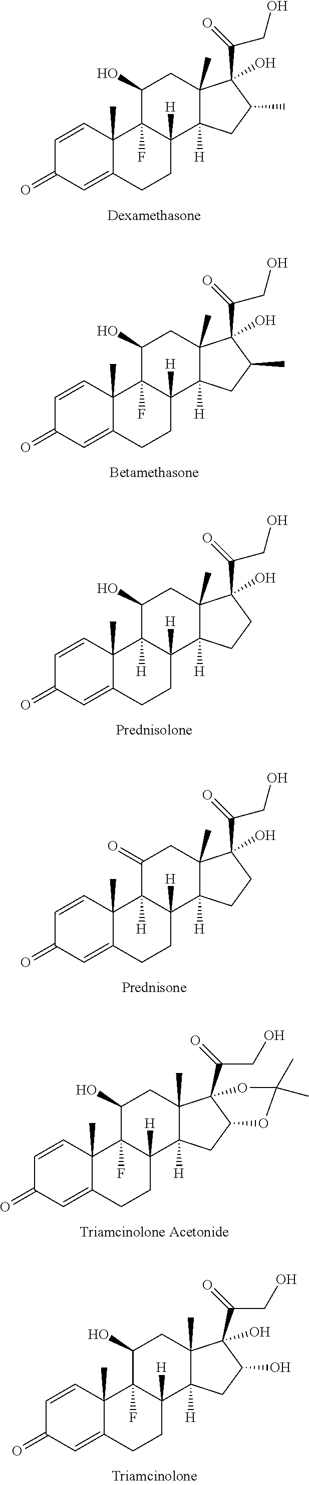

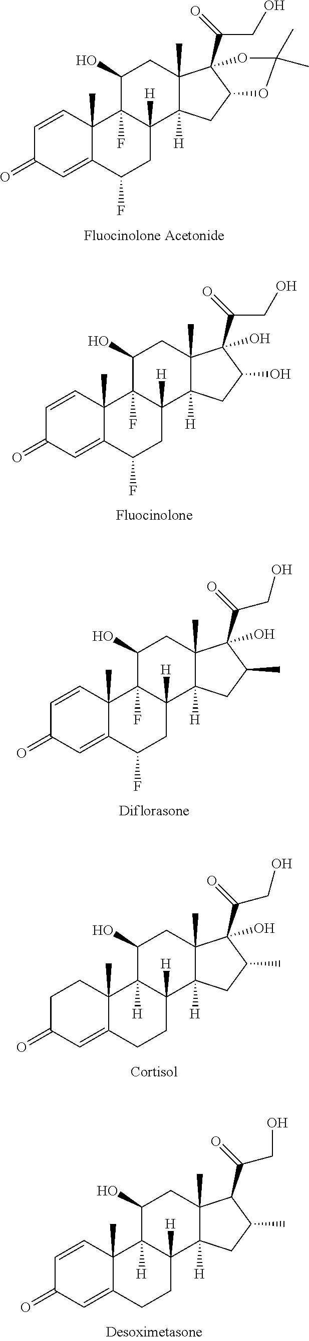

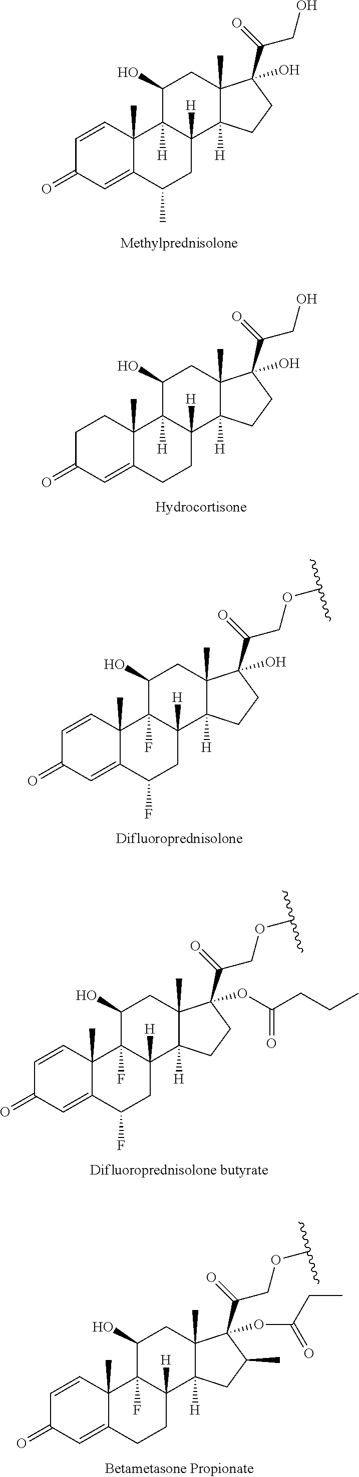

linked through their primary alcohols to form an ester linkage.

In another aspect, provided herein are methods of treating an eye disease or disorder in a subject in need thereof, comprising administering to the subject an effective amount of a compound or composition provided herein.

In another aspect, provided herein are methods of reducing intraocular pressure in an eye of a subject in need thereof, comprising administering to the subject an effective amount of a compound or composition provided herein.

In another aspect, provided herein are methods of modulating kinase activity in a cell, comprising contacting the cell with an amount of a compound or composition provided herein effective to modulate kinase activity.

In another aspect, provided herein are methods of treating an ocular inflammatory disease or disorder in a subject in need thereof, comprising administering to the subject an effective amount of a compound or composition provided herein. In a particular embodiment of such a method, intraocular pressure is not increased. In even more particular embodiments of such a method, intraocular pressure is maintained at physiological intraocular pressure, or reduced.

### BRIEF DESCRIPTION OF THE DRAWINGS

FIG. 1 shows a synthetic scheme for the synthesis of 4-((S)-3-amino-1-(isoquinolin-6-ylamino)-1-oxopropan-2-yl)benzyl (2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl) glutarate dihydrochloride (E5 2HCl).

FIG. 2 shows a synthetic scheme for the synthesis of 4-((S)-3-amino-1-(isoquinolin-6-ylamino)-1-oxopropan-2-yl)benzyl (2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl) adipate dihydrogen chloride (E8 2HCl).

FIG. 3 shows a synthetic scheme for the synthesis of 4-((S)-3-amino-1-(isoquinolin-6-ylamino)-1-oxopropan-2-yl)benzyl 4-(2-(2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)-2-oxoethoxy)benzoate dihydrochloride (E12 2HCl).

FIG. 4 shows a synthetic scheme for the synthesis of 4-((S)-3-amino-1-(isoquinolin-6-ylamino)-1-oxopropan-2-yl)benzyl (2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl) carbonate dihydrochloride (E102, 2HCl).

FIG. 5 shows the difference in stability of compounds described herein with different linker moieties (▾ glutamate, pH 6; ● glutarate, pH 6; ▴ carbonate, pH 5; and ▪ carbonate, pH 6) in a buffered solution.

FIG. 6 shows the difference in hydrolytic stability of compounds described herein in a buffered solution as described in Example 5.

FIG. 7 shows the difference in hydrolytic stability of compounds described herein in a buffered solution as described in Example 5.

FIG. 8 shows the difference in esterase stability of compounds described herein as described in Example 6.

FIG. 9 shows the clinical scoring following treatment with vehicle, loteprednol etabonate gel, and E5 in an in vivo PK/PD ocular surface model for Meibomian gland dysfunction as described in Example 7.

FIG. 10 shows the Meibomian gland orifice plugging following treatment with vehicle, loteprednol etabonate gel, and E5 in an in vivo PK/PD ocular surface model for Meibomian gland dysfunction as described in Example 7.

FIG. 11 shows the neutrophil levels in the meibomian gland and cunjunctiva following treatment with vehicle, loteprednol etabonate gel, and E5 in an in vivo PK/PD ocular surface model for Meibomian gland dysfunction as described in Example 7.

FIG. 12 shows re-epithelialization in an in vivo PK/PD ocular surface model for corneal wound healing (CWH.0023) following treatment with PBS vehicle, 0.1% dexamethasone in PBS, 0.5% loteprednol etabonate, 3.5% SBEβCD vehicle, and 0.1% E5 in SBEβCD as described in Example 8.

FIG. 13 shows myeloperoxidase (MPO) levels (eye surface inflammation) in an in vivo PK/PD ocular surface model for corneal wound healing (CWH.0023) following treatment with PBS vehicle, 0.1% dexamethasone in PBS, 0.5% loteprednol etabonate, 3.5% SBEβCD vehicle, and 0.1% E5 in SBEβCD as described in Example 8.

FIG. 14 shows the change in intraocular pressure (IOP) from baseline in Dutch belted rabbits following a single dose treatment with E5 at 0.1% or 0.25%.

FIG. 15 shows the average intraocular pressure (IOP) in Dutch belted rabbits following a single dose treatment with E5 at 0.1% or 0.25%.

### DETAILED DESCRIPTION

Listed below are definitions of various terms used to describe the present disclosure. These definitions apply to the terms as they are used throughout this specification and claims, unless otherwise limited in specific instances, either individually or as part of a larger group.

As used herein, the articles “a” and “an” refer to one or to more than one (i.e. to at least one) of the grammatical object of the article. By way of example, “an element” means one element or more than one element. Furthermore, use of the term “including” as well as other forms, such as “include”, “includes,” and “included,” is not limiting.

As used herein, the term “about” will be understood by one of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

As used herein, the term “administering” refers to administration of the compounds provided herein to a cell or a subject as needed to achieve the desired effect.

As used herein, the term “alkyl,” by itself or as part of another substituent means, unless otherwise stated, a straight or branched chain hydrocarbon having the number of carbon atoms designated (i.e., C₁₋₆ means one to six carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. In some embodiments the alkyl is C₁₋₆ alkyl, such as ethyl, methyl, isopropyl, isobutyl, n-pentyl, n-hexyl, or cyclopropylmethyl.

As used herein, the term “alkylene” by itself or as part of another substituent means, unless otherwise stated, a divalent alkyl.

As used herein, the term “aryl,” employed alone or in combination with other terms, means, unless otherwise stated, a carbocyclic aromatic system containing one or more rings (typically one, two or three rings), wherein such rings may be attached together in a pendent manner, such as a biphenyl, or may be fused, such as naphthalene. Examples of aryl groups include phenyl, anthracyl, and naphthyl. In some embodiments, the aryl is phenyl or naphthyl. In some embodiments, the aryl is phenyl.

As used herein, the terms “composition” or “pharmaceutical composition” refer to a mixture of at least one compound—useful as described herein—with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

As used herein, the term “contacting a cell” refers to contacting a cell in vitro or in vivo, i.e. in a subject, such as a mammal, including humans, livestock, rabbits, cats, dogs, and mice.

As used herein, the term “controlling the disease or disorder” is used to mean changing the activity of one or more kinases to affect the disease or disorder.

As used herein, the term “cycloalkyl” refers to a mono cyclic or polycyclic non-aromatic radical, wherein each of the atoms forming the ring (i.e., skeletal atoms) is a carbon atom. In one embodiment, the cycloalkyl group is saturated or partially unsaturated. In another embodiment, the cycloalkyl group is fused with an aromatic ring. Cycloalkyl groups include groups having from 3 to 10 ring atoms. Monocyclic cycloalkyls include, but are not limited to, cyclopropyl, cyclobutyl, cyclopentyl, cyclohexyl, cycloheptyl, and cyclooctyl. Bicyclic cycloalkyls include, but are not limited to, tetrahydronaphthyl, indanyl, and tetrahydropentalenyl. Polycyclic cycloalkyls include adamantine and norbornane. The term cycloalkyl includes “unsaturated nonaromatic carbocyclyl” or “nonaromatic unsaturated carbocyclyl” groups, both of which refer to a nonaromatic carbocycle, which contains at least one carbon-carbon double bond or one carbon-carbon triple bond.

As used herein, the term “disease or disorder associated with kinase activity” refers to a disease, condition or disorder treatable, in whole or in part, by inhibition of one or more kinases.

As used herein, the terms “effective amount,” “pharmaceutically effective amount” and “therapeutically effective amount” refer to a nontoxic but sufficient dosage amount of an agent (e.g., the compounds or compositions provided herein) to provide the desired biological result, which result may be reduction or alleviation, or both, of the signs, symptoms, or causes of a disease, or any other desired alteration of a biological system including influencing, reducing or inhibiting the activity of or preventing activation of a kinase (e.g., modulating kinase activity). An appropriate therapeutic amount in any individual case may be determined by one of ordinary skill in the art using routine experimentation. These terms as used herein may also refer to an amount effective at bringing about a desired in vivo effect in an animal—where in some embodiments, the animal is a human—including, but not limited to, uveitis, reduction in intraocular pressure, or dry eye.

As used herein, the term “excipient” refers to physiologically compatible additives useful in preparation of a pharmaceutical composition. Examples of pharmaceutically acceptable carriers and excipients can, for example, be found in Remington Pharmaceutical Science, 16^{th} Ed.

As used herein, the term “eye disease or disorder” refers to, but is not limited to, glaucoma, allergy, cancers of the eye, neurodegenerative diseases or disorders of the eye, such as diabetic eye disease, macular degeneration (AMD), an ocular inflammatory disease or disorder, and dry eye.

As used herein, the term “ocular inflammatory disease or disorder” refers to, but is not limited to uveitis, a non-infectious corneal ulcer, endophthalmitis, an autoimmune disease of the cornea or ocular surface, an ophthalmic manifestation of HIV disease, or any combination thereof, including inflammatory processes associated with dry eye disease.

As used herein, the term “halo” or “halogen” alone or as part of another substituent (e.g., haloalkyl, haloalkylene, haloaryl, halocycloalkyl, and the like) means, unless otherwise stated, a fluorine, chlorine, bromine, or iodine atom. In some embodiments, the halo or halogen is fluorine, chlorine, or bromine. In some embodiments, the halo or halogen is fluorine or chlorine. When used as part of another substituent, examples may include more than one halogen (e.g., 2, 3, 4, 5, 6, 7, 8, 9, 10, or 11 or more halogens) wherein each halogen is independently fluorine, chlorine, bromine, or iodine.

As used herein, the terms “subject,” “patient” or “individual” refer to a human or a non-human mammal. Non-human mammals include, for example, livestock and pets, such as ovine, bovine, porcine, canine, feline, and murine mammals. In some embodiments, the patient, subject, or individual is human.

As used herein, the term “pharmaceutically acceptable” refers to a material that does not abrogate the biological activity or properties of the compound, and is relatively non-toxic, i.e. the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound, and not injurious to the patient. As used herein, “pharmaceutically acceptable carrier” also includes any and all coatings, antibacterial and antifungal agents, and absorption delaying agents, and the like that are compatible with the activity of the compound described herein and are physiologically acceptable to the patient. The term “pharmaceutically acceptable carrier” may further include a pharmaceutically acceptable salt of a compound provided herein. Other additional ingredients that may be included in the pharmaceutical compositions provided herein are described, for example, in Remington's Pharmaceutical Sciences (Genaro, Ed., Mack Publishing Co., 1985, Easton, Pa.), which is incorporated herein by reference. The “pharmaceutically acceptable carrier” is useful for the preparation of a pharmaceutical composition that is: generally compatible with the other ingredients of the composition, not deleterious to the recipient, and neither biologically nor otherwise undesirable. “A pharmaceutically acceptable carrier” includes one or more than one carrier. Embodiments include carriers for topical, ocular, parenteral, intravenous, intraperitoneal intramuscular, sublingual, nasal or oral administration. “Pharmaceutically acceptable carrier” also includes agents for preparation of aqueous dispersions and sterile powders for injection or dispersions.

As used herein, the term “pharmaceutically acceptable salt” refers to derivatives of the compounds provided herein wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the compounds provided herein include the conventional non-toxic salts of the parent compound formed, for example, from non-toxic inorganic or organic acids. The pharmaceutically acceptable salts of the compounds provided herein can be synthesized from the parent compound which contains a basic or acidic moiety by conventional chemical methods. Generally, such salts can be prepared by combining the free acid or base forms of these compounds with a stoichiometric amount of the appropriate base or acid in water or in an organic solvent, or in a mixture of the two; generally, nonaqueous media such as ether, ethyl acetate, ethanol, isopropanol, or acetonitrile may be used. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

As used herein, the terms “prevent” or “prevention” refer to no disorder or disease development if none had occurred, or no further disorder or disease development if there had already been development of the disorder or disease. Also considered is the ability of one to prevent some or all of the symptoms associated with the disorder or disease.

As used herein, the term “protecting group” refers to a chemical moiety used to control the reactivity of a chemical functional group that is attached to a parent molecule while the parent molecule is involved in a multi-step synthetic procedure. Protecting groups may be sensitive to specific chemical environments, wherein the protecting group will cleave upon exposure to the chemical environment thereby producing the chemical functional group of the parent molecule. For example, exposure of a parent molecule comprising an amine—that is protected with an acid-labile protecting group—to an acidic environment will cleave the acid-labile protecting group and produce a molecule comprising an amine (i.e. an unprotected amine, i.e. a primary amine or a secondary amine). Protecting groups are described in Greene and Wuts, Protective Groups in Organic Synthesis 3^{rd} Ed., (Wiley 1999), which is incorporated herein by reference. Examples of acid-labile protecting groups include, but are not limited to, t-butyloxycarbonyl (Boc), benzyloxycarbonyl (Cbz), o-chlorobenzyloxycarbonyl, biphenylisopropyloxycarbonyl, t-amyloxycarbonyl (Amoc), isobornyloxycarbonyl, α,α-dimethyl-3,5-dimethoxybenzyloxy-carbonyl, o-nitrophenylsulfenyl, 2-cyano-t-butoxycarbonyl, 9-fluorenyl-methoxycarbonyl (Fmoc) and the like. In some embodiments, the acid-labile protecting group is Boc or Fmoc.

As used herein, the terms “treatment” or “treating” refer to the application or administration of a therapeutic agent, i.e. a compound provided herein, to a patient, or application or administration of a therapeutic agent to an isolated tissue or cell line from a patient (e.g., for diagnosis or ex vivo applications), who has a disease, a symptom of the disease or the potential to develop the disease, with the purpose to heal, alleviate, relieve, alter, remedy, ameliorate, improve or affect the disease, the symptoms of the disease, or the potential to develop the disease. Such treatments may be specifically tailored or modified, based on knowledge obtained from the field of pharmacogenomics.

As used herein, the term “physiological intraocular pressure” refers to the intraocular pressure found in individuals not suffering from a disease or disorder that increases intraocular pressure, such as glaucoma. For most of the population, physiological intraocular pressure ranges between about 10 mm Hg and about 21 mm Hg, inclusive.

### Compounds

In one aspect, provided herein are compounds of Formula (I):

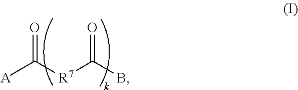

or a pharmaceutically acceptable salt thereof;
wherein
k is 0 or 1;
A is a corticosteroid moiety, linked through its primary alcohol to form an ester linkage.
B is a primary alcohol-containing rho kinase inhibitor, linked through its primary alcohol to form an ester linkage; and
R⁷ is

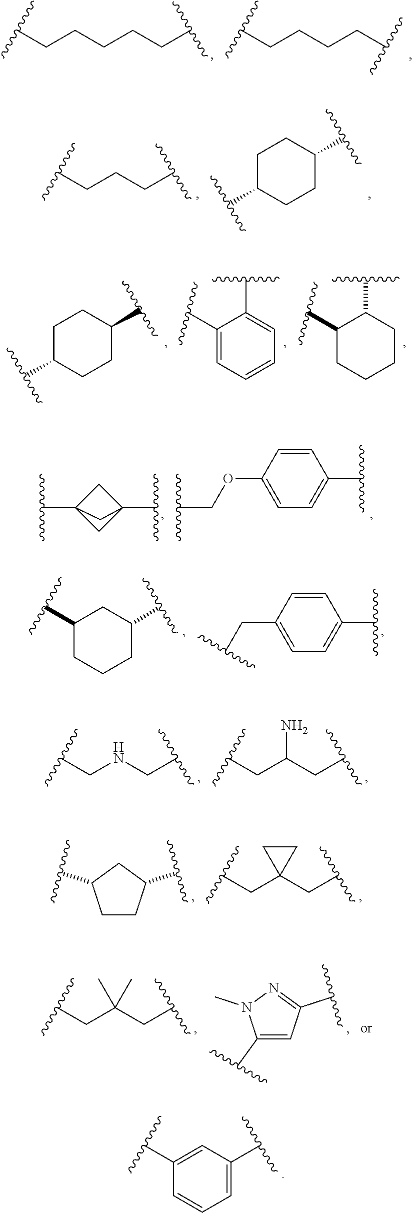

In some embodiments, the compound of Formula (I) is a compound of Formula (Ia):

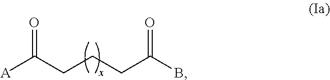

or a pharmaceutically acceptable salt thereof;
wherein
A is a corticosteroid moiety, linked through its primary alcohol to form an ester linkage.
B is a primary alcohol-containing rho kinase inhibitor, linked through its primary alcohol to form an ester linkage; and
x is 1 or 2 or 3.

In some embodiments, the compound of Formula (I) is a compound of Formula (Ib):

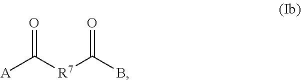

or a pharmaceutically acceptable salt thereof.

In some embodiments, the compound of Formula (I) is a compound of Formula (Ic):

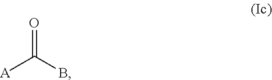

or a pharmaceutically acceptable salt thereof.

In some embodiments, A is R⁸ as defined herein, and B is R¹¹ as defined herein.

In some embodiments, Formula (I) is of Formula (II):

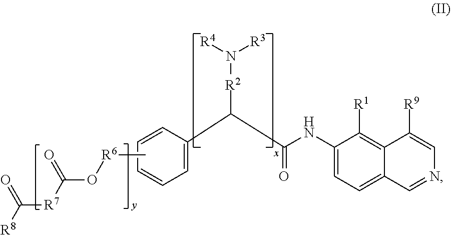

or a pharmaceutically acceptable salt thereof,
wherein
R¹ is H, —C₁₋₃ alkyl, —C₁₋₃ haloalkyl, or halogen;
R² is C₁₋₃ alkylene or C₁₋₃ haloalkylene;
R³ is H, —C₁₋₆ alkyl, —C₁₋₆ haloalkyl, or an acid-labile protecting group;
R⁴ is H, —C₁₋₆ alkyl, or —C₁₋₆ haloalkyl;
R⁶ is C₁₋₆ alkylene or C₁₋₆ haloalkylene;
R⁷ is C₁₋₆ alkyl, C₁₋₆ heteroalkyl, C₁₋₆ haloalkyl, C₃₋₁₀ cycloalkyl, C₃₋₁₀-cyclohaloalkyl aryl, heteroaryl, C₃₋₈ alkylene or a polyglycol of 5 to 10 units; and
R⁸ is a corticosteroidal moiety containing a primary alcohol, wherein R⁸ and the carbonyl to which it is attached form an ester linkage via that primary alcohol;
R⁹ is H, —C₁₋₃ alkyl, —C₁₋₃ haloalkyl, or halogen;
x is 0 or 1; and
y is 0 or 1.

In some embodiments, Formula (II) is of Formula (V):

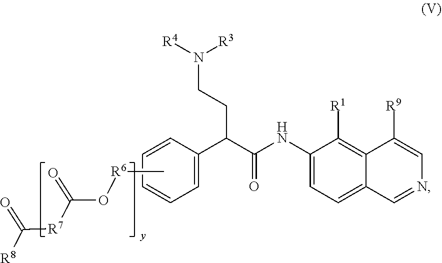

or a pharmaceutically acceptable salt thereof.

In some embodiments, Formula (II) is of Formula (VI):

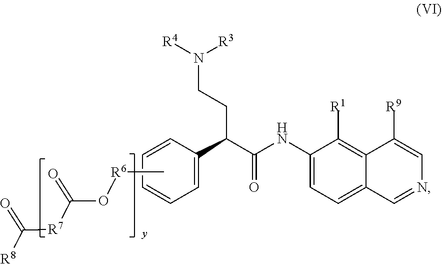

or a pharmaceutically acceptable salt thereof.

In some embodiments, Formula (II) is of Formula (VII):

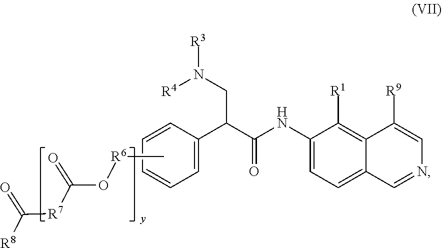

or a pharmaceutically acceptable salt thereof.

In some embodiments, Formula (II) is of Formula (VIII):

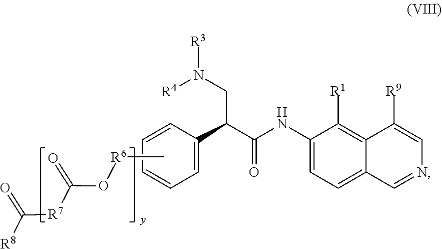

or a pharmaceutically acceptable salt thereof.

In some embodiments, Formula (I) is of Formula (IX):

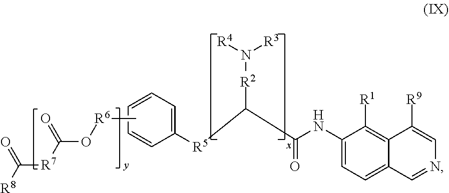

or a pharmaceutically acceptable salt thereof,
wherein
R¹ is H, —C₁₋₃ alkyl, —C₁₋₃ haloalkyl, or halogen;
R² is C₁₋₃ alkylene or C₁₋₃ haloalkylene;
R³ is H, —C₁₋₆ alkyl, —C₁₋₈ haloalkyl, or an acid-labile protecting group;
R⁴ is H, —C₁₋₈ alkyl, or —C₁₋₈ haloalkyl;
R⁵ is C₁₋₃ alkylene or C₁₋₃ haloalkylene;
R⁶ is C₁₋₆ alkylene or C₁₋₈ haloalkylene;
R⁷ is C₁₋₆ alkyl, C₁₋₆ heteroalkyl, C₁₋₆ haloalkyl, C₃₋₁₀ cycloalkyl, C₃₋₁₀-cyclohaloalkyl aryl, heteroaryl, C₃₋₈ alkylene or a polyglycol of 5 to 10 units; and
R⁸ is a corticosteroidal moiety containing a primary alcohol, wherein R⁸ and the carbonyl to which it is attached form an ester linkage via that primary alcohol;
R⁹ is H, —C₁₋₃ alkyl, —C₁₋₃ haloalkyl, or halogen;
x is 0 or 1; and
y is 0 or 1.

In some embodiments, Formula (I) is of Formula (X):

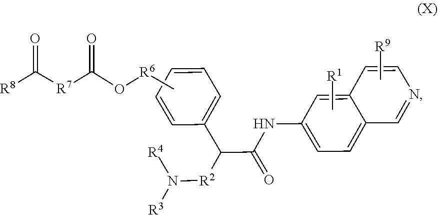

or a pharmaceutically acceptable salt thereof,
wherein
R¹ is H, —C₁₋₃ alkyl, —C₁₋₃ haloalkyl, or halogen;
R² is C₁₋₃ alkylene or C₁₋₃ haloalkylene;
R³ is H, —C₁₋₆ alkyl, —C₁₋₆ haloalkyl, or an acid-labile protecting group;
R⁴ is H, —C₁₋₆ alkyl, or —C₁₋₆ haloalkyl;
R⁶ is Cis alkylene or C₁₋₆ haloalkylene;
R⁷ is C₁₋₆ alkyl, C₁₋₆ heteroalkyl, C₁₋₈ haloalkyl, C₃₋₁₀ cycloalkyl, C₃₋₁₀-cyclohaloalkyl aryl, heteroaryl, C₃₋₈ alkylene or a polyglycol of 5 to 10 units; and
R⁸ is a corticosteroidal moiety containing a primary alcohol, wherein R⁸ and the carbonyl to which it is attached form an ester linkage via that primary alcohol; and
R⁹ is H, —C₁₋₃ alkyl, —C₁₋₃ haloalkyl, or halogen.

In some embodiments of the formulae herein, C₃₋₁₀ cycloalkyl or C₃₋₁₀-cyclohaloalkyl may be C₃₋₆ cycloalkyl or C₃₋₆-cyclohaloalkyl, respectively, e.g., monocycloalkyl or monocyclohaloalkyl moieties. In some embodiments of the formulae herein, C₃₋₁₀ Cycloalkyl or C₃₋₁₀-cyclohaloalkyl may be C₆₋₁₀ cycloalkyl or C₆₋₁₀-cyclohaloalkyl, respectively, e.g., bicycloalkyl or bicyclohaloalkyl moieties.

In some embodiments of the formulae herein, R⁸ is a corticosteroidyl moiety or a derivative thereof.

In some embodiments of the formulae herein, R⁸ is dexamethasonyl, prednisolonyl, fluocinolonyl, or triamcinolonyl.

In some embodiments of the formulae herein, R⁷ is

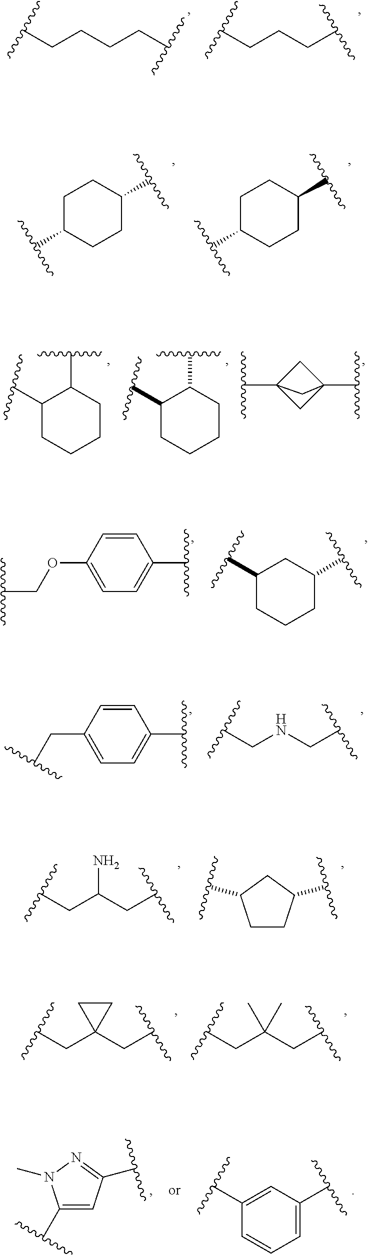

In some embodiments of the formulae herein, R⁸ is

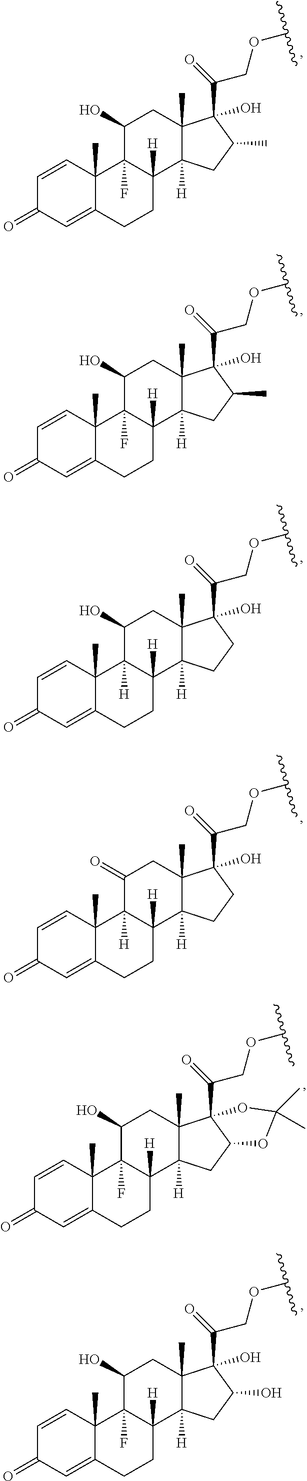

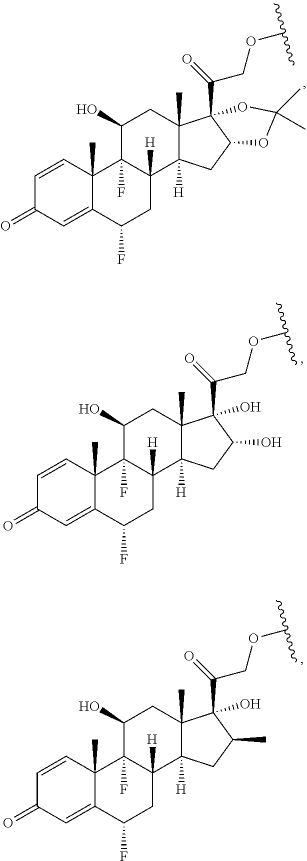

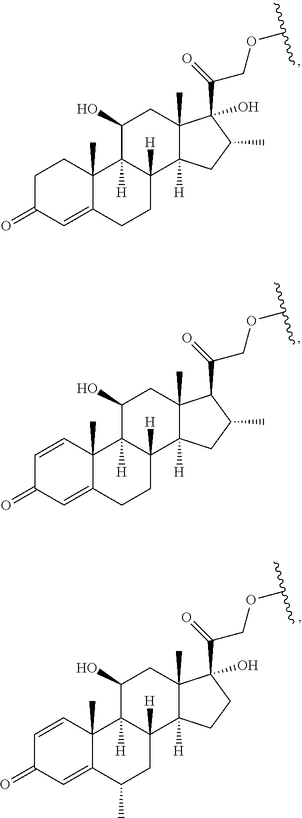

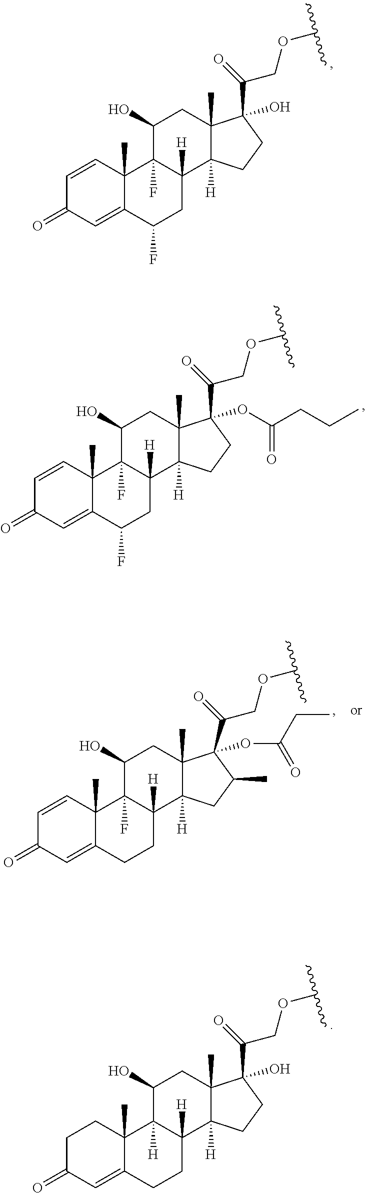

In some embodiments, R¹⁰ is

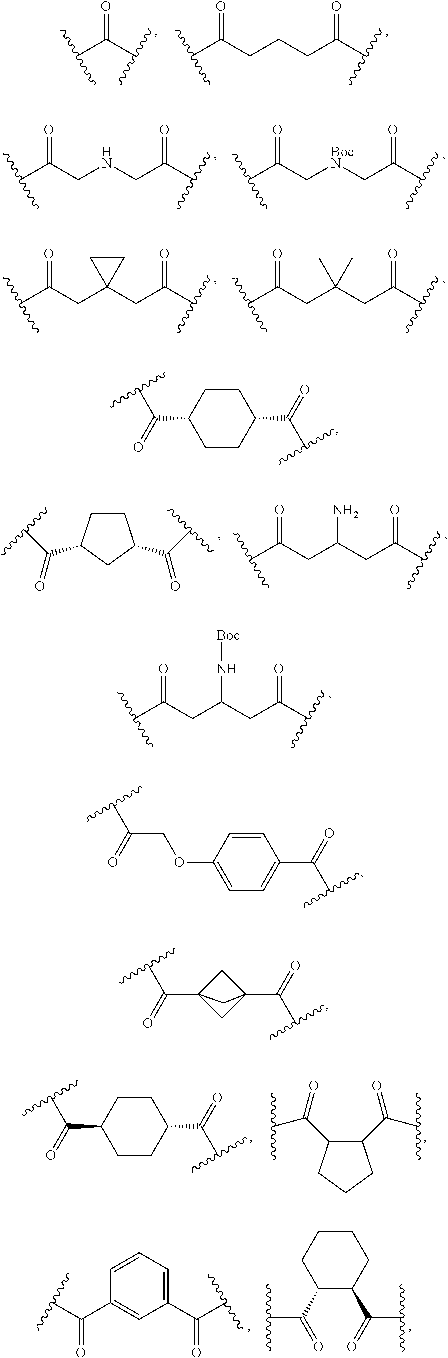

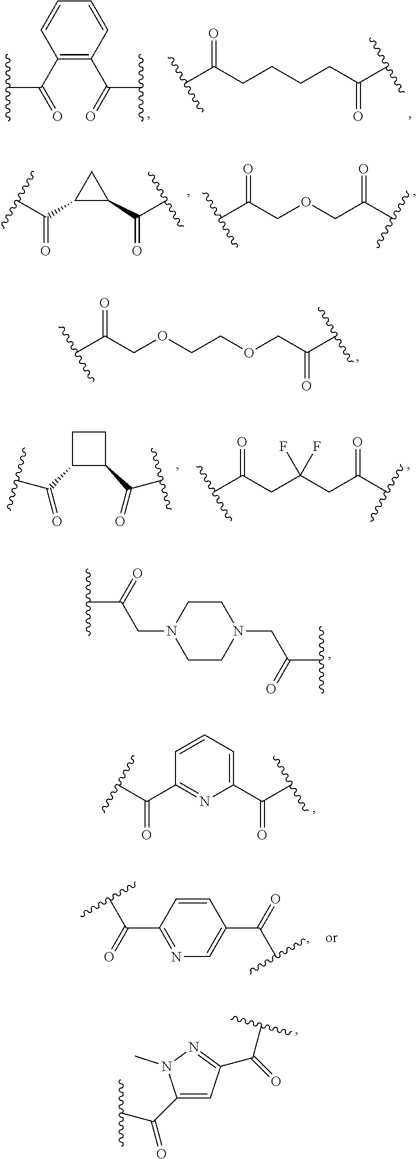

in some embodiments of the formulae provided herein, R¹¹ is

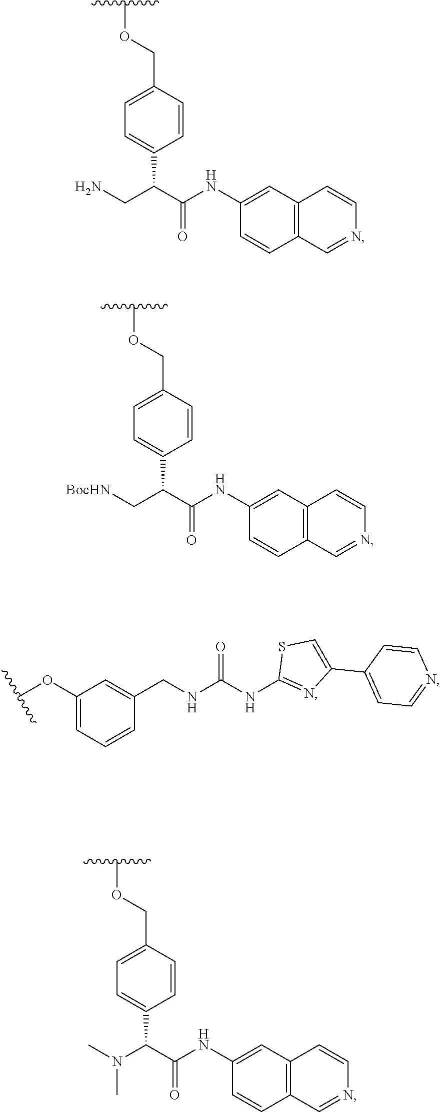 [SMILES: *OCc1ccc([C@@H](CN)C(=O)Nc2ccc3cnccc3c2)cc1 ; *OCc1ccc([C@@H](CNC(=O)OC(C)(C)C)C(=O)Nc2ccc3cnccc3c2)cc1 ; *Oc1cccc(CNC(=O)Nc2nc(-c3ccncc3)cs2)c1 ; *OCc1ccc([C@H](C(=O)Nc2ccc3cnccc3c2)N(C)C)cc1]

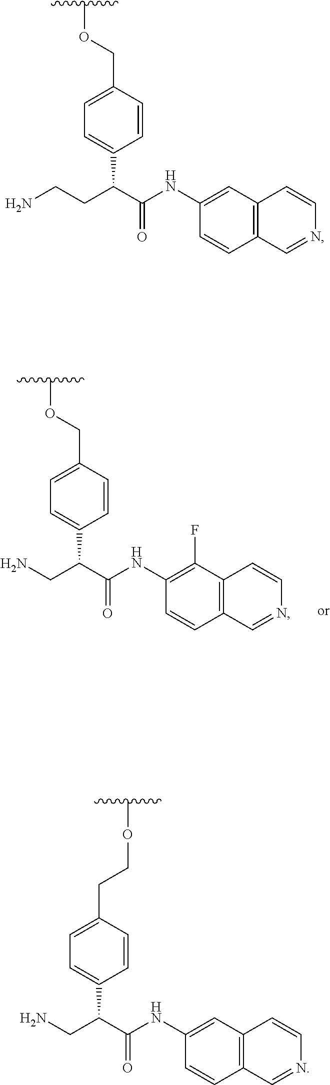 [SMILES: *OCc1ccc([C@@H](CCN)C(=O)Nc2ccc3cnccc3c2)cc1 ; *OCc1ccc([C@@H](CN)C(=O)Nc2ccc3cnccc3c2F)cc1 ; *OCCc1ccc([C@@H](CN)C(=O)Nc2ccc3cnccc3c2)cc1]

In some embodiments, the compound is a compound of Table 1, or a pharmaceutically acceptable salt thereof.

TABLE 1

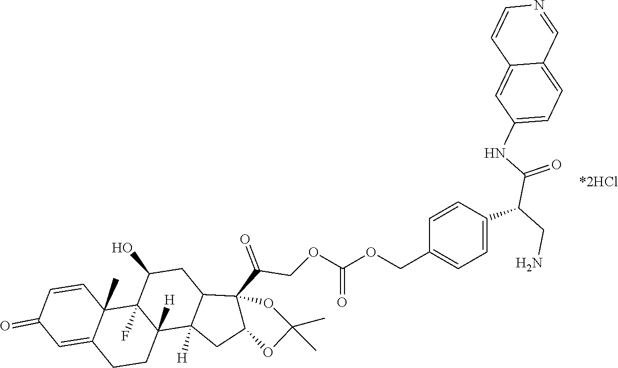

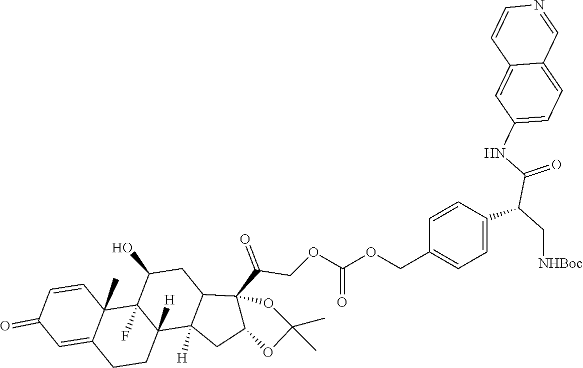

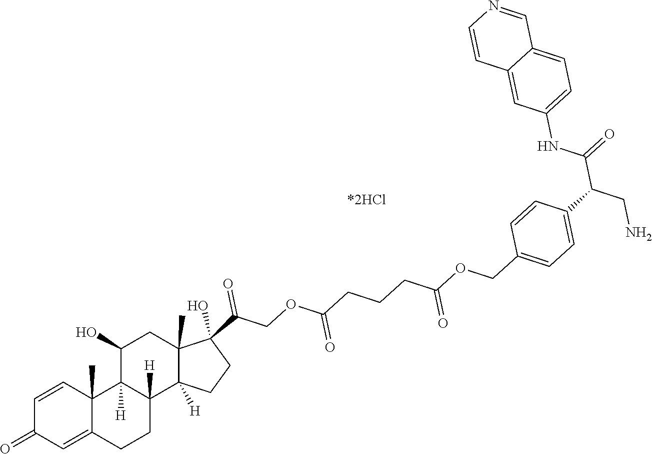

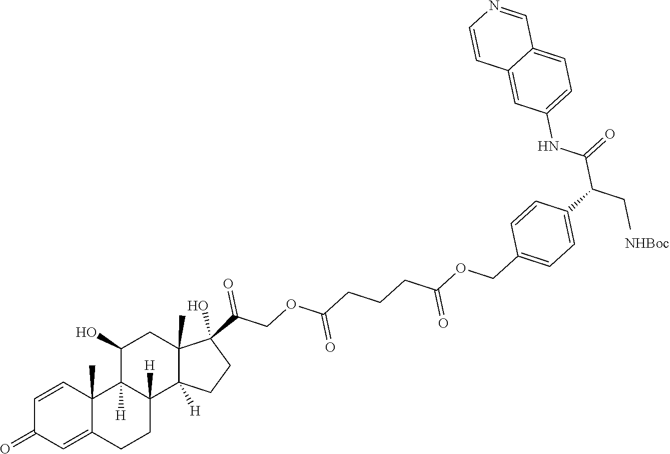

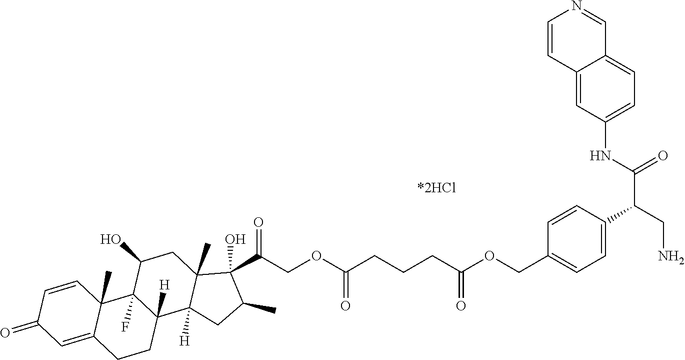

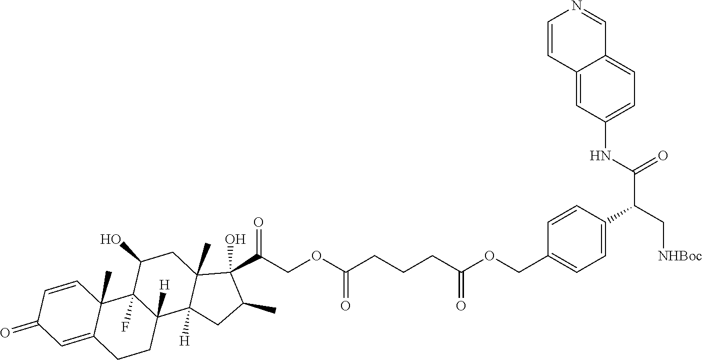

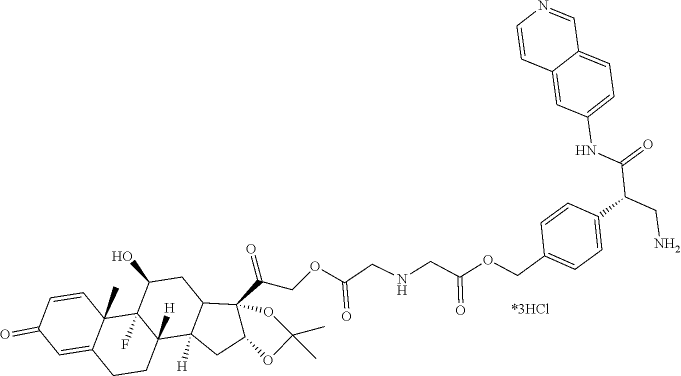

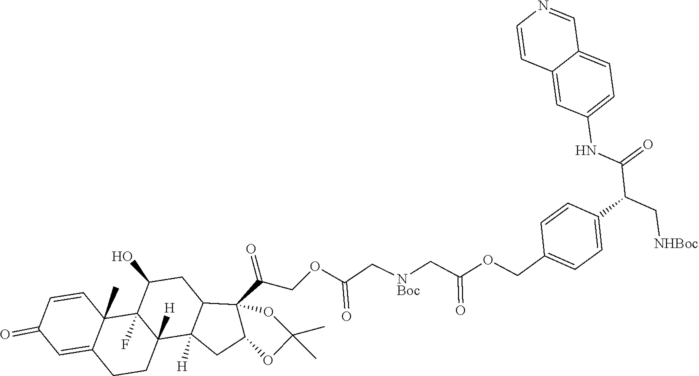

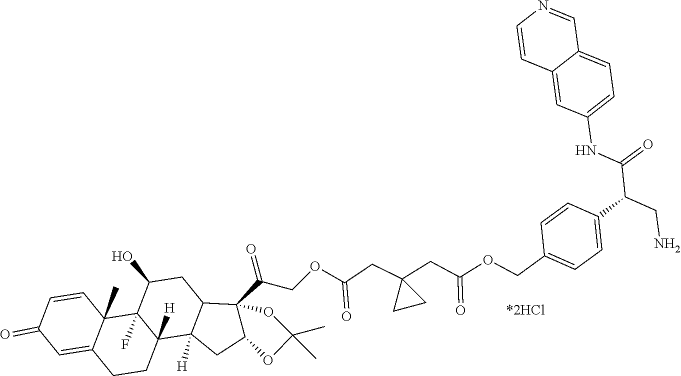

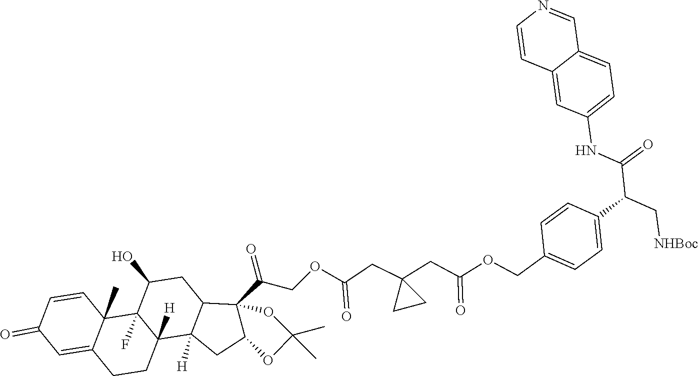

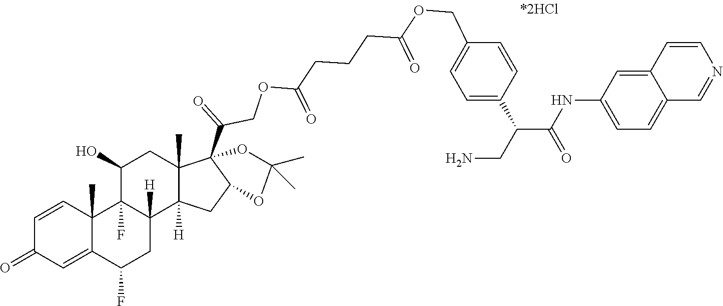

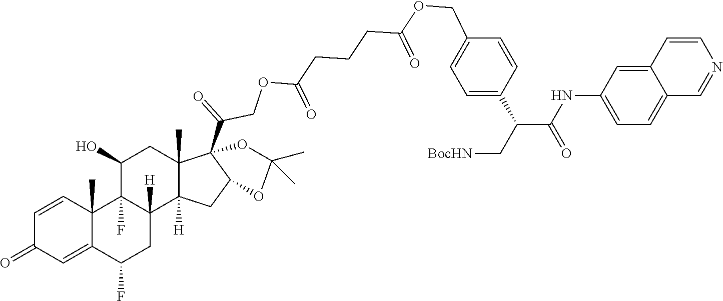

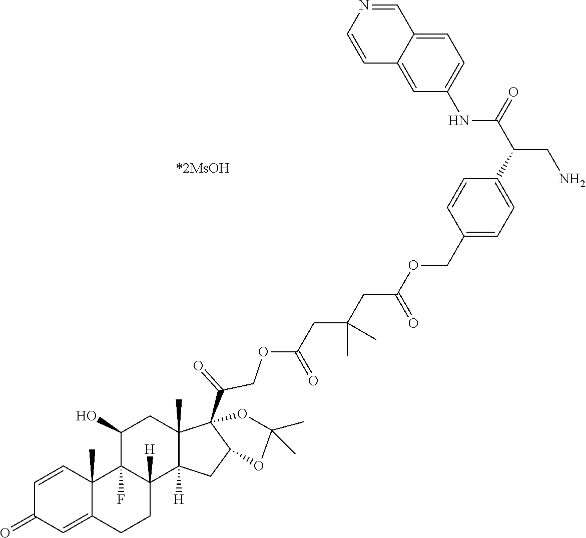

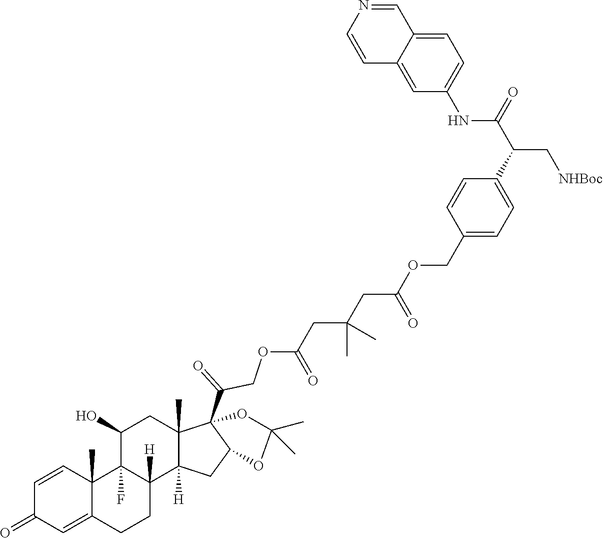

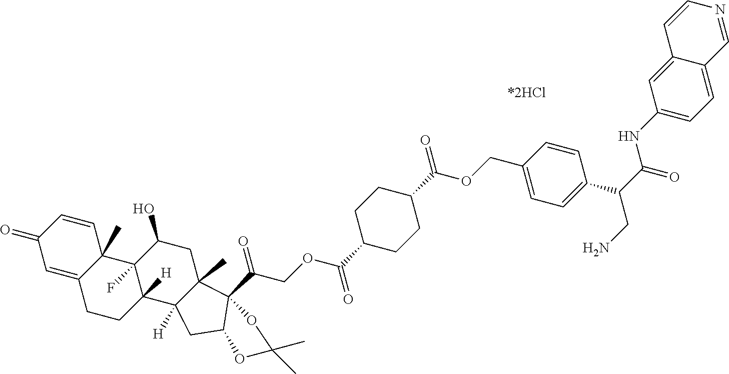

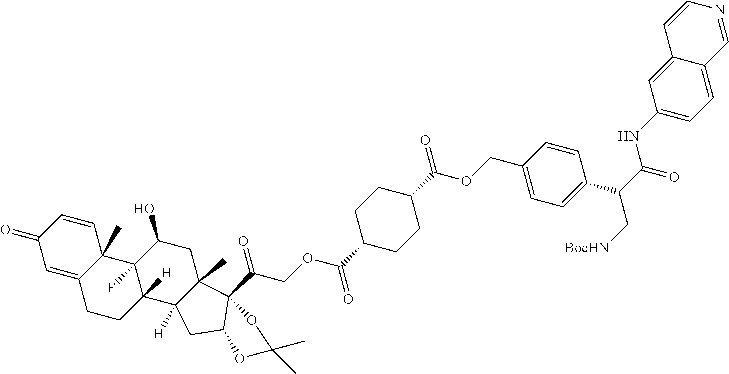

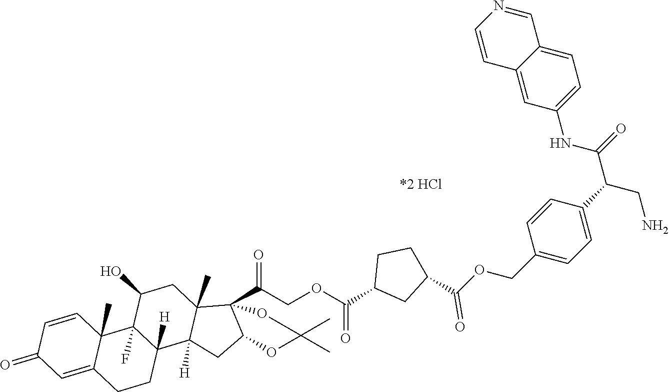

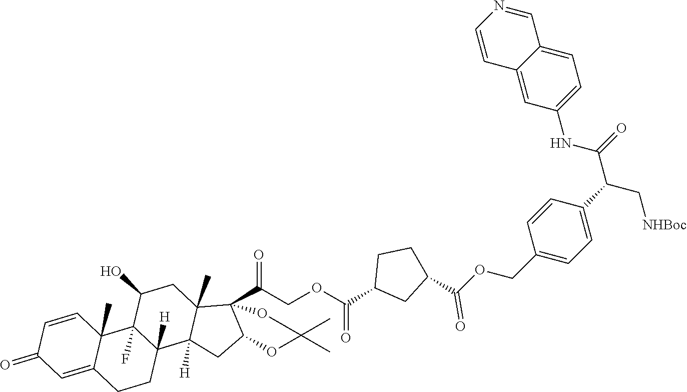

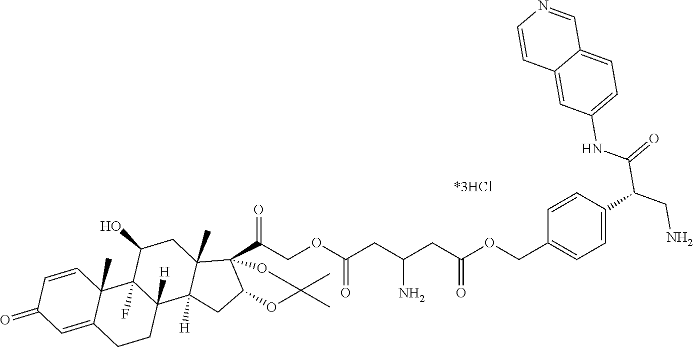

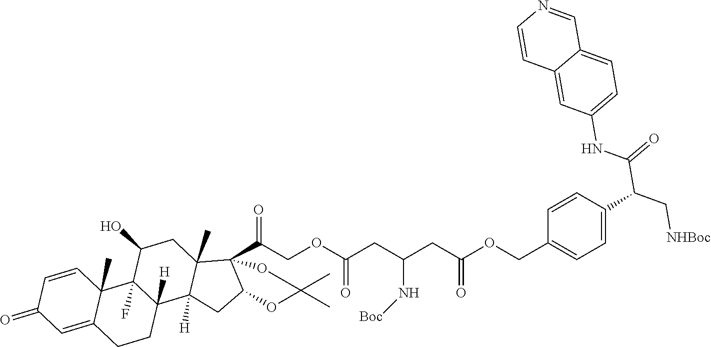

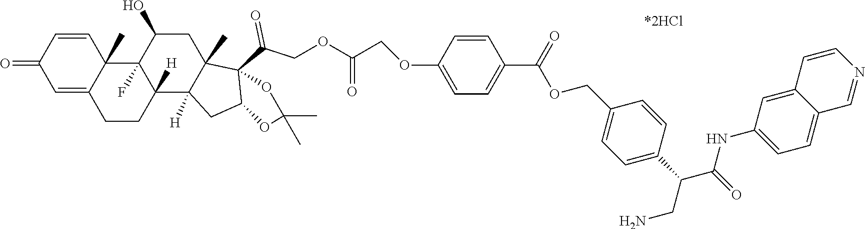

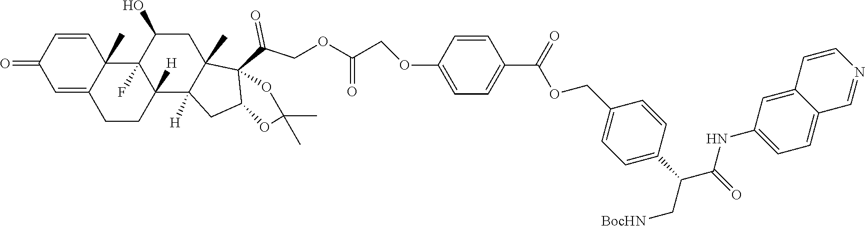

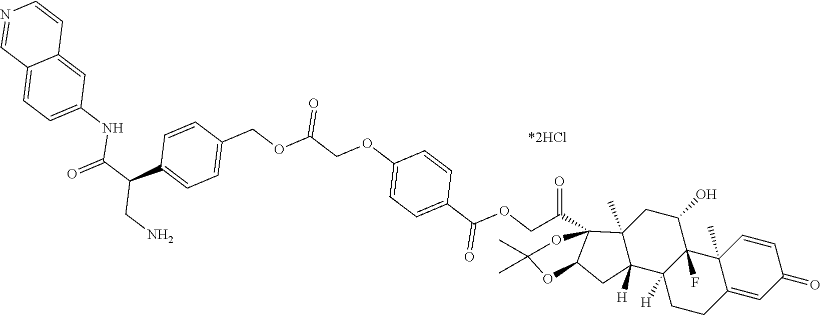

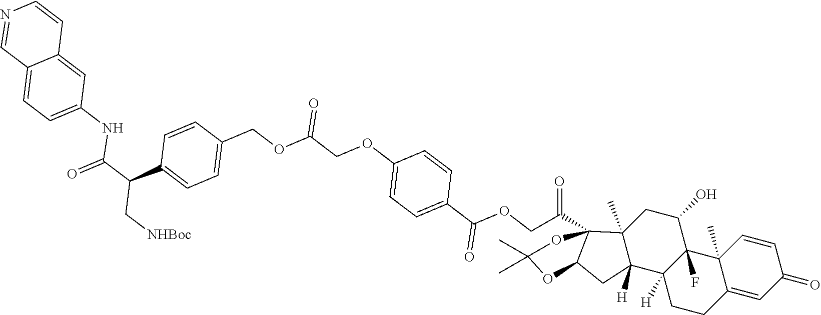

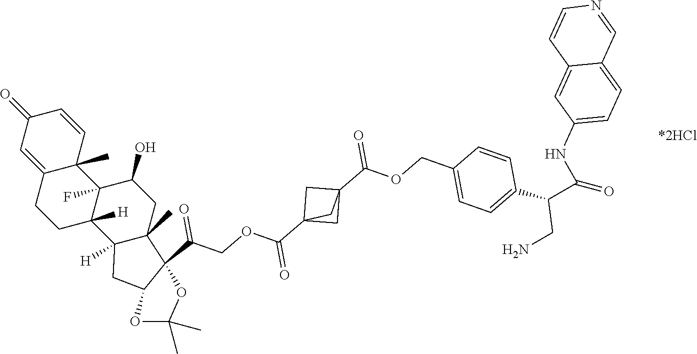

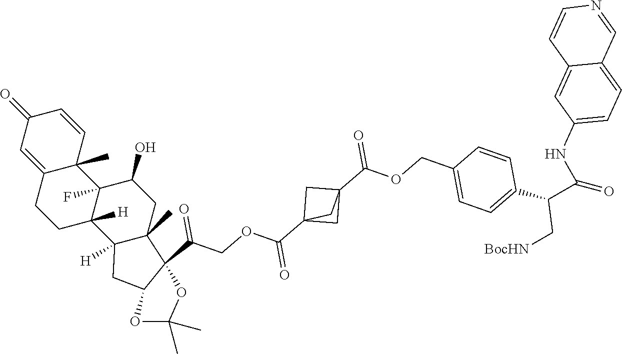

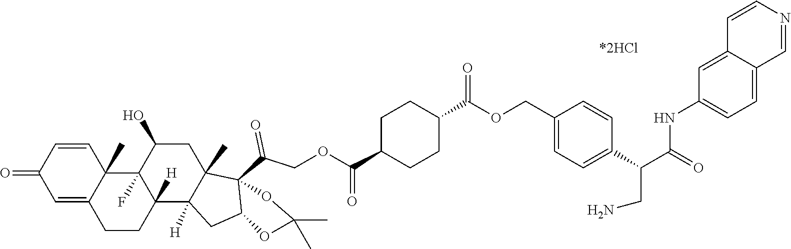

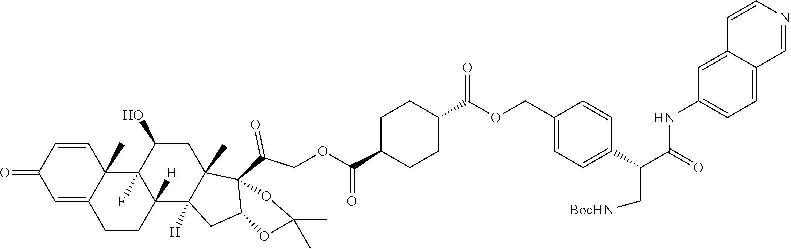

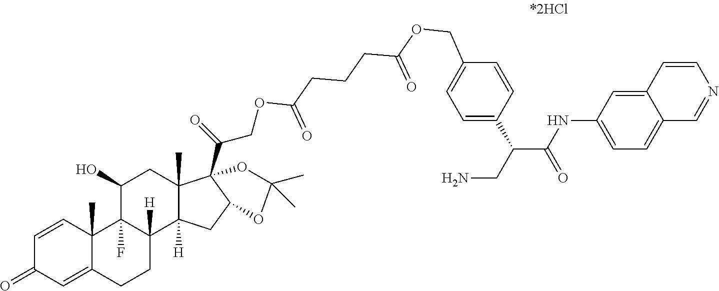

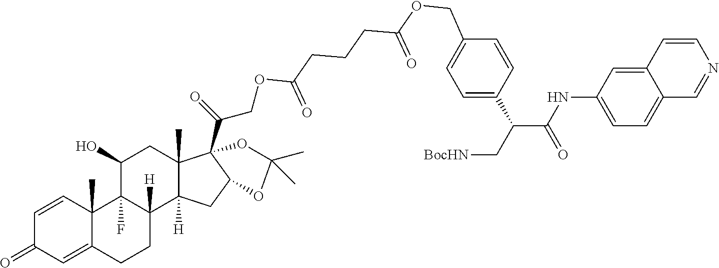

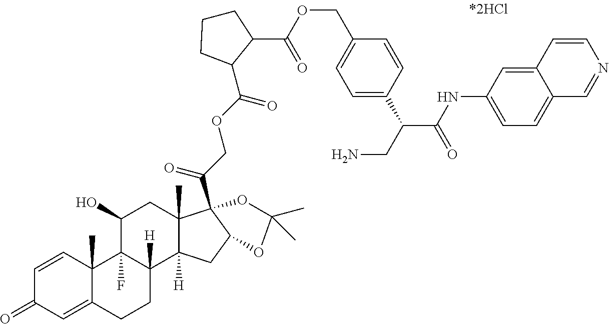

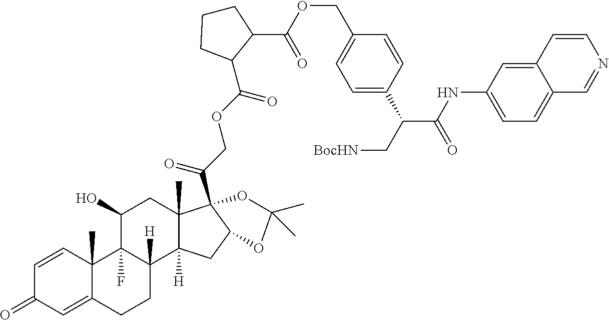

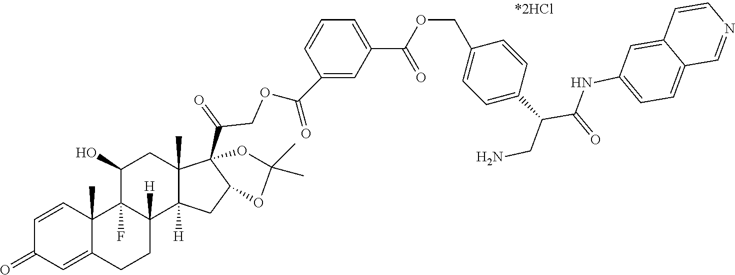

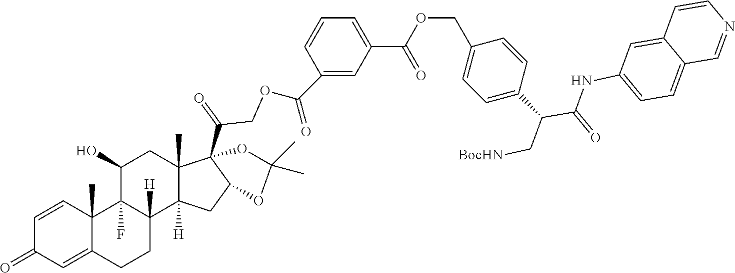

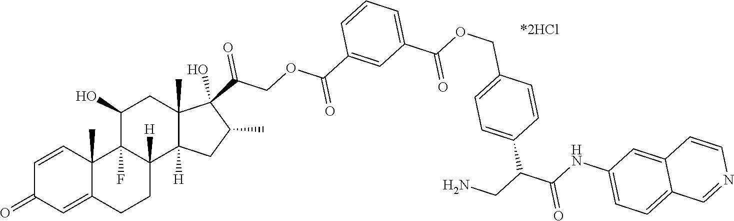

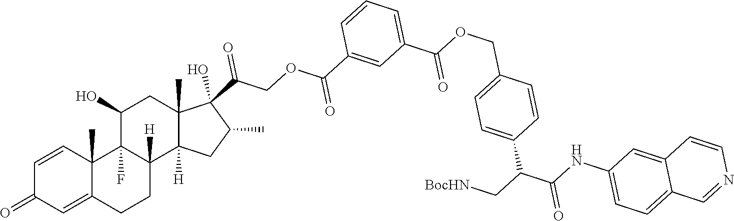

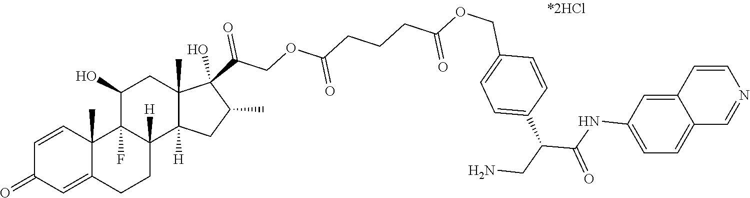

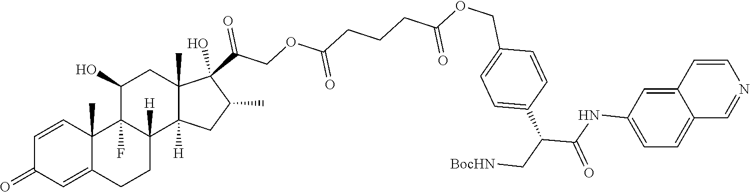

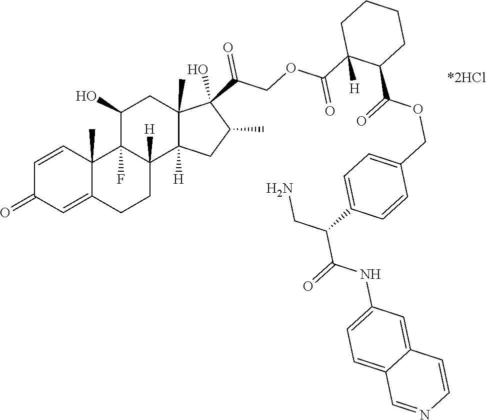

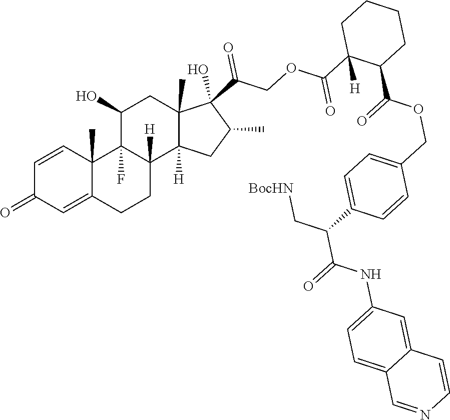

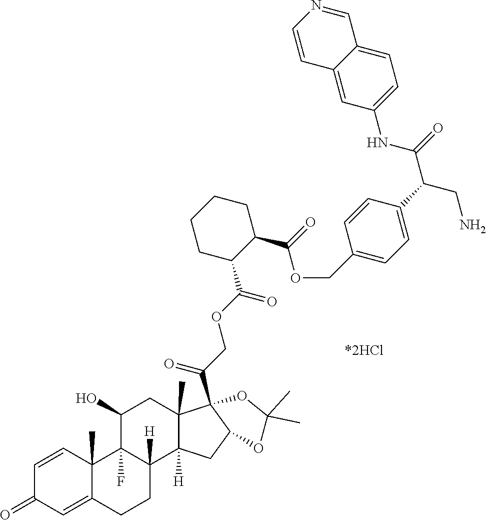

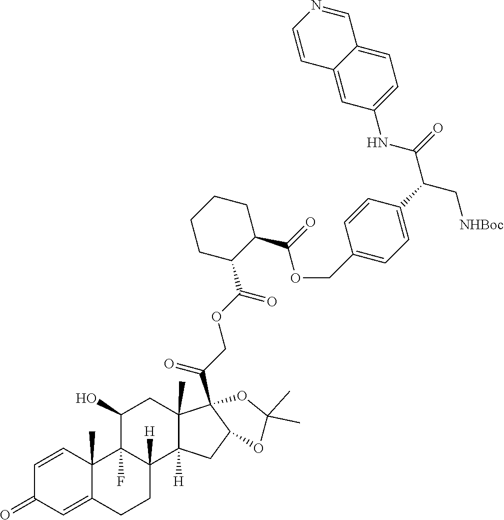

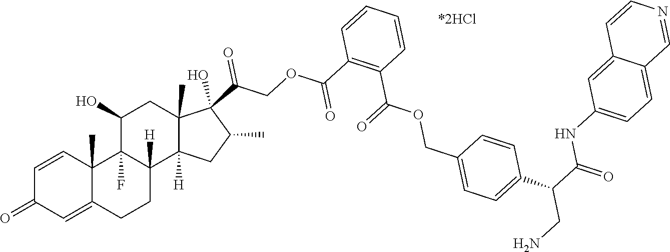

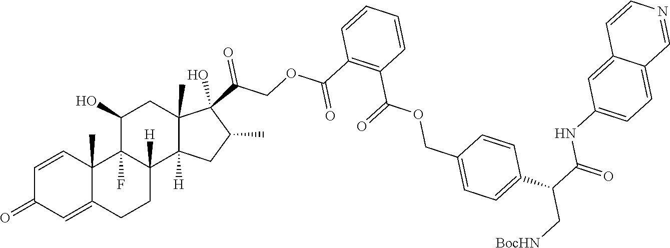

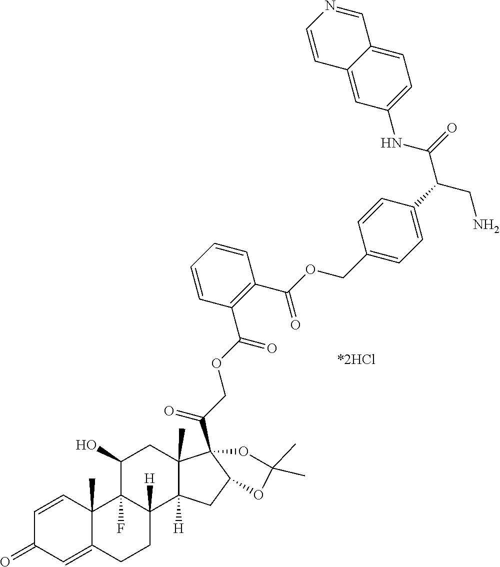

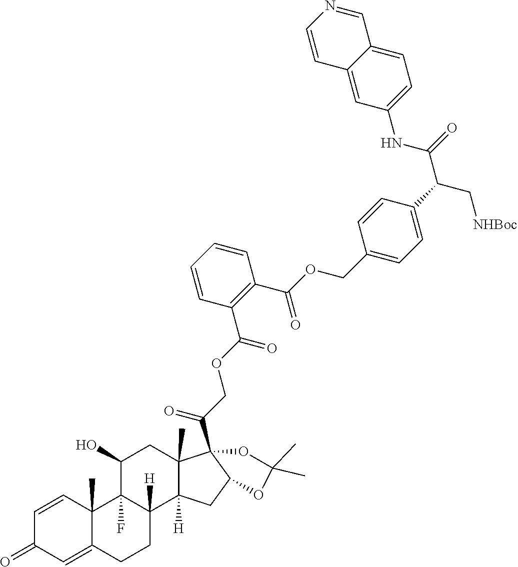

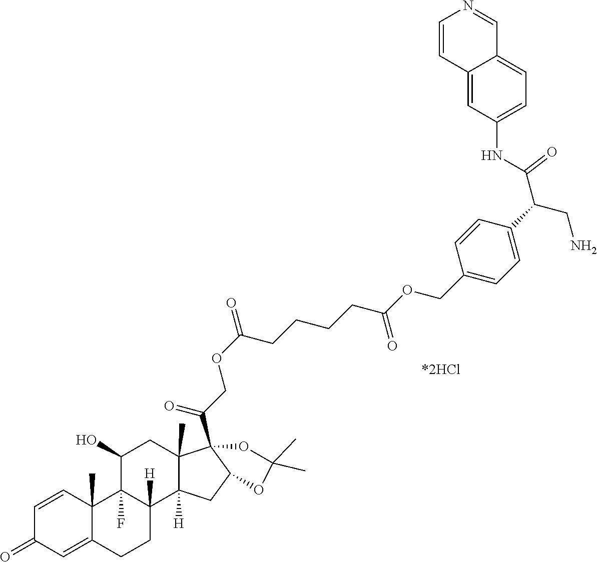

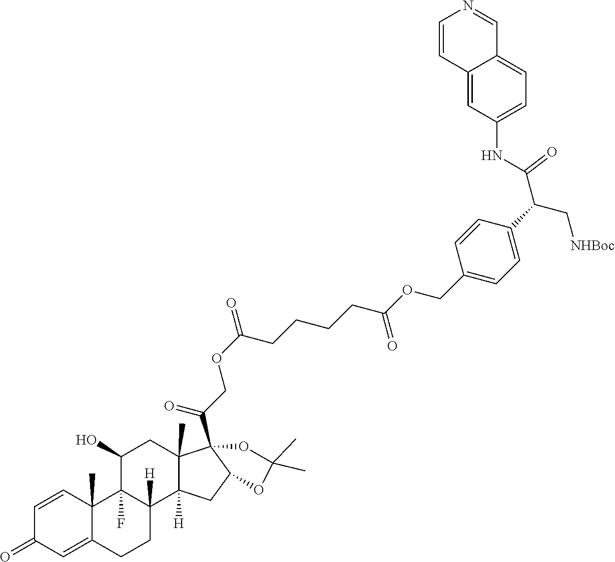

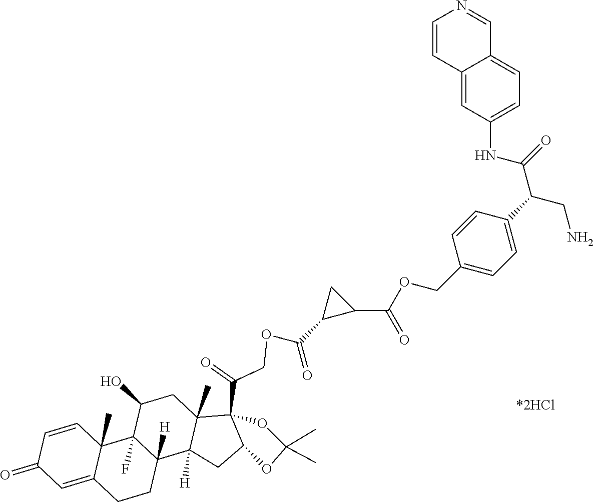

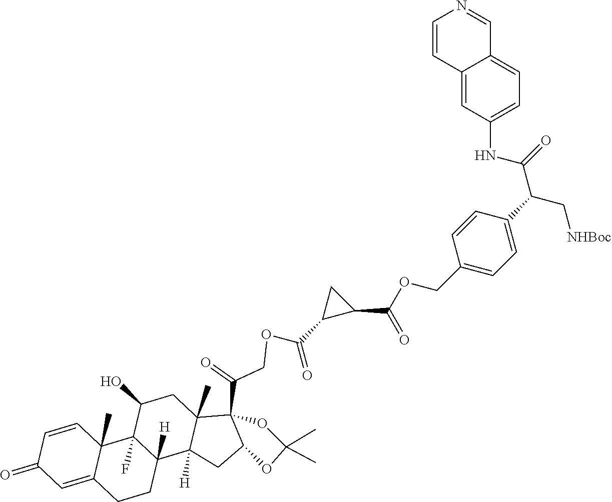

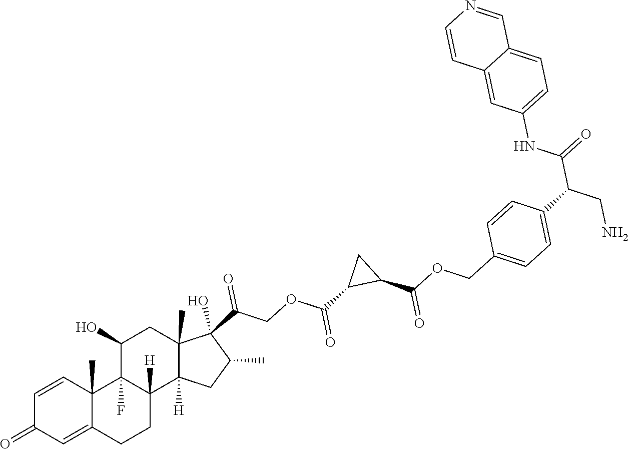

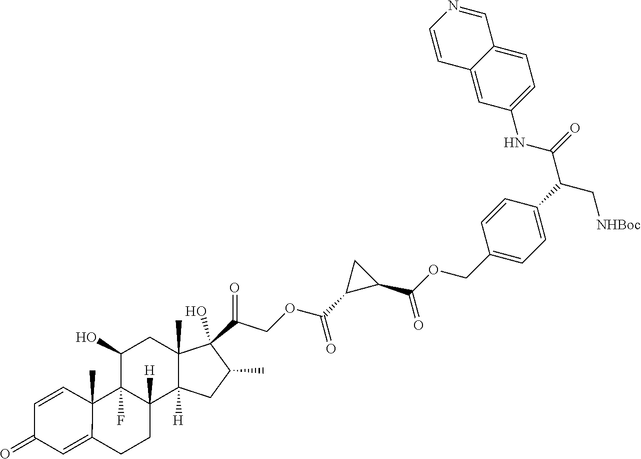

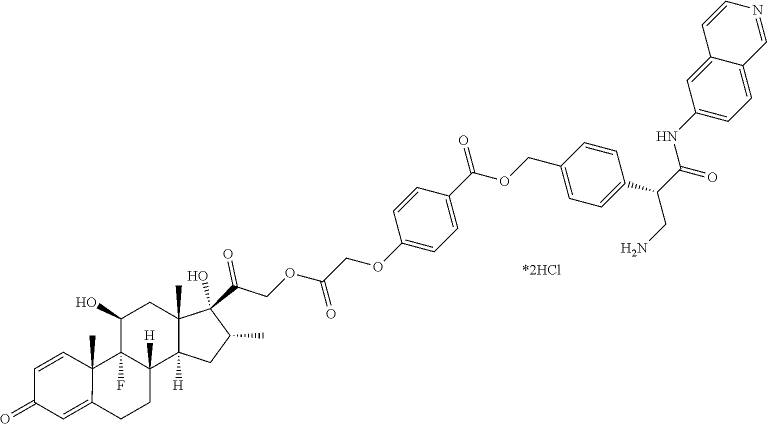

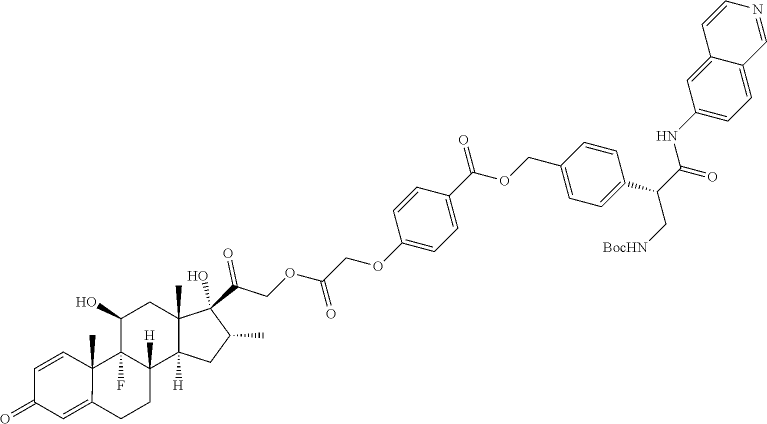

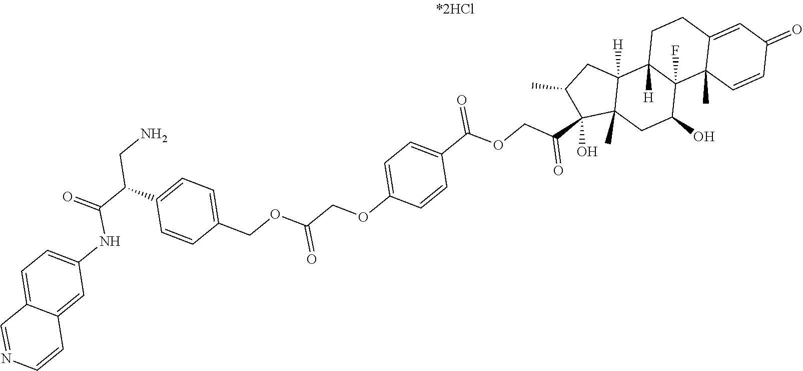

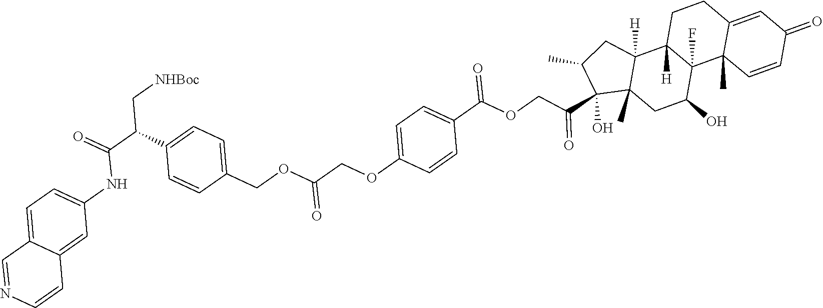

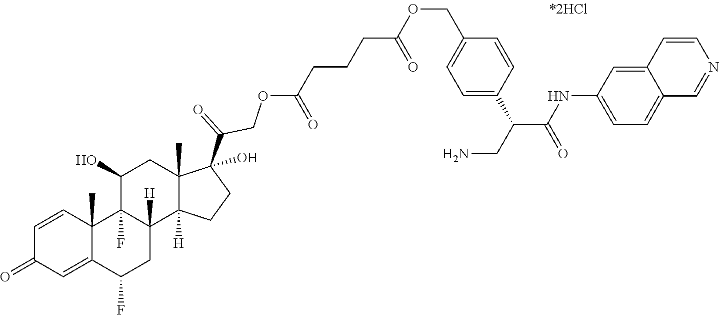

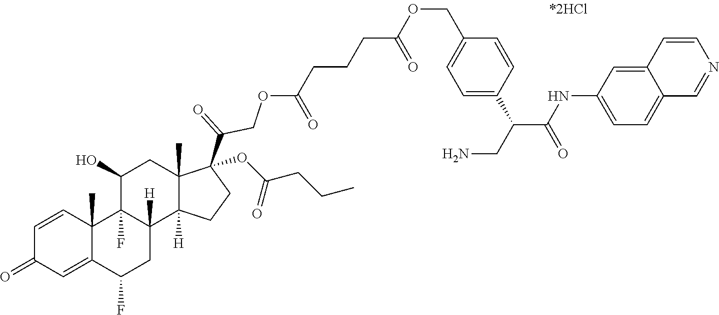

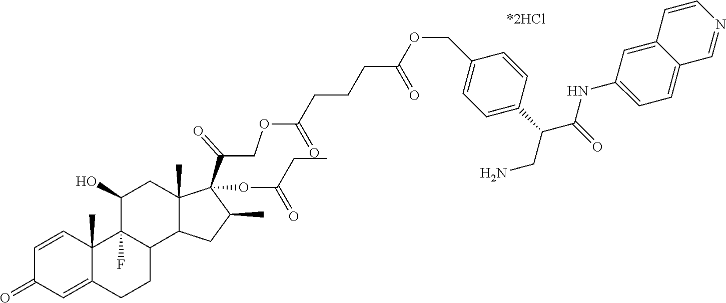

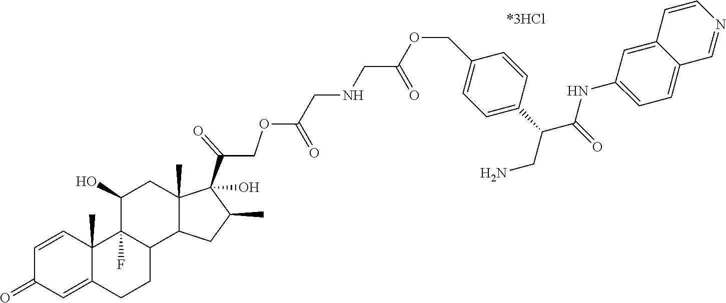

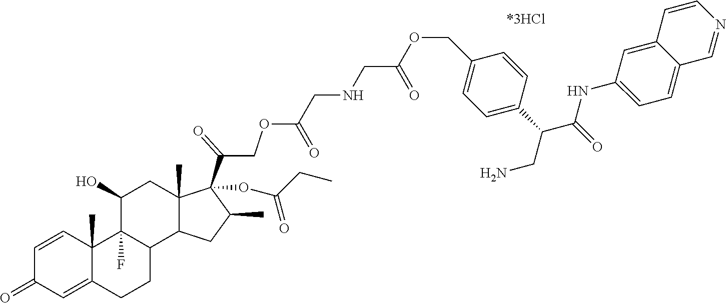

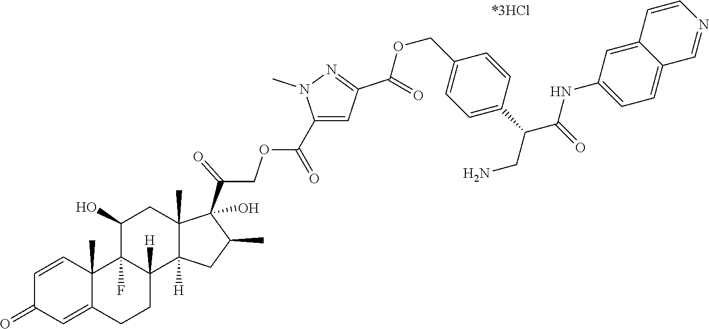

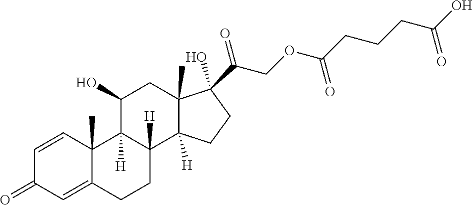

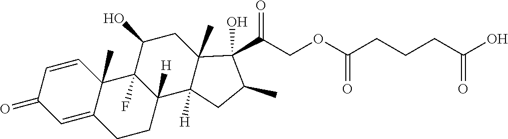

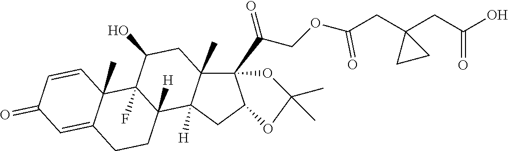

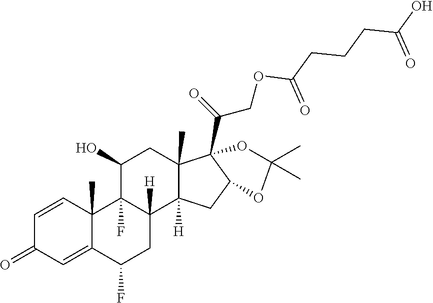

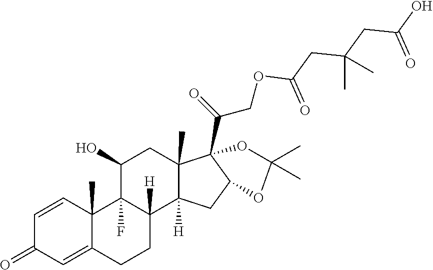

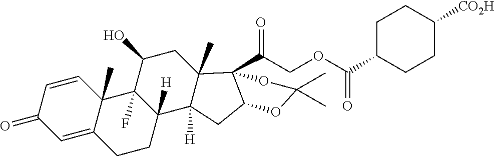

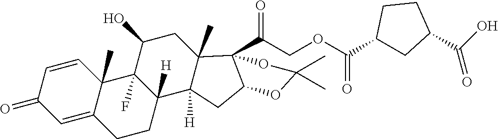

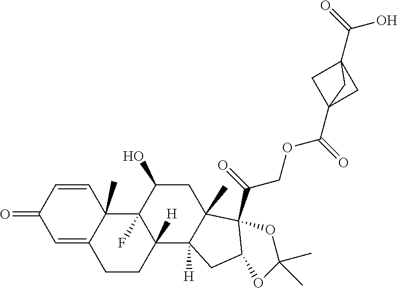

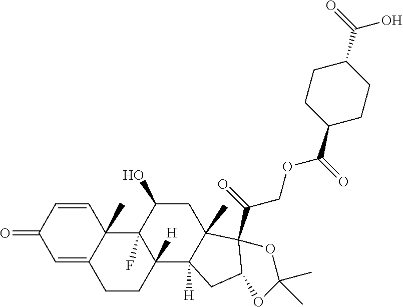

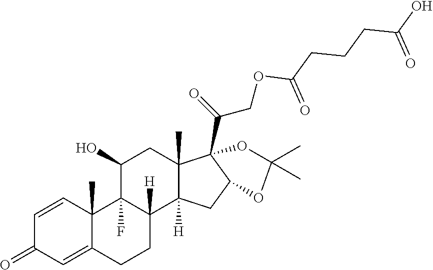

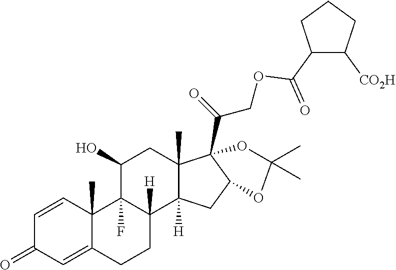

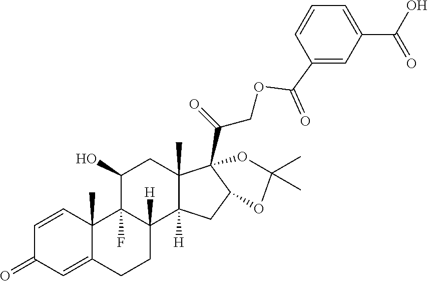

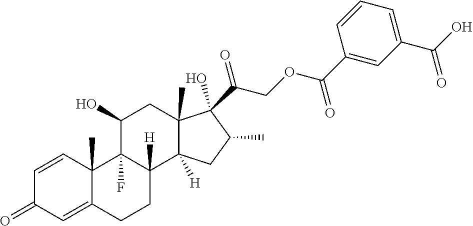

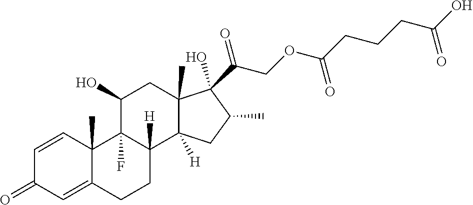

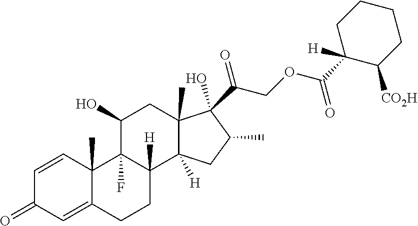

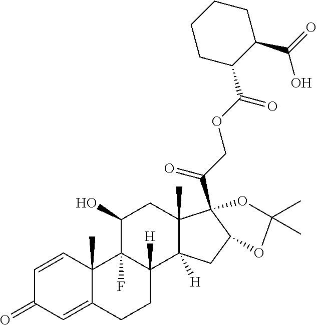

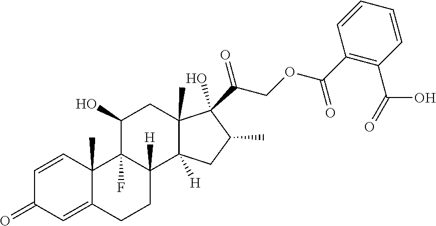

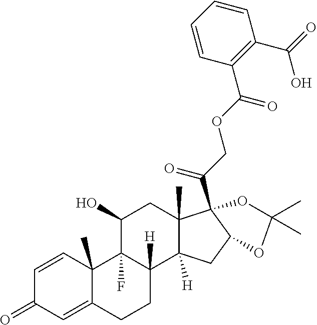

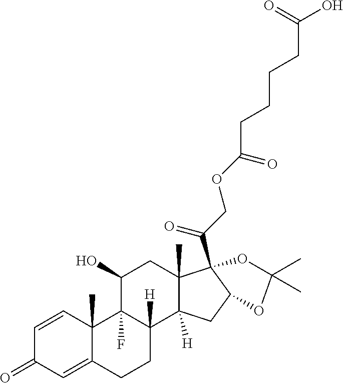

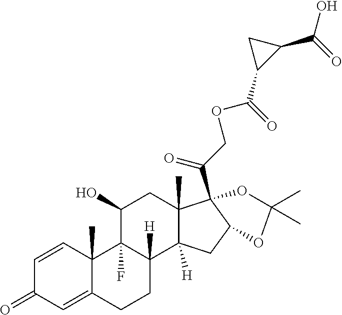

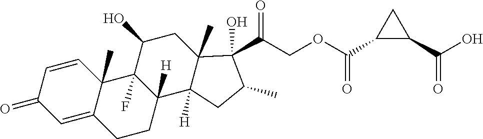

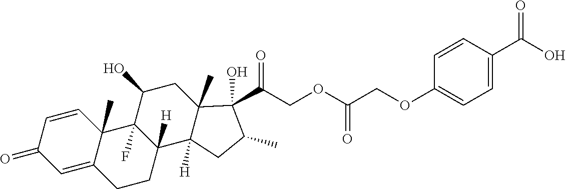

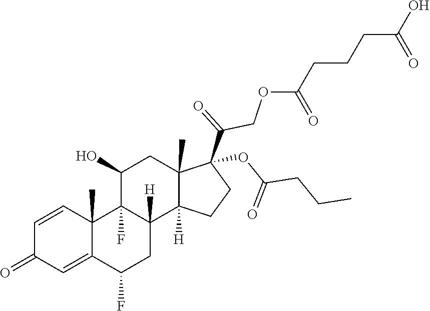

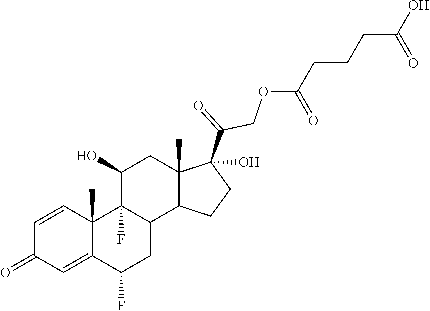

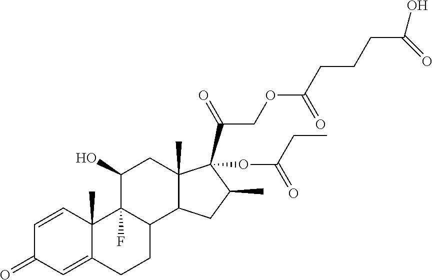

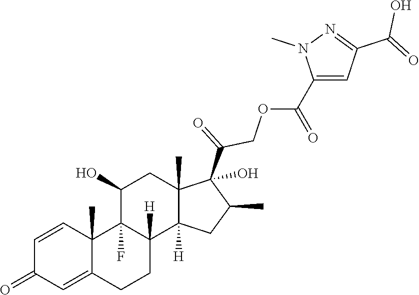

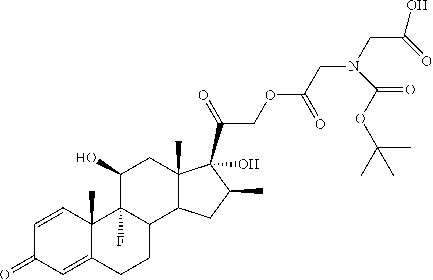

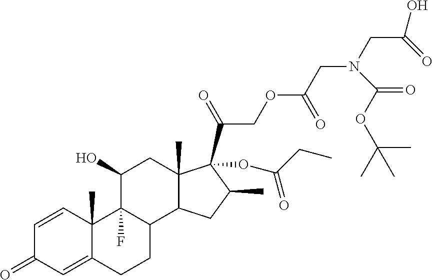

or a pharmaceutically acceptable salt thereof.

In some embodiments, the compound of Formula (I) is a compound of Formula (IX):

R⁸—R¹⁰—R¹¹ (IX)
of a pharmaceutically acceptable salt thereof,
wherein
R⁸ is

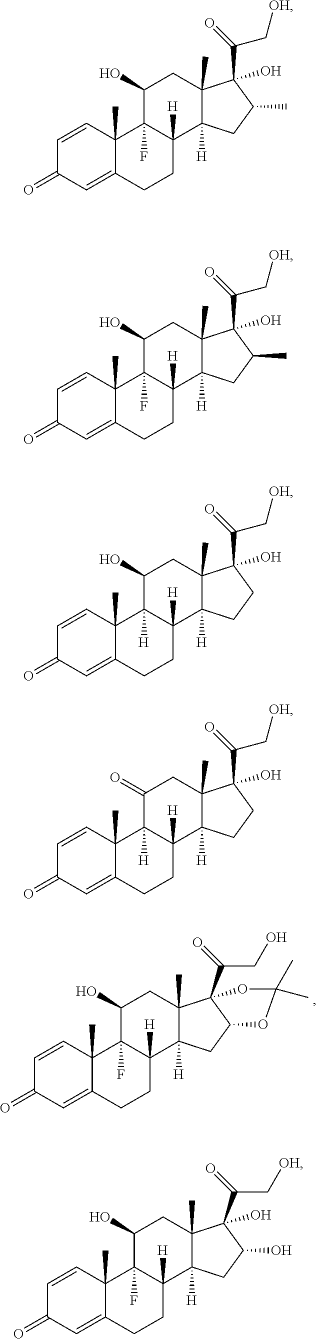

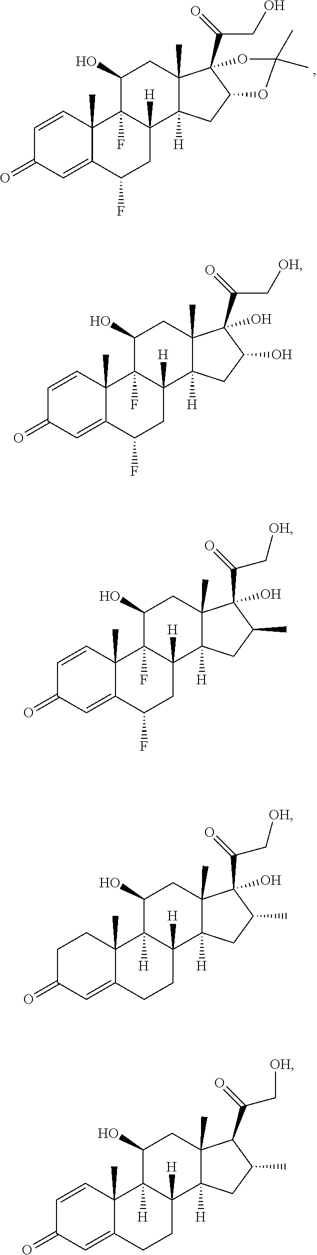

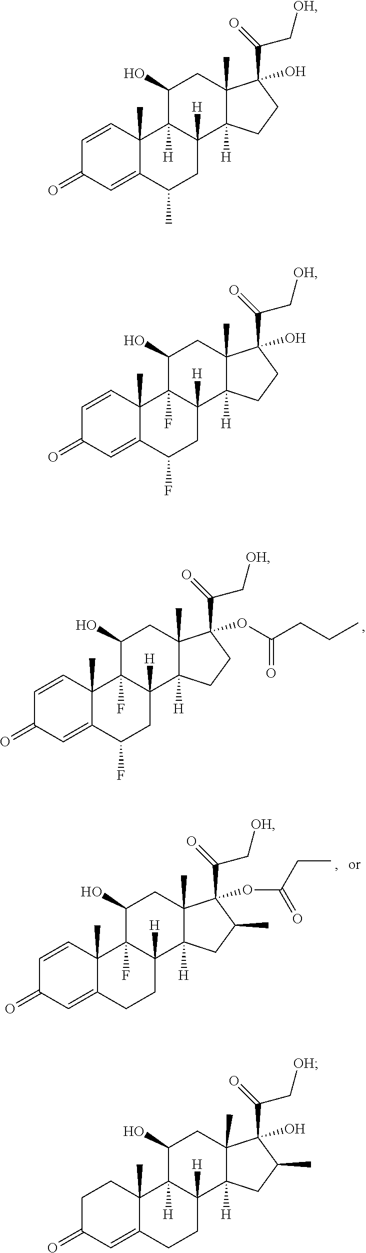

wherein the covalent linkage of the primary alcohol of R⁸ with R¹⁰ forms an ester;
R¹⁰ is

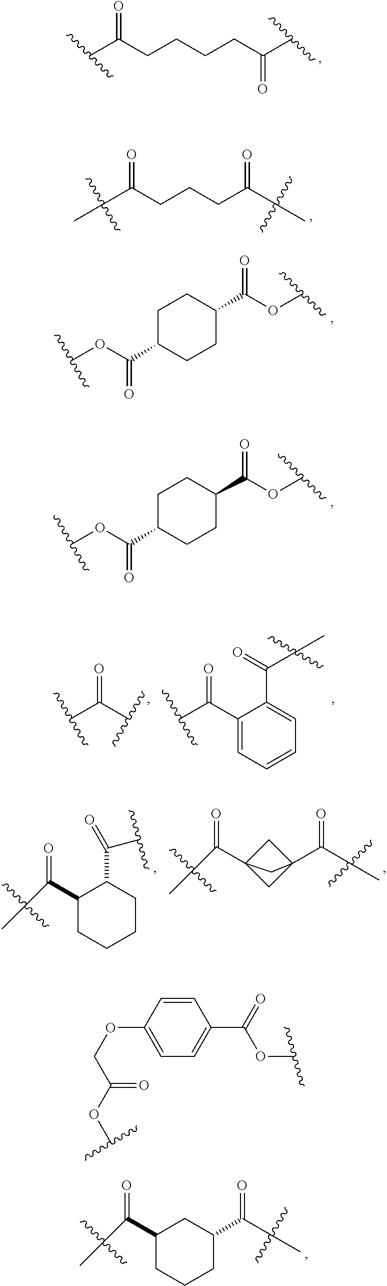

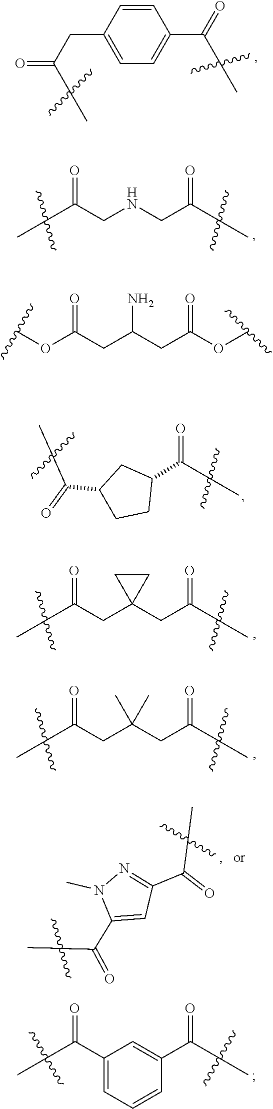

and
R¹¹ is

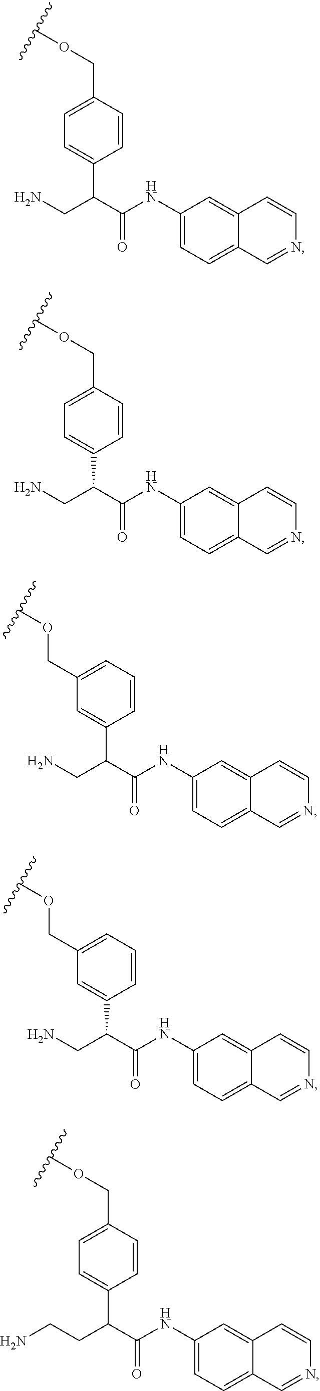 [SMILES: *OCc1ccc(C(CN)C(=O)Nc2ccc3cnccc3c2)cc1 ; *OCc1ccc([C@@H](CN)C(=O)Nc2ccc3cnccc3c2)cc1 ; *OCc1cccc(C(CN)C(=O)Nc2ccc3cnccc3c2)c1 ; *OCc1cccc([C@@H](CN)C(=O)Nc2ccc3cnccc3c2)c1 ; *OCc1ccc(C(CCN)C(=O)Nc2ccc3cnccc3c2)cc1]

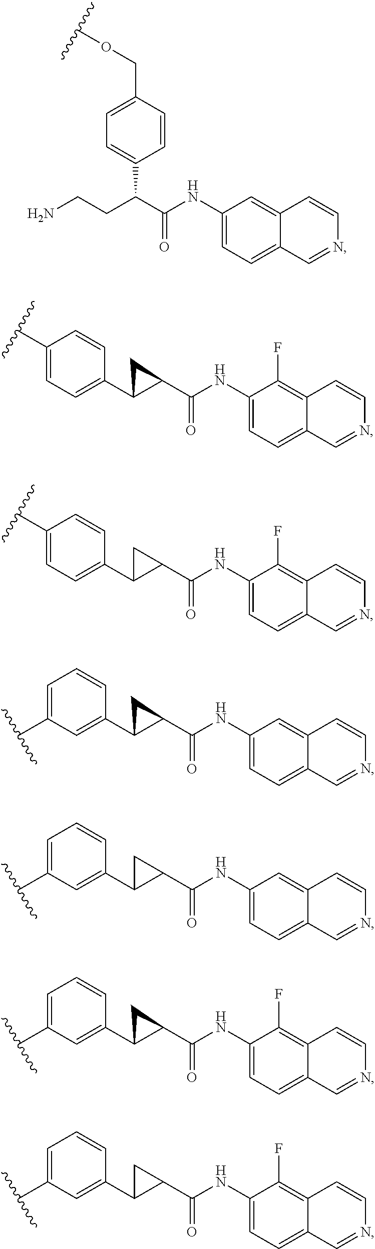

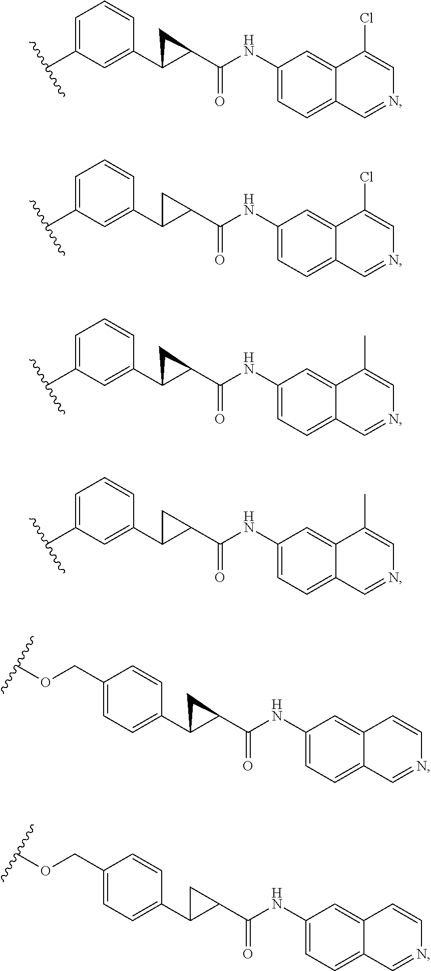 [SMILES: *c1cccc([C@@H]2C[C@H]2C(=O)Nc2ccc3cncc(Cl)c3c2)c1 ; *c1cccc(C2CC2C(=O)Nc2ccc3cncc(Cl)c3c2)c1 ; *c1cccc([C@@H]2C[C@H]2C(=O)Nc2ccc3cncc(C)c3c2)c1 ; *c1cccc(C2CC2C(=O)Nc2ccc3cncc(C)c3c2)c1 ; *OCc1ccc([C@@H]2C[C@H]2C(=O)Nc2ccc3cnccc3c2)cc1 ; *OCc1ccc(C2CC2C(=O)Nc2ccc3cnccc3c2)cc1]

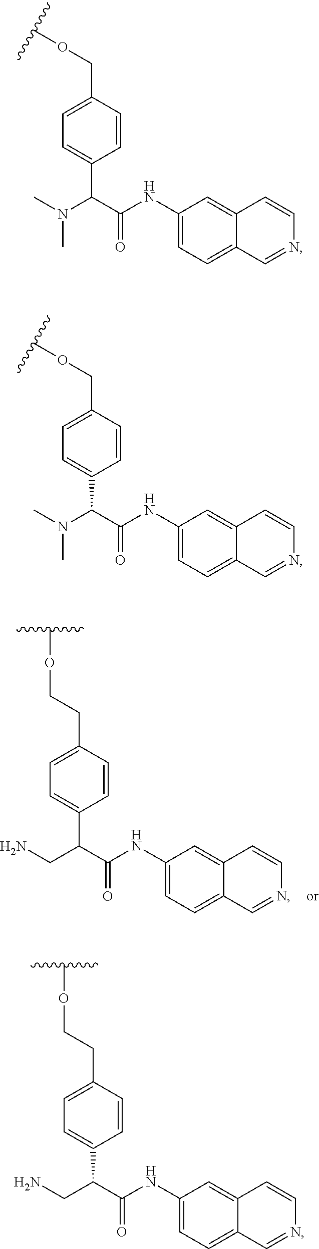 [SMILES: *OCc1ccc(C(C(=O)Nc2ccc3cnccc3c2)N(C)C)cc1 ; *OCc1ccc([C@H](C(=O)Nc2ccc3cnccc3c2)N(C)C)cc1 ; *OCCc1ccc(C(CN)C(=O)Nc2ccc3cnccc3c2)cc1 ; *OCCc1ccc([C@@H](CN)C(=O)Nc2ccc3cnccc3c2)cc1]

In some embodiments:
R¹⁰ is

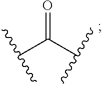 [SMILES: *C(*)=O]

and
R¹¹ is

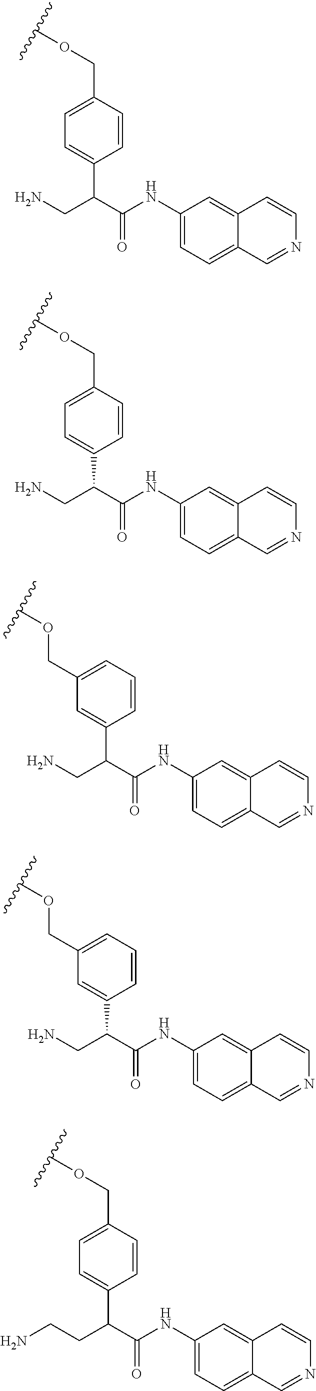 [SMILES: *OCc1ccc(C(CN)C(=O)Nc2ccc3cnccc3c2)cc1 ; *OCc1ccc([C@@H](CN)C(=O)Nc2ccc3cnccc3c2)cc1 ; *OCc1cccc(C(CN)C(=O)Nc2ccc3cnccc3c2)c1 ; *OCc1cccc([C@@H](CN)C(=O)Nc2ccc3cnccc3c2)c1 ; *OCc1ccc(C(CCN)C(=O)Nc2ccc3cnccc3c2)cc1]

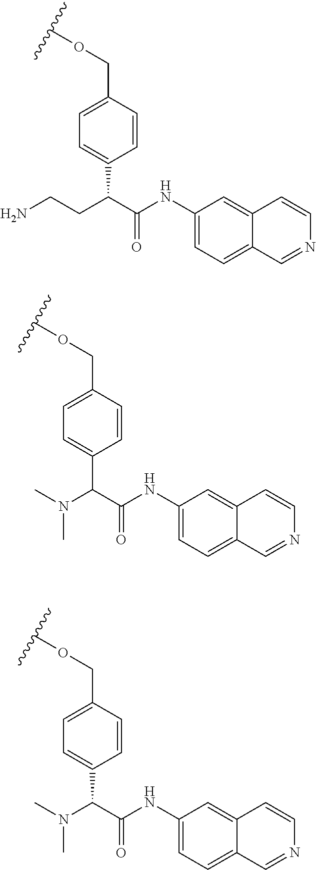 [SMILES: *OCc1ccc([C@@H](CCN)C(=O)Nc2ccc3cnccc3c2)cc1 ; *OCc1ccc(C(C(=O)Nc2ccc3cnccc3c2)N(C)C)cc1 ; *OCc1ccc([C@H](C(=O)Nc2ccc3cnccc3c2)N(C)C)cc1]

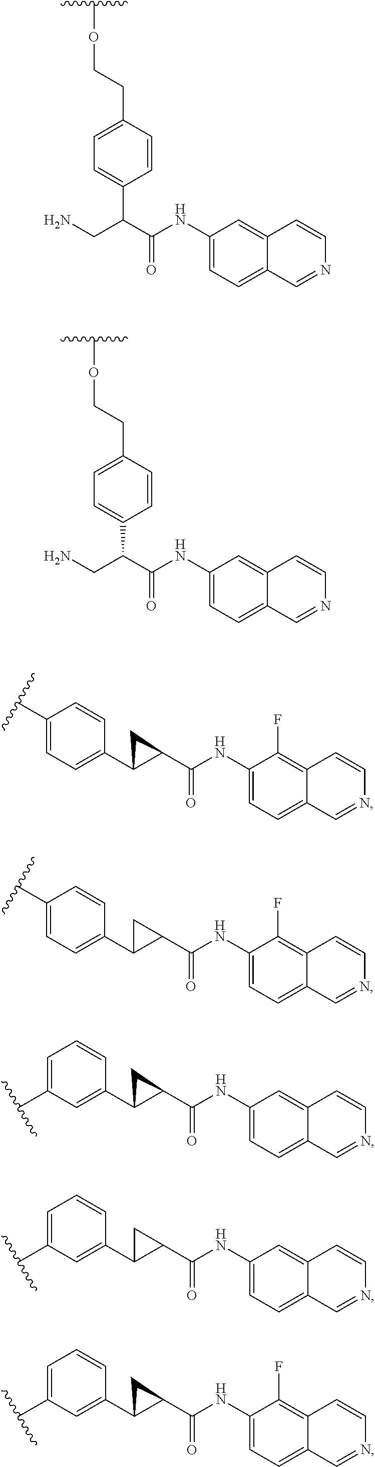

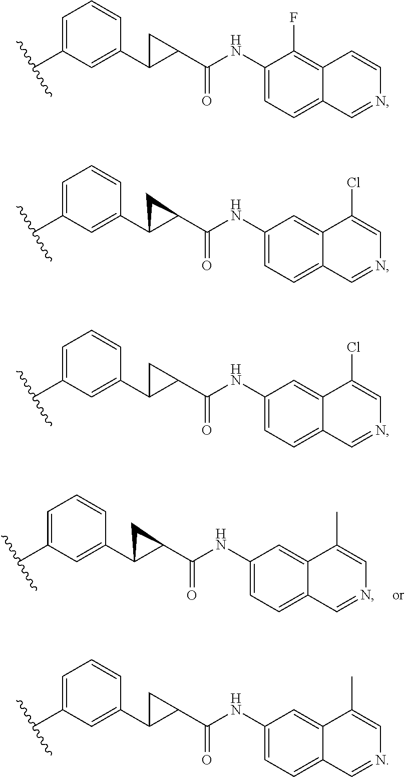 [SMILES: *c1cccc(C2CC2C(=O)Nc2ccc3cnccc3c2F)c1 ; *c1cccc([C@@H]2C[C@H]2C(=O)Nc2ccc3cncc(Cl)c3c2)c1 ; *c1cccc(C2CC2C(=O)Nc2ccc3cncc(Cl)c3c2)c1 ; *c1cccc([C@@H]2C[C@H]2C(=O)Nc2ccc3cncc(C)c3c2)c1 ; *c1cccc(C2CC2C(=O)Nc2ccc3cncc(C)c3c2)c1]

In some embodiments, the compound is a compound of Table 1, Table 2, Table 3, Table 4, Table 5, or Table 6 or a pharmaceutically acceptable salt thereof.

TABLE 2

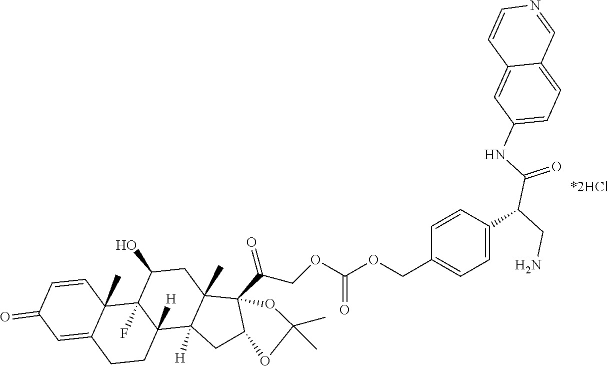

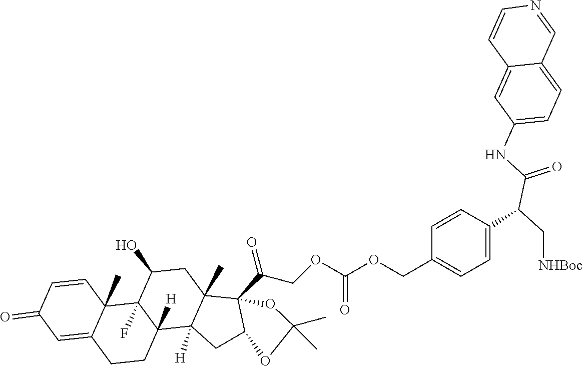

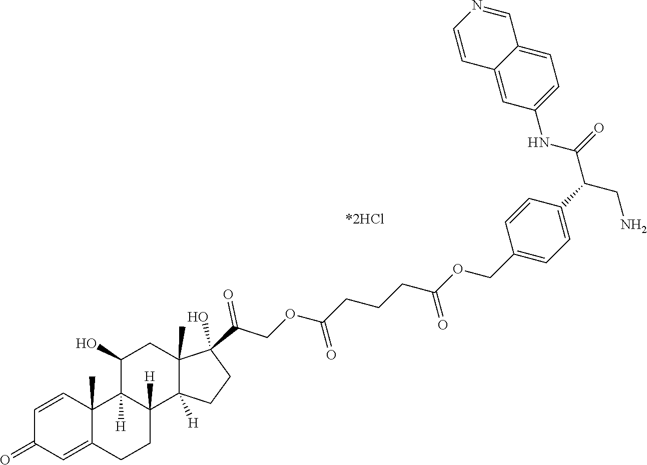

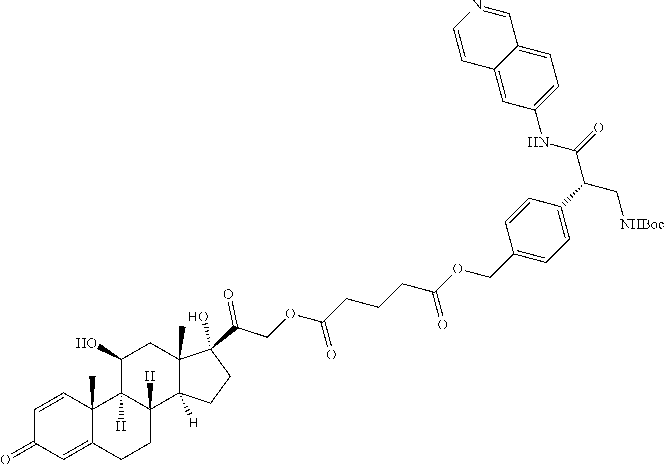

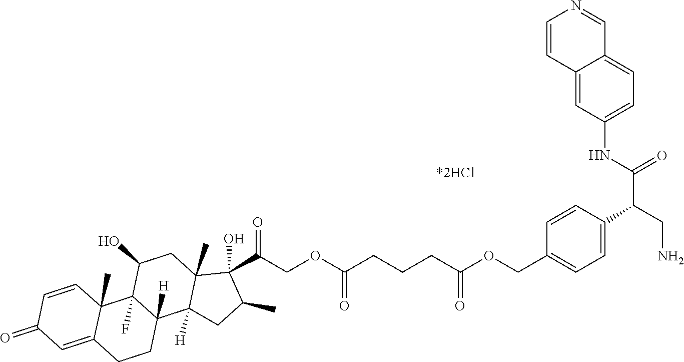

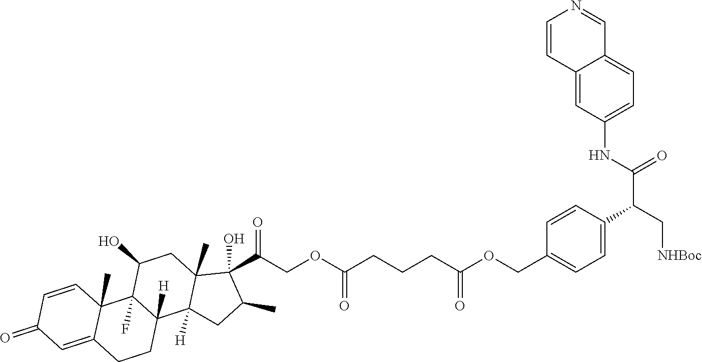

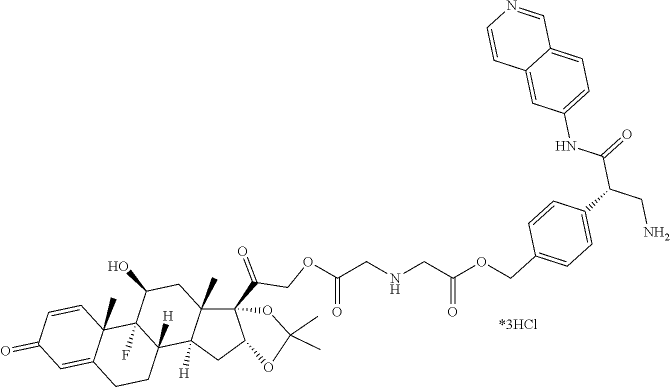

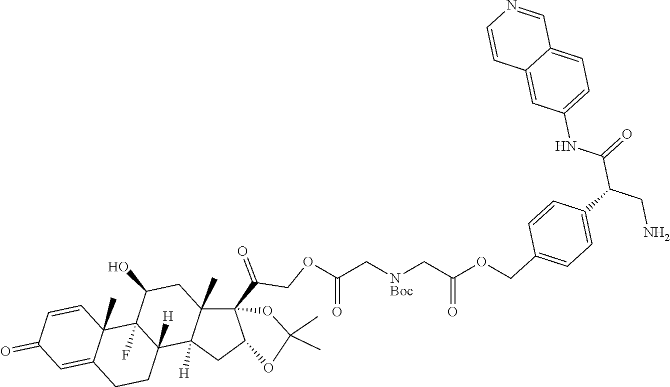

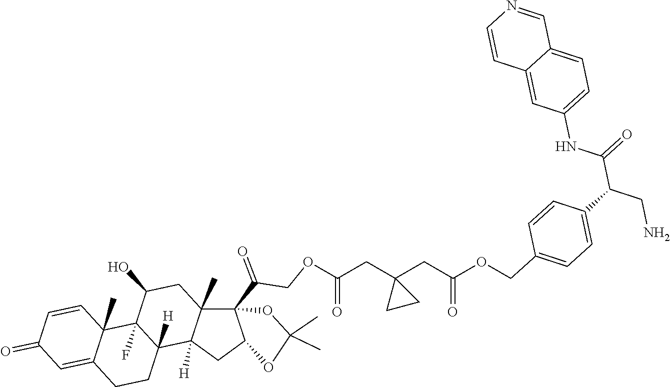

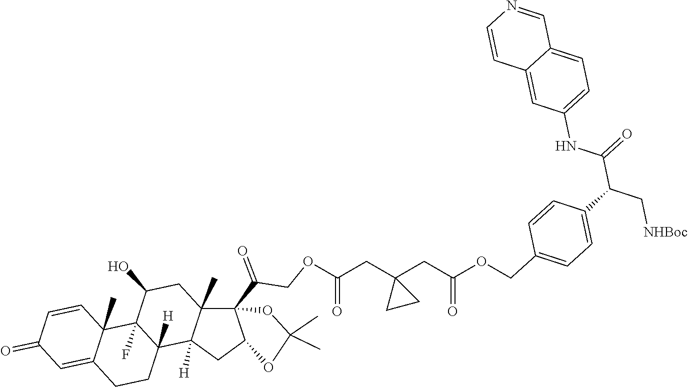

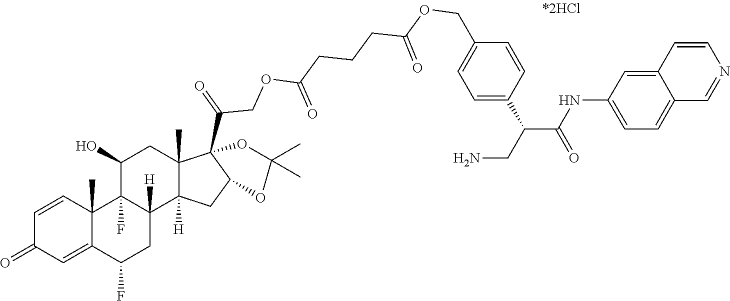

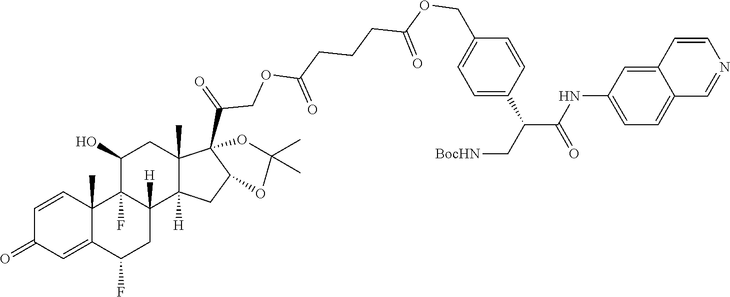

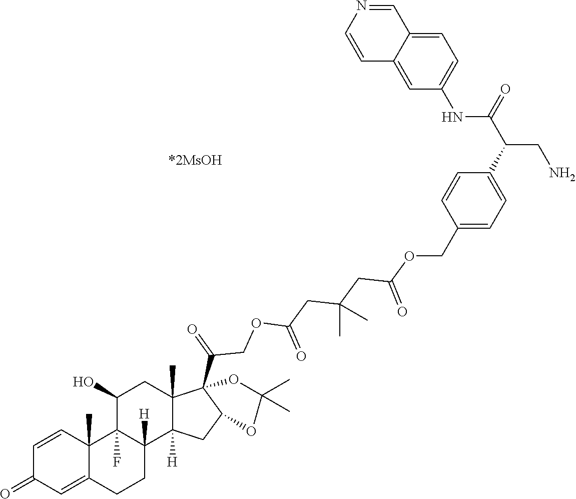

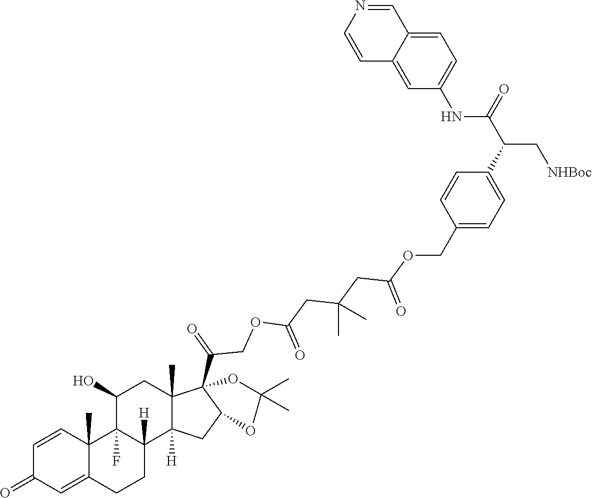

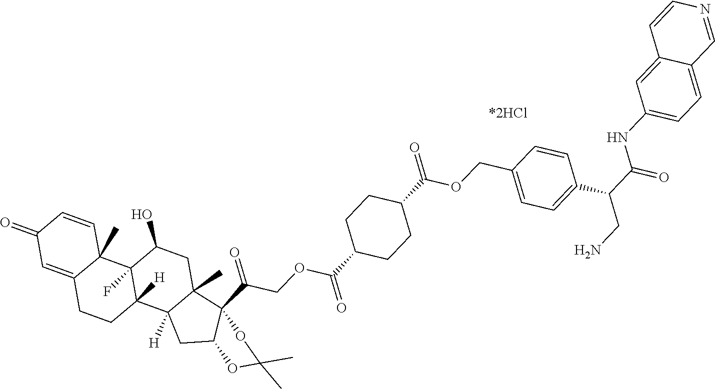

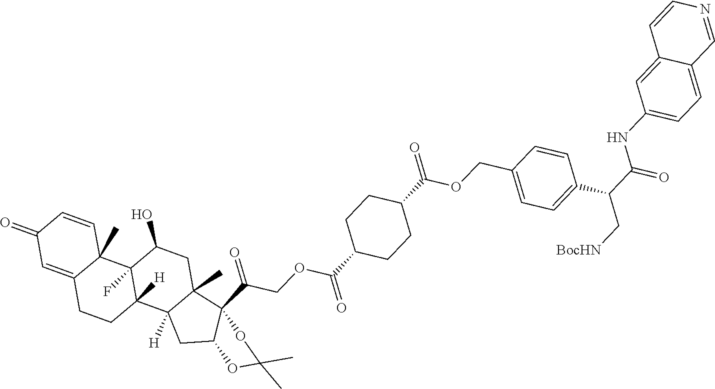

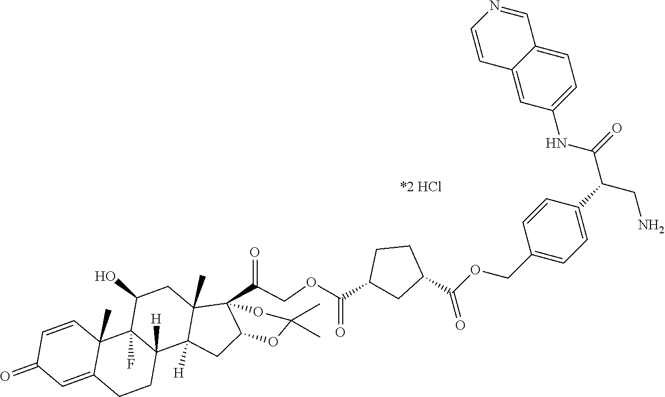

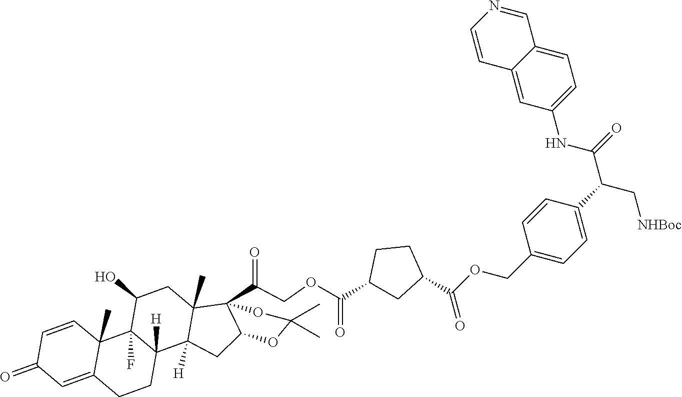

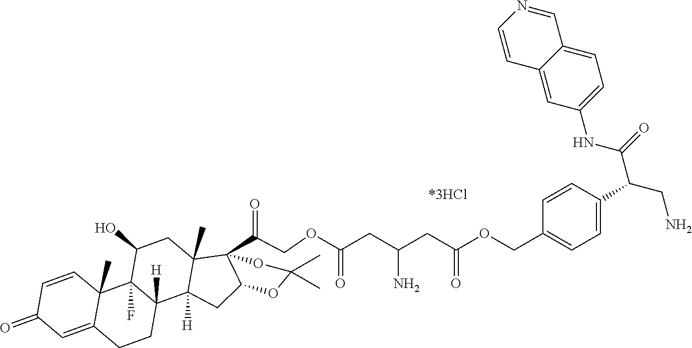

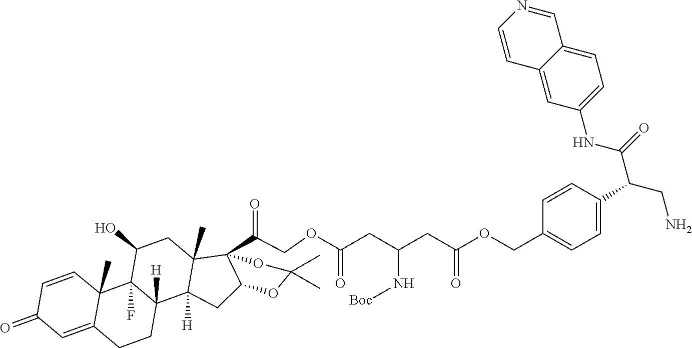

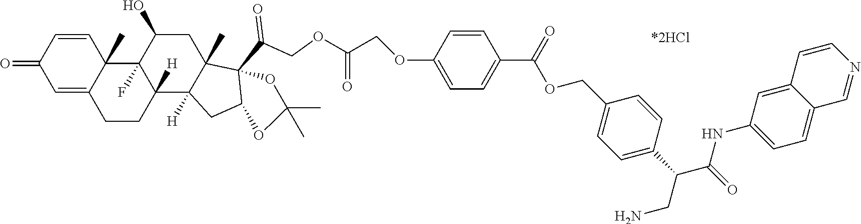

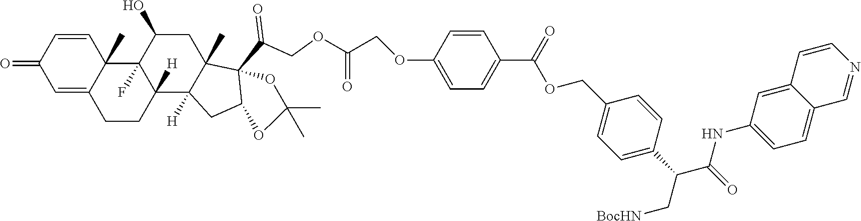

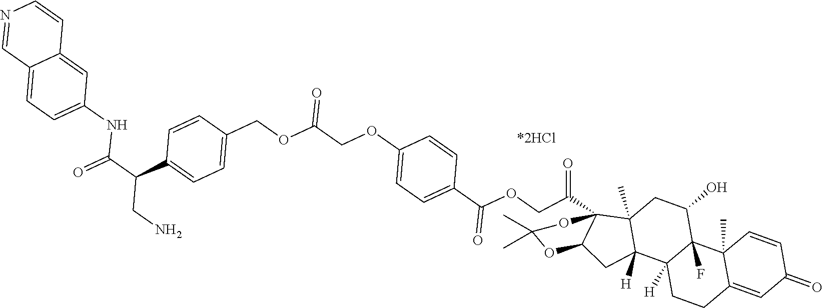

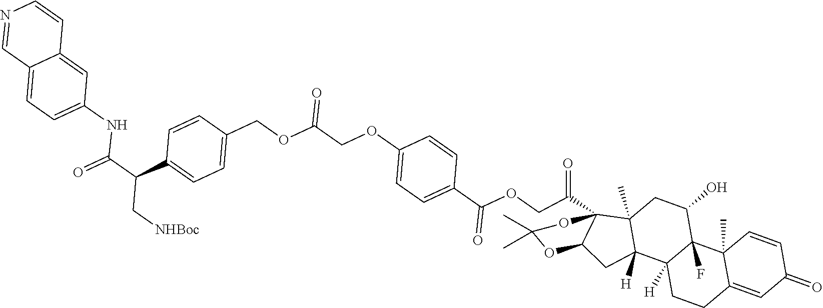

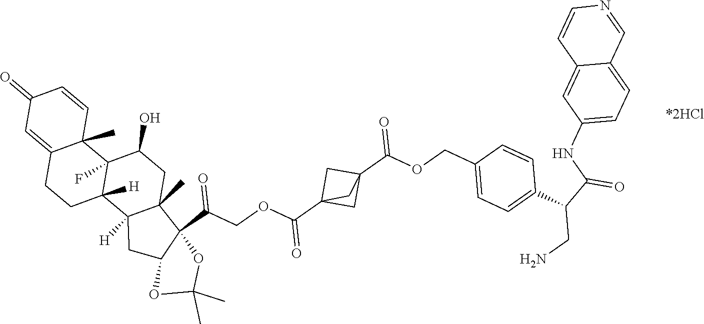

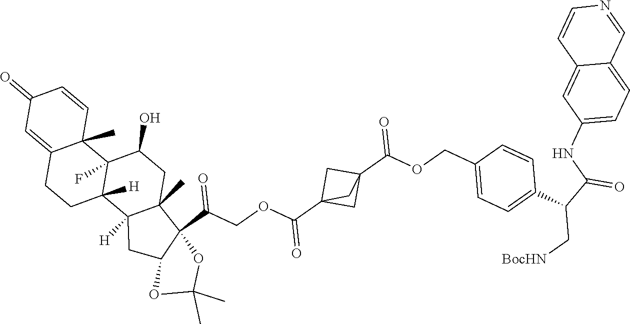

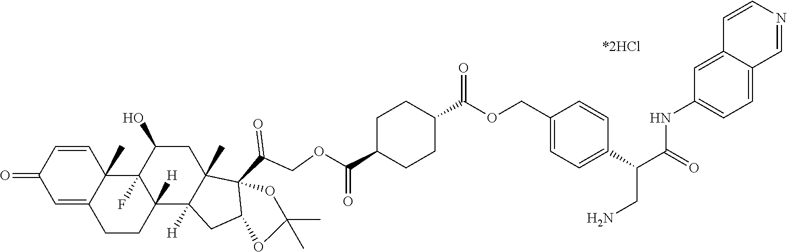

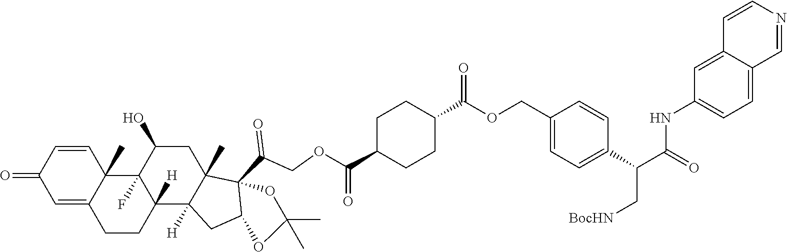

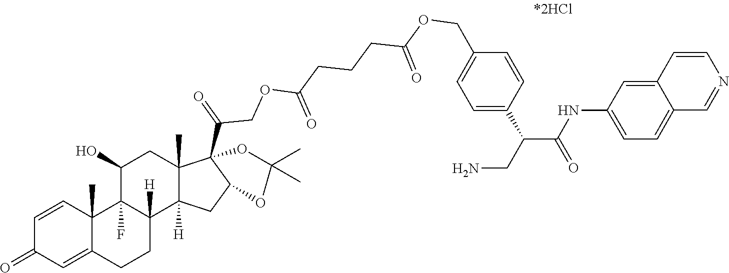

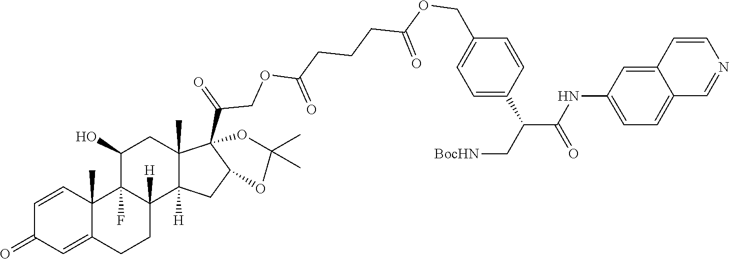

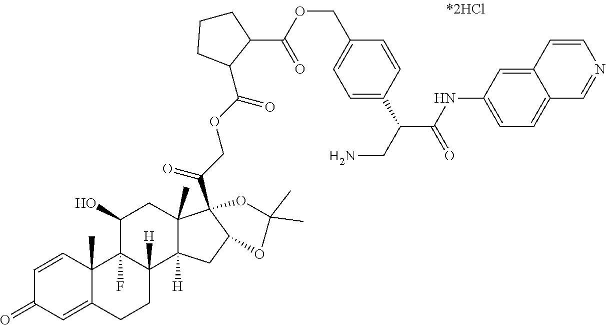

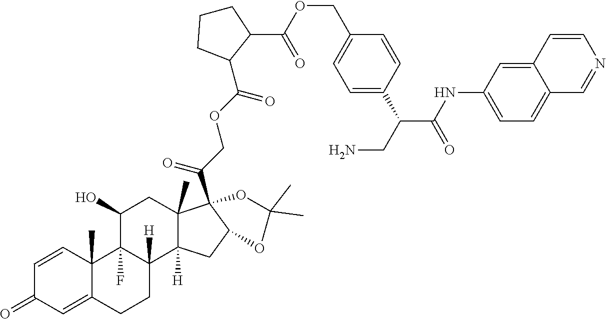

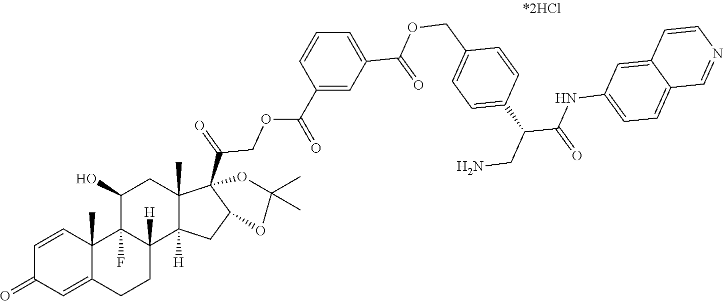

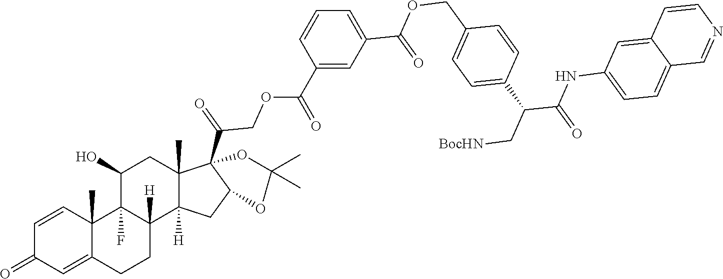

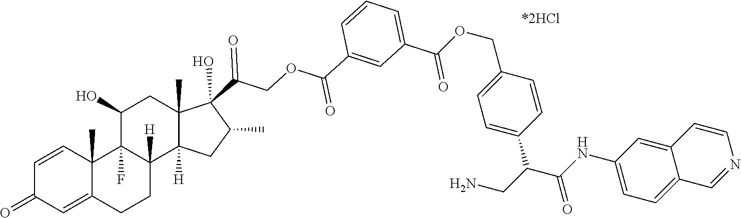

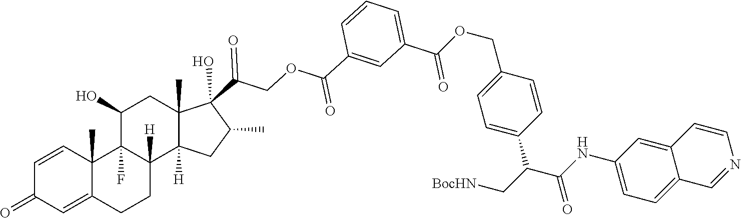

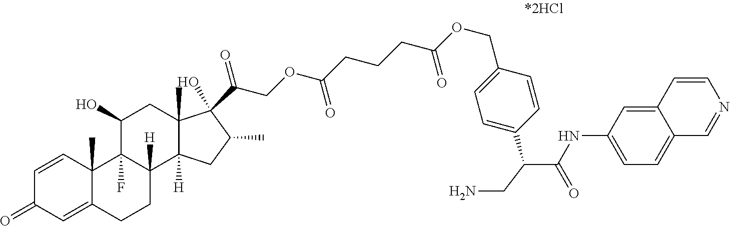

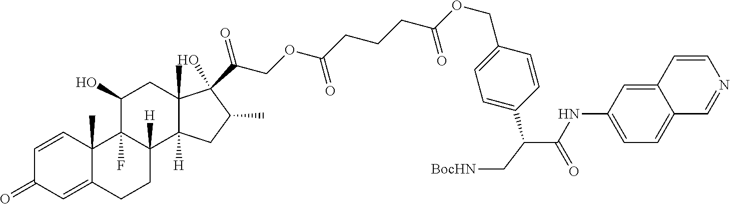

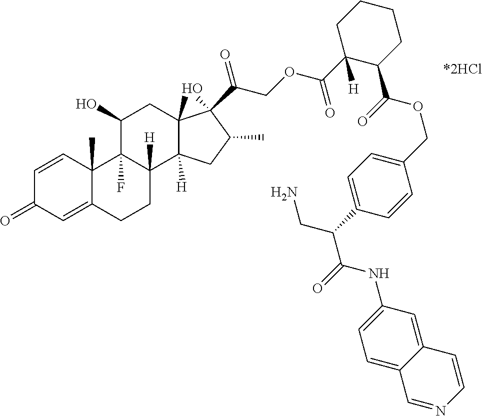

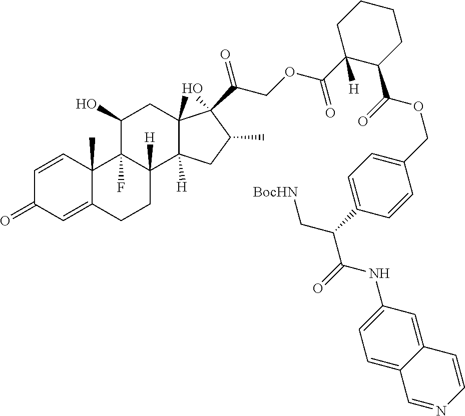

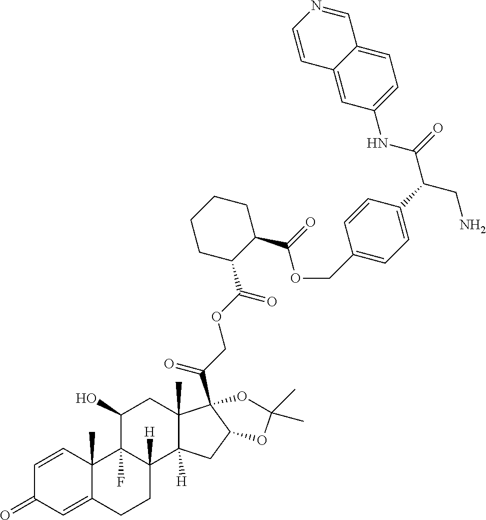

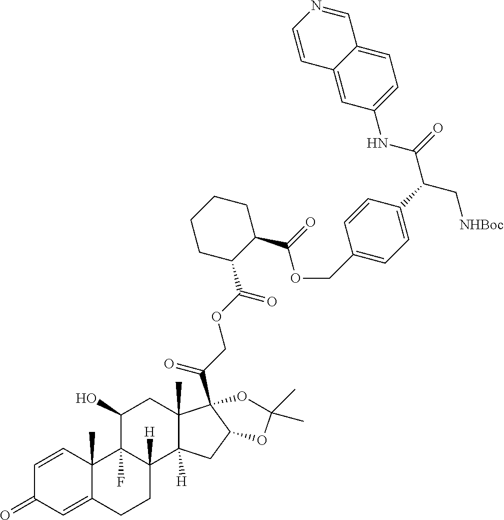

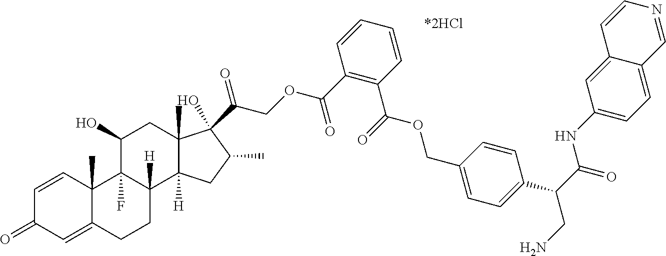

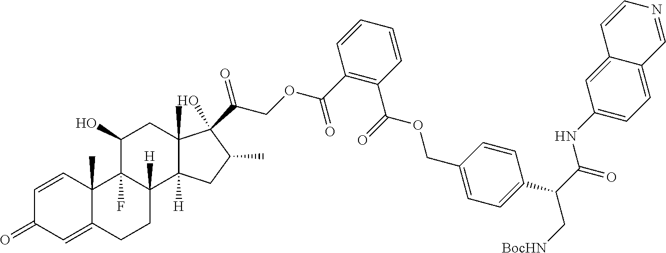

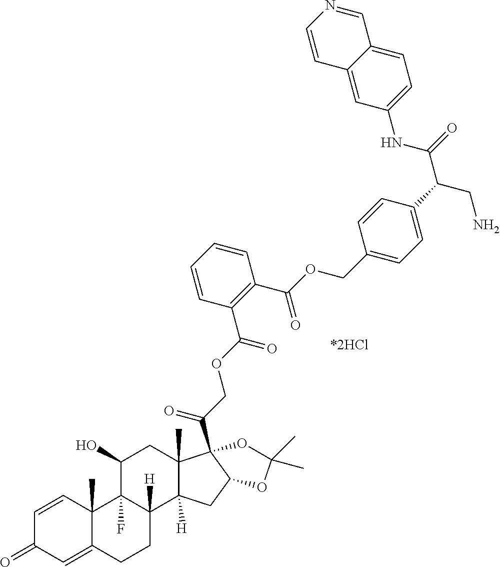

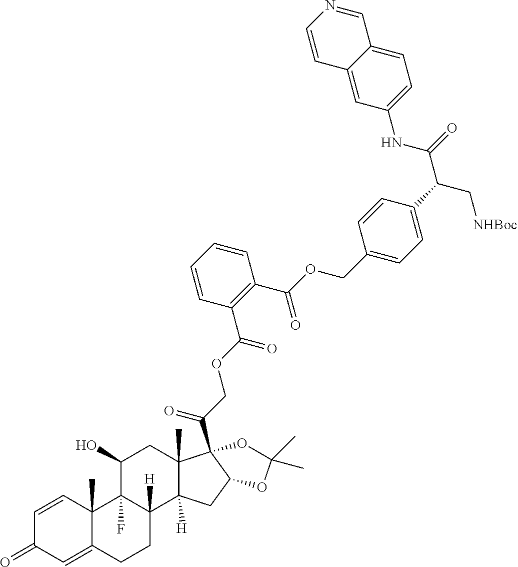

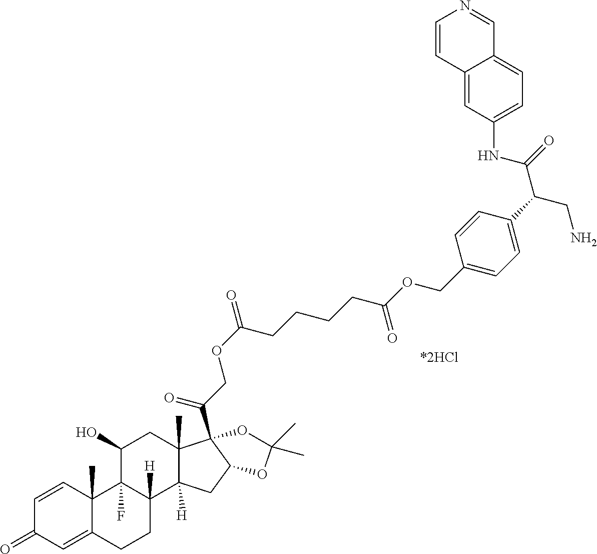

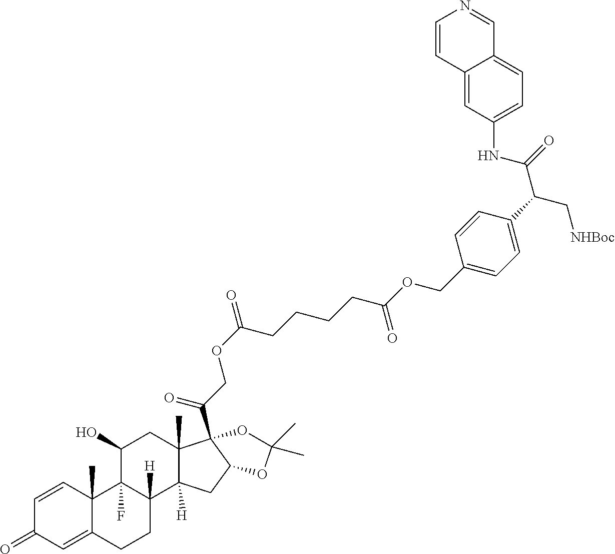

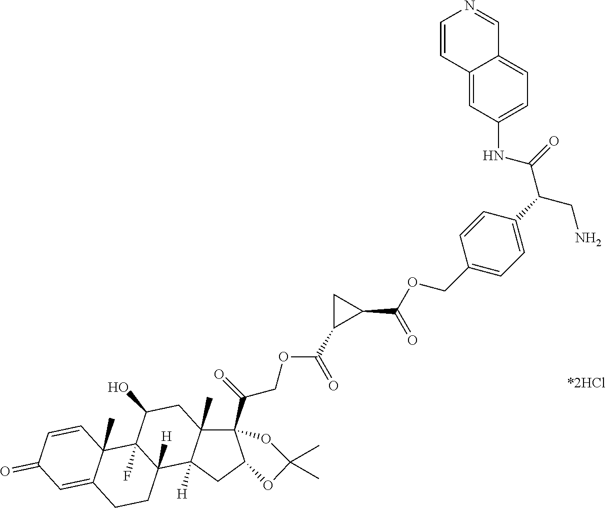

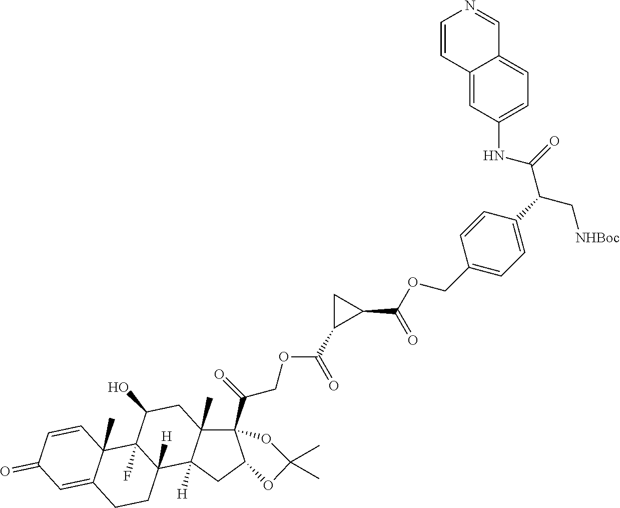

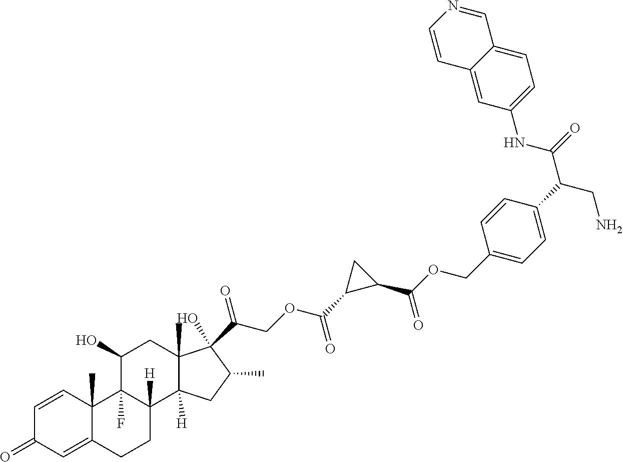

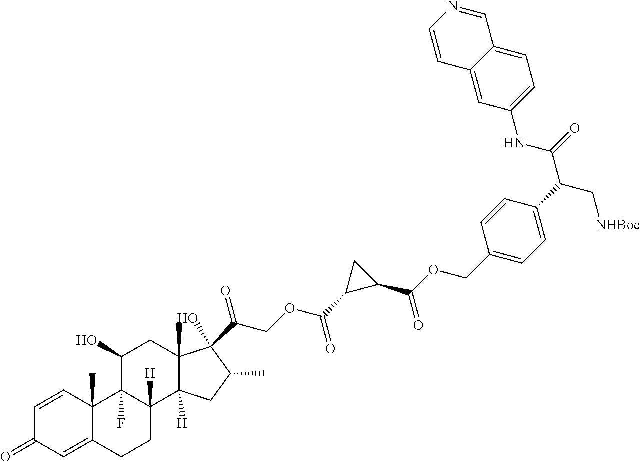

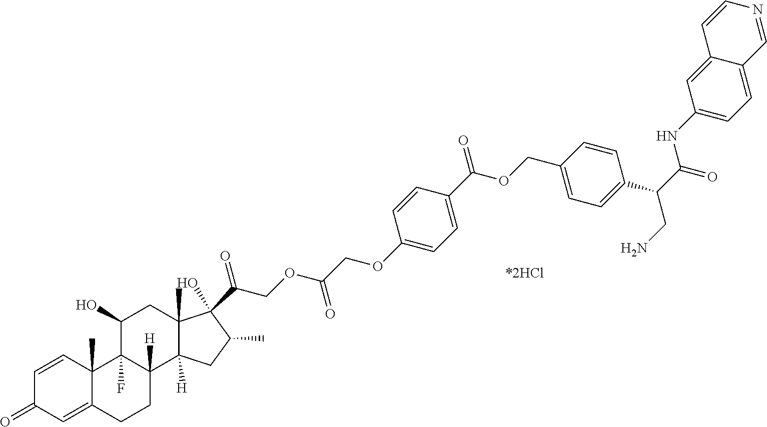

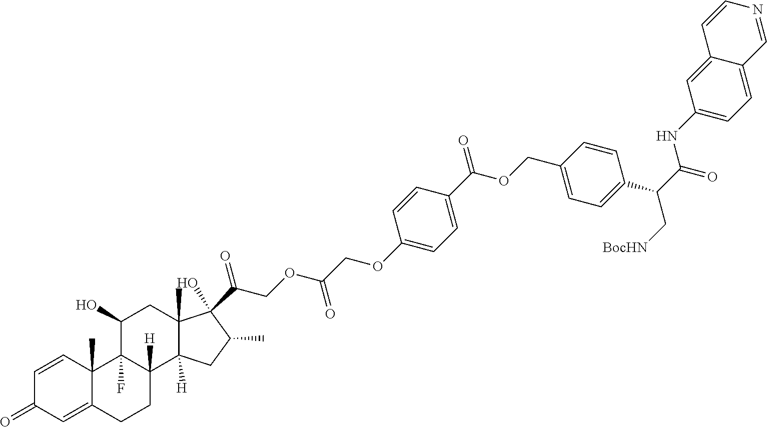

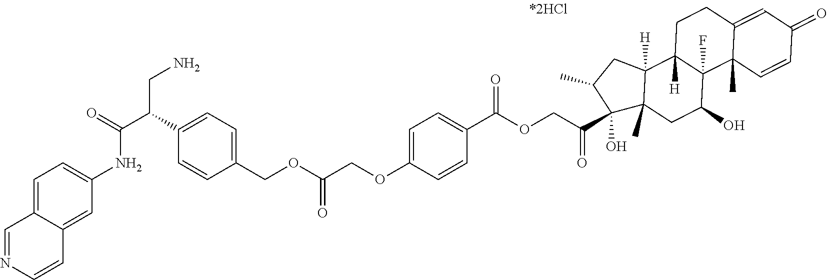

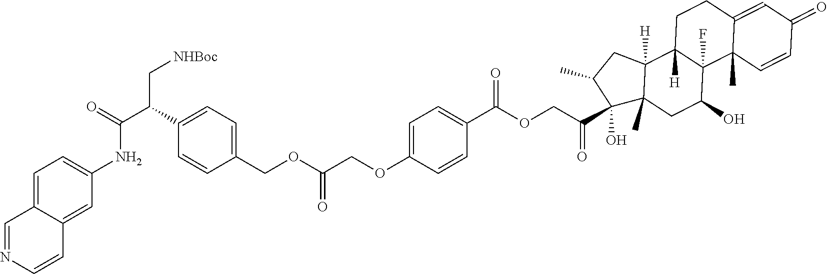

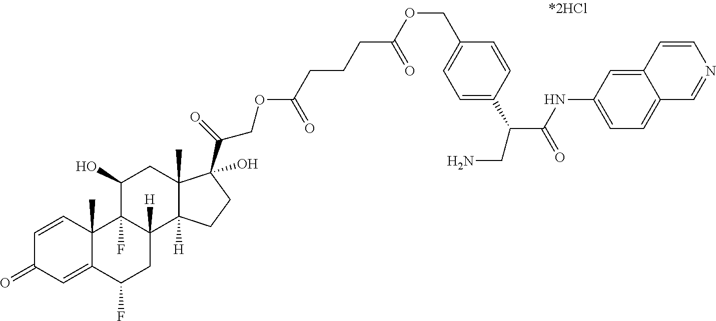

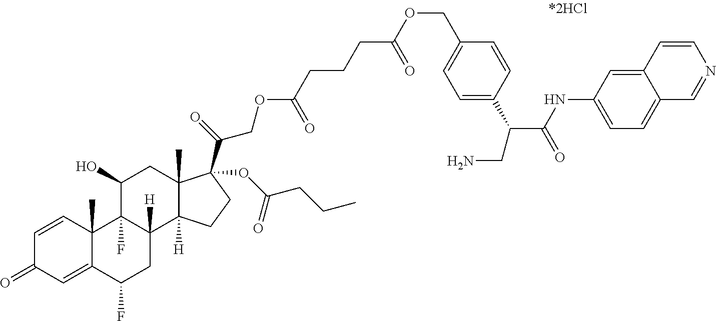

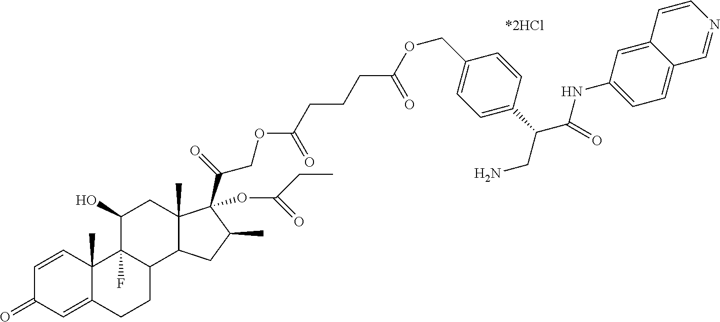

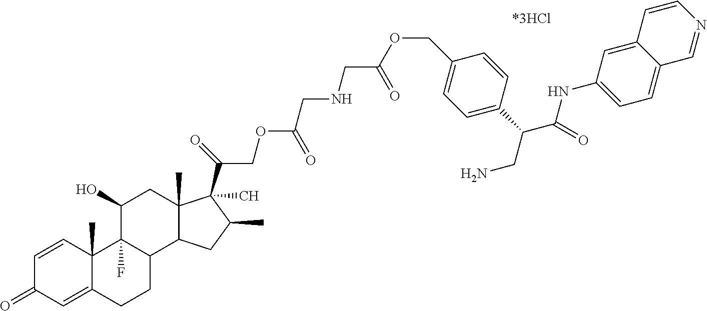

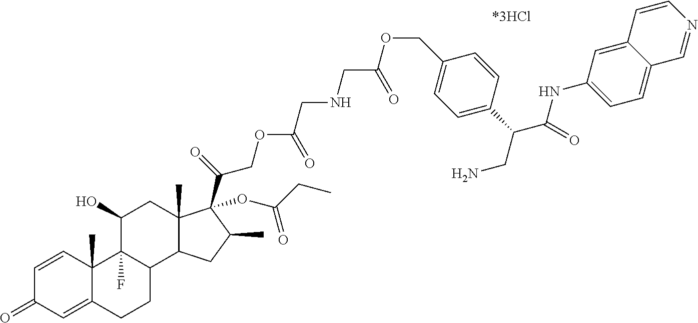

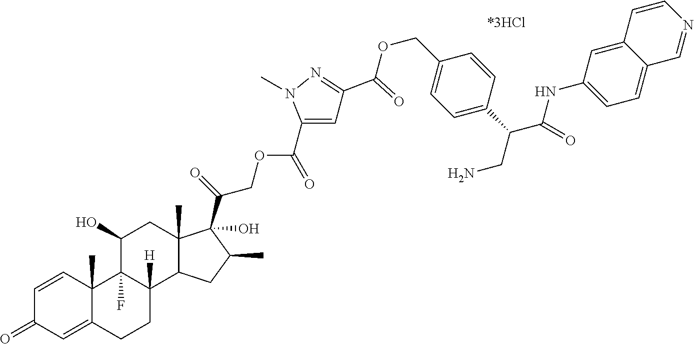

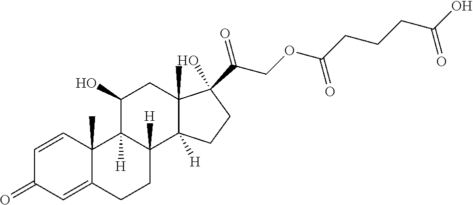

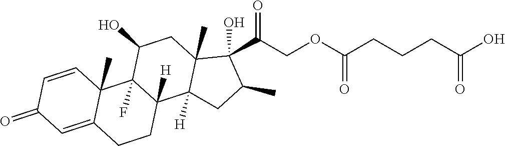

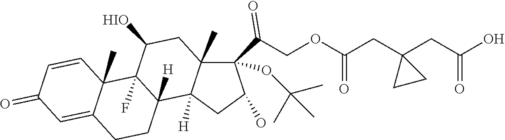

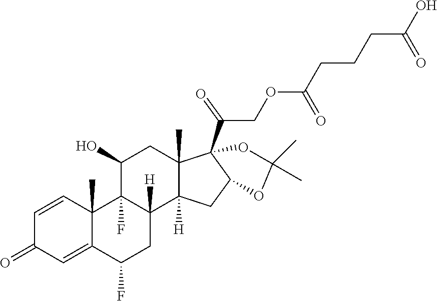

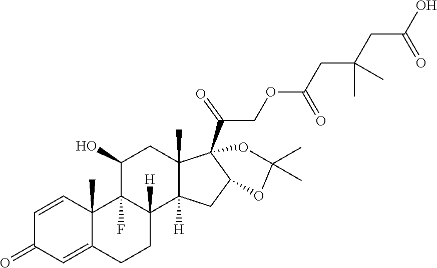

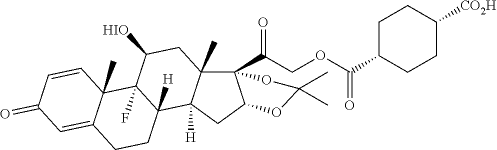

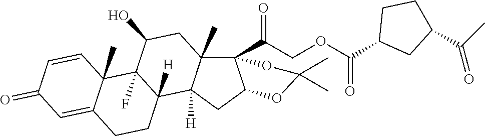

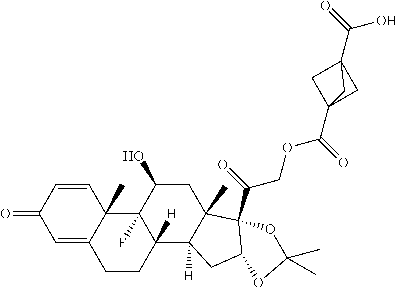

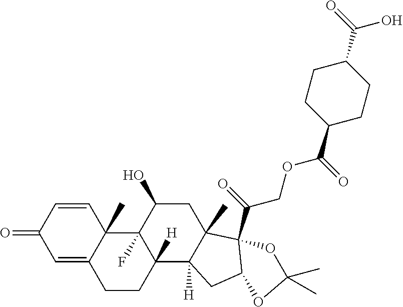

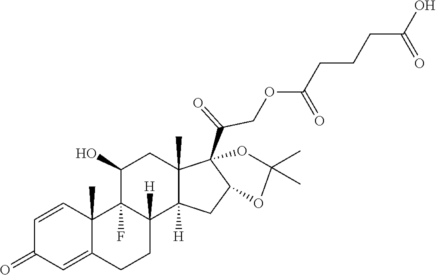

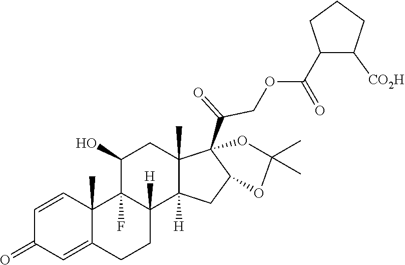

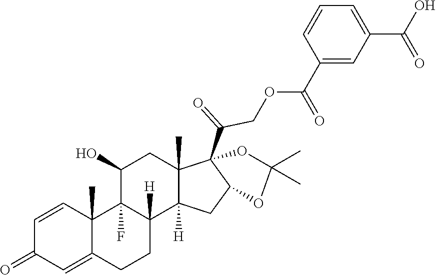

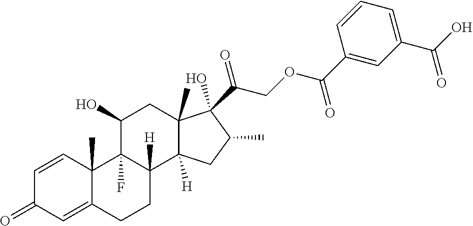

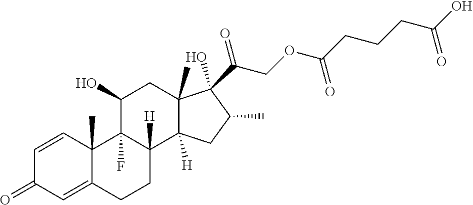

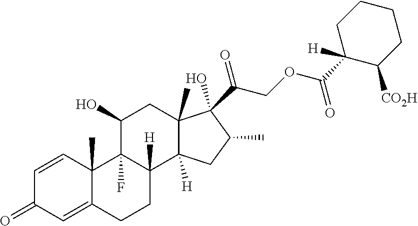

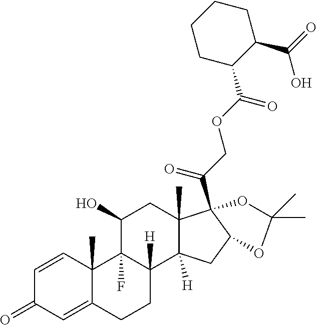

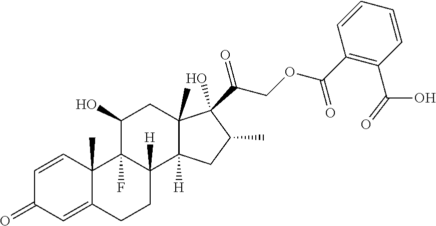

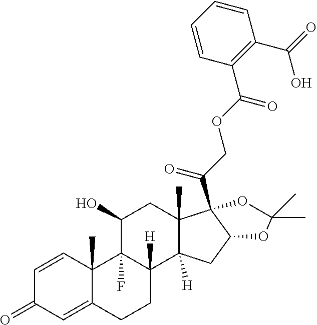

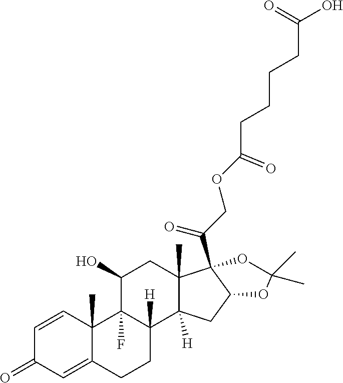

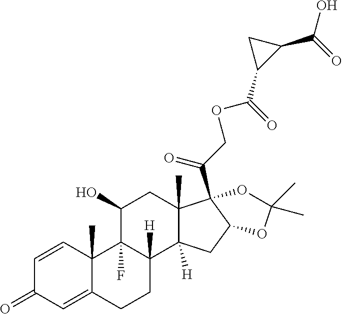

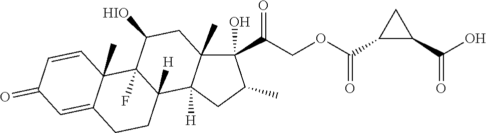

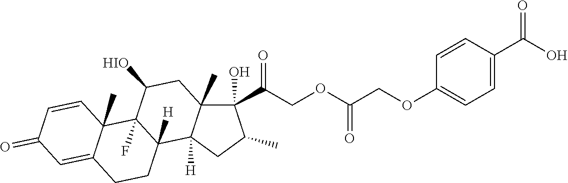

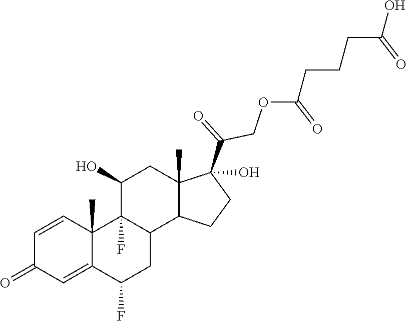

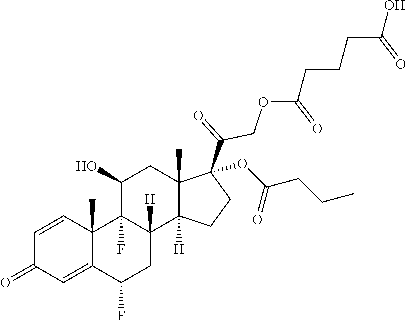

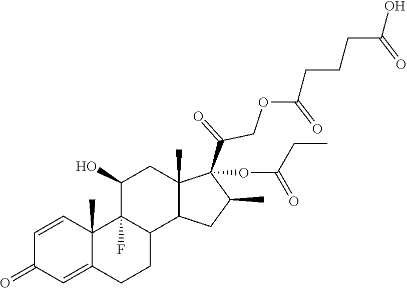

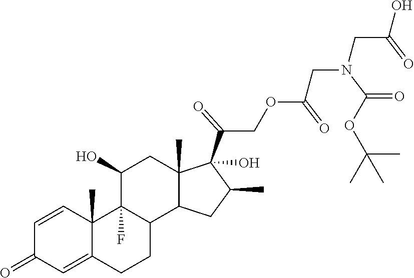

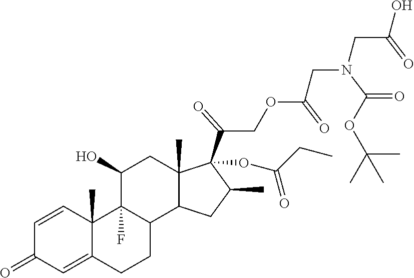

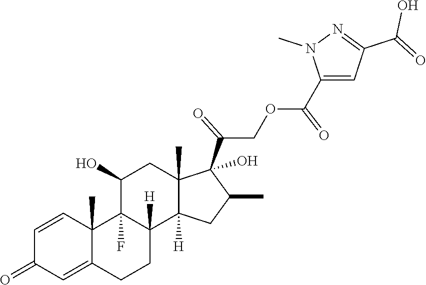

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁶O, ³²P, and ³⁵S. In one embodiment, isotopically-labeled compounds are useful in drug and/or substrate tissue distribution studies. In another embodiment, substitution with heavier isotopes such as deuterium affords greater metabolic stability (for example, increased in vivo half-life or reduced dosage requirements). In yet another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In one embodiment, the compounds described herein are labeled by other means, including, but not limited to, the use of chromophores or fluorescent moieties, bioluminescent labels, or chemiluminescent labels.

It will be appreciated that the description of the compounds provided herein should be construed in congruity with the laws and principals of chemical bonding. In some instances, it may be necessary to remove a hydrogen atom in order to accommodate a substituent at any given location. Some compounds shown in the tables provided herein may not include hydrogens on hydroxyl groups or amine groups (i.e. primary or secondary amines); it is understood that hydrogen is present at these positions if not shown, just as a carbon may not always explicitly show every hydrogen attached thereto.

The compounds described herein, and other related compounds having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons. 1991), LaRock's Comprehensive Organic Transformations (VCH Publishers Inc., 1989), March, Advanced Organic Chemistry 4^{th} Ed., (Wiley 1992); Carey and Sundberg, Advanced Organic Chemistry 4^{th} Ed., Vols. A and B (Plenum 2000, 2001), and Greene and Wuts, Protective Groups in Organic Synthesis 3^{rd} Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources or are prepared using procedures described herein.

### Methods

Provided herein are methods of treating a disease in a subject in need thereof, comprising administering to the subject an effective amount of a compound provided herein.

Thus, in one aspect, provided herein is a method of treating an eye disease or disorder in a subject in need thereof, comprising administering to the subject an effective amount of a compound of Formulae I-X or a pharmaceutically acceptable salt thereof.

In some embodiments, provided herein is a method of treating an eye disease or disorder in a subject in need thereof, comprising administering to the subject an effective amount of a compound of Table 1, Table 2, Table 3, Table 4, Table 5, or Table 6, or a pharmaceutically acceptable salt thereof.

In some embodiments, the eye disease or disorder comprises glaucoma, a neurodegenerative eye disease or disorder, dry eye, ocular hypertension, or an ocular inflammatory disease or disorder.

Also provided herein are methods of reducing intraocular pressure in an eye of a subject in need thereof, comprising administering to the subject an effective amount of a compound provided herein.

Thus, in another aspect, provided herein is a method of reducing intraocular pressure in an eye of a subject in need thereof, comprising administering to the subject an effective amount of a compound of Formulae I-X or a pharmaceutically acceptable salt thereof.

In some embodiments, provided herein is a method of reducing intraocular pressure in an eye of a subject in need thereof, comprising administering to the subject an effective amount of a compound of Table 1, Table 2, Table 3, Table 4, Table 5, or Table 6, or a pharmaceutically acceptable salt thereof.

In some embodiments, the subject is suffering from glaucoma or ocular hypertension.

Also provided herein are methods of modulating kinase activity in a cell, comprising contacting the cell with an amount effective to modulate kinase activity of a compound provided herein.

Thus, in yet another aspect, provided herein is a method of modulating kinase activity in a cell, comprising contacting the cell with an amount effective to modulate kinase activity of a compound of Formulae I-X or a pharmaceutically acceptable salt thereof.

In some embodiments, provided herein is a method of modulating kinase activity in a cell, comprising contacting the cell with an amount effective to modulate kinase activity of a compound of Table 1, Table 2, Table 3, Table 4, Table 5, or Table 6, or a pharmaceutically acceptable salt thereof.

In some embodiments, the cell is in a subject.

In some embodiments of these methods, the subject is a human.

In some embodiments of these methods, the administration is topical administration. In some embodiments, the topical administration is topical administration to an eye, or both eyes, of the subject.

In some embodiments of these methods, the administration is ocular administration.

In some embodiments of these methods, the administration is systemic administration,

### Administration/Dosage/Formulations

In another aspect, provided herein are compositions comprising a compound provided herein.

In another aspect, provided herein are pharmaceutical compositions comprising a compound provided herein and a pharmaceutically acceptable carrier.

Actual dosage levels of an active ingredient in the pharmaceutical compositions provided herein may be varied so as to obtain an amount of the active ingredient that is effective to achieve a desired therapeutic response for a particular subject, composition, or mode of administration, without being toxic to the subject.

In some embodiments, it is especially advantageous to formulate the compound in dosage unit form for ease of administration and uniformity of dosage. Dosage unit form as used herein refers to physically discrete units suited as unitary dosages for the subject to be treated; each unit containing a predetermined quantity of therapeutic compound calculated to produce the desired therapeutic effect in association with the required pharmaceutical vehicle. The dosage unit forms of the present disclosure are dictated by and directly dependent on (a) the unique characteristics of the therapeutic compound and the particular therapeutic effect to be achieved, and (b) the limitations inherent in the art of compounding/formulating such a therapeutic compound for the treatment of the diseases referred to herein in a subject in need thereof.

In one embodiment, the compounds or compositions provided herein are formulated using one or more pharmaceutically acceptable excipients or carriers. In one embodiment, the pharmaceutical compositions provided herein comprise a therapeutically effective amount of a compound provided herein and a pharmaceutically acceptable carrier.

In one embodiment, the present disclosure provides packaged pharmaceutical compositions comprising a container holding at least one therapeutically effective amount of a compound provided herein, and instructions for using the compound to treat one or more symptoms of a disease referred to herein in a subject in need thereof.

Routes of administration of any of the compositions provided herein include oral, nasal, rectal, intravaginal, parenteral, buccal, sublingual, topical, or ocular. The compounds for use as provided herein may be formulated for administration by any suitable route, such as for ocular, oral or parenteral, for example, transdermal, transmucosal (e.g., sublingual, lingual, (trans) buccal, (trans) urethral, vaginal (e.g., trans- and perivaginally), (intra) nasal and (trans) rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for ocular or intravesical administration and the like. It should be understood that the formulations and compositions that would be useful as provided herein are not limited to the particular formulations and compositions that are described herein.

Those skilled in the art will recognize or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size or volume, and experimental reagents, such as solvents, catalysts, pressures, atmospheric conditions, e.g., nitrogen atmosphere, and reducing or oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or present disclosure as set forth herein.

### EXAMPLES

The present disclosure is now described with reference to the following Examples. These Examples are provided for the purpose of illustration only, and the present disclosure is not limited to these Examples, but rather encompasses all variations that are evident as a result of the teachings provided herein.

### Example 1. 4-((S)-3-amino-1-(isoquinolin-6-ylamino)-1-oxopropan-2-yl)benzyl (2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl) glutarate dihydrochloride (E4 2HCl)

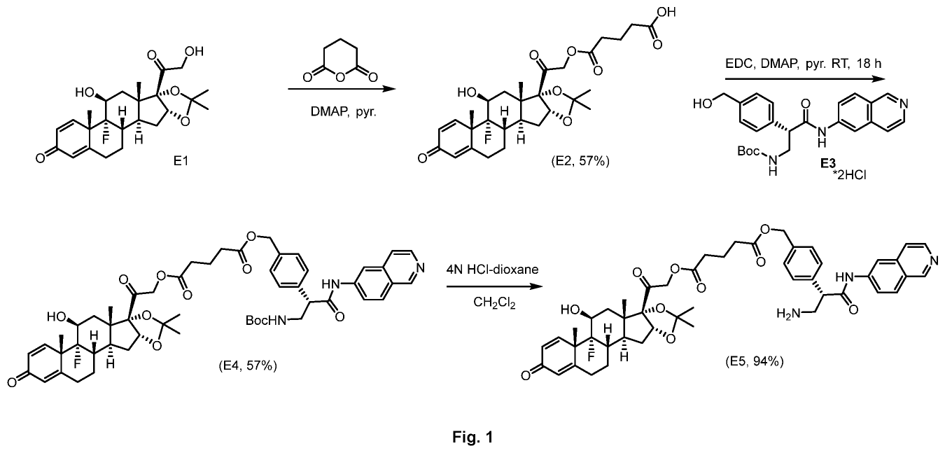

4-((S)-3-amino-1-(isoquinolin-6-ylamino)-1-oxopropan-2-yl)benzyl (2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl) glutarate dihydrochloride (E5 2HCl) was prepared according to the synthetic scheme shown in FIG. 1.

### Preparation of 5-(2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)-5-oxopentanoic acid (E2)

To a solution of triamcinolone acetonide (E1) in anhydrous pyridine was added DMAP and glutaric anhydride. The reaction mixture was stirred overnight under N₂ at room temperature. The mixture was poured into EtOAc and HCl (1N) and extracted. The organic layer was dried over Na₂SO₄, filtered and concentrated to give 5-(2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)-5-oxopentanoic acid (E2, 57%).

### Preparation of 4-((S)-3-((tert-butoxycarbonyl)amino)-1-(isoquinolin-6-ylamino)-1-oxopropan-2-yl)benzyl (2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl) glutarate (E3)

To a solution of 5-(2-((6aS,6bR,7S,8aS,8bS,11aR,12aS,12bS)-6b-fluoro-7-hydroxy-6a,8a, 10. 10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)-5-oxopentanoic acid (E2) in anhydrous pyridine was added EDC, DMAP and tert-butyl(S)-(2-(4-(hydroxymethyl)phenyl)-3-(isoquinolin-6-ylamino)-3-oxopropyl) carbamate (E3) and the solution was stirred under N₂ at room temperature overnight. The reaction was poured into EtOAc/NaHCO₃ (sat) and extracted with EtOAc, dried over Na₂SO₄, filtered and concentrated. Column chromatography 0-5% MeOH/CH₂Cl₂ gave pure 4-((S)-3-((tert-butoxycarbonyl)amino)-1-(isoquinolin-6-ylamino)-1-oxopropan-2-yl)benzyl (2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl) glutarate (E4, 57%).

### Preparation of 4-((S)-3-amino-1-(isoquinolin-6-ylamino)-1-oxopropan-2-yl)benzyl (2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl) glutarate dihydrogicoride (E5 2HCl)

To a solution of 4-((S)-3-((tert-butoxycarbonyl)amino)-1-(isoquinolin-6-ylamino)-1-oxopropan-2-yl)benzyl (2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl) glutarate (E4). in CH₂Cl₂ was added 4M HCl solution in dioxane. The reaction was stirred at room temperature for 5 hours then evaporated. The precipitate was filtered and washed with CH₂Cl₂ to isolate pure 4-((S)-3-amino-1-(isoquinolin-6-ylamino)-1-oxopropan-2-yl)benzyl (2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl) glutarate dihydrogicoride (E5 2HCl, 94%).

### Example 2. Synthesis of 4-((S)-3-amino-1-(isoquinolin-6-ylamino)-1-oxopropan-2-yl)benzyl (2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl) adipate dihydrogen chloride (E8 2HCl)

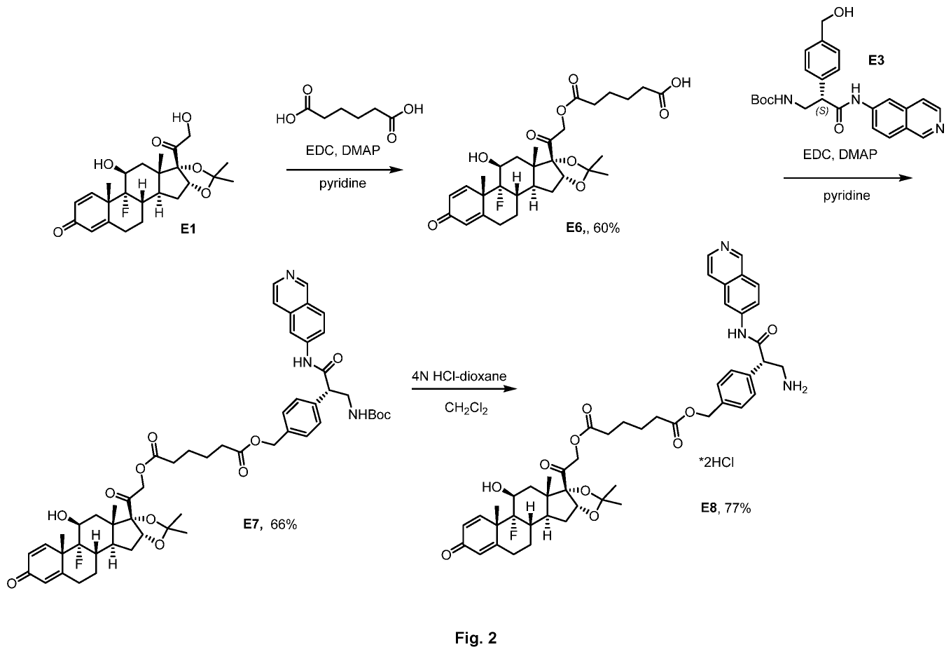

4-((S)-3-amino-1-(isoquinolin-6-ylamino)-1-oxopropan-2-yl)benzyl (2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl) adipate dihydrogen chloride (E8, 2HCl). was prepared according to the synthetic scheme shown in FIG. 2.

### Preparation of -(2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)-6-oxohexanoic acid (E6)

To Triamcinolone acetonide (E1) in pyridine was added adipic acid, EDC and DMAP and the solution stirred overnight at room temperature. The mixture was poured into EtOAc and HCL (1N) and extracted, dried (Na₂SO₄), filtered and evaporated. Column chromatography 0-5% MeOH·CH₂Cl₂ gave pure 6-(2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4.5]indeno[1,2-d][1,3]dioxol-8b-yl)-2-oxoethoxy)-6-oxohexanoic acid (E6, 60%).

### Preparation of 4-((S)-3-((tert-butoxycarbonyl)amino)-1-(isoquinolin-6-ylamino)-1-oxopropan-2-yl)benzyl (2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl) adipate (E7)

To 6-(2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)-6-oxohexanoic acid (E6) in pyridine was added EDC, DMAP and tert-butyl(S)-(2-(4-(hydroxymethyl)phenyl)-3-(isoquinolin-6-ylamino)-3-oxopropyl) carbamate (E3) and the solution was stirred at room temperature overnight. The mixture was poured into EtOAc and NaHCO₃ (sat) and extracted. The combined organics were dried (Na₂SO₄), filtered and evaporated. Column chromatography 0-3% MeOH—CH₂Cl₂ gave pure 4-((S)-3-((tert-butoxycarbonyl)amino)-1-(isoquinolin-6-ylamino)-1-oxopropan-2-yl)benzyl (2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl) adipate (E7, 66%).

### Preparation of 4-((S)-3-amino-1-(isoquinolin-6-ylamino)-1-oxopropan-2-yl)benzyl (2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl) adipate dihydrogen chloride (E8)

To 4-((S)-3-((tert-butoxycarbonyl)amino)-1-(isoquinolin-6-ylamino)-1-oxopropan-2-yl)benzyl (2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl) adipate (E7) in CH₂Cl₂ was added HCl (4N in dioxane) and the solution was stirred at room temperature for 5 hours. The solvents were filtered and the solids washed with CH₂Cl₂, dried to give 4-((S)-3-amino-1-(isoquinolin-6-ylamino)-1-oxopropan-2-yl)benzyl (2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl) adipate dihydrogen chloride (E8, 77%)

### Example 3. 4-((S)-3-amino-1-(isoquinolin-6-ylamino)-1-oxopropan-2-yl)benzyl 4-(2-(2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)-2-oxoethoxy)benzoate dihydrochloride (E12 2HCl)

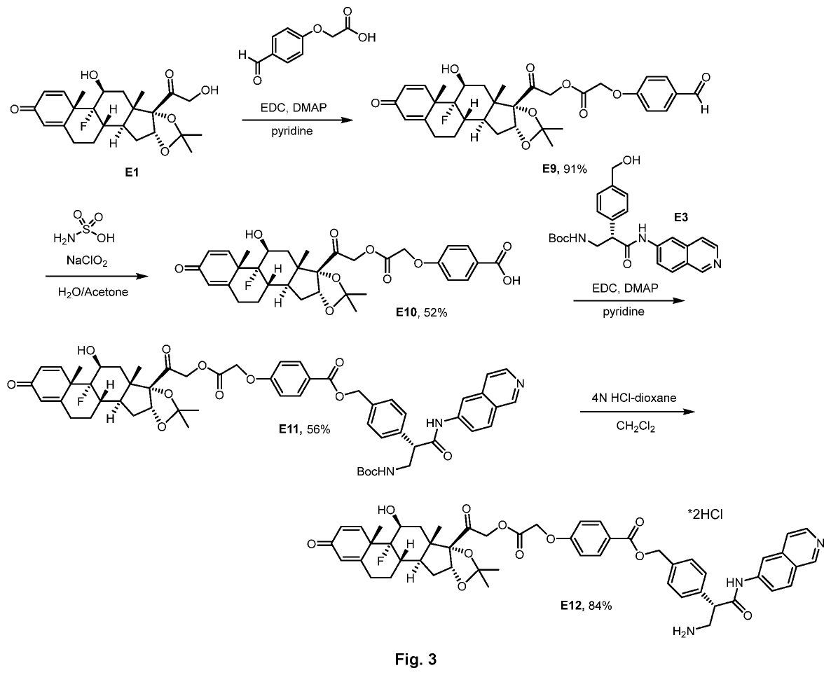

4-((S)-3-amino-1-(isoquinolin-6-ylamino)-1-oxopropan-2-yl)benzyl 4-(2-(2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)-2-oxoethoxy)benzoate dihydrochloride (E12, 2HCl). was prepared according to the synthetic scheme shown in FIG. 3.

### Preparation of 2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-86H-naphtho[2′,1′:4,5]indeno[1,2-d][1,3]dioxol-8b-yl)-2-oxoethyl 2-(4-formylphenoxy)acetate (E9)

To Triamcinolone acetonide (E1) was added 2-(4-formylphenoxy) acetic acid, EDC, and DMAP in pyridine and the solution was stirred overnight at room temperature. The mixture was poured into EtOAc and NaHCO₃ (sat) extracted, dried (Na₂SO₄) filtered and evaporated. Column chromatography 0-5% MeOH·CH₂Cl₂ gave pure 2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl 2-(4-formylphenoxy)acetate (E9, 91%).

### Preparation of 4-(2-(2-((6aS,66R,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)-2-oxoethoxy)benzoic acid (E10)

To 2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl 2-(4-formylphenoxy)acetate (E9) in H₂O and Acetone was added sulfamic acid and sodium chlorite ant the solution was stirred at room temperature overnight. The solution was then extracted with EtOAc, dried (Na₂SO₄), filtered and evaporated. Column chromatography 0-2% MeOH·CH₂Cl₂ gave pure 4-(2-(2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)-2-oxoethoxy)benzoic acid (E10, 62%).

### Preparation of 4-((S)-3-((tert-butoxycarbonyl)amino)-1-(isoquinolin-6-ylamino)-1-oxopropan-2-yl)benzyl 4-(2-(2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)-2-oxoethoxy)benzoate (E11)

To 4-(2-(2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)-2-oxoethoxy)benzoic acid (E10) in pyridine was added EDC, DMAP and tert-butyl(S)-(2-(4-(hydroxymethyl)phenyl)-3-(isoquinolin-6-ylamino)-3-oxopropyl) carbamate (E3) and the solution was stirred overnight. The mixture was poured into EtOAc and NaHCO₃ (sat) and extracted, dried (Na₂SO₄), filtered and evaporated. Column chromatography 0-5% MeOH·CH₂Cl₂ gave pure 4-((S)-3-((tert-butoxycarbonyl)amino)-1-(isoquinolin-6-ylamino)-1-oxopropan-2-yl)benzyl 4-(2-(2-((6aS,6bR,7S,8aS,8bS,11aR,12aS, 12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)-2-oxoethoxy)benzoate (E11, 56%).

### Preparation of 4-((S)-3-amino-1-(isoquinolin-6-ylamino)-1-oxopropan-2-yl)benzyl 4-(2-(2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)-2-oxoethoxy)benzoate dihydrochloride (E12)

To 4-((S)-3-((tert-butoxycarbonyl)amino)-1-(isoquinolin-6-ylamino)-1-oxopropan-2-yl)benzyl 4-(2-(2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)-2-oxoethoxy)benzoate (E11) in CH₂Cl₂ was added HCl (4N in dioxane) and the mixture was stirred at room temperature for 5 hours. Then the solids were filtered and washed with CH₂Cl₂ and dried to give 4-((S)-3-amino-1-(isoquinolin-6-ylamino)-1-oxopropan-2-yl)benzyl 4-(2-(2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy)-2-oxoethoxy)benzoate dihydrochloride (E12 84%).

Using commercially available compounds and largely the procedures set forth in E2-E5 and substituting the appropriate starting materials, E2-E77 (Table 3) were made and E78-E99 (Table 4) and compound A and compound B are synthesized according to the methods provided herein.

TABLE 3

| | | Molecular |
|---|---|---|
| Example | | Weight |
| number | Structure | (g/mol) |
| E2 | 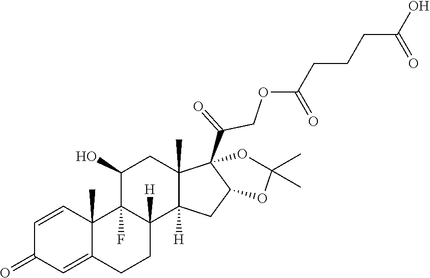 | MW: 548.6 |
| E4 | 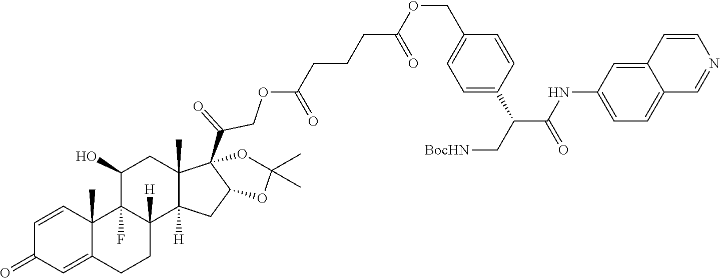 | MW: 952.1 |
| ES | 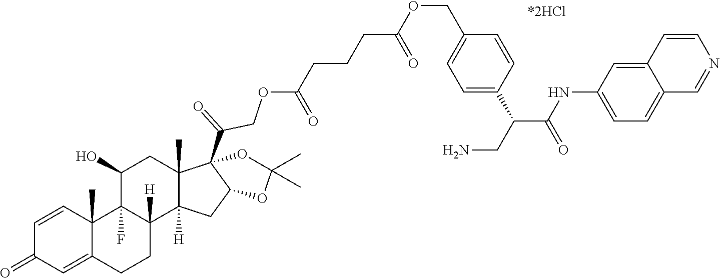 | MW: 851.9 MW 2HCl: 924.9 |
| E6 | 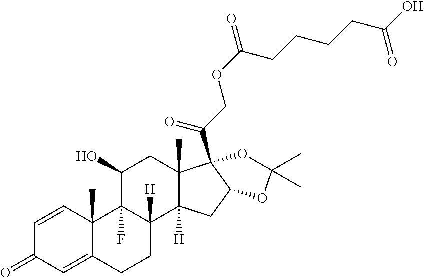 | MW: 562.6 |
| E7 | 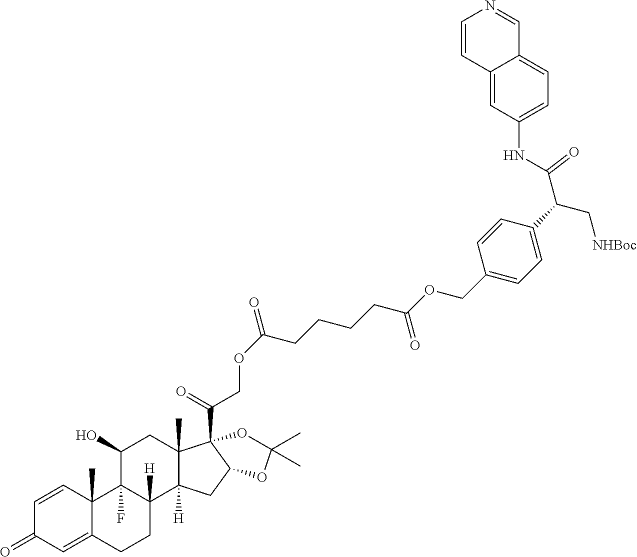 | MW: 966.1 |
| E8 | 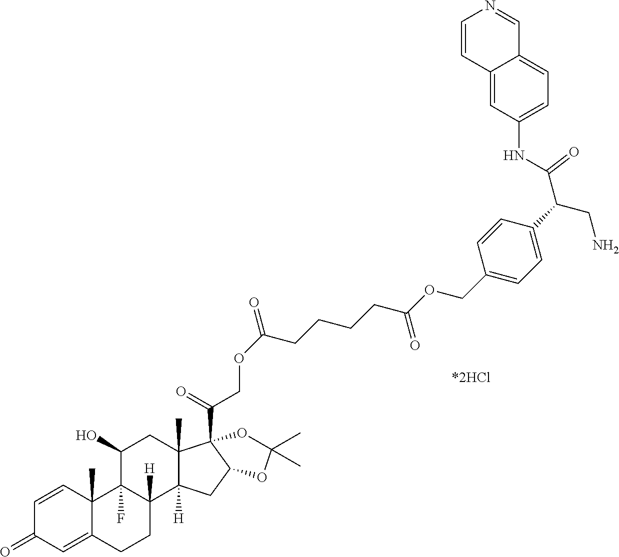 | MW: 866.0 MW 2HCl: 938.9 |
| E9 | 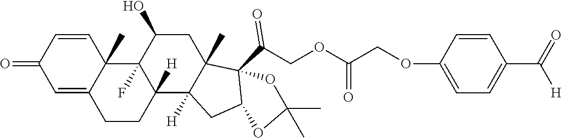 | MW: 596.6 |
| E10 | 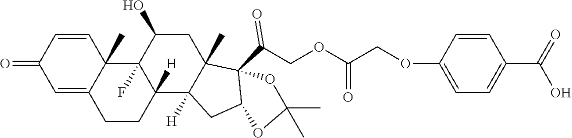 | MW: 612.6 |
| E11 | 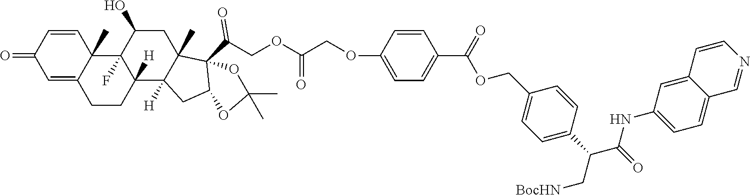 | MW: 1016.1 |
| E12 | 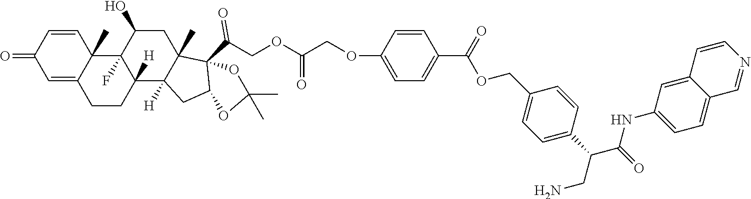 | MW: 916.0 MW 2HCl: 988.9 |
| E13 | 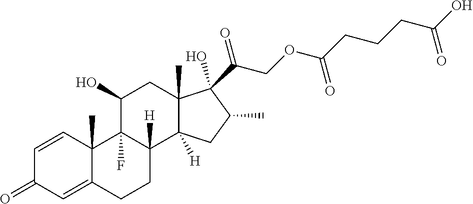 | MW: 506.6 |
| E14 | 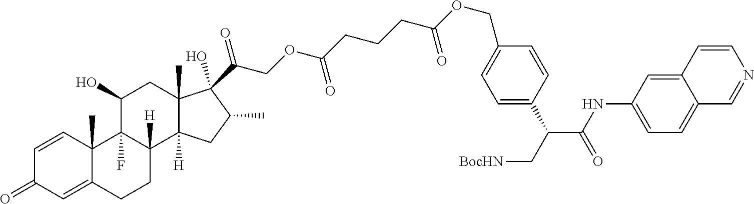 | MW: 910.1 |
| E15 | 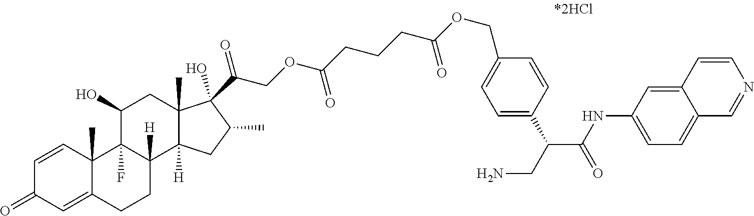 | MW = 809.2 MW 2HCl: 882.8 |
| E16 | 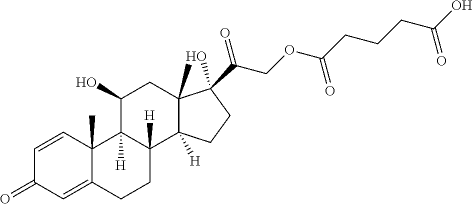 | 474.6 |
| E17 | 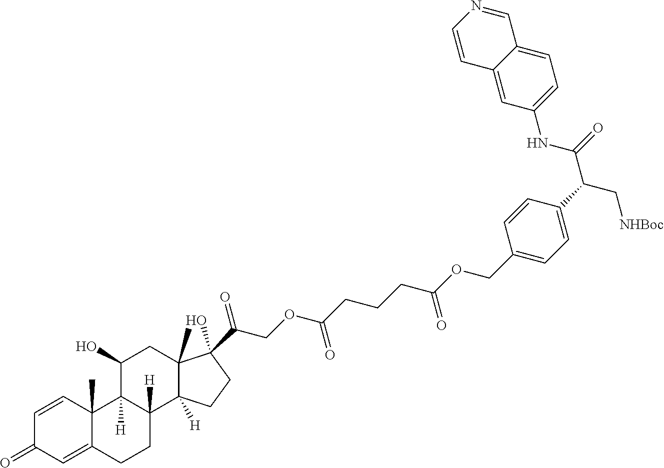 | MW: 878.0 |
| E18 | 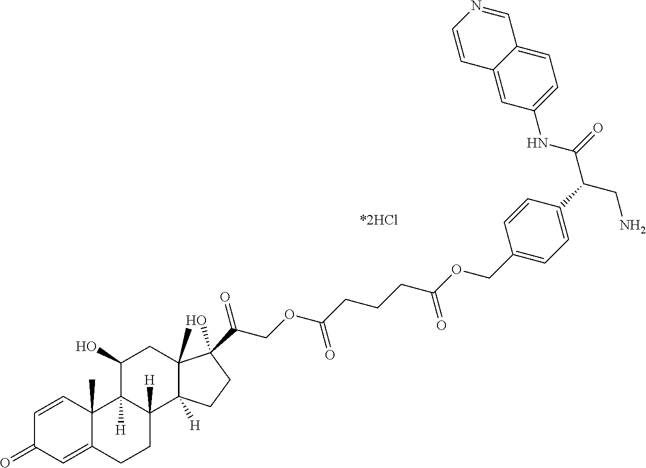 | MW: 777.9 MW 2HCl: 850.8 |
| E19 | 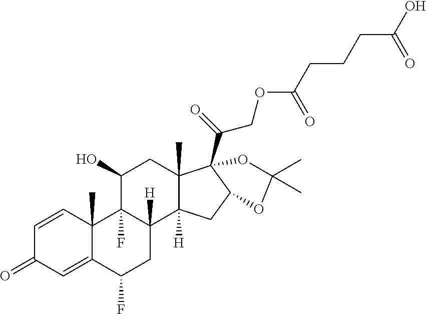 | MW: 566.6 |
| E20 | 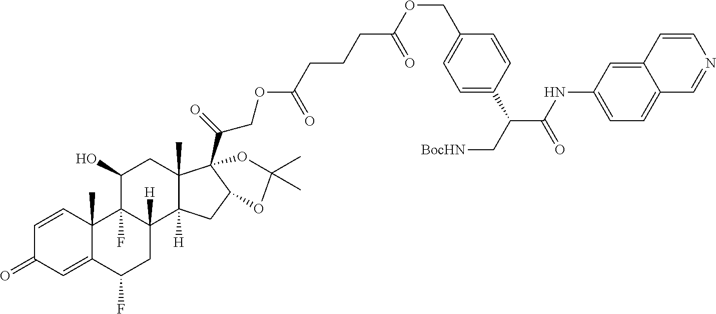 | MW: 970.1 |
| E21 | 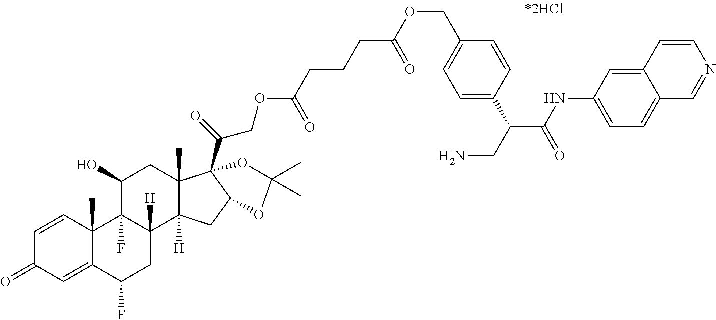 | MW: 869.9 MW 2HCl: 942.9 |
| E22 | 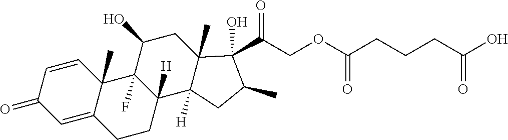 | MW: 506.6 |
| E23 | 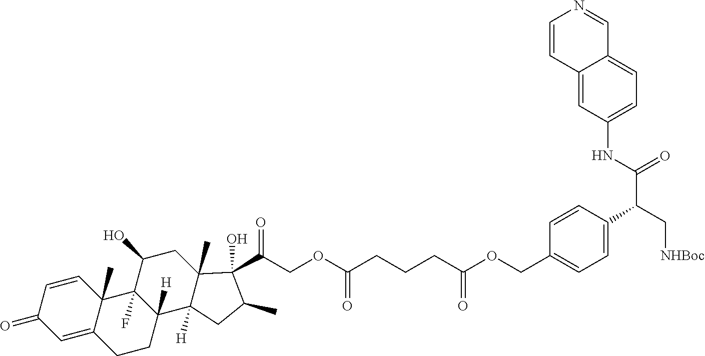 | MW: 910.1 |
| E24 | 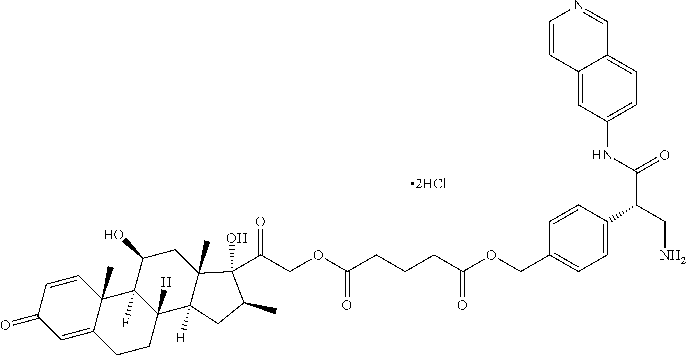 | MW: 809.9 MW 2HCl: 882.84 |
| E25 | 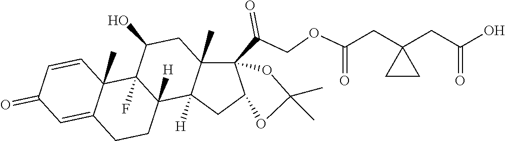 | MW: 574.6 |
| E26 | 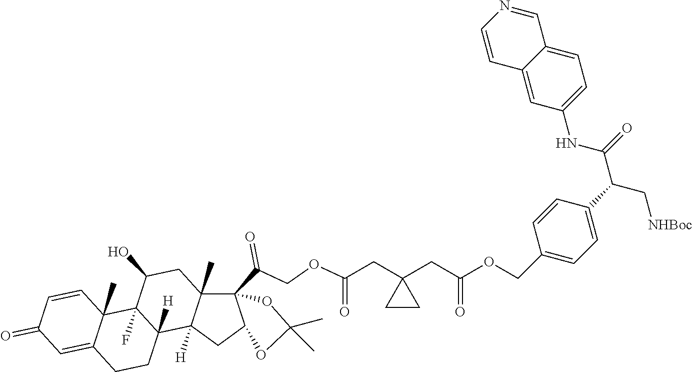 | MW: 978.1 |
| E27 | 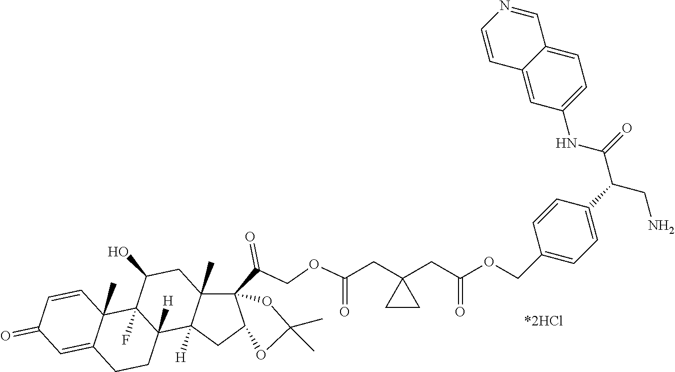 | MW: 878.0 MW 2HCl: 950.9 |
| E28 | 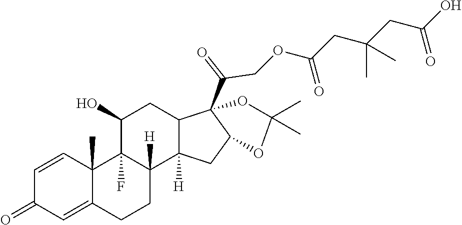 | MW: 576.7 |
| E29 | 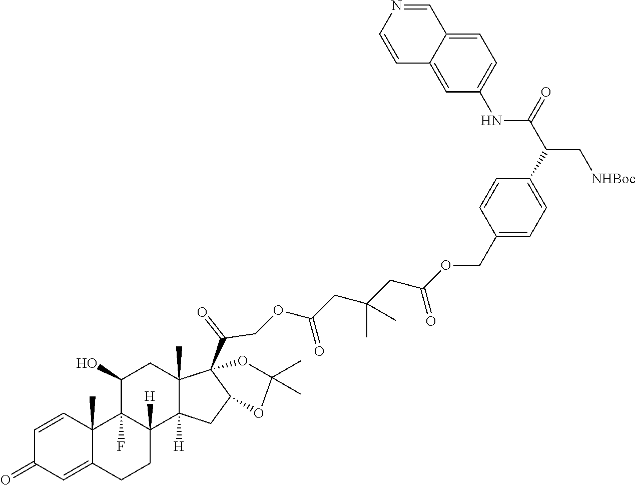 | MW: 980.4 |
| E30 | 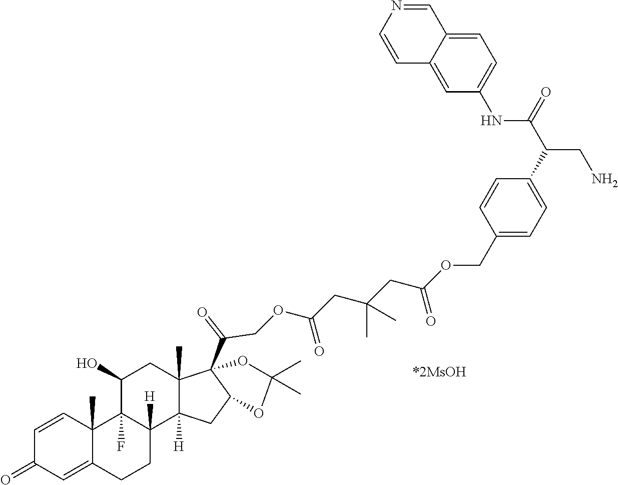 | MW: 880.0 MW 2 MsOH: 1072.2 |
| E31 | 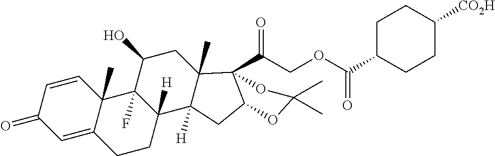 | MW: 588.7 |
| E32 | 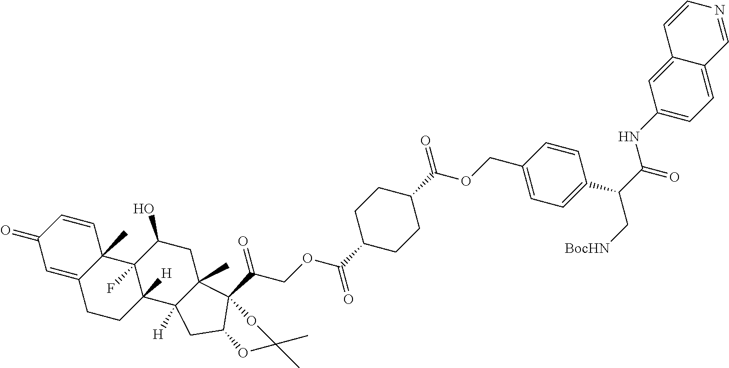 | MW: 992.2 |
| E33 | 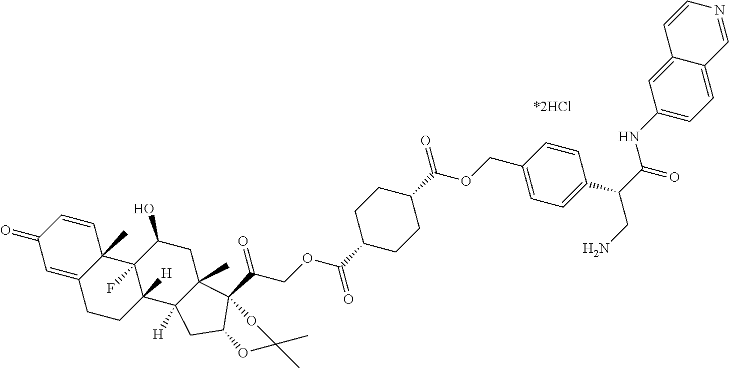 | MW: 892.02 MW 2HCl: = 964.94 |
| E34 | 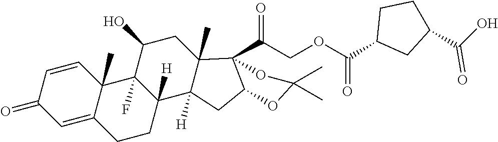 | MW: 574.6 |
| E35 | 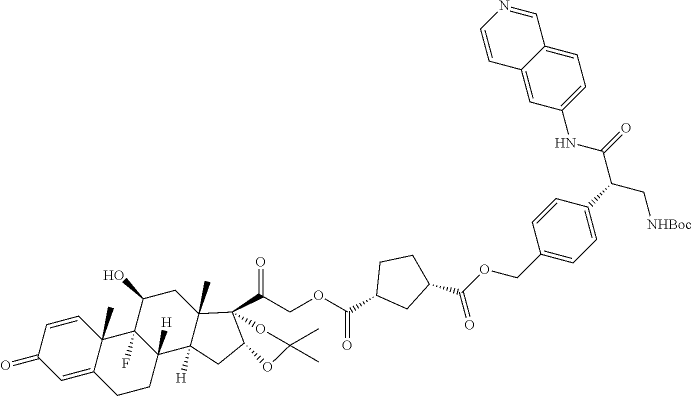 | MW: 978.1 |
| E36 | 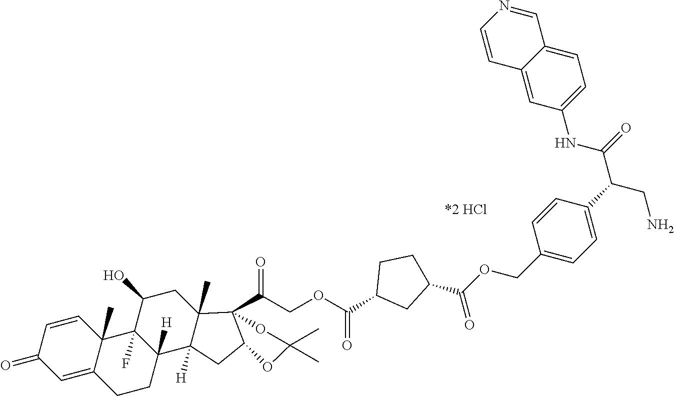 | MW: 878.0 MW 2HCl: 950.91 |
| E37 | 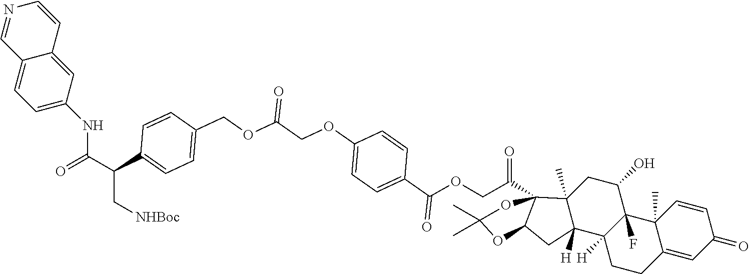 | MW: 1016.1 |
| E38 | 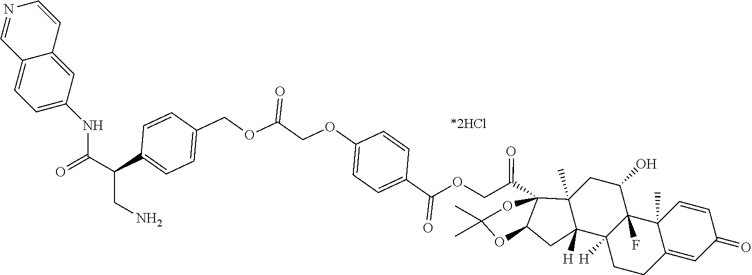 | MW = 916.0 MW 2HCl: 988.9 |
| E39 | 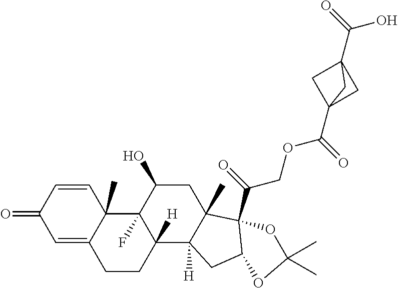 | MW: 572.6 |
| E40 | 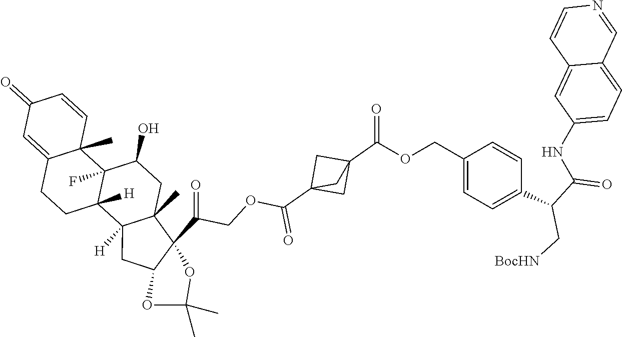 | MW: 976.1 |
| E41 | 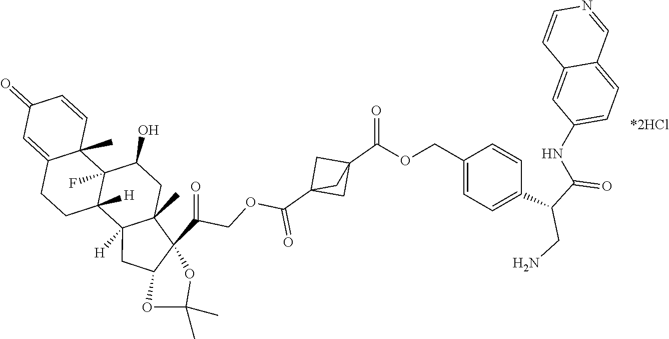 | MW = 875.9 MW 2HCl: 948.9 |
| E42 | 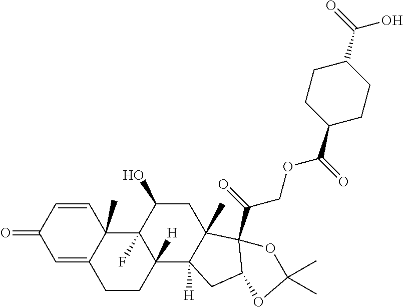 | MW: 588.7 |
| E43 | 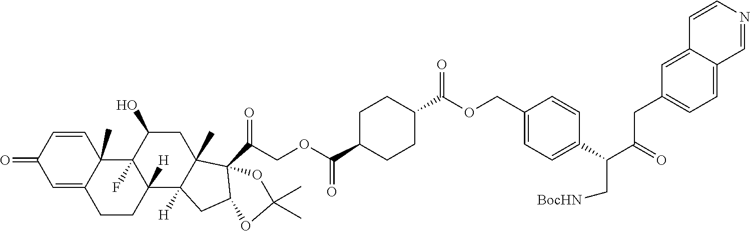 | MW: 992.2 |
| E44 | 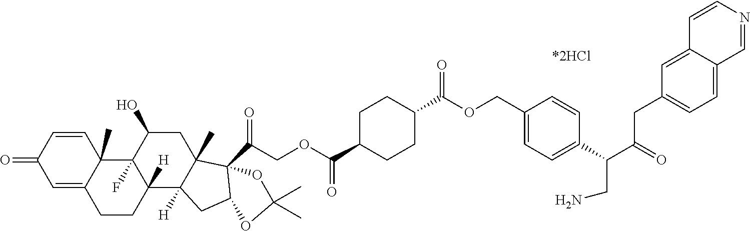 | MW: 892.0 MW 2HCl: 964.9 |
| E45 | 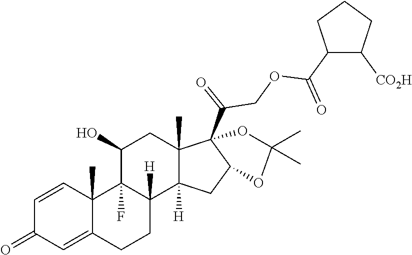 | MW: 574.6 |
| E46 | 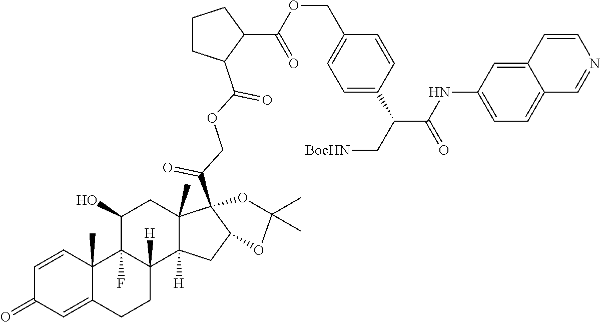 | MW:978.1 |
| E47 | 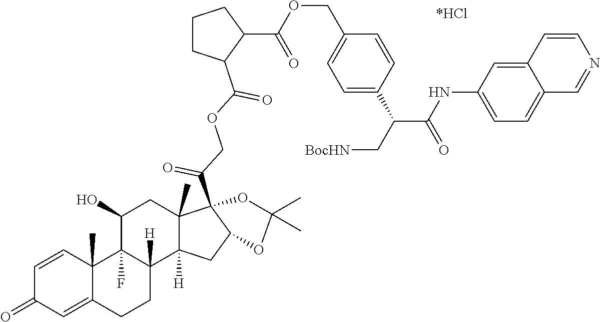 | MW: 878.0 MW 2HCl: 950.92 |
| E48 | 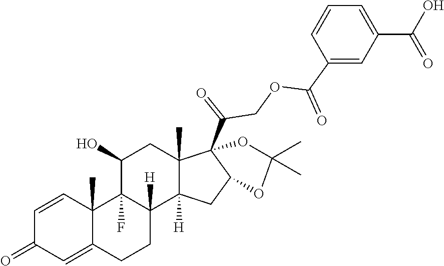 | MW: 582.6 |
| E49 | 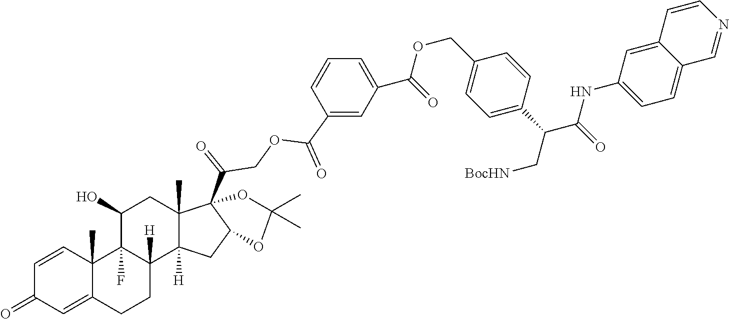 | MW: 986.1 |
| E50 | 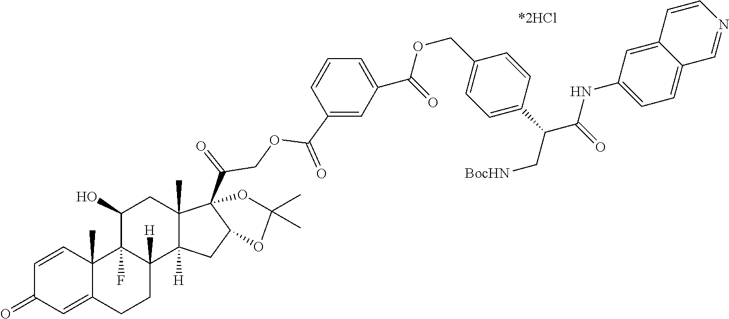 | MW: 885.9 MW: 2HCl: 958.9 |
| E51 | 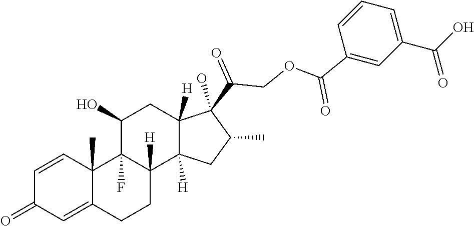 | MW: 540.6 |
| E52 | 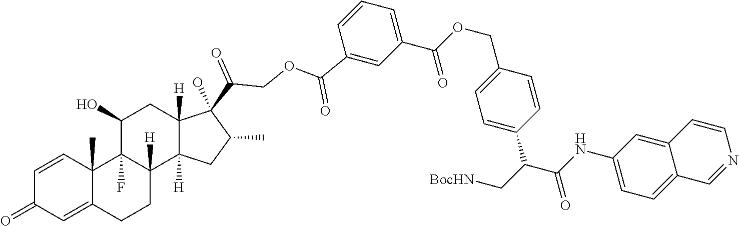 | MW: 944.1 |
| E63 | 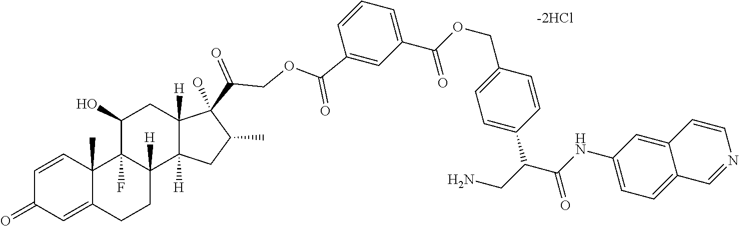 | MW: 843.9 MW 2HCl: 916.9 |
| E54 | 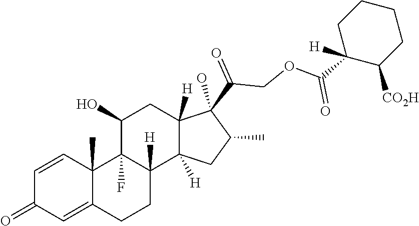 | MW: 546.6 |
| E55 | 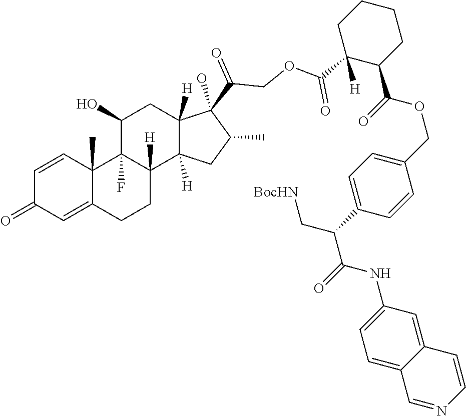 | MW: 950.1 |
| E56 | 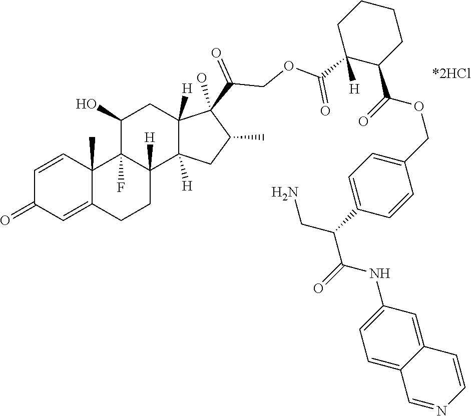 | MW: 850.0 MW 2HCl: 922.9 |
| E57 | 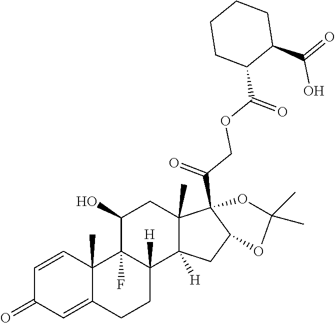 | MW: 588.7 |
| E58 | 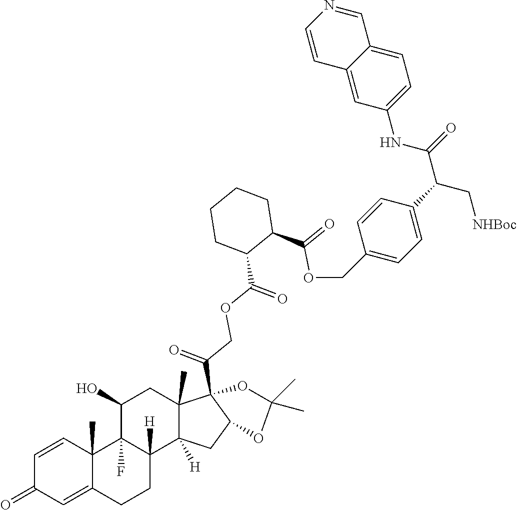 | MW: 992.2 |
| E59 | 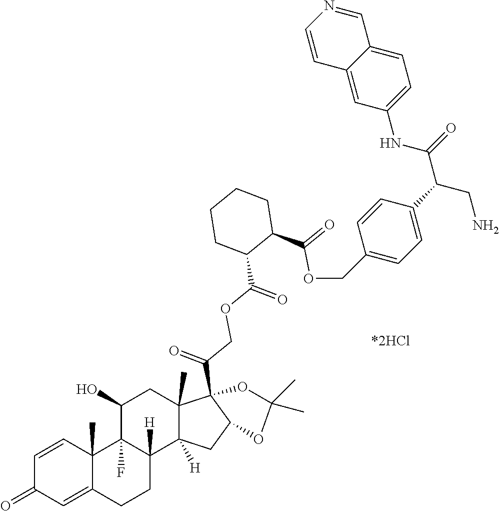 | MW: 892.0 MW 2HCl: 964.9 |
| E60 | 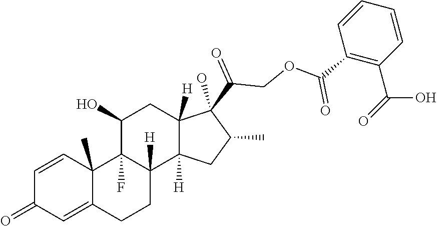 | MW: 540.6 |
| E61 | 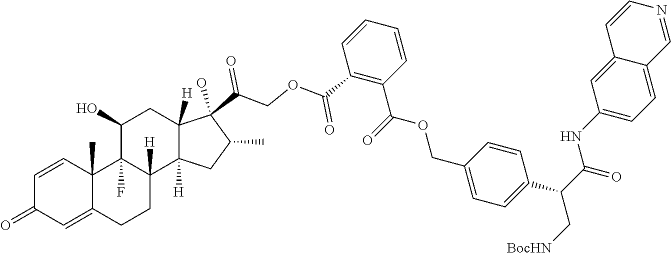 | MW: 944.1 |
| E62 | 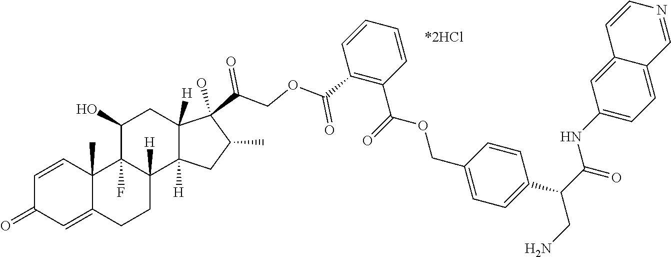 | MW: 843.9 MW 2HCl: 916.9 |
| E63 | 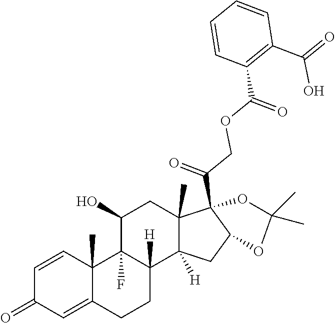 | MW: 582.6 |
| E64 | 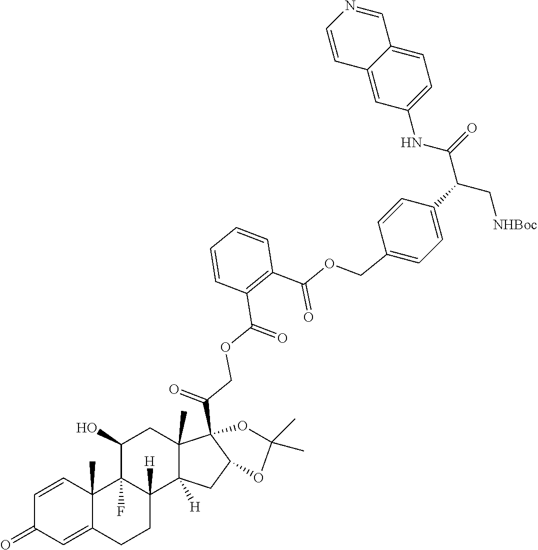 | MW: 986.1 |
| E65 | 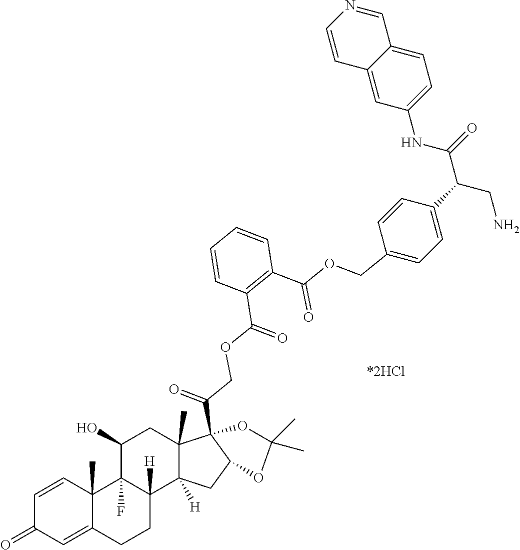 | MW: 885.9 MW 2HCl: 958.9 |
| E66 | 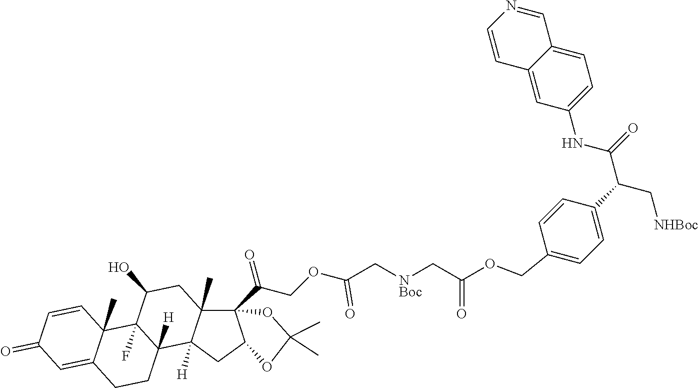 | MW: 1053.2 |
| E67 | 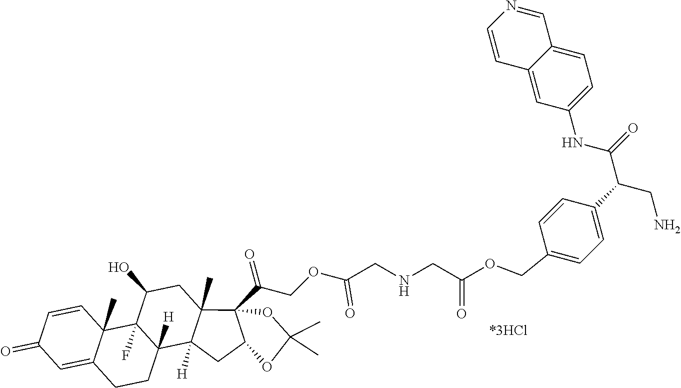 | MW: 852.9 MW 3HCl salt: 962.3 |
| E68 | 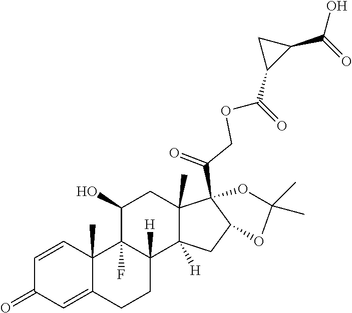 | MW: 546.6 |
| E69 | 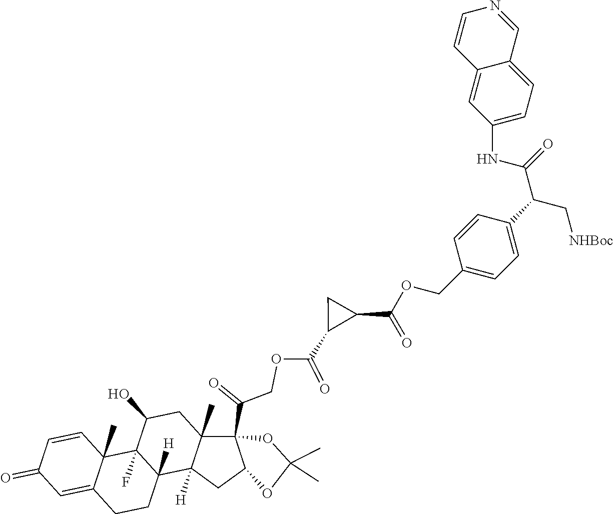 | MW: 950.1 |
| E70 | 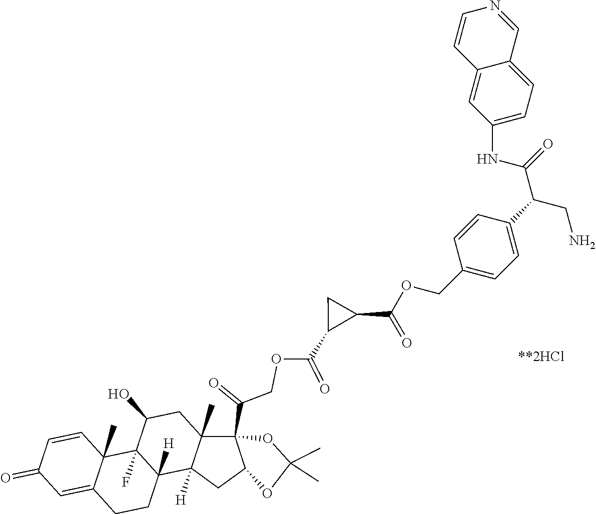 | MW: 849.94 MW 2HCl: 922.86 |
| E71 | 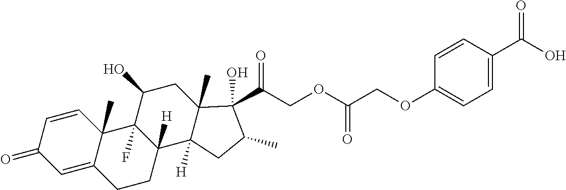 | MW: 570.6 |
| E72 | 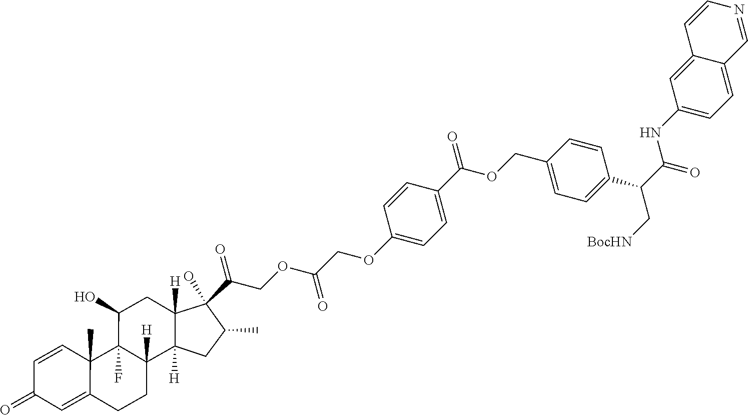 | MW: 974.1 |
| E73 | 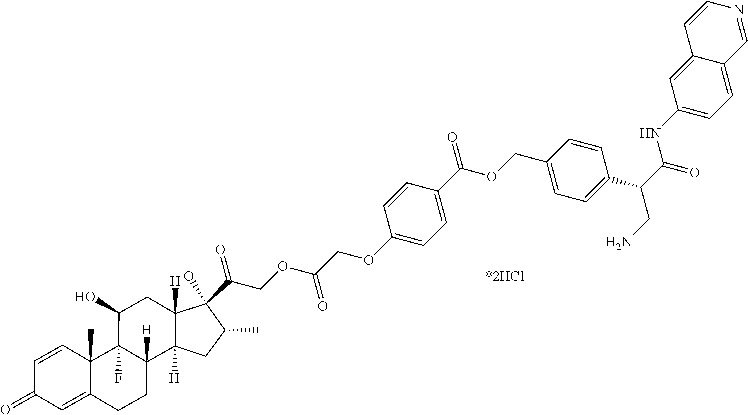 | MW: 873.96 MW 2HCl: 946.88 |
| E74 | 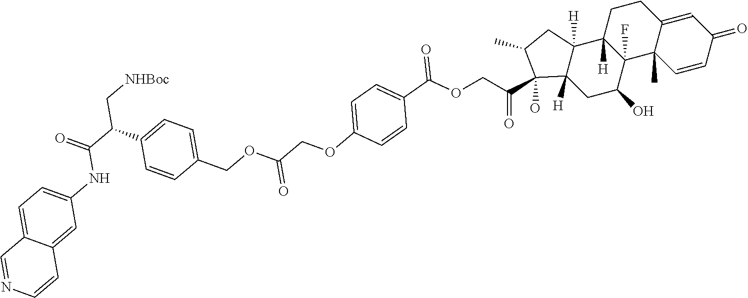 | MW: 974.1 |
| E76 | 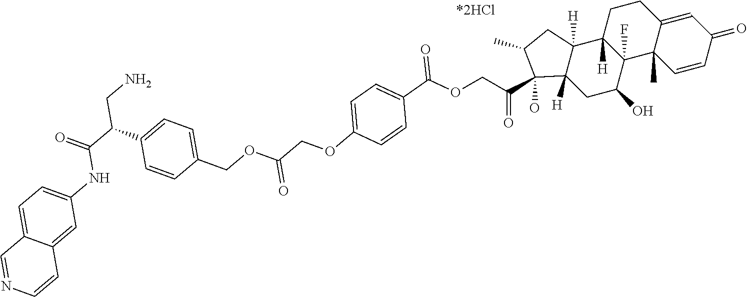 | MW: 873.9 MW 2HCl: 946.9 |
| E76 | 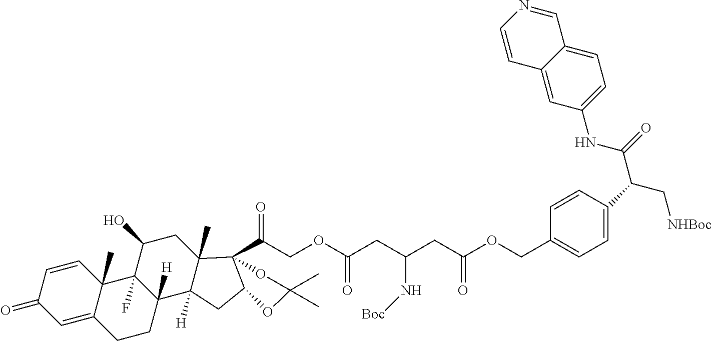 | MW: 1067.2 |
| E77 | 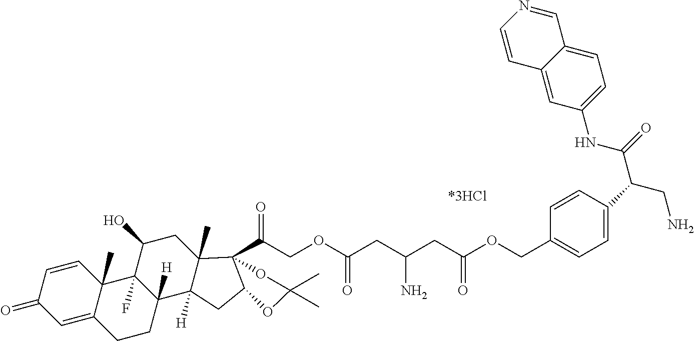 | MW = 866.96 MW 3HCl salt = 976.35 |
| E77B | 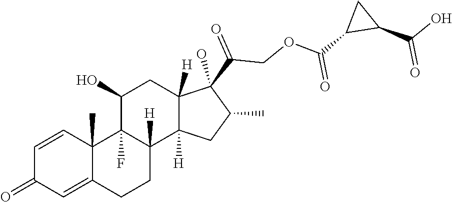 | MW = 504.5 |
| E77C | 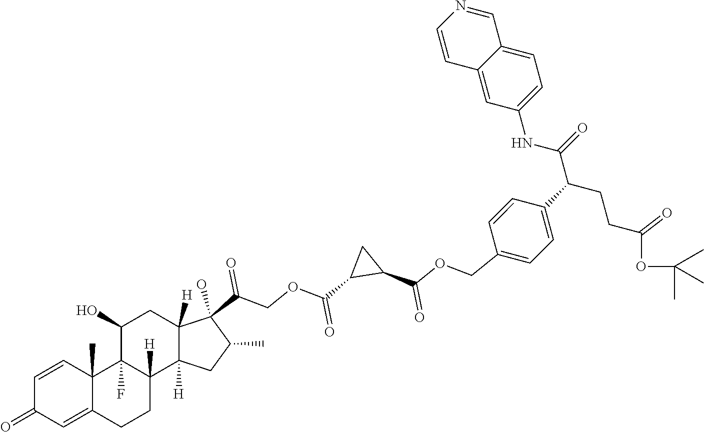 | MW = 908.0 |
| E77D | 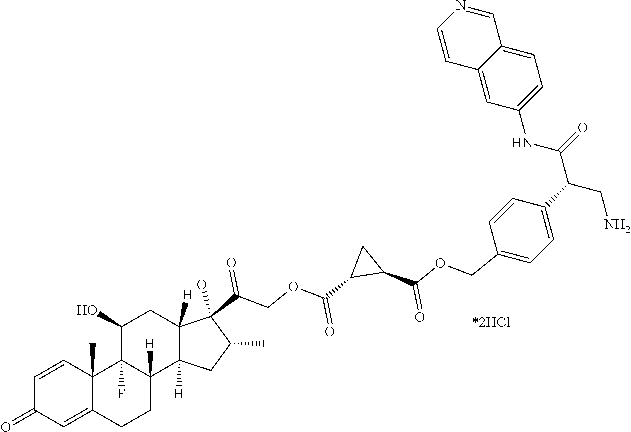 | MW = 807.9 MW diHCl = 880.8 |
| E77E | 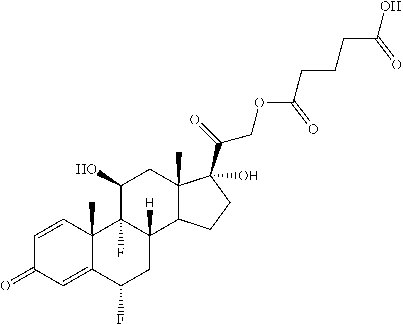 | MW = 510.5 |
| E77F | 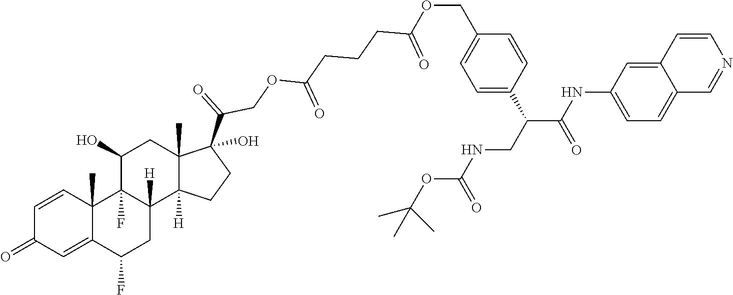 | MW = 913.9 |
| E77G | 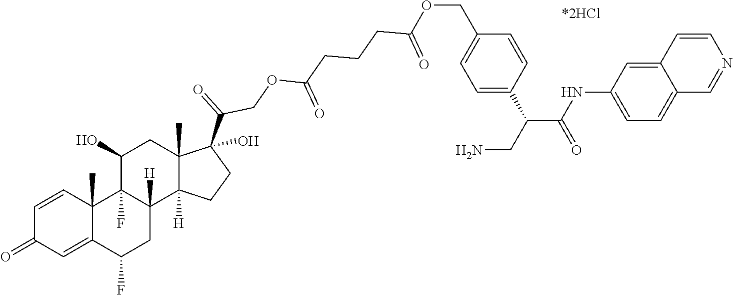 | MW = 813.9 MW 2HCl = 886.8 |
| E77H | 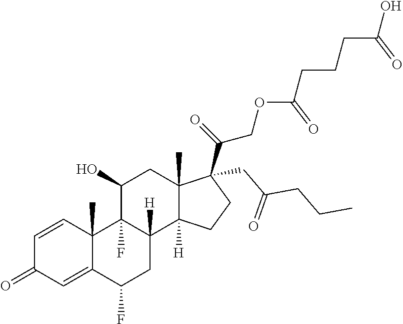 | MW = 580.6 |
| E771 | 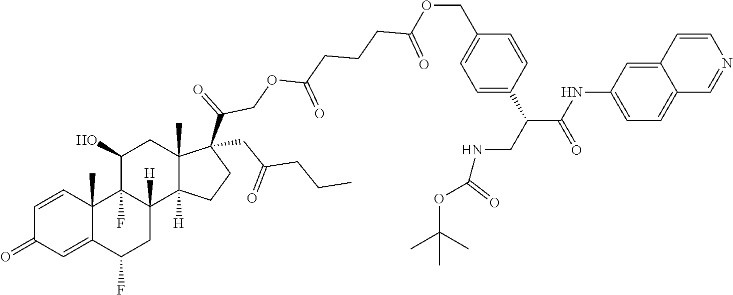 | MW = 984.1 |
| E77J | 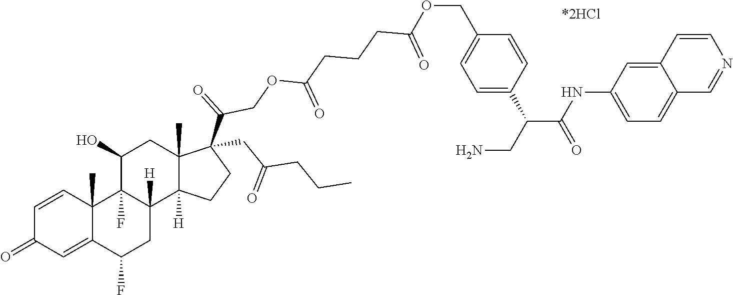 | MW = 883.9 MW 2HCl = 956.9 |
| E77K | 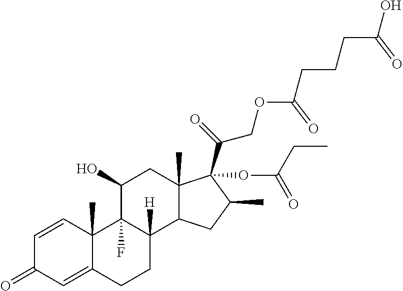 | MW = 562.6 |
| E77L | 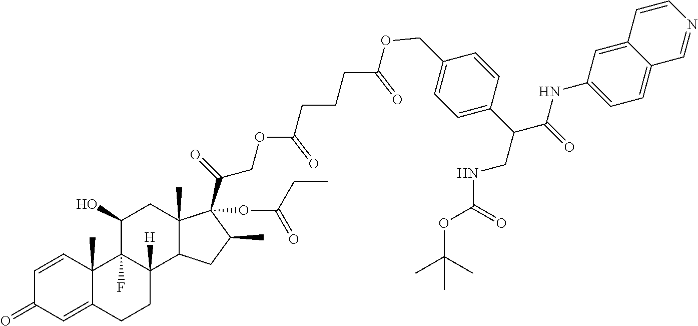 | MW = 966.1 |
| E77M | 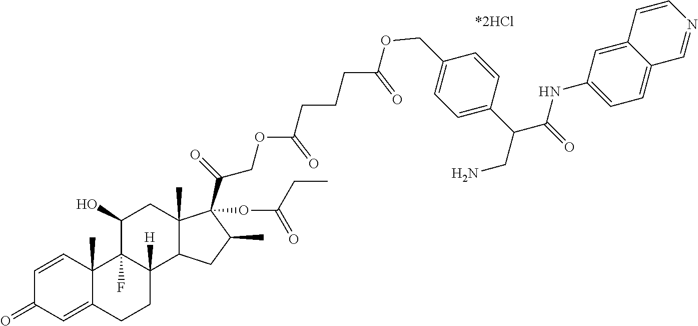 | MW = 866.0 MW 2HCl = 938.9 |
| E77N | 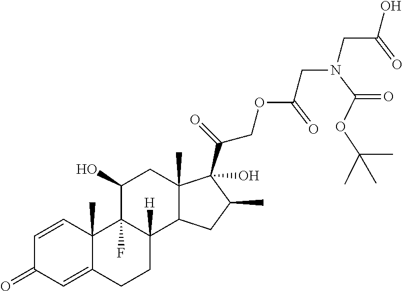 | MW = 607.6 |
| E770 | 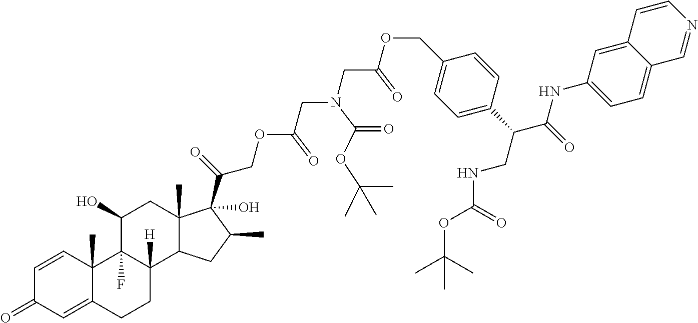 | MW = 1011.1 |
| E77P | 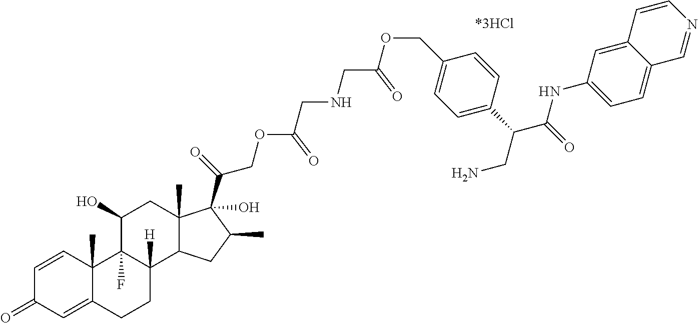 | MW = 810.9 MW 3HCl = 920.3 |
| E77Q | 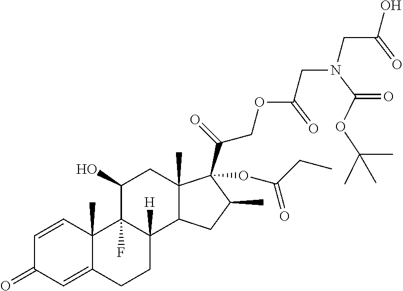 | MW = 663.7 |
| E77R | 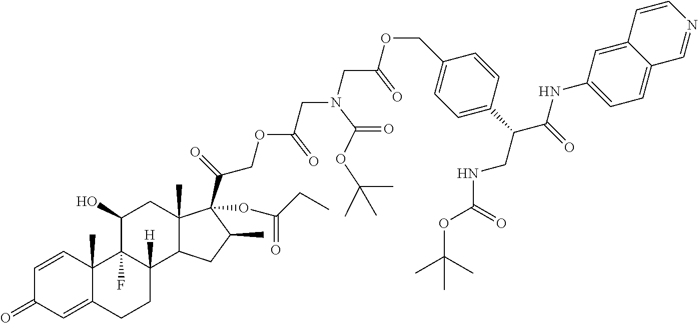 | MW = 1067.2 |
| E77S | 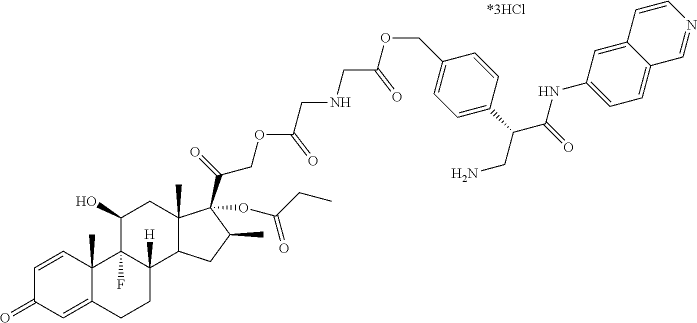 | MW = 866.9 MW 3 HCl = 976.4 |
| E77T | 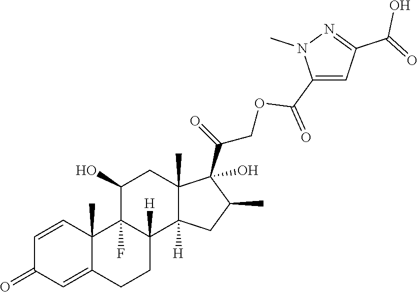 | MW = 544.6 |
| E77U | 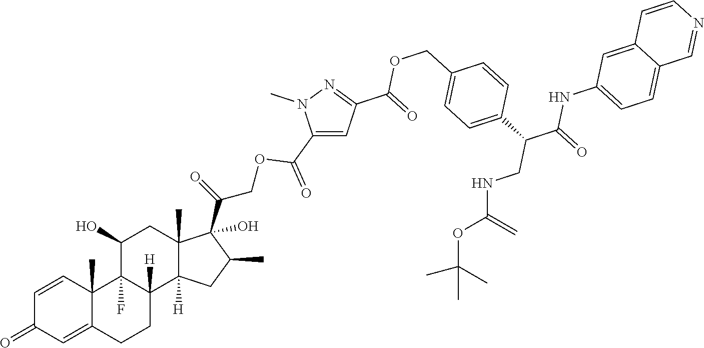 | MW= 948.0 |
| E77V | 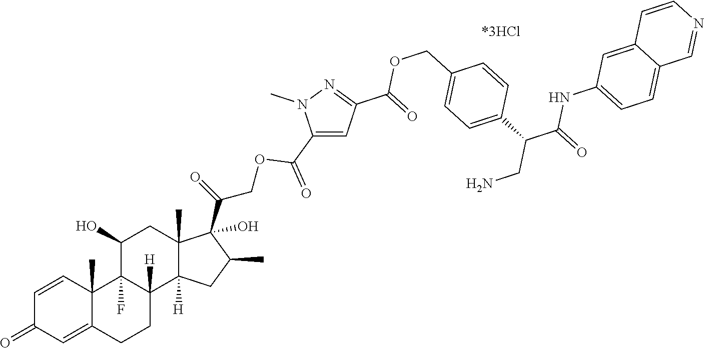 | MW = 847.9 MW 3HCl = 957.3 |

TABLE 4
Additional exemplary compounds that are synthesized using the methods described herein.

| Example | Structure |
|---|---|
| E78 | 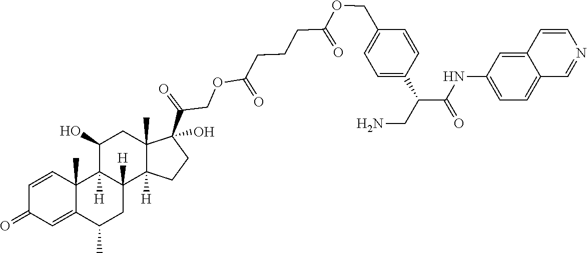 |
| E79 | 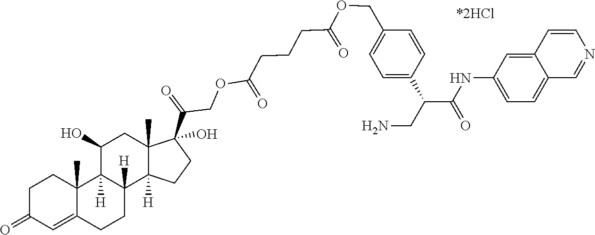 |
| E80 | 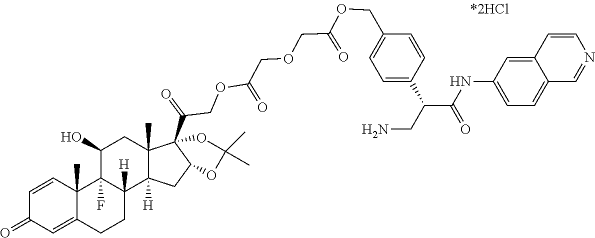 |
| E81 | 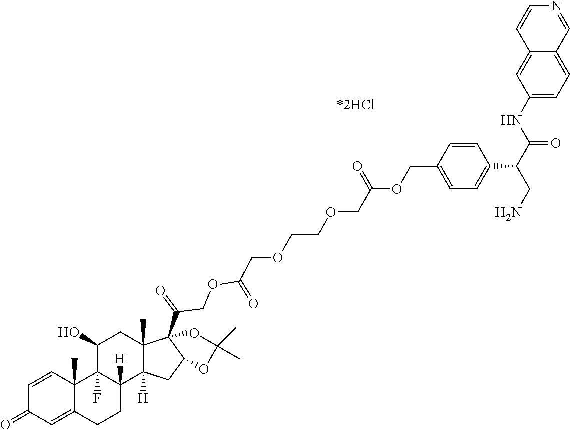 |
| E82 | 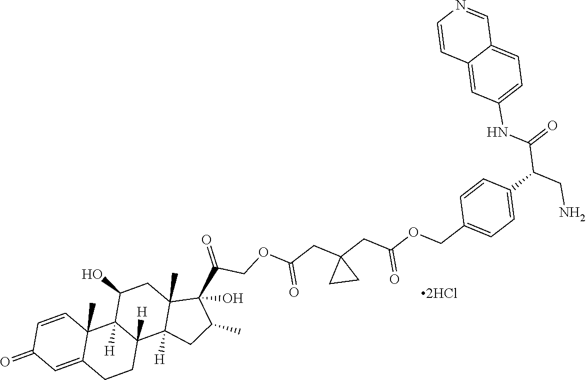 |
| E83 | 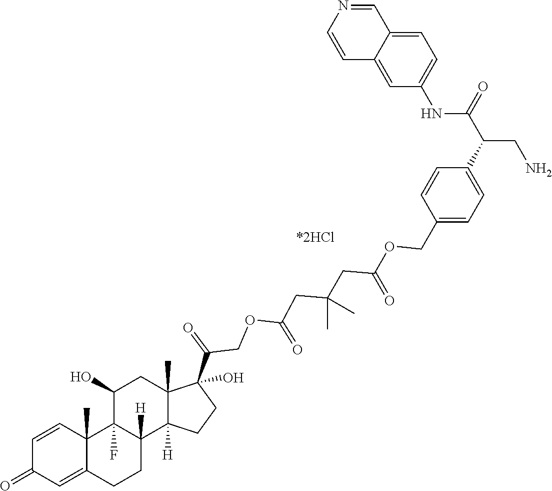 |
| E84 | 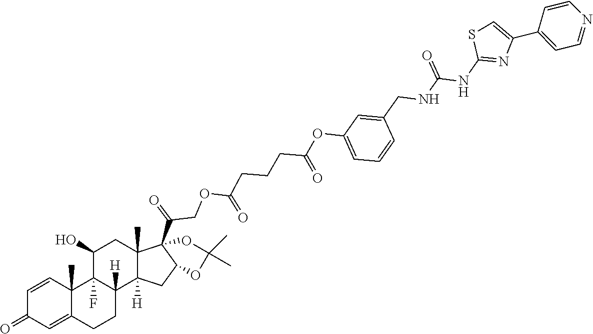 |
| E85 | 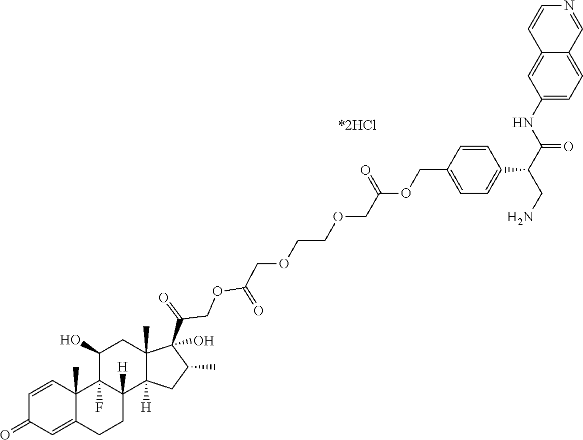 |
| E86 | 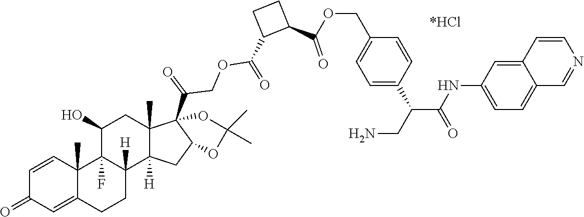 |
| E87 | 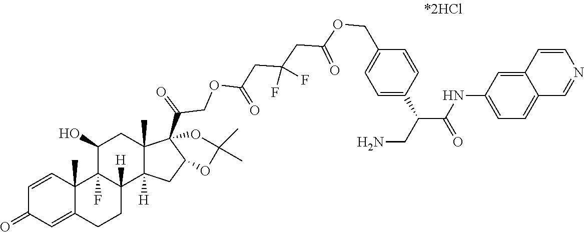 |
| E88 | 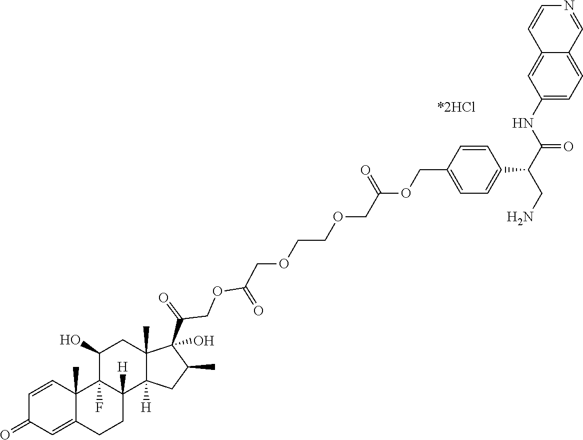 |
| E89 | 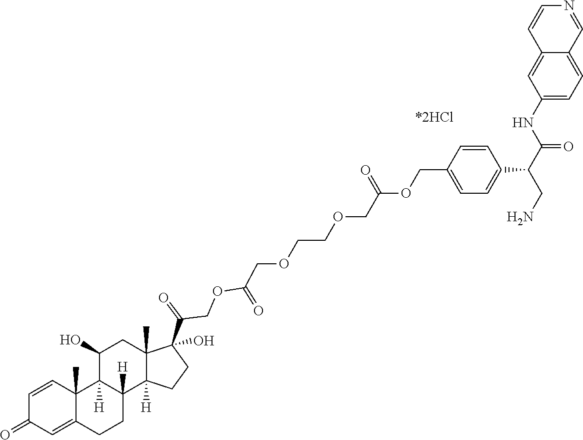 |
| E90 | 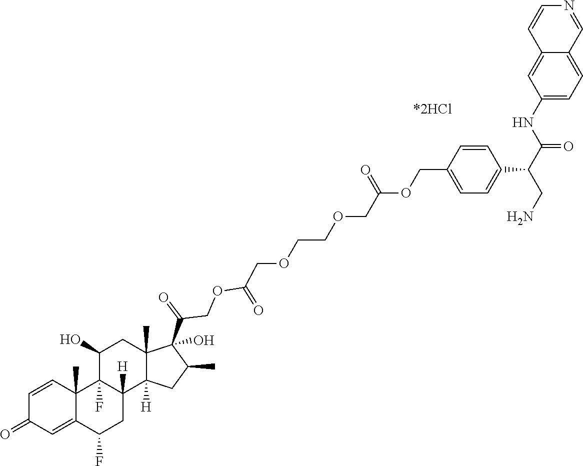 |
| E91 | 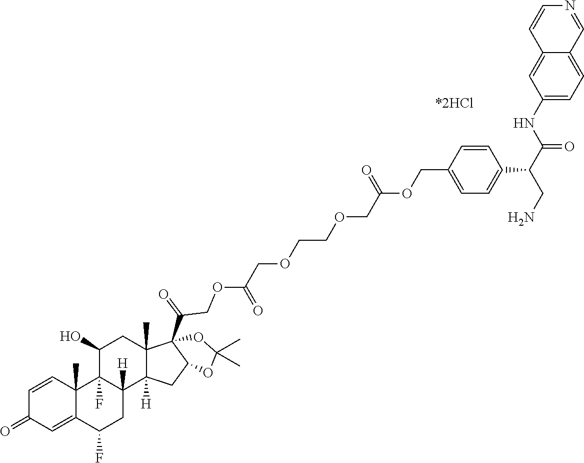 |
| E92 | 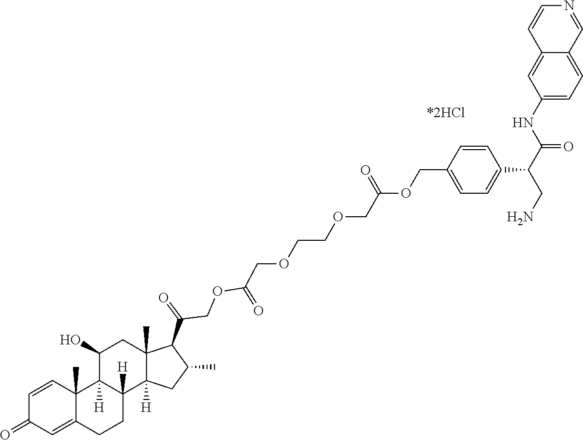 |
| E93 | 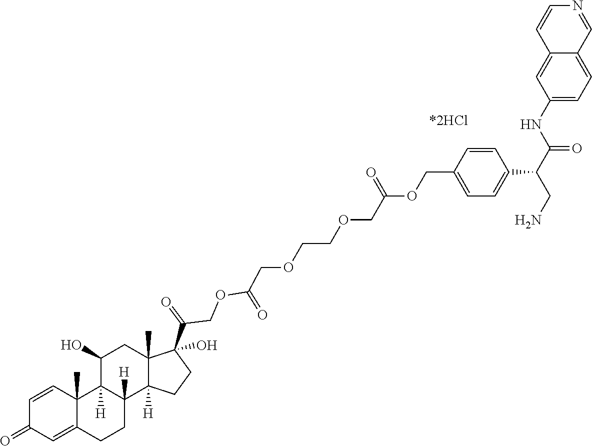 |
| E94 | 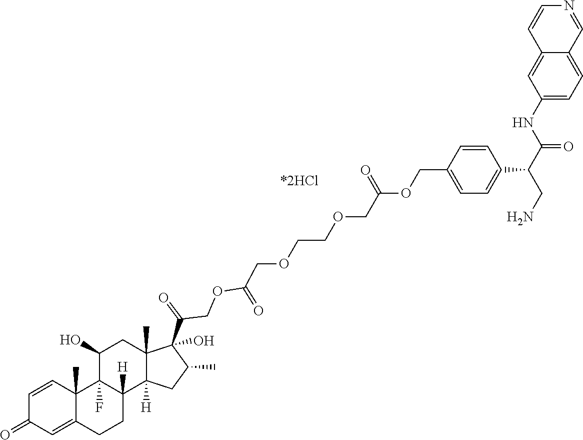 |
| E95 | 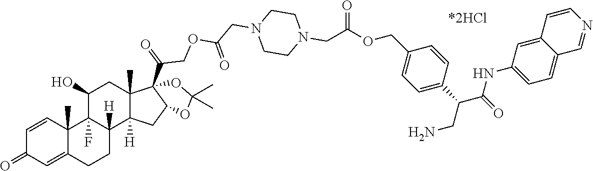 |
| E96 | 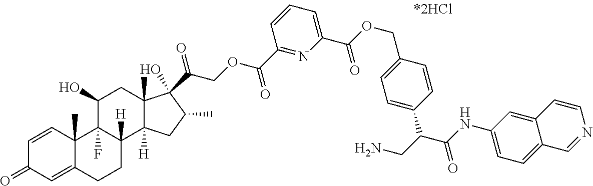 |
| E97 | 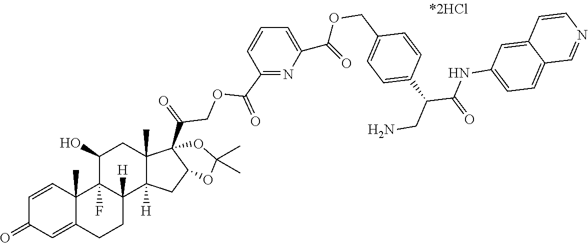 |
| E98 | 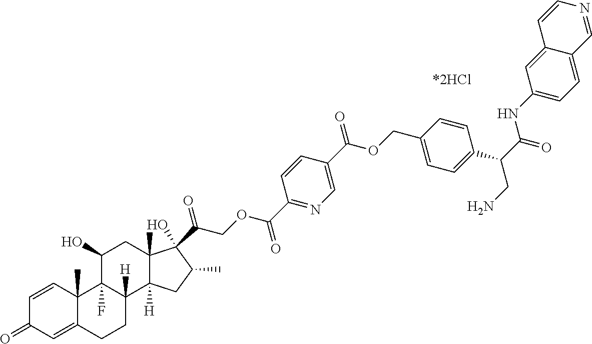 |
| E99 | 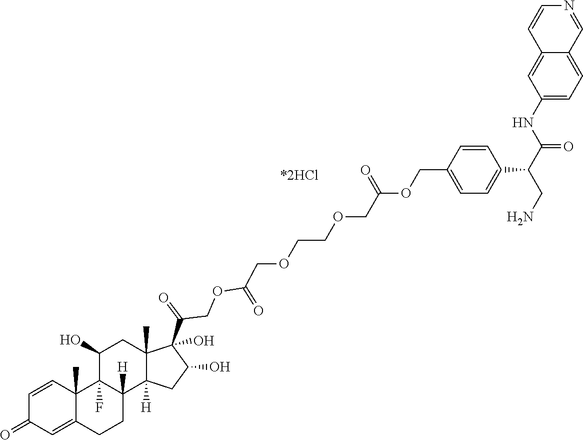 |
| Compound A | 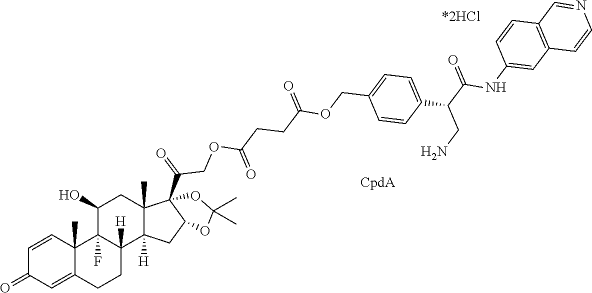 |

### Example 3. 4-((S)-3-amino-1-(isoquinolin-6-ylamino)-1-oxopropan-2-yl)benzyl (2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl) carbonate dihydrochloride (E102, 2HCl)

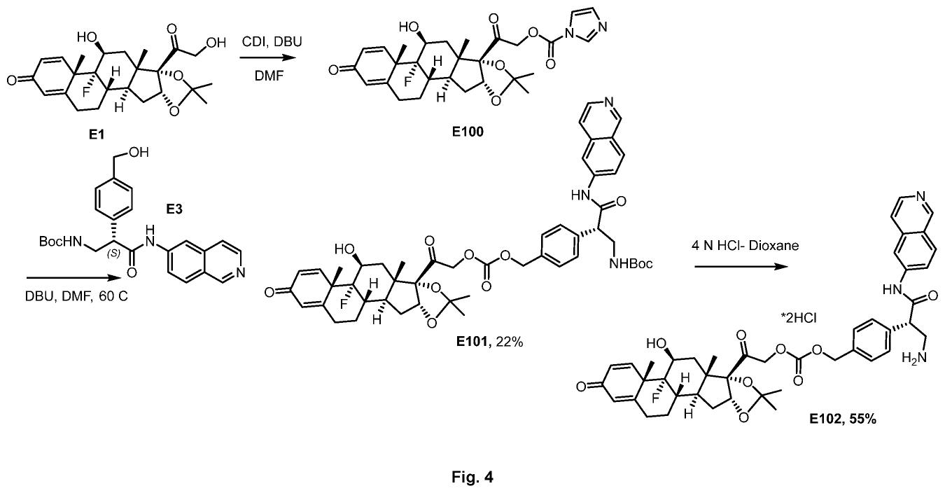

4-((S)-3-amino-1-(isoquinolin-6-ylamino)-1-oxopropan-2-yl)benzyl (2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl) carbonate dihydrochloride (E102, 2HCl) was prepared according to the synthetic scheme shown in FIG. 4.

### Preparation of 2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl 1H-imidazole-1-carboxylate (E100)

To Triamcinolone acetonide (E1) in DMF was added CDI, DMAP and pyridine and the solution was heated to 40° C. overnight. The mixture was poured into EtOAc and water and extracted and dried (Na₂SO₄), filtered and evaporated to give 2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl 1H-imidazole-1-carboxylate (E100).

### Preparation of tert-butyl ((S)-2-(4-((((2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy) carbonyl)oxy) methyl)phenyl)-3-(isoquinolin-6-ylamino)-3-oxopropyl) carbamate (E101)

To 2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl 1H-imidazole-1-carboxylate (E100) in DMF was added tert-butyl(S)-(2-(4-(hydroxymethyl)phenyl)-3-(isoquinolin-6-ylamino)-3-oxopropyl) carbamate (E3) and DBU and the solution was heated at 60° C. for 2 hours. The mixture was cooled to room temperature and poured into EtOAc and NaHCO₃ (saturated) and extracted, dried (Na₂SO₄), filtered and evaporated. Column chromatography 0-8% MeOH·CH₂Cl₂ gave pure tert-butyl ((S)-2-(4-((((2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy) carbonyl)oxy) methyl)phenyl)-3-(isoquinolin-6-ylamino)-3-oxopropyl) carbamate (E101, 22%).

### Preparation of 4-((S)-3-amino-1-(isoquinolin-6-ylamino)-1-oxopropan-2-yl)benzyl (2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl) carbonate dihydrochloride (E102)

To tert-butyl ((S)-2-(4-((((2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethoxy) carbonyl)oxy) methyl)phenyl)-3-(isoquinolin-6-ylamino)-3-oxopropyl) carbamate (E101)

in CH₂Cl₂ was added HCl (4N in dioxane) and the solution was stirred 4 hours at room temperature. The liquids were decanted and the solids were filtered and washed with CH₂Cl₂ and dried to give 4-((S)-3-amino-1-(isoquinolin-6-ylamino)-1-oxopropan-2-yl)benzyl (2-((6aS,6bR,7S,8aS,8bS,11aR,12aS,12bS)-6b-fluoro-7-hydroxy-6a,8a,10,10-tetramethyl-4-oxo-1,2,4,6a,6b,7,8,8a,11a,12,12a,12b-dodecahydro-8bH-naphtho[2′,1′:4,5]indeno[1,2-d][1,3]dioxol-8b-yl)-2-oxoethyl) carbonate dihydrochloride (E102, 55%).

Using commercially available compounds and largely the procedures set forth in E100-E102 and substituting the appropriate starting materials, E102 (Table 5) was made and E103-E114 (Table 6) are synthesized according to the methods described herein.

TABLE 5

| Example | Structure | MW |
|---|---|---|
| E102 | 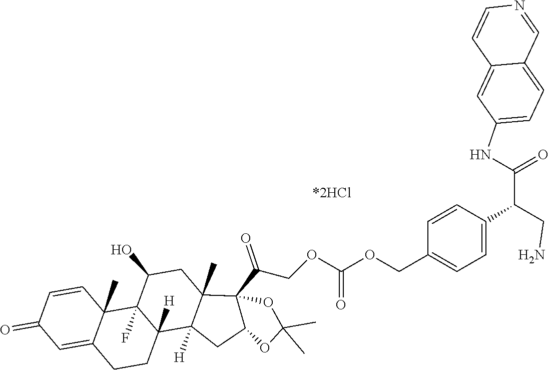 | MW= 781.9 MW diHCI = 854.8 |

TABLE 6

| Example | Structure |
|---|---|
| E103 | 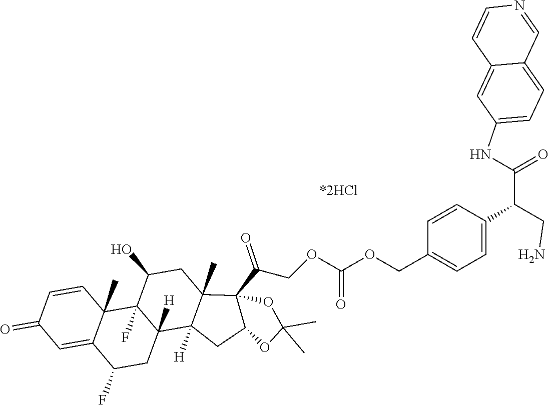 |
| E104 | 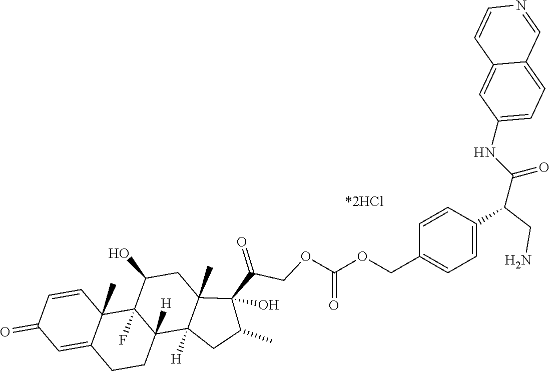 |
| E105 | 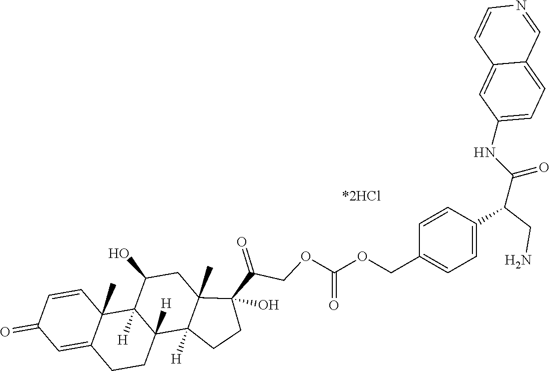 |
| E106 | 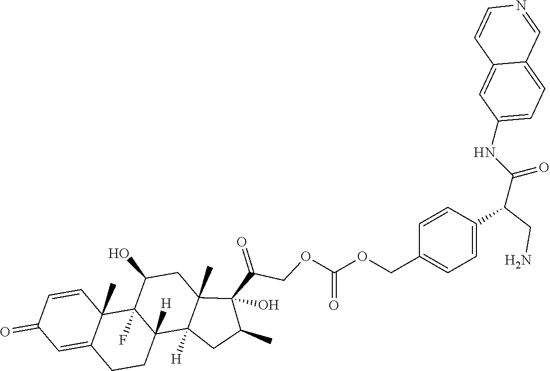 |
| E107 | 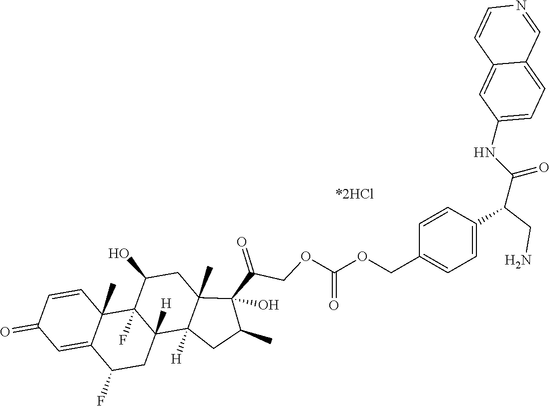 |
| E108 | 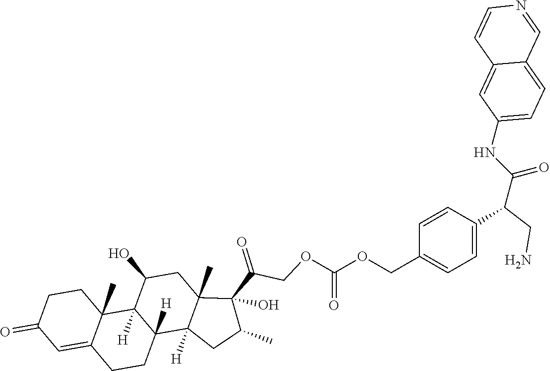 |
| E109 | 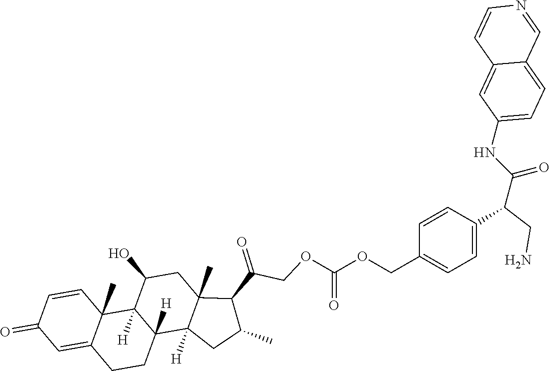 |
| E110 | 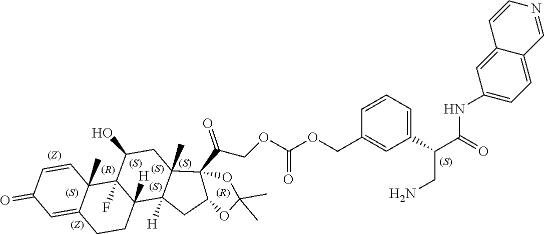 |
| E111 | 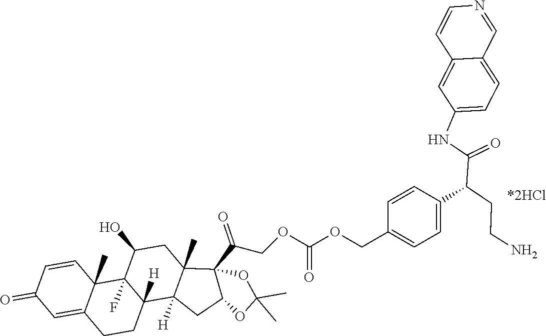 |
| E112 | 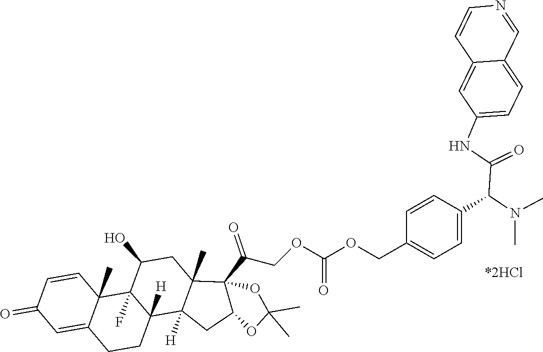 |
| E113 | 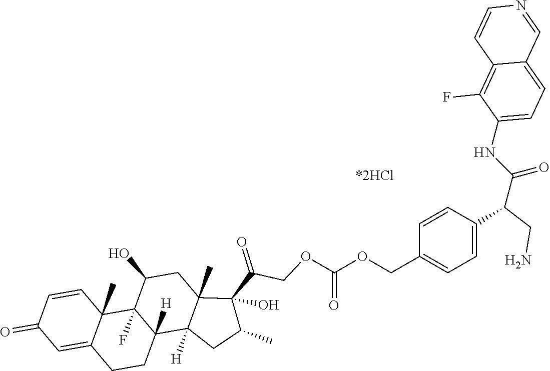 |
| E114 | 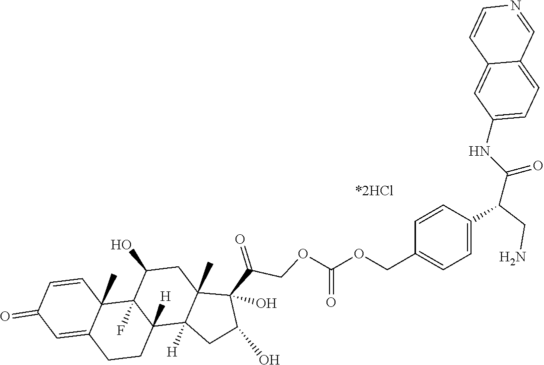 |

### Example 5. In Vitro Buffer Stability

The in vitro stability effect of certain of the compounds provided herein is assessed in a 10 mM phosphate buffer solution at pH 5 or pH 6 at a targeted concentration of approximately 1 mg/mL.

Initial preparations of compounds are made in 10 mM Phosphate buffer (pH 6.0). Each compound (1.0-1.5 mg) was weighed in a 4 mL glass vial and 10 mM phosphate buffer pH 6.0 was added targeting API concentration of 0.1% followed by vortex. The typical initial pH was in the range between 4.1 and 4.5, then the pH was adjusted to 6.00±0.10 with 1N NaOH (or 1N HCl if necessary). In order to assess intrinsic solubility of each compound, no solubilizing agents were added. Solubility of the compound is evaluated at this point, including assessment of the purity of the compound at the initial time point using HPLC-MS. Stability is evaluated at day 5 and day 12. Stability evaluation is normalized to the initial purity from day 0. Total concentration is observed over the time period.

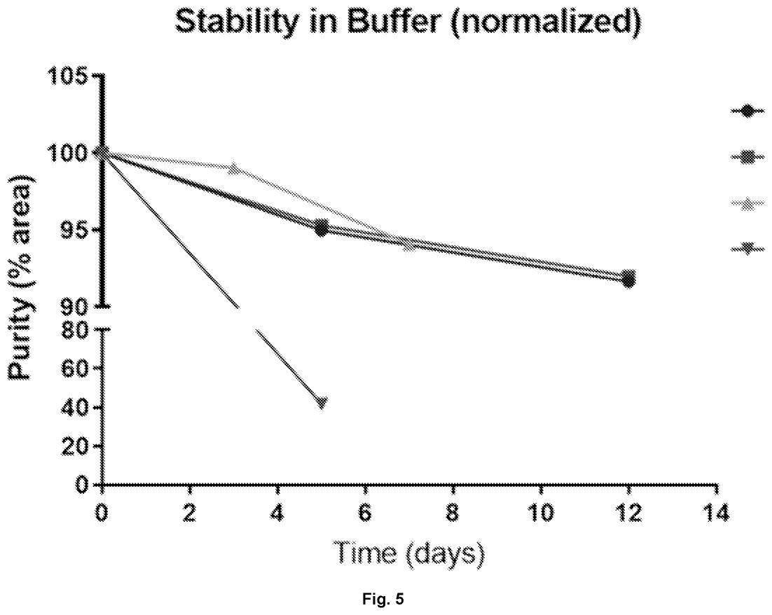

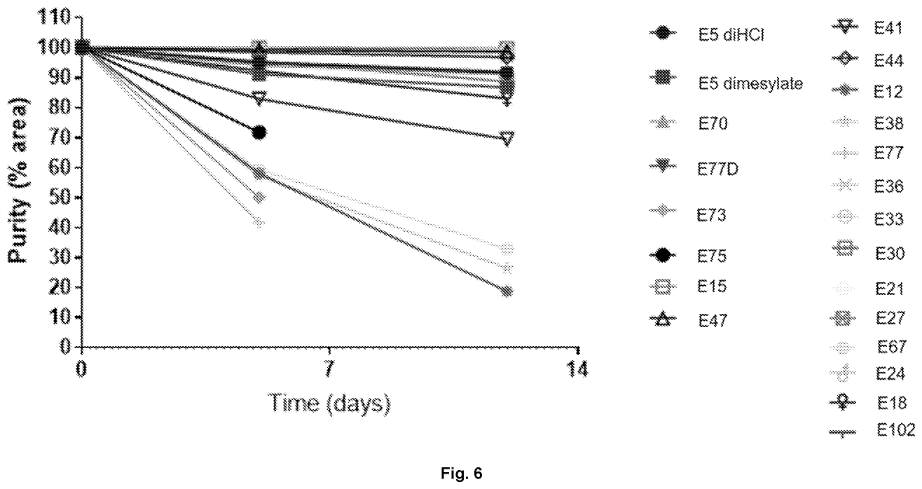

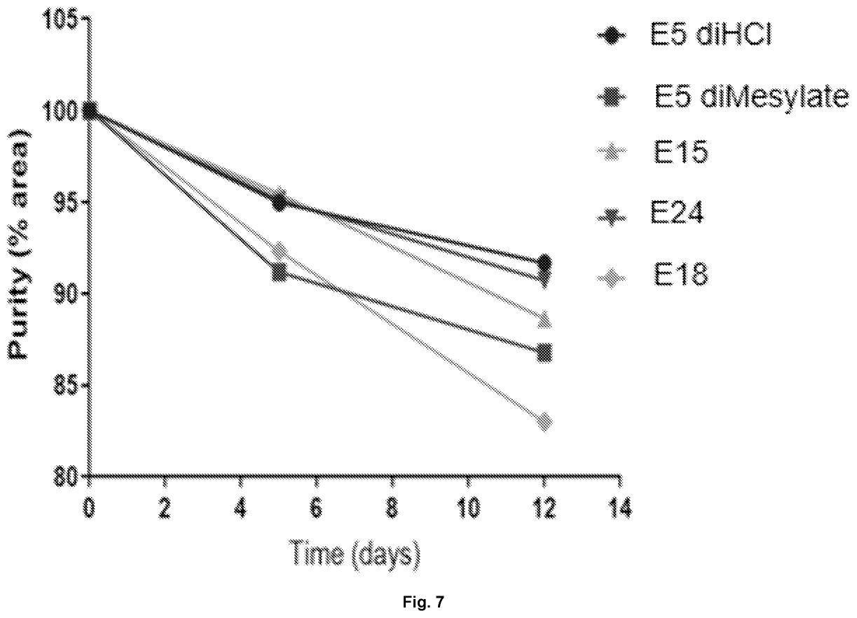

The difference in stability can be seen in FIG. 5. Compounds including a glutarate or carbonate linker were both more stable than a compound including a glutamate linker. Removing an amine from the linker, when comparing glutarate and glutamate linkers, provided a surprisingly more stable compound. A more stable compound—one that paces hydrolysis-permits a likewise pacing of continuity in treatment as compared to a compound that hydrolyses according to a more rapid exponential decay—one having a rapid initial hydrolysis followed by a slower tailing hydrolysis. FIG. 6 shows the hydrolysis of the compounds varies in a 10 mM phosphate buffer solution at pH 6 depending on the linker and steroid used. FIG. 7 shows a number of compounds having a glutarate linker that demonstrate a hydrolysis profile within about 10% of one another after 12 days in buffer.

### Example 6. In Vitro Pig Liver Esterase (PLE) Stability

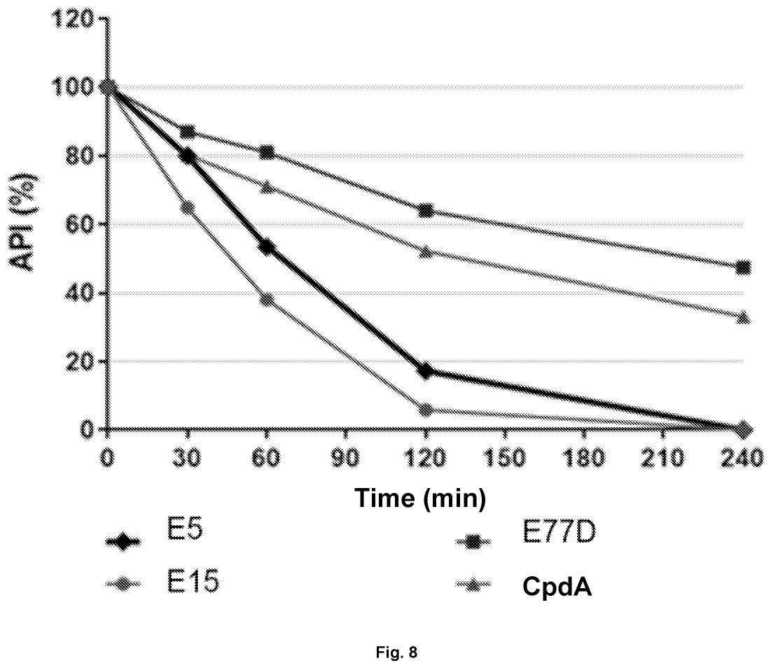

The in vitro enzymatic hydrolysis of compounds was evaluated using pig liver esterase (PLE) enzyme. In a time course study, each compound at 50 UM was incubated with 0.5 U PLE at room temperature in aqueous buffer pH 7 and samples taken at 0, 30, 60, 120, and 240 minutes for subsequent analytical assessment. Sample in the absence of PLE enzyme was run as a control measure of non-enzymatic hydrolysis of the compounds. Samples were analyzed by HPLC-MS for the change in parent compounds well as increase of the predicted metabolites. FIG. 8 shows compounds with a glutarate linker are more susceptible to faster pig liver esterase degradation (cell-free, in vitro enzyme metabolism time course assay) as compared to compounds with a cyclopropyl or succinate linker.

### Example 7. Chronic/Severe Allergic Eye Disease (AED) Model

E5 was formulated in 5% sulfobutylether-β-cyclodextrin (SBEβCD), 10 mM PBS (0.1% NaCl) solution, pH 6 and filtered with a 0.2 μm PVDF filter. AED was induced in wildtype C57Bl/6 mice. Animals were immunized with one intraperitoneal (i.p) injection of 100 μg of OVA (InvivoGen) in 300 ng of pertussis toxin (List Biologicals) and 4 mg of aluminum hydroxide (Alum, Thermo Fisher Scientific Pierce) on Day 0. Two weeks later, mice were treated topically QD with 5 μL OVA (50 μg/μL) in sterile saline for 5 days. Mice were treated topically BID with 5 uL of vehicle (10% SBEβCD), Lotemax® (Bausch & Lomb; loteprednol etabonate ophthalmic gel 0.5%) or 0.1% E5 in SBEβCD (50:1). The first treatment was administered on Day 14 (2 weeks after initial immunization), 10 minutes prior to OVA instillation followed by a second dose >7 hrs thereafter.

Assessment was carried out following euthanasia. All four lids were gently inverted under a dissection microscope to expose the posterior lid margin. The number of plugs on the upper and lower eyelids are analyzed and scored for severity as follows: 0 indicates no plug, 1+ indicates appearance of plug. 2+ is assigned to a plug that obstructs the entire MG (Meibomian gland) orifice, 3+ is a plug with a dome that rises above the plane of the eyelid margin, and a 4 is given to a plug that covers a larger surface than the MG orifice (usually with irregular circumference edges).

Clinical parameters including eyelid edema, chemosis, conjunctival redness, and tearing were each scored on a scale of 0-3+. A score of 0 indicated no evidence of the respective parameter, 1+ is assigned if the response is mild, but distinctly greater than the naive controls; 2+ for a moderate change in the respective parameters that could be noted by slit-lamp microscopy, but not with naked eye; and 3+ if the response is severe enough that could be perceived with naked eye.

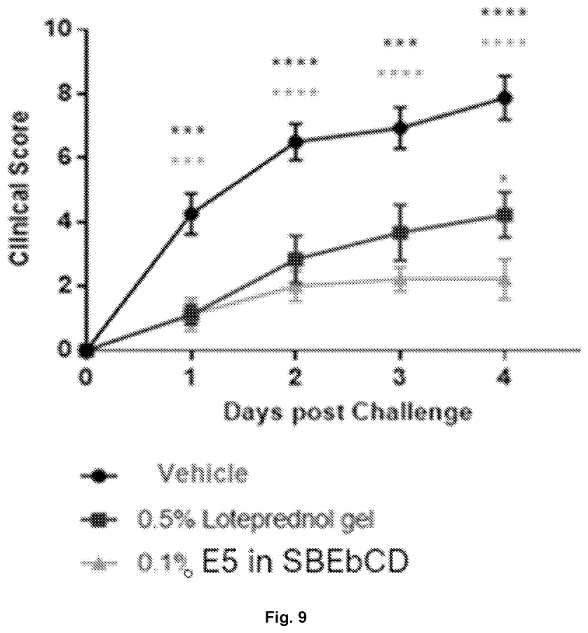

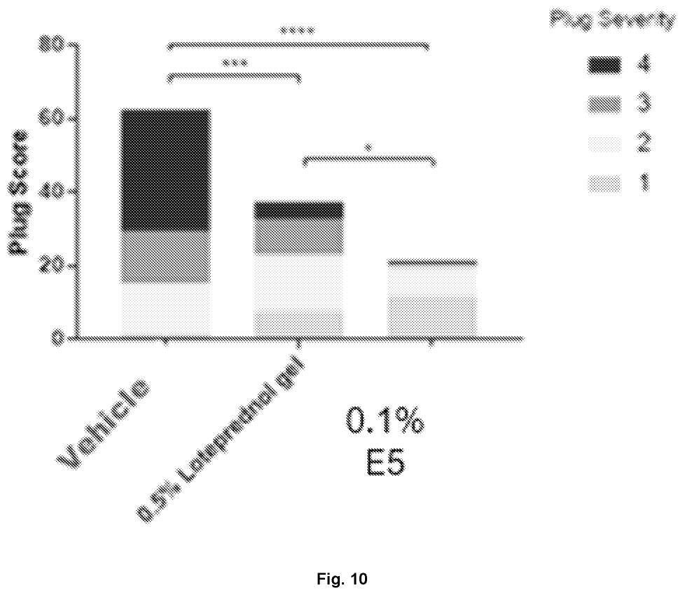

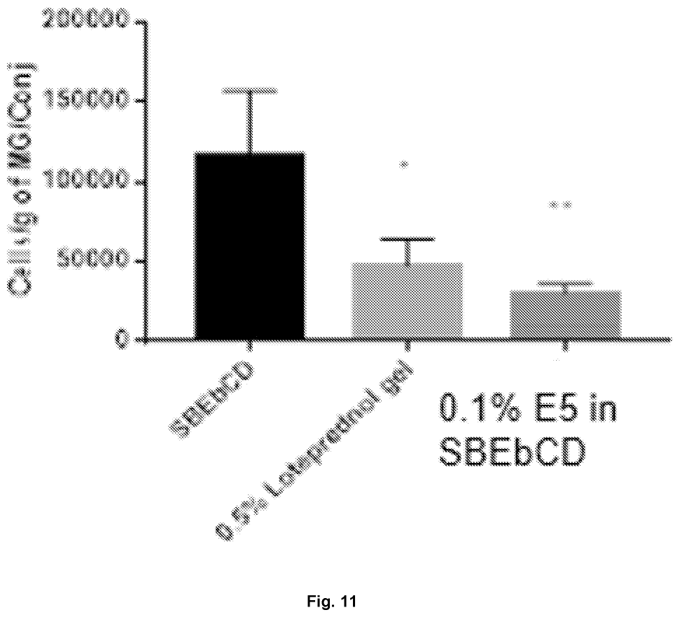

Results are shown in FIG. 9, FIG. 10, and FIG. 11. Clinical scoring shows 0.1% E5 is statistically significantly improved compared to vehicle performed better than loteprednol gel demonstrating statistical significance on day 4 (FIG. 9). 0.1% E5 was effective in lowering both the number and severity of the MG plugs compared to both vehicle and loteprednol (FIG. 10). 0.1% E5 reduced the number of neutrophils in the Meibomian gland and conjunctiva (FIG. 11).

### Example 8. In Vivo PK/PD Ocular Surface Corneal Wound Healing Model

E5 was formulated in 3.5% sulfobutylether-β-cyclodextrin (SBEβCD), 10 mM PBS, pH 6 and filtered with a 0.2 μm PVDF filter.

Anesthetized animals (8-10 weeks old male C57/B1/6 mice) were placed under a dissecting microscope and eyes proposed. Corneas of OD eyes were demarcated by centrally, and softly, marking the perimeters of the debridement area with a 2 mm trephine. A 10 μL drop of PBS was placed on the eye and an Algerbrush with a 0.5 mm burr attachment was utilized to carefully remove the epithelium within the demarcated area of the cornea. Topical fluorescein (Sigma) was applied, and excess fluorescein flushed prior to capture of a baseline image of the wound (time=0). Topical treatments were administered at t=0 and t=6 hours post-injury by applying 5 UL to the surface of the debrided eye. Fluorescein-stained corneas were imaged again 24 h after injury. Percent re-epithelialization was assessed by analysis of imaged corneas with Image J (NIH, Bethesda, MD).

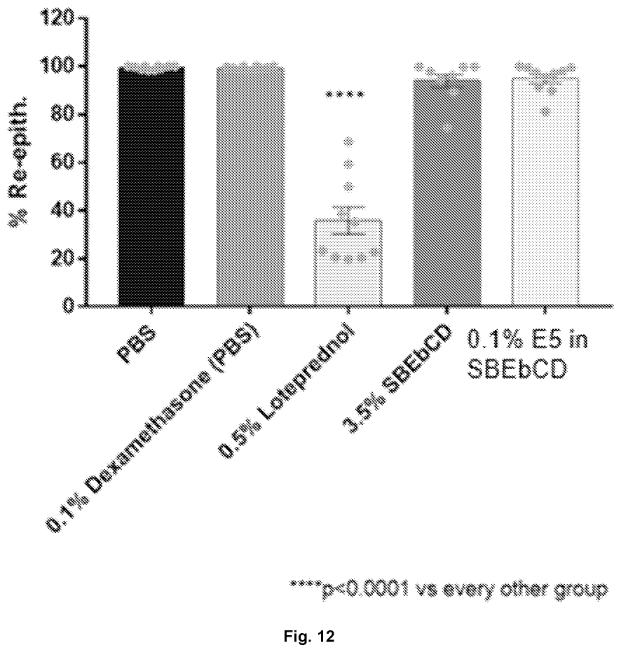

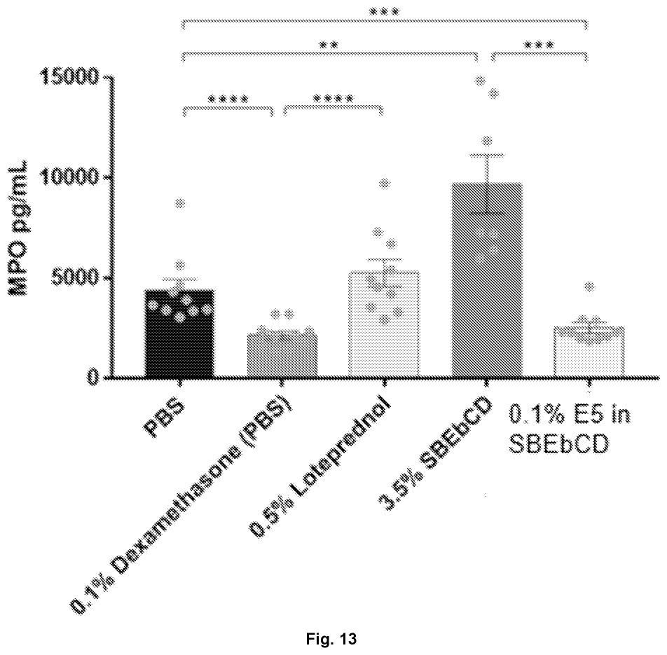

Results are shown in FIG. 12 and FIG. 13. The corneal wound healing model shows the ability of 0.1% E5 to reduce eye surface inflammation (MPO; myeloperoxidase) without having the negative effect on epithelium regrowth/healing 24 hrs after epithelium debridement. 0.1% dexamethasone and 0.5% loteprednol were used as positive clinically relevant controls. 0.1% E5 did not affect the recovery of epithelium in this model (FIG. 12). 0.1% E5 significantly reduced the cornea inflammation in comparison to PBS (phosphate-buffered saline) vehicle and loteprednol groups (FIG. 13).

### Example 9. Intraocular Pressure Study in Normotensive Dutch Belted Rabbits

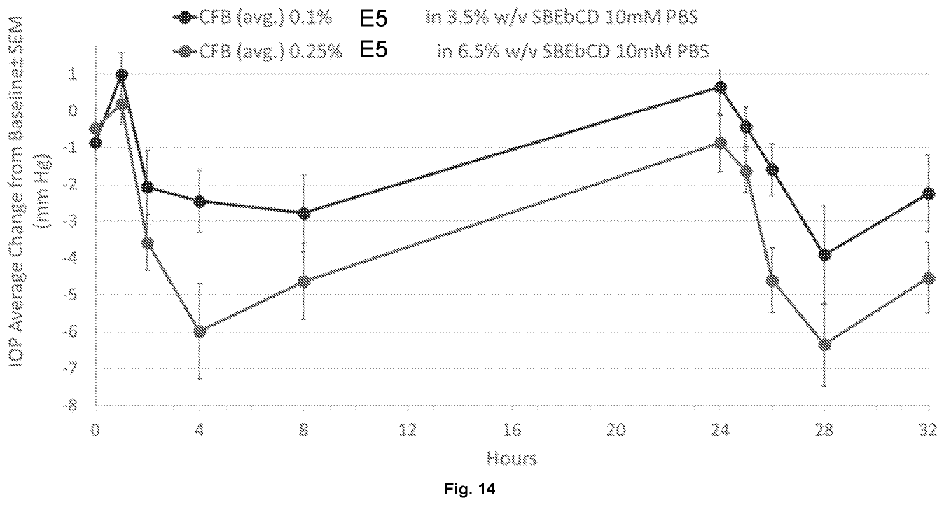

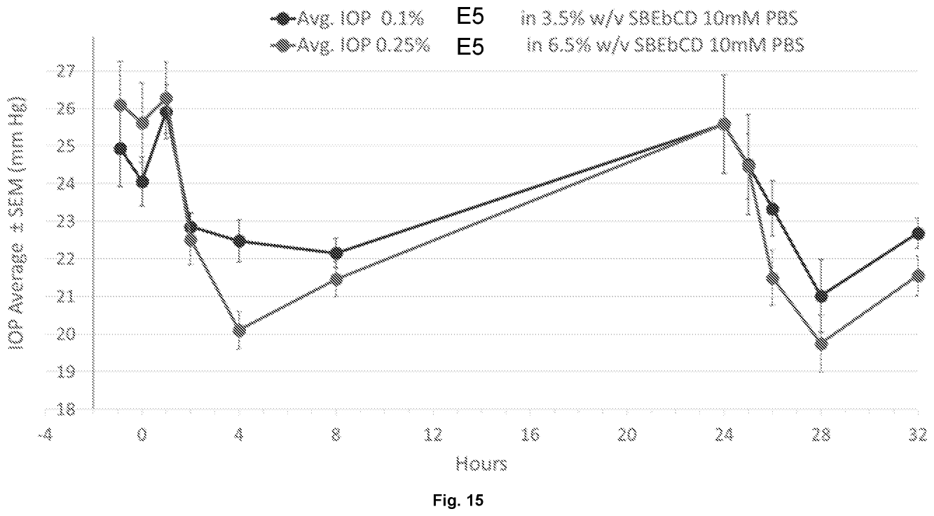

E5 was formulated in 3.5% or 6.5% sulfobutylether-β-cyclodextrin (SBEβCD), 10 mM PBS, pH 6 and filtered with a 0.2 μm PVDF filter. A single dose was administered and the intraocular pressure was measured, e.g., with a pneumatonometer, for 32 hours post-dose. Results are shown in FIG. 14 and FIG. 15. A single dose of E5 lowered IOP in a dose-dependent manner with no hyperemia observed (FIG. 14 and FIG. 15).

### Example 10. In Vitro Steroid and ROCKi Activity

Activities of certain compounds and components of compounds described herein were assayed. Results are shown in Table 7. It was observed that E5 and E15 possess both ROCKi and steroid potency and activity. Rho-associated protein kinase 2=ROCK2; Rho-associated protein kinase 1=ROCK1; Porcine Trabecular Meshwork cells=PTM; Human Trabecular Meshwork cells=HTM; Splenic Interleukin-23=SPNL IL23; and Tumor Necrosis Factor α=TNFα.

TABLE 7
IC₅₀ (nM)

| | | | | | | | SPLN | |
|---|---|---|---|---|---|---|---|---|
| No. | Steroid | Linkage | ROCK2 | ROCK1 | PTM | HTM | IL23 | TNFα |
| A | None | None | 0.99 | 1.7 | 13 | 5 | | |
| B | Dexamethasone | None | 50000 | 50000 | 3000 | 3000 | 4.6 | 47 |
| C | Triamcinolone | None | 50000 | 50000 | | | 0.93 | 0.51 |
| E5 | Triamcinolone | Glutarate | 9 | 9 | 55 | | 1 | 10 |
| E15 | Dexamethasone | Glutarate | 4 | 7 | 40 | | 12 | 147 |

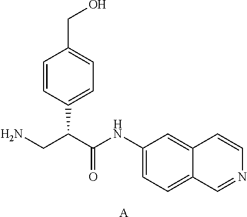

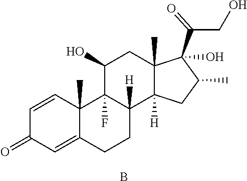

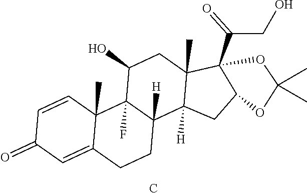

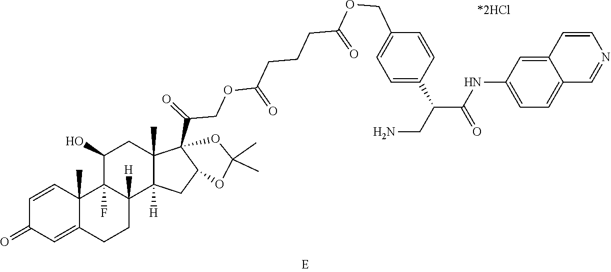

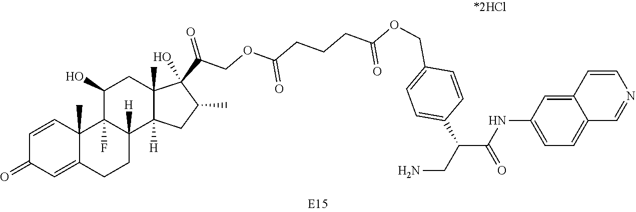

Where publications and patents are referred to throughout this disclosure, all U.S. Patents cited herein are hereby incorporated by reference. All percentages, ratios, and proportions used herein are percent by weight unless otherwise specified.

Unless defined otherwise, all technical and scientific terms used herein generally have the same meaning as commonly understood by one of ordinary skill in the art to which this disclosure relates. Generally, the nomenclature and laboratory procedures used herein are those commonly employed by one of ordinary skill in the art.

1. A compound of the formula: 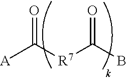
or a pharmaceutically acceptable salt thereof,
wherein:
k is 0 or 1;
A is a corticosteroid moiety, linked through its primary alcohol to form an ester linkage;
B is a primary alcohol-containing rho kinase inhibitor, linked through its primary alcohol to form an ester linkage; and
R⁷ is

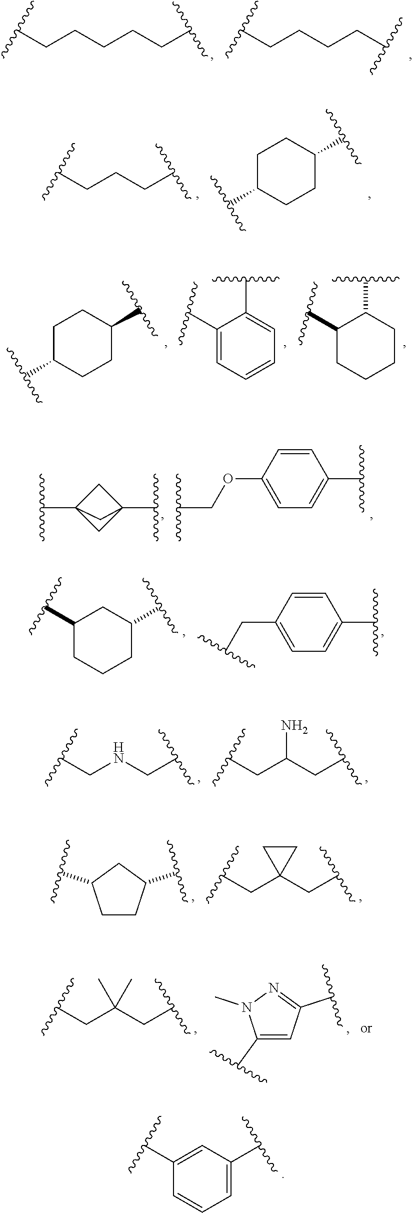

2. The compound of claim 1, having the formula: 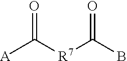
or a pharmaceutically acceptable salt thereof.

3. The compound of claim 1, having the formula: 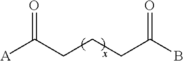
or a pharmaceutically acceptable salt thereof,
wherein:
x is 1 or 2 or 3.

4. The compound of claim 1, having the formula: 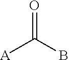
or a pharmaceutically acceptable salt thereof.

5. The compound of claim 1, wherein:
A is 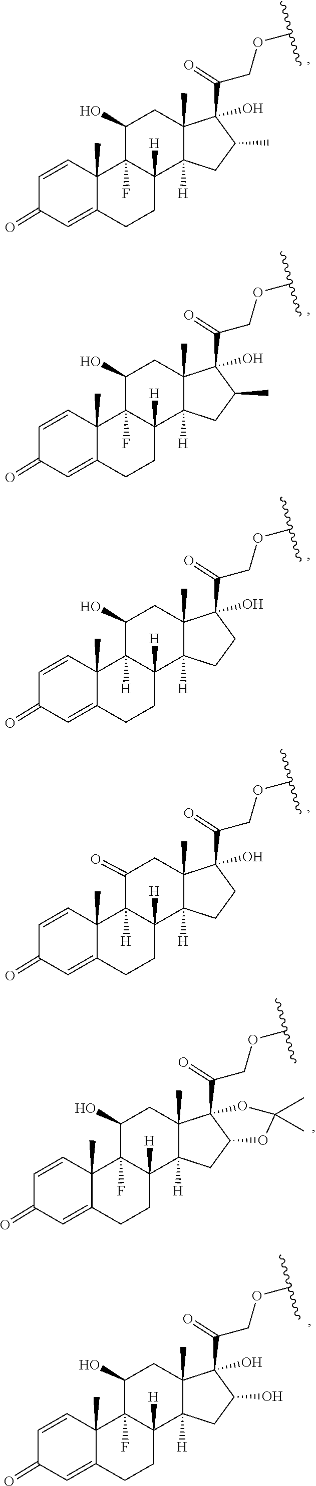 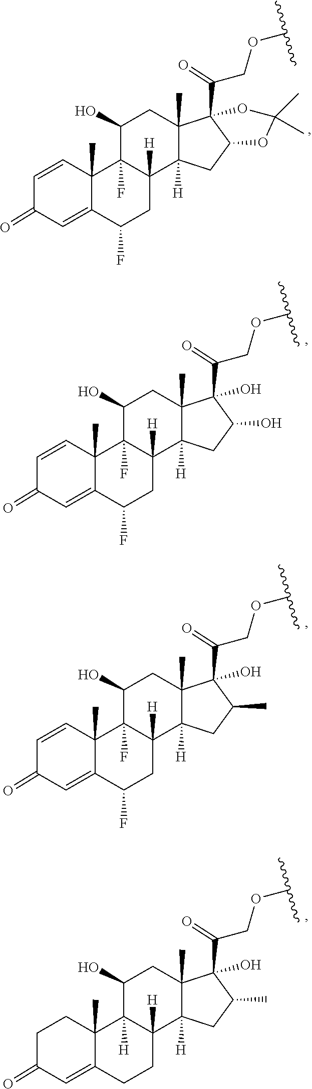 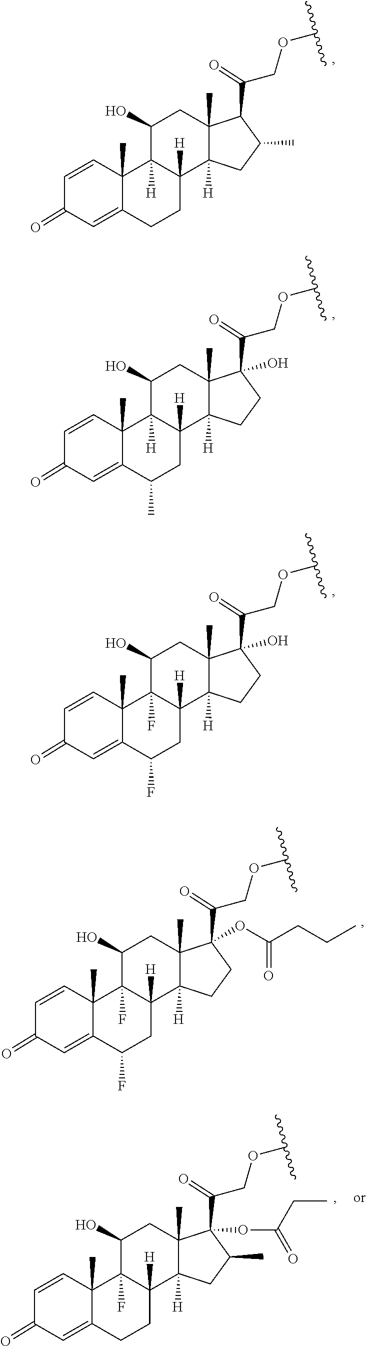 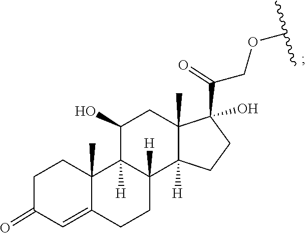
and
B is

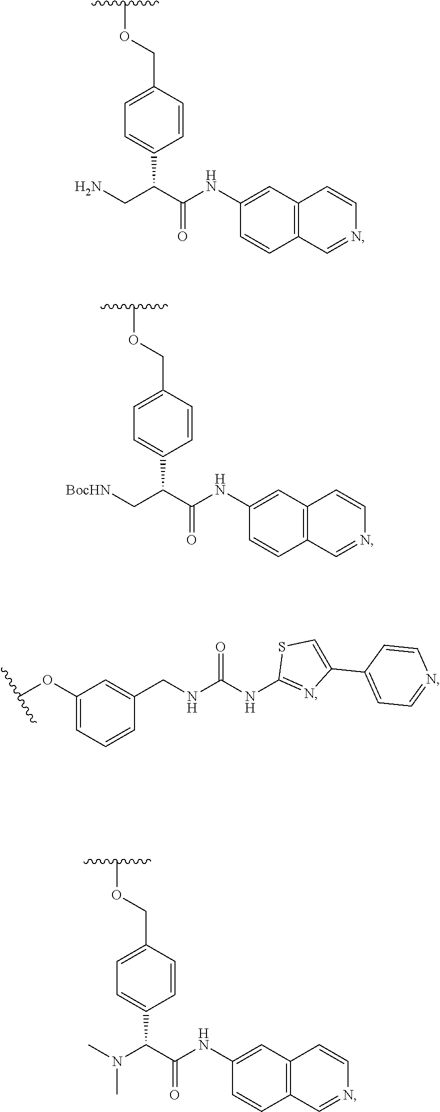 [SMILES: *OCc1ccc([C@@H](CN)C(=O)Nc2ccc3cnccc3c2)cc1 ; *OCc1ccc([C@@H](CNC(=O)OC(C)(C)C)C(=O)Nc2ccc3cnccc3c2)cc1 ; *Oc1cccc(CNC(=O)Nc2nc(-c3ccncc3)cs2)c1 ; *OCc1ccc([C@H](C(=O)Nc2ccc3cnccc3c2)N(C)C)cc1]

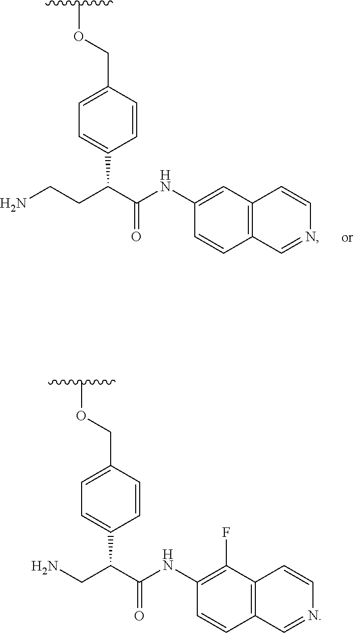 [SMILES: *OCc1ccc([C@@H](CCN)C(=O)Nc2ccc3cnccc3c2)cc1 ; *OCc1ccc([C@@H](CN)C(=O)Nc2ccc3cnccc3c2F)cc1]

6. The compound of claim 1, having the formula:
R⁸—R¹⁰—R¹¹
or a pharmaceutically acceptable salt thereof,
wherein:
R⁸ is 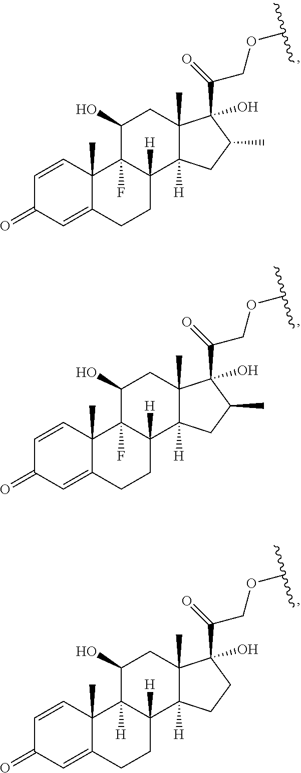 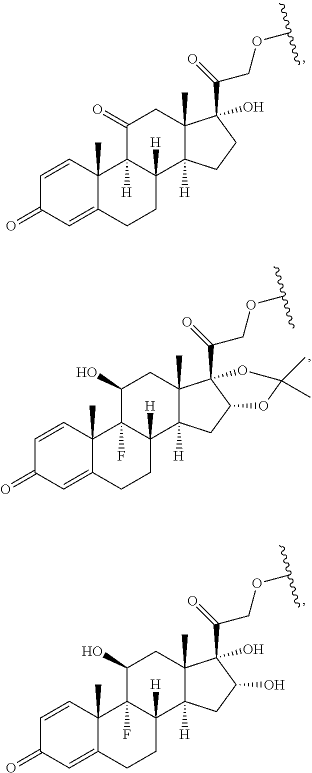 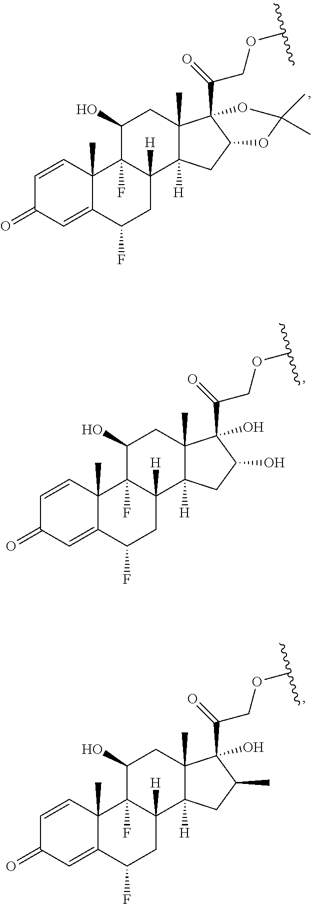 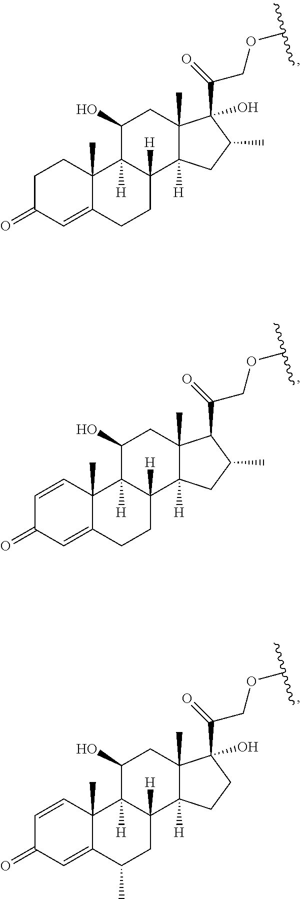 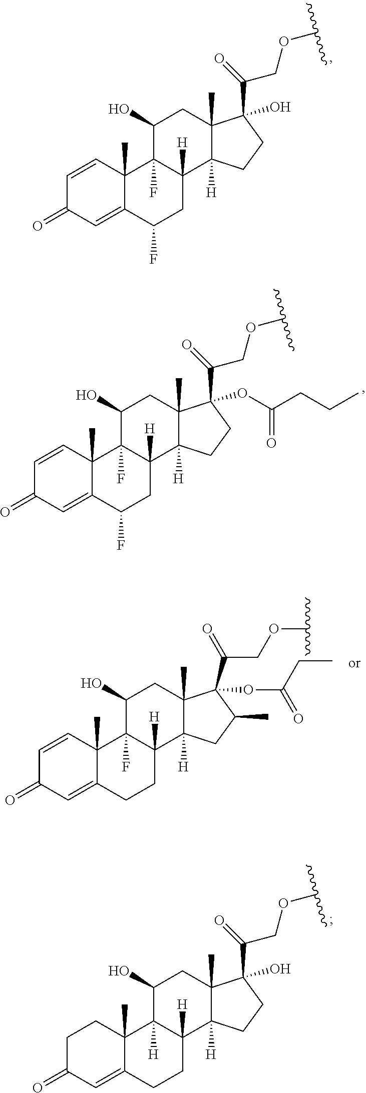
and
R¹⁰ is 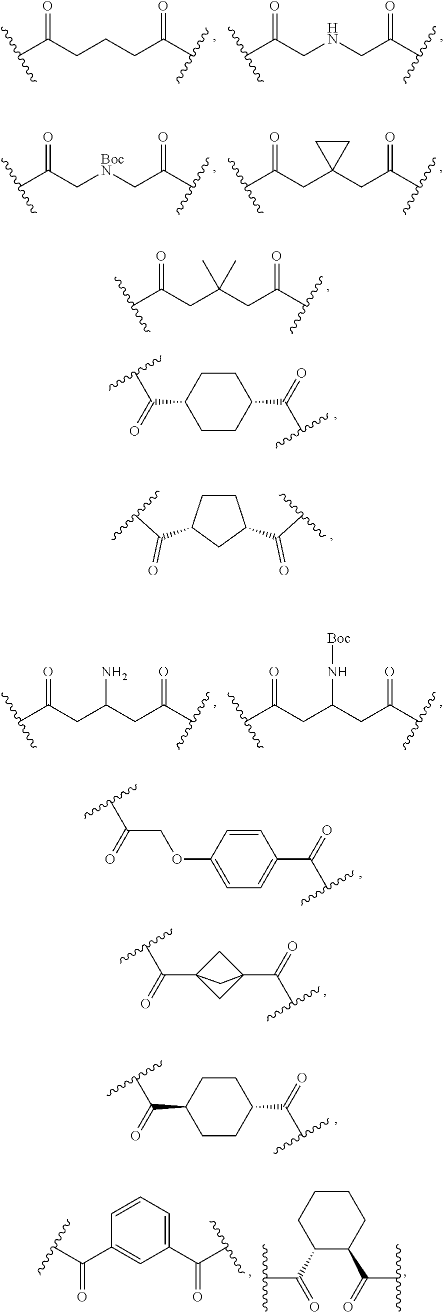 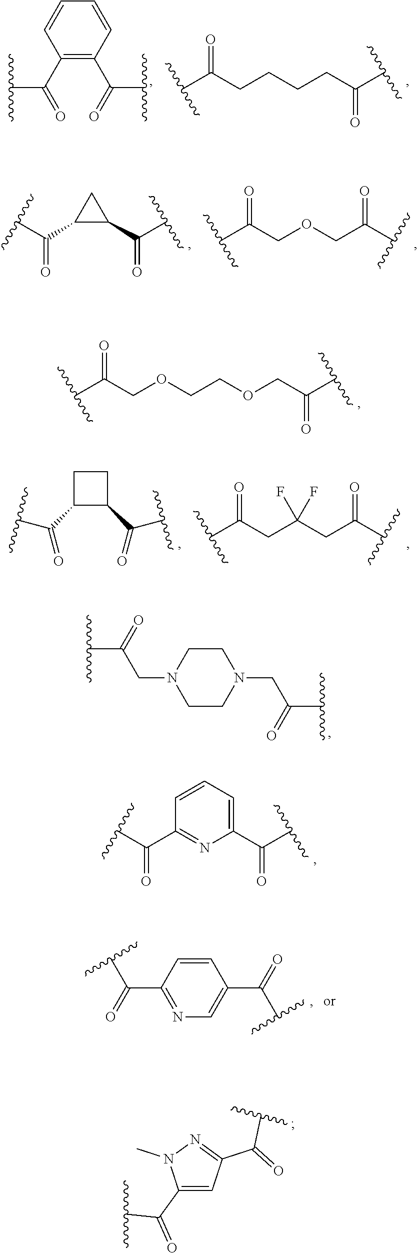
and
R¹¹ is

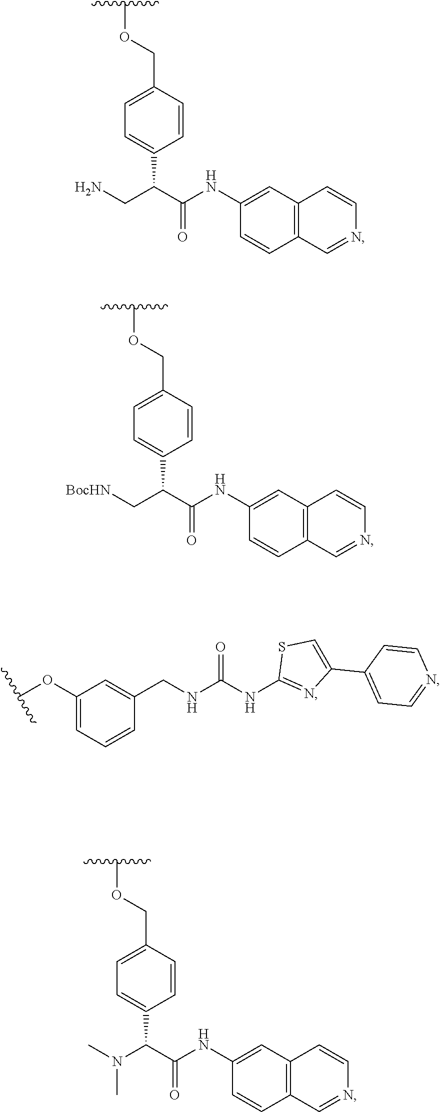 [SMILES: *OCc1ccc([C@@H](CN)C(=O)Nc2ccc3cnccc3c2)cc1 ; *OCc1ccc([C@@H](CNC(=O)OC(C)(C)C)C(=O)Nc2ccc3cnccc3c2)cc1 ; *Oc1cccc(CNC(=O)Nc2nc(-c3ccncc3)cs2)c1 ; *OCc1ccc([C@H](C(=O)Nc2ccc3cnccc3c2)N(C)C)cc1]

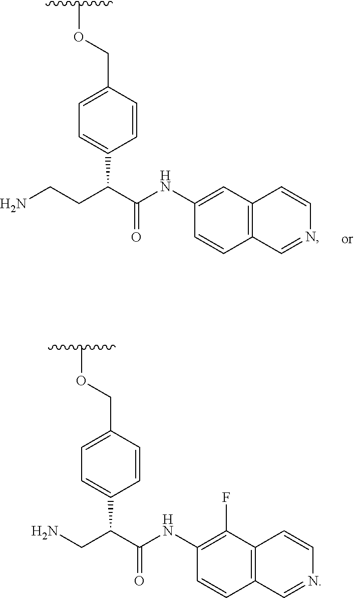 [SMILES: *OCc1ccc([C@@H](CCN)C(=O)Nc2ccc3cnccc3c2)cc1 ; *OCc1ccc([C@@H](CN)C(=O)Nc2ccc3cnccc3c2F)cc1]

7. The compound of claim 1, having the formula:
R⁸—R¹⁰—R¹¹
or a pharmaceutically acceptable salt thereof,
wherein:
R⁸ is 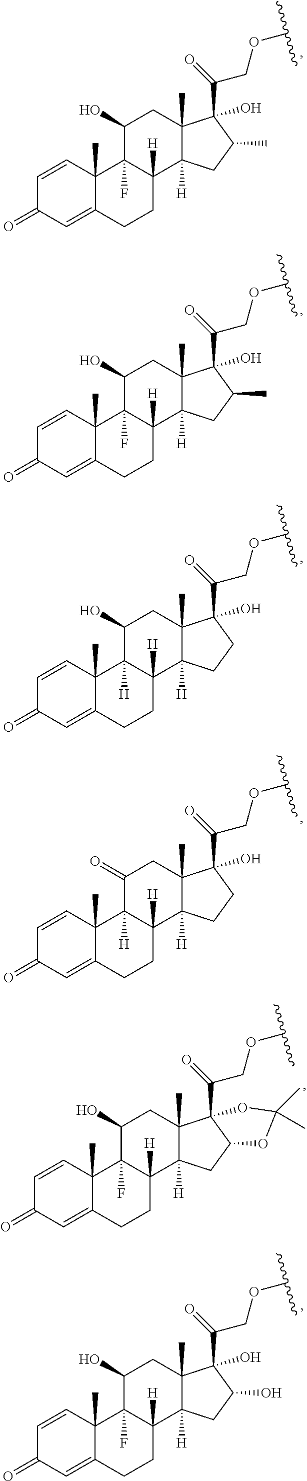 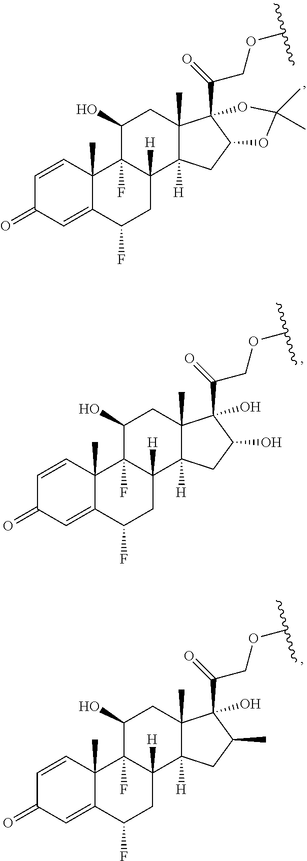 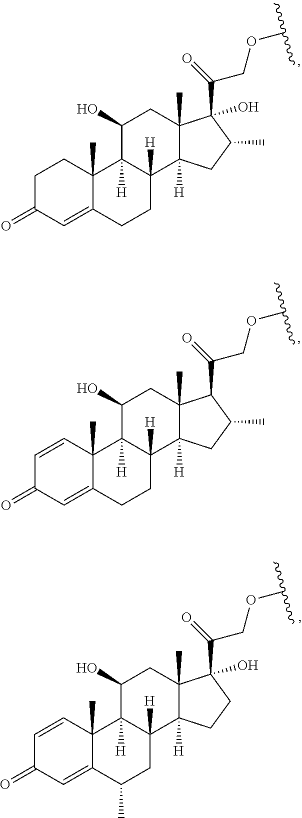 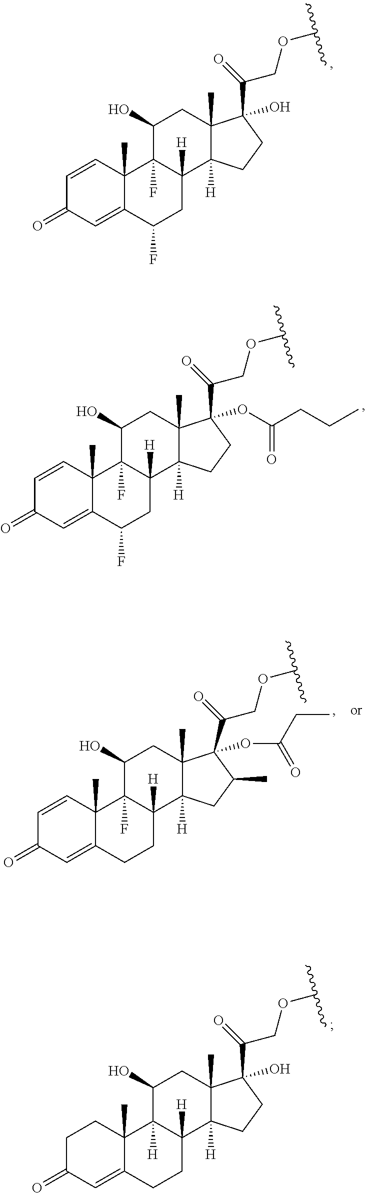
and
R¹⁰ is 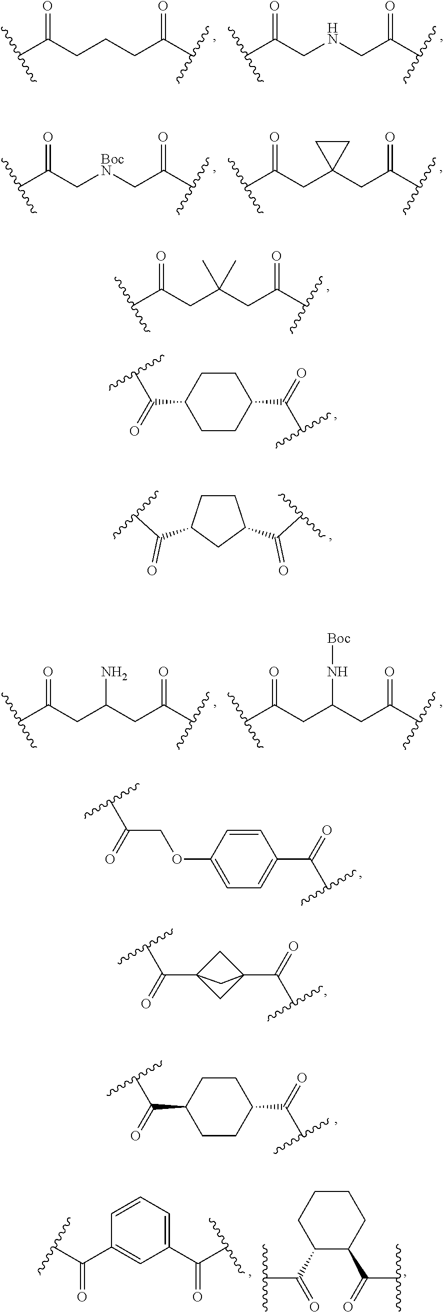 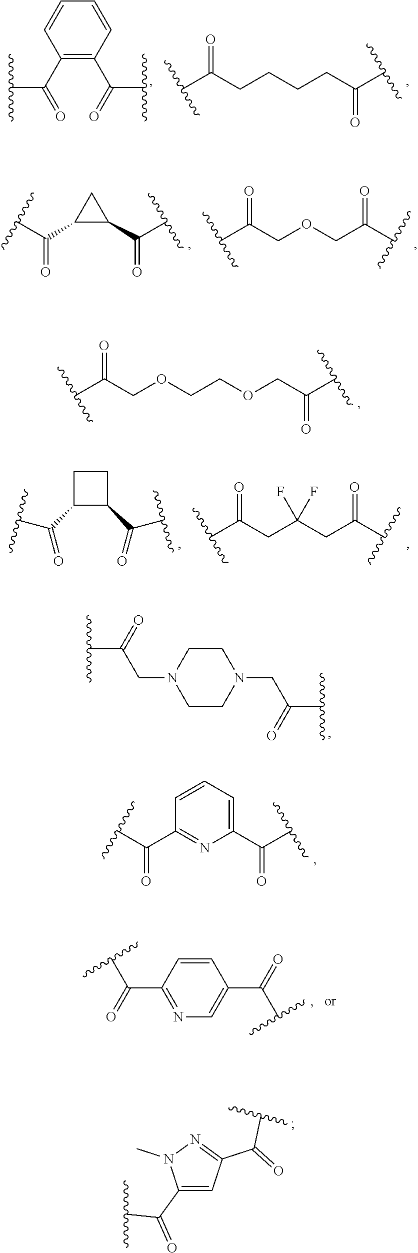
and
R¹¹ is

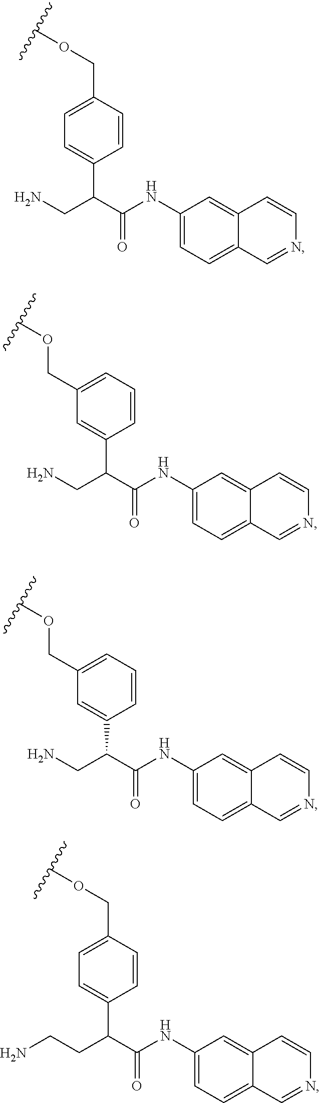 [SMILES: *OCc1ccc(C(CN)C(=O)Nc2ccc3cnccc3c2)cc1 ; *OCc1cccc(C(CN)C(=O)Nc2ccc3cnccc3c2)c1 ; *OCc1cccc([C@@H](CN)C(=O)Nc2ccc3cnccc3c2)c1 ; *OCc1ccc(C(CCN)C(=O)Nc2ccc3cnccc3c2)cc1]

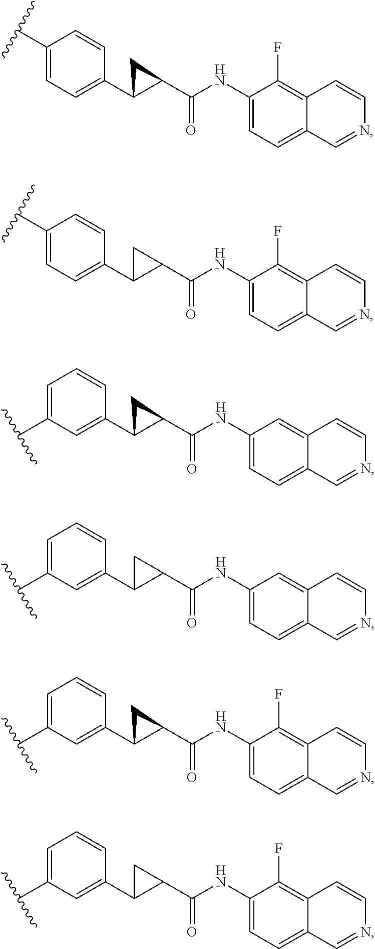 [SMILES: *c1ccc([C@@H]2C[C@H]2C(=O)Nc2ccc3cnccc3c2F)cc1 ; *c1ccc(C2CC2C(=O)Nc2ccc3cnccc3c2F)cc1 ; *c1cccc([C@@H]2C[C@H]2C(=O)Nc2ccc3cnccc3c2)c1 ; *c1cccc(C2CC2C(=O)Nc2ccc3cnccc3c2)c1 ; *c1cccc([C@@H]2C[C@H]2C(=O)Nc2ccc3cnccc3c2F)c1 ; *c1cccc(C2CC2C(=O)Nc2ccc3cnccc3c2F)c1]

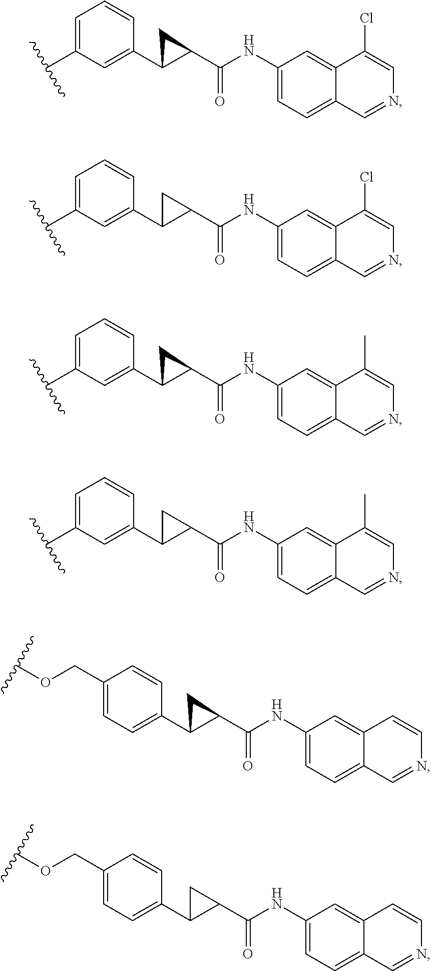 [SMILES: *c1cccc([C@@H]2C[C@H]2C(=O)Nc2ccc3cncc(Cl)c3c2)c1 ; *c1cccc(C2CC2C(=O)Nc2ccc3cncc(Cl)c3c2)c1 ; *c1cccc([C@@H]2C[C@H]2C(=O)Nc2ccc3cncc(C)c3c2)c1 ; *c1cccc(C2CC2C(=O)Nc2ccc3cncc(C)c3c2)c1 ; *OCc1ccc([C@@H]2C[C@H]2C(=O)Nc2ccc3cnccc3c2)cc1 ; *OCc1ccc(C2CC2C(=O)Nc2ccc3cnccc3c2)cc1]

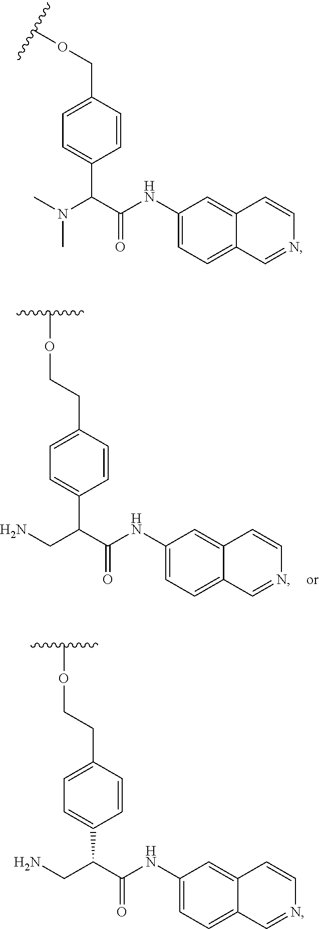 [SMILES: *OCc1ccc(C(C(=O)Nc2ccc3cnccc3c2)N(C)C)cc1 ; *OCCc1ccc(C(CN)C(=O)Nc2ccc3cnccc3c2)cc1 ; *OCCc1ccc([C@@H](CN)C(=O)Nc2ccc3cnccc3c2)cc1]

8. The compound of claim 1, having a formula selected from: 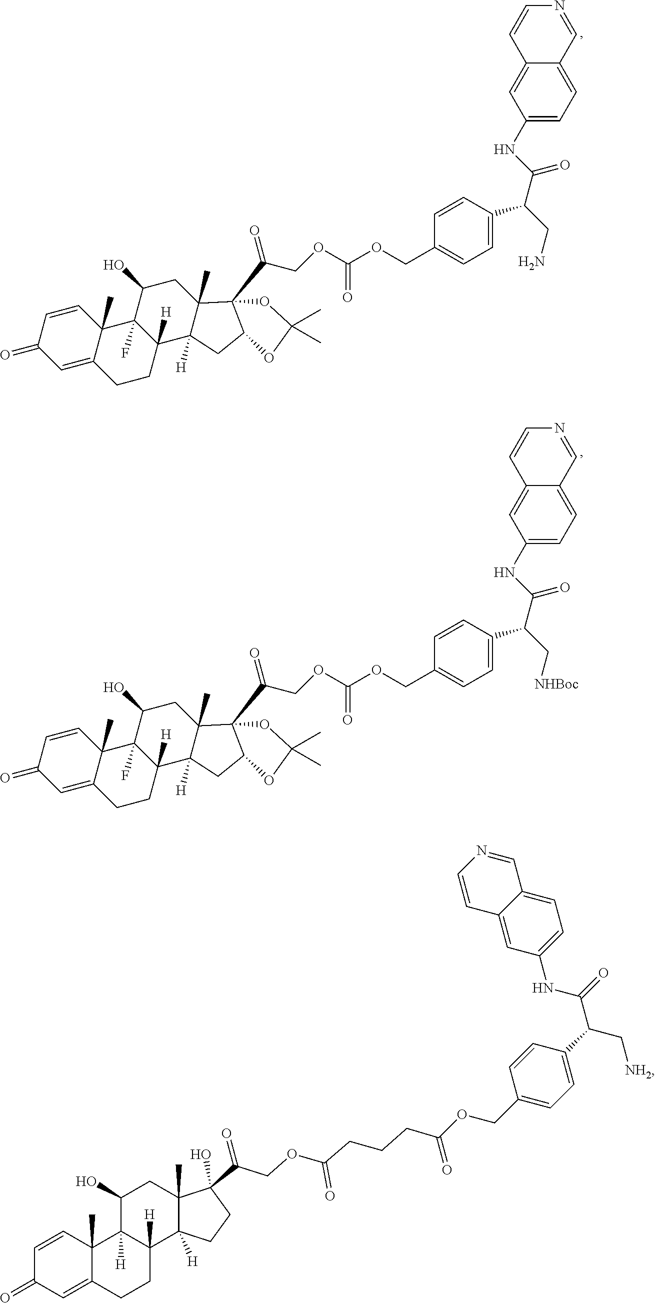 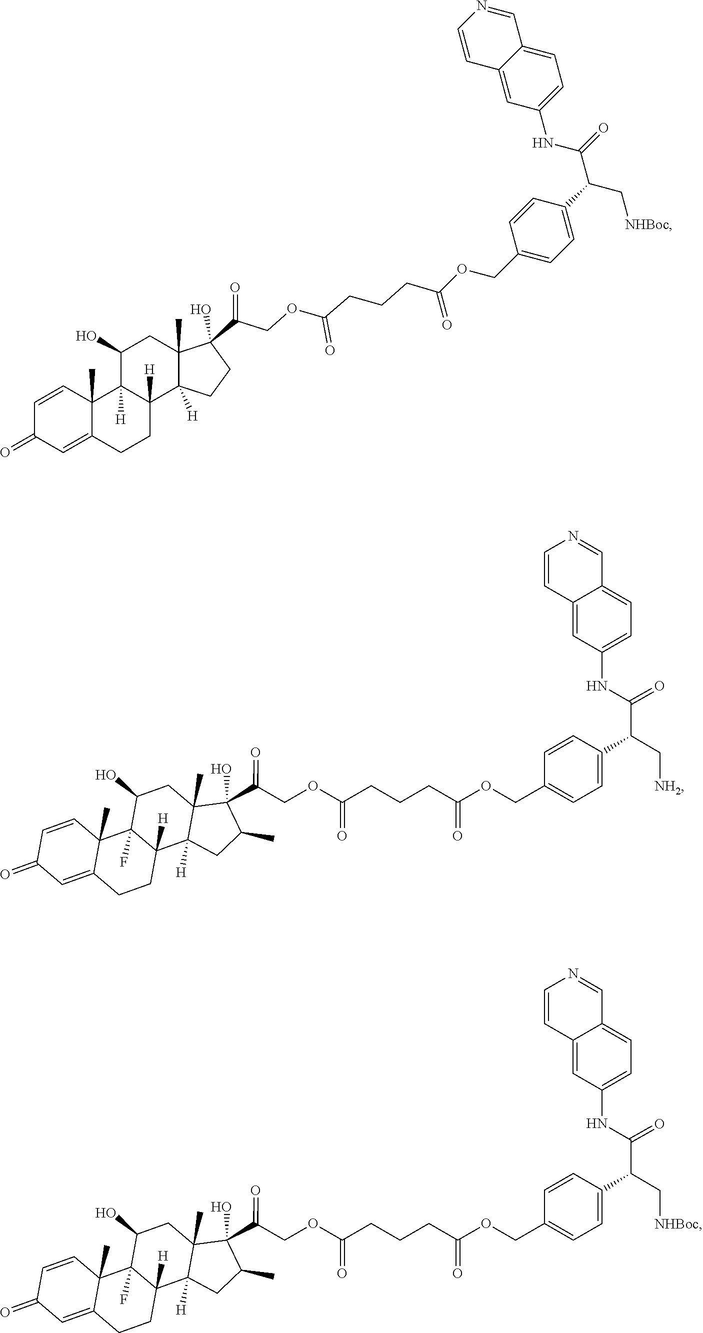 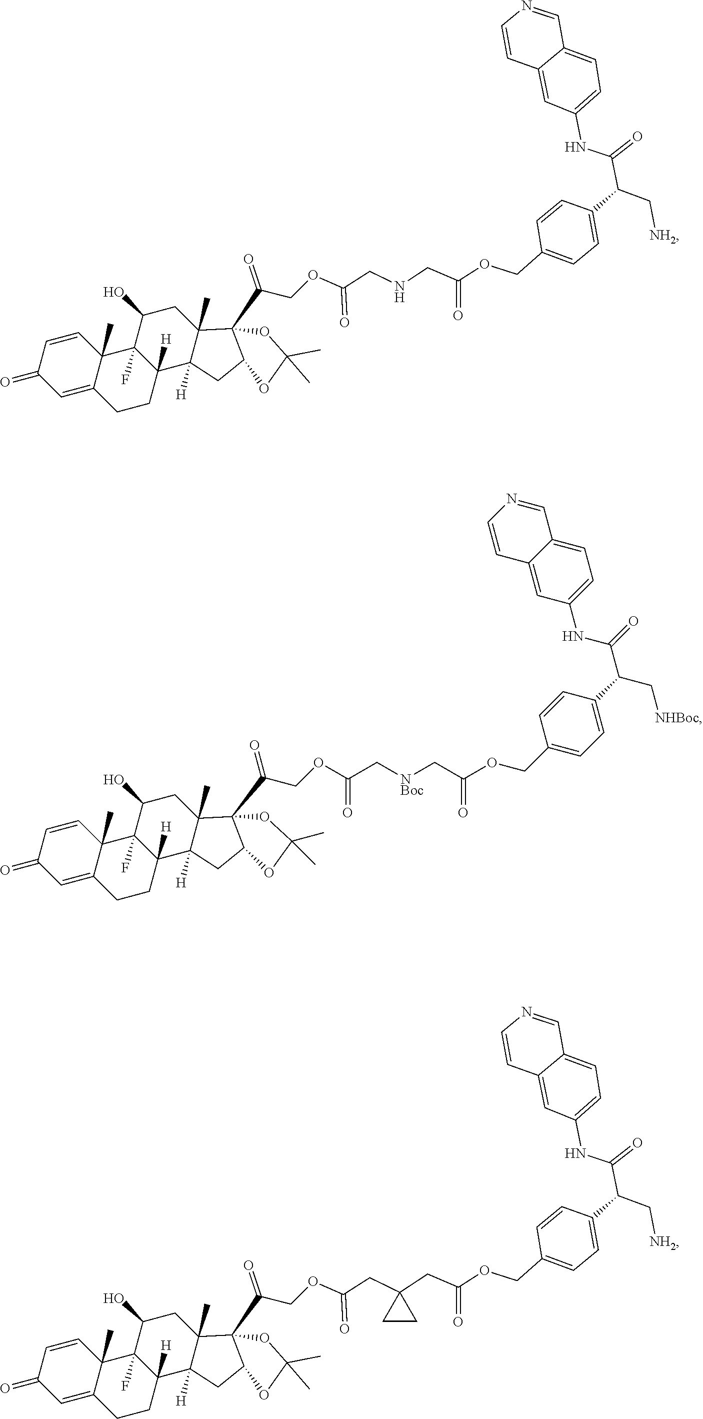 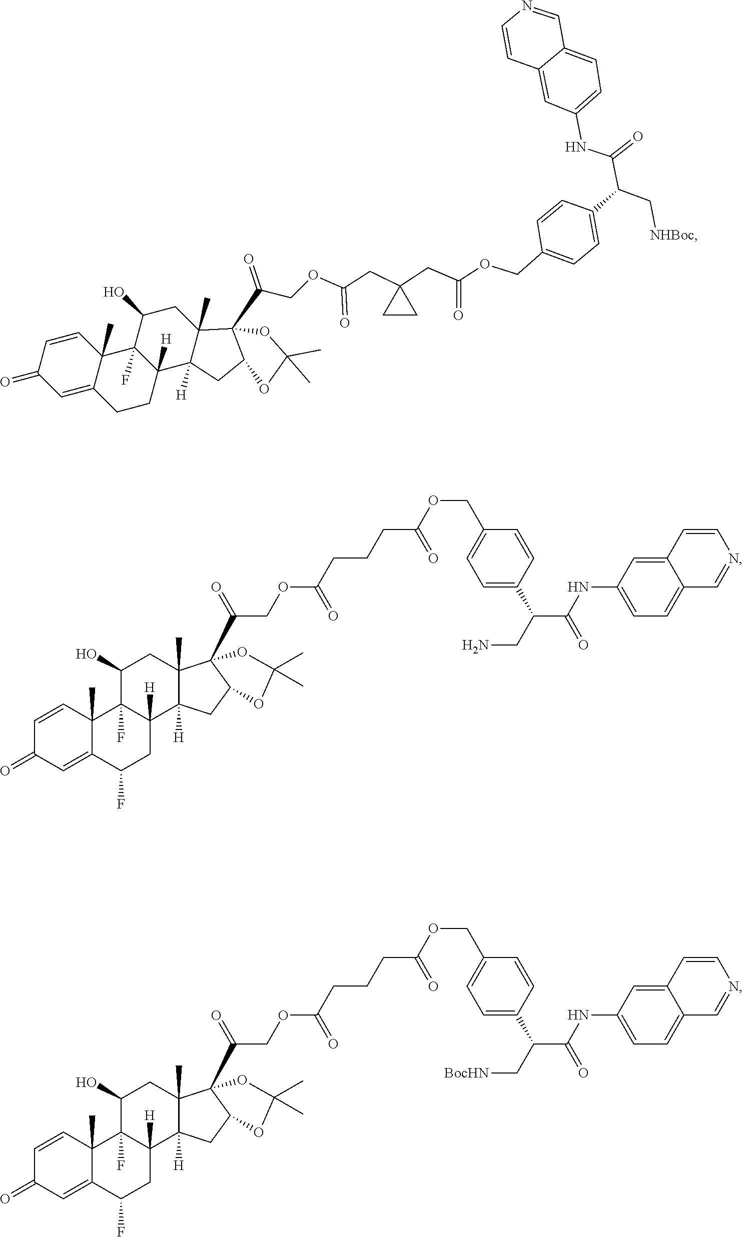 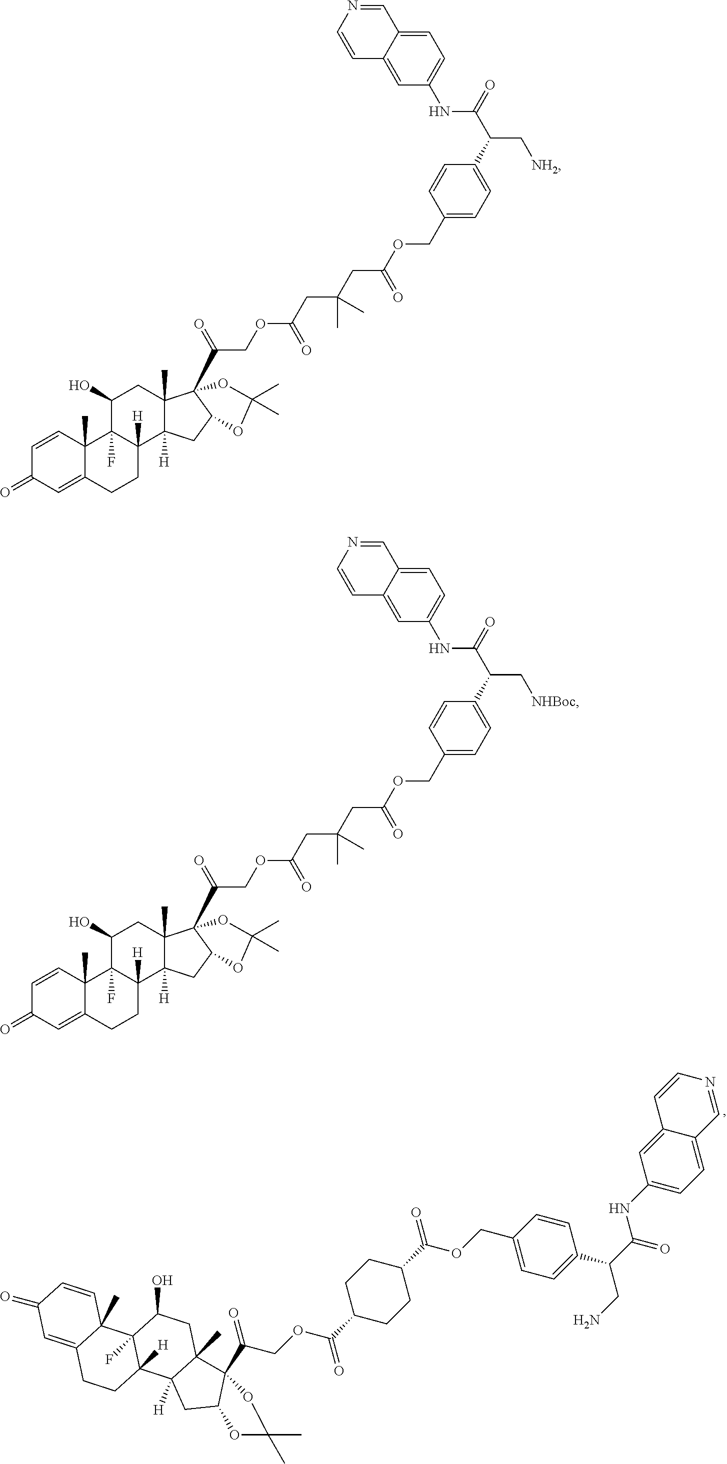 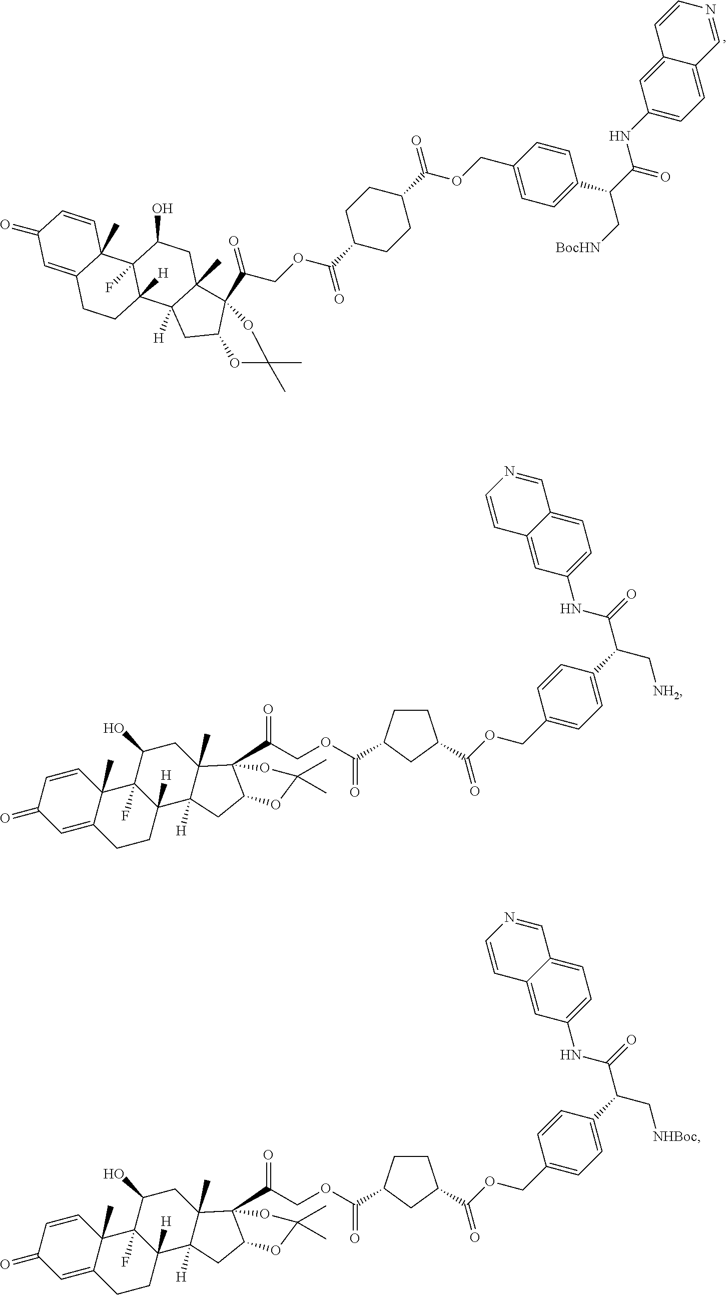 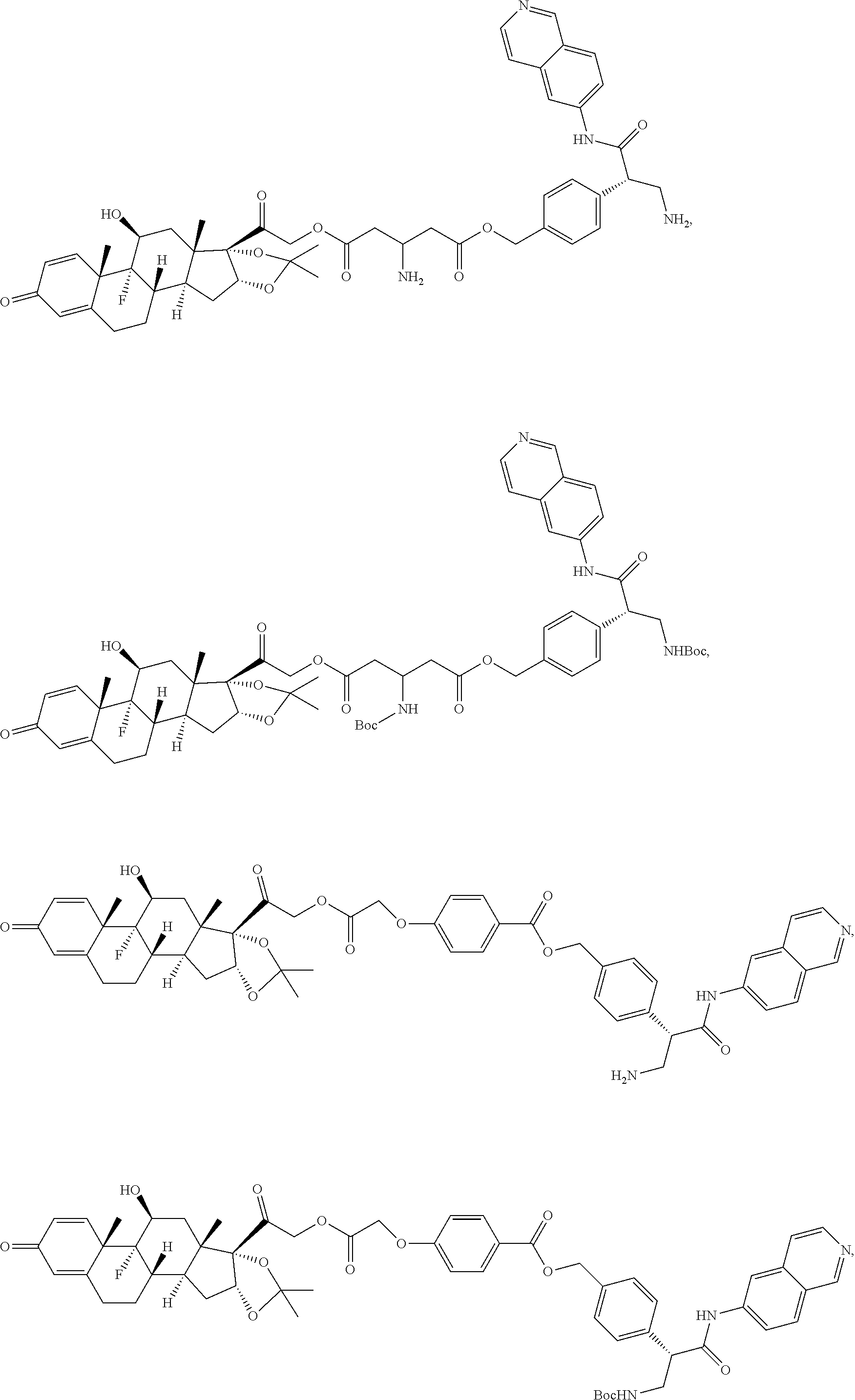 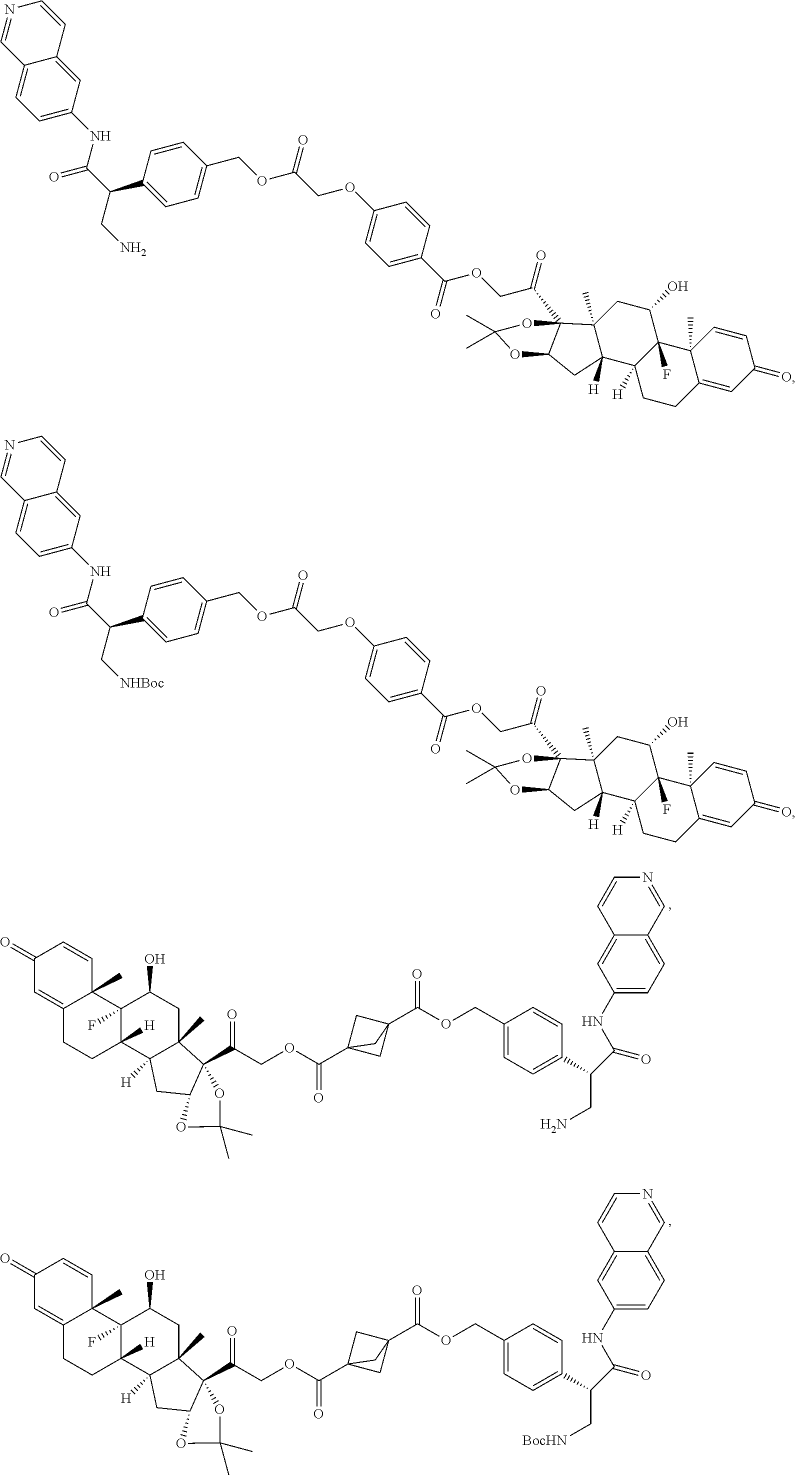 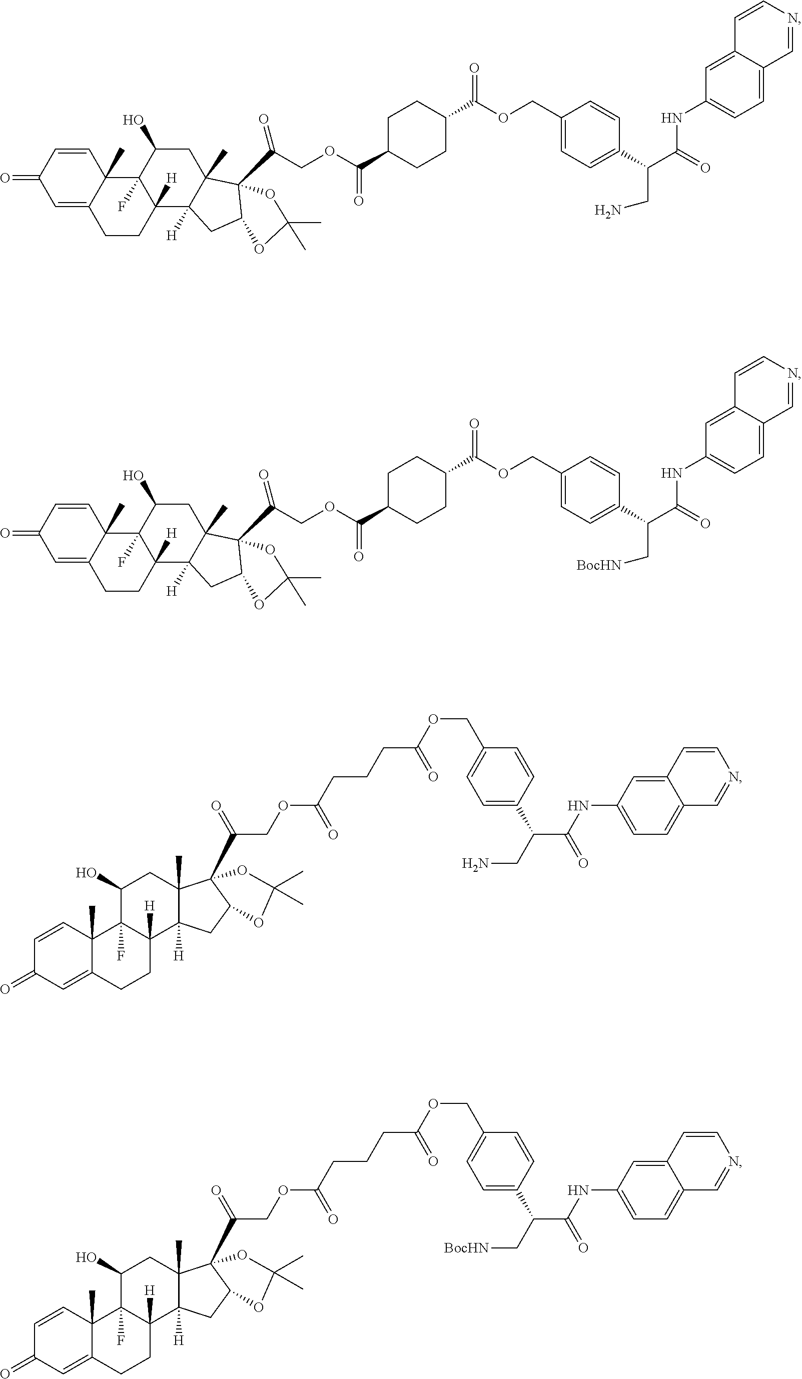 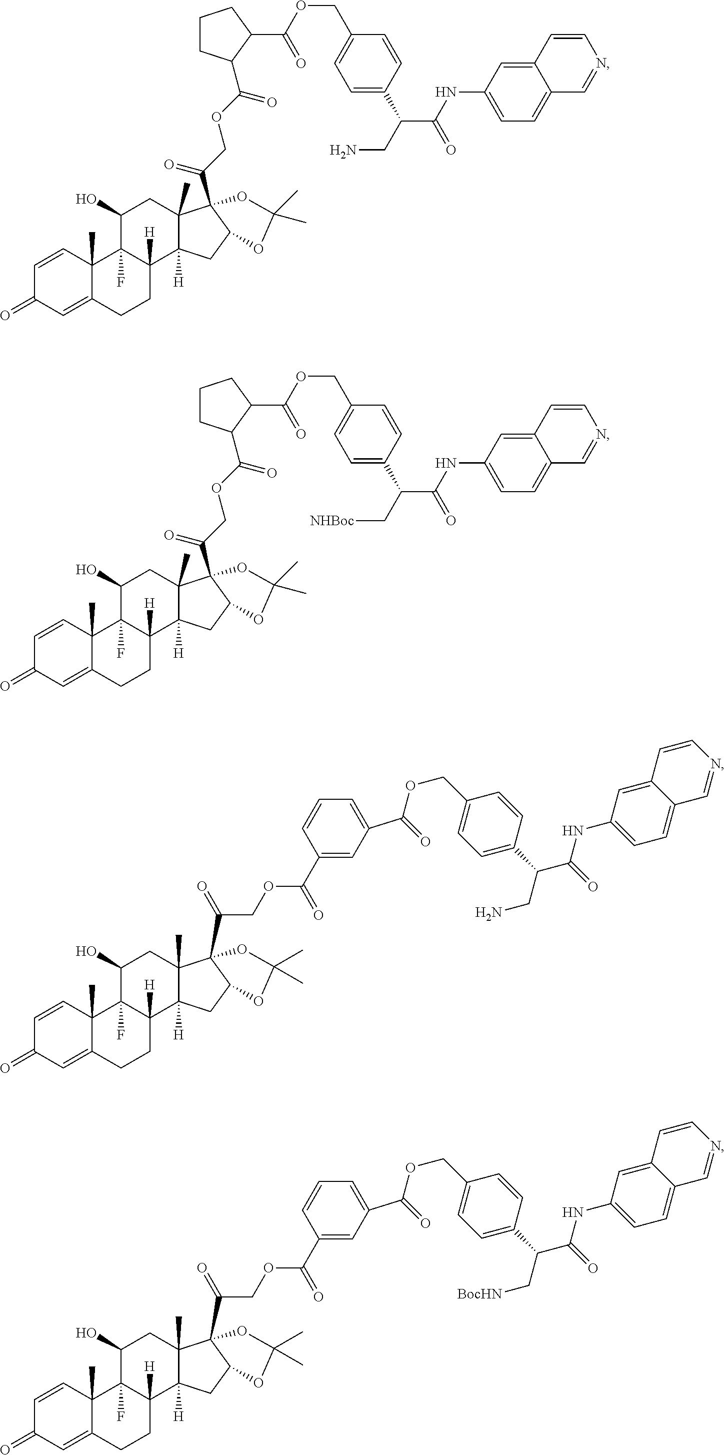 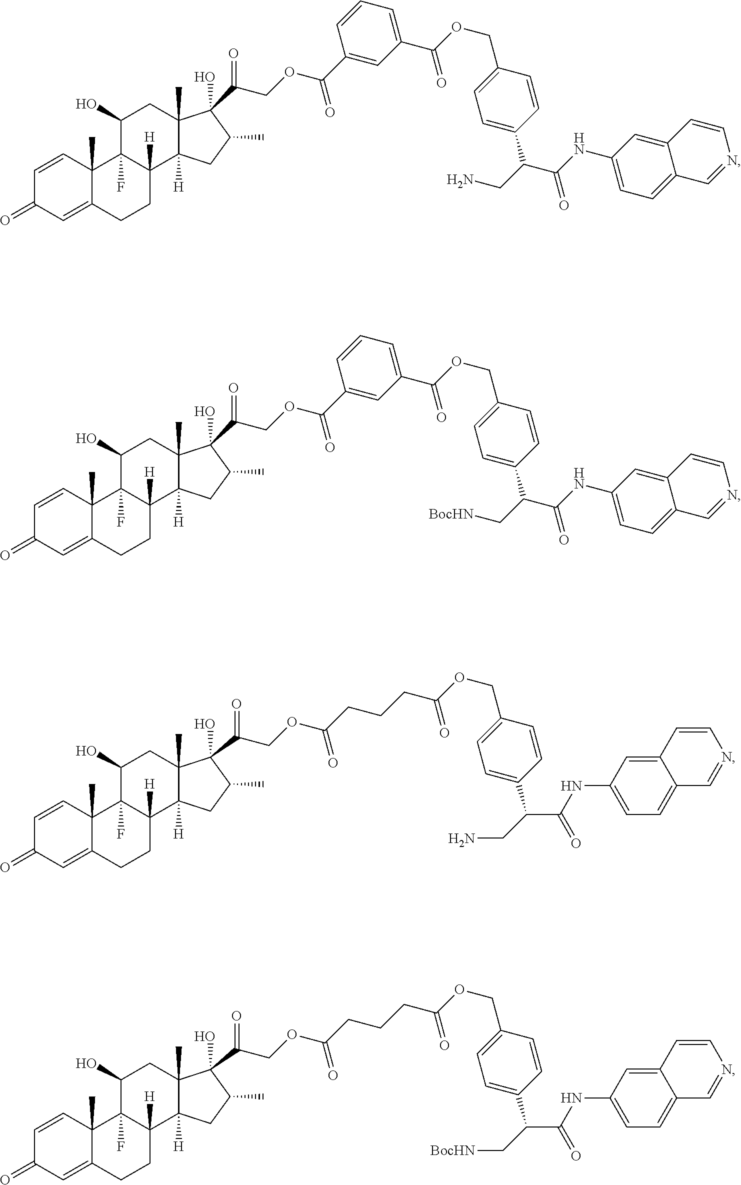 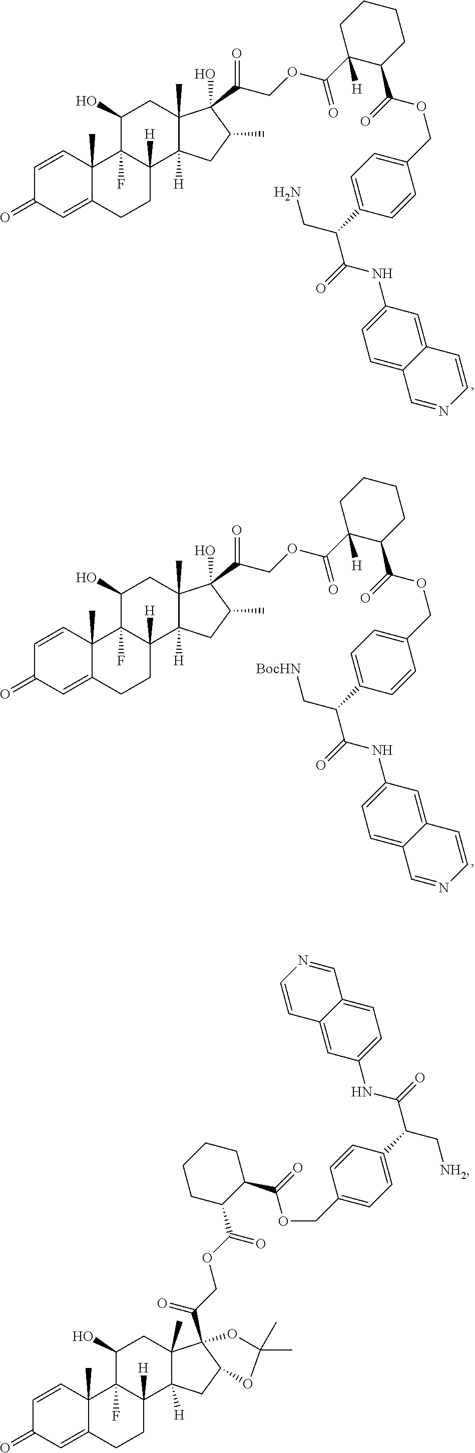 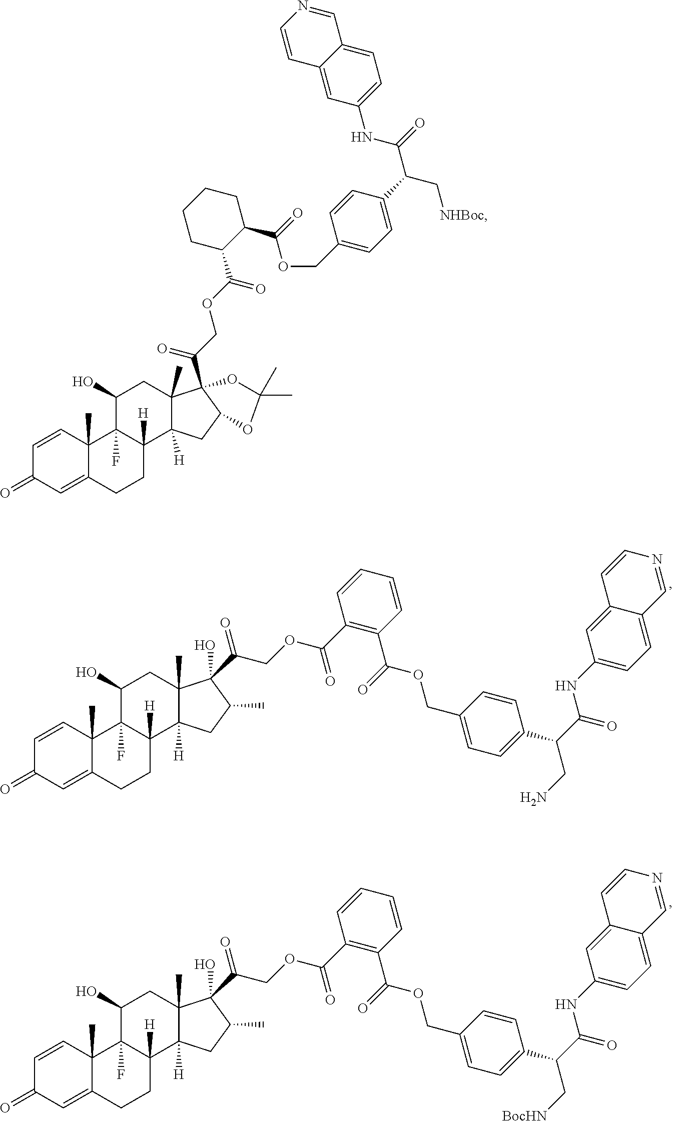 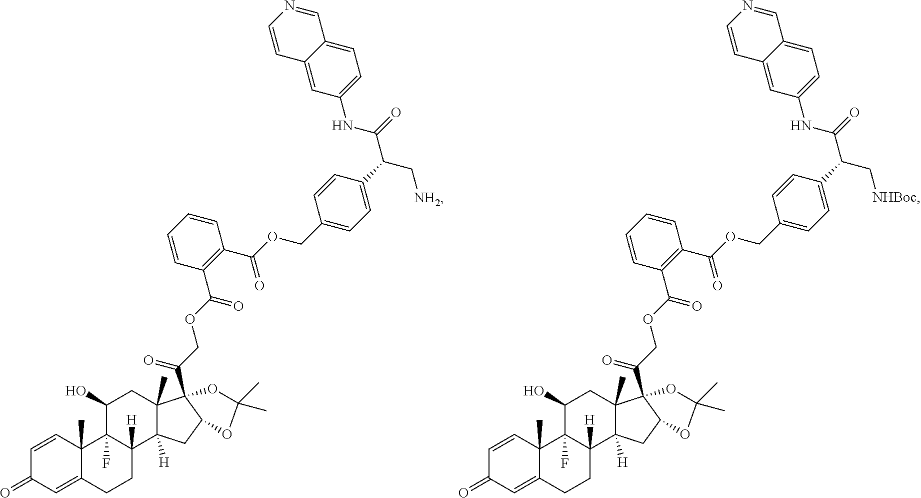 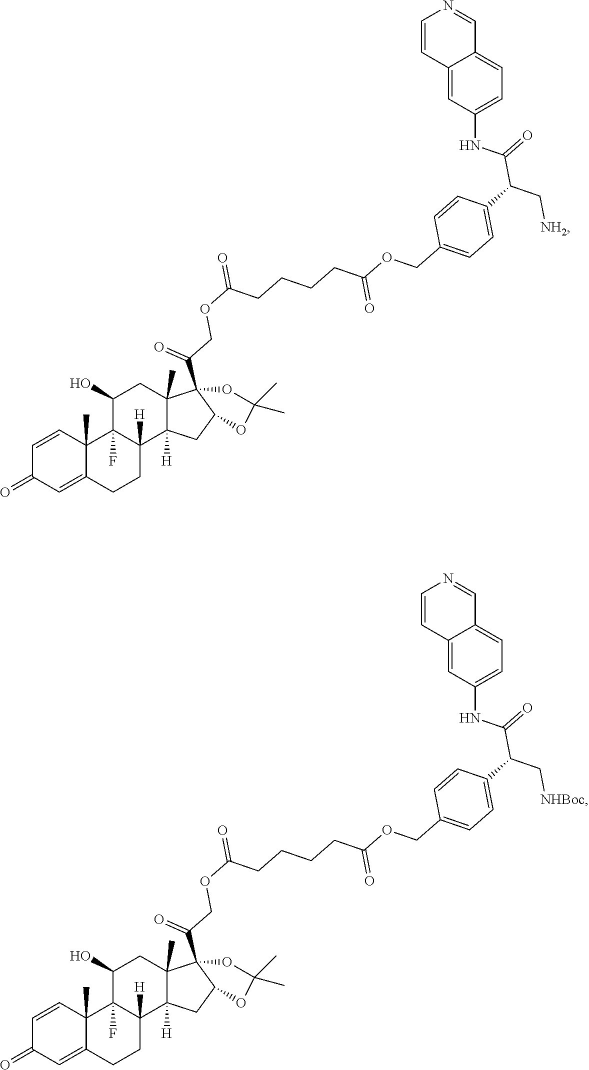 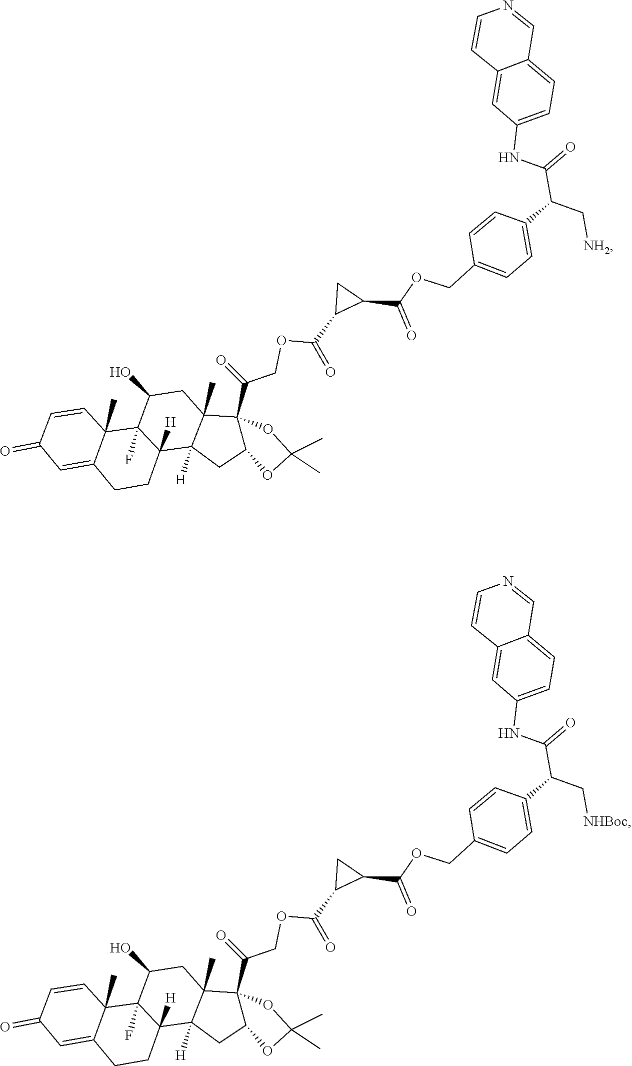 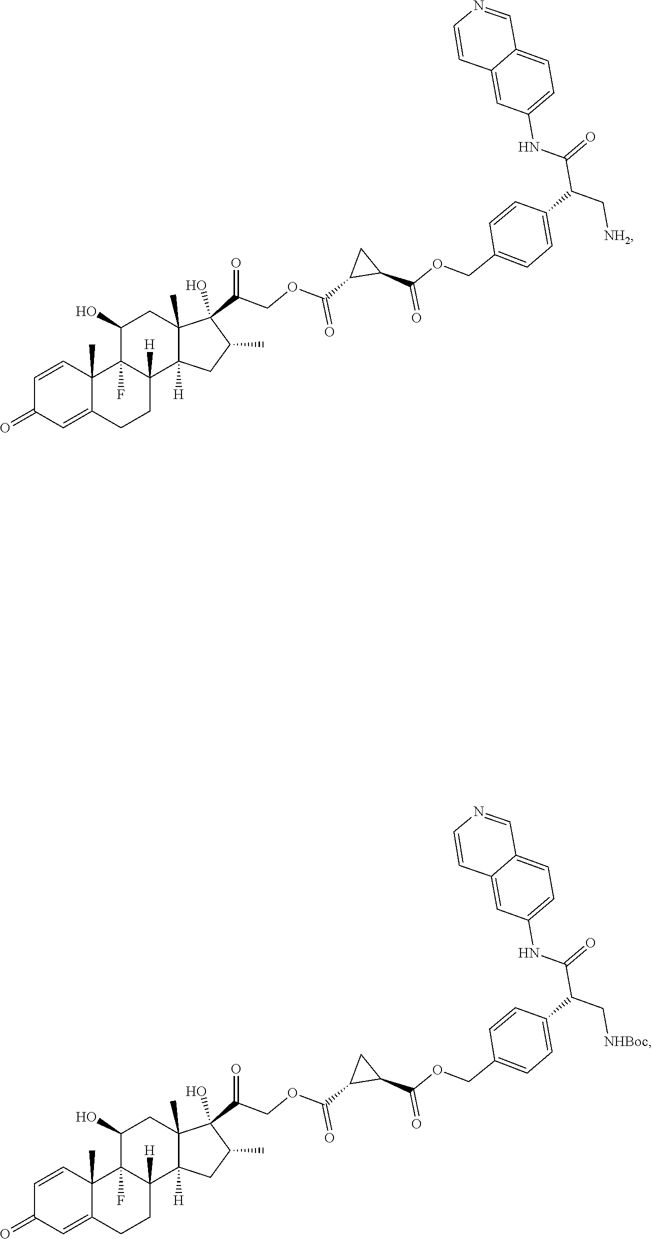 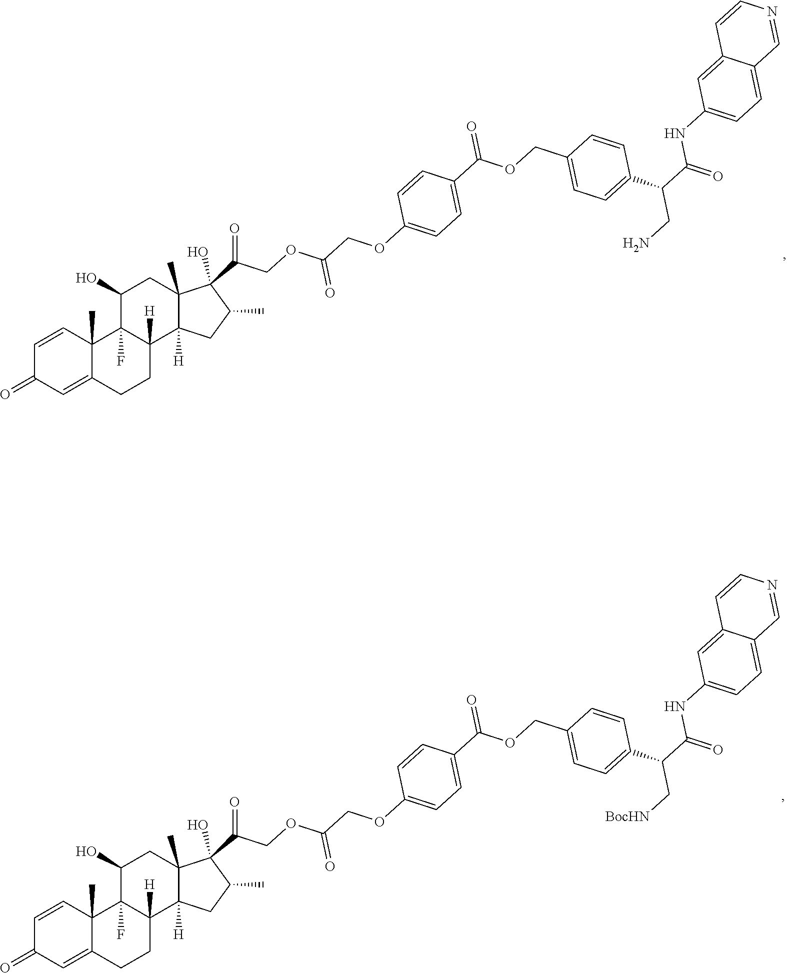 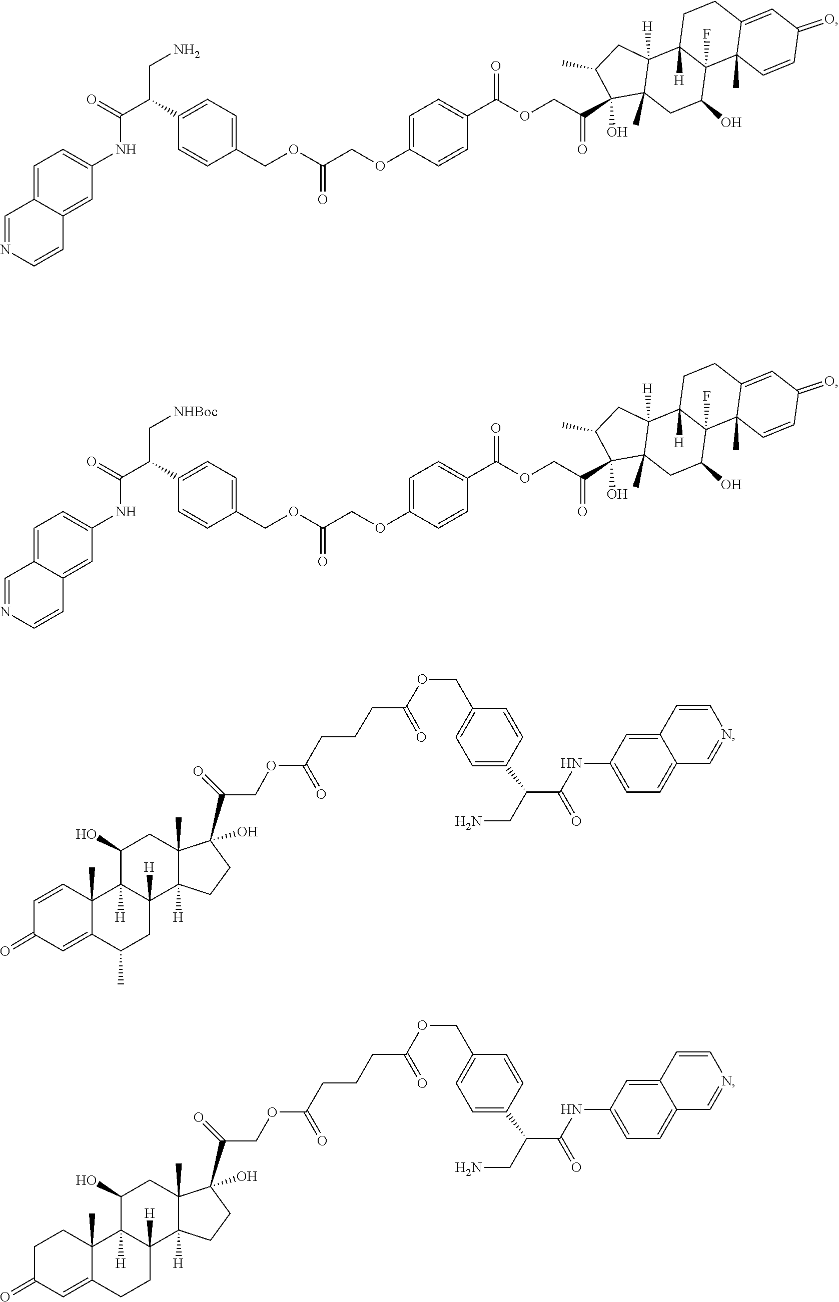 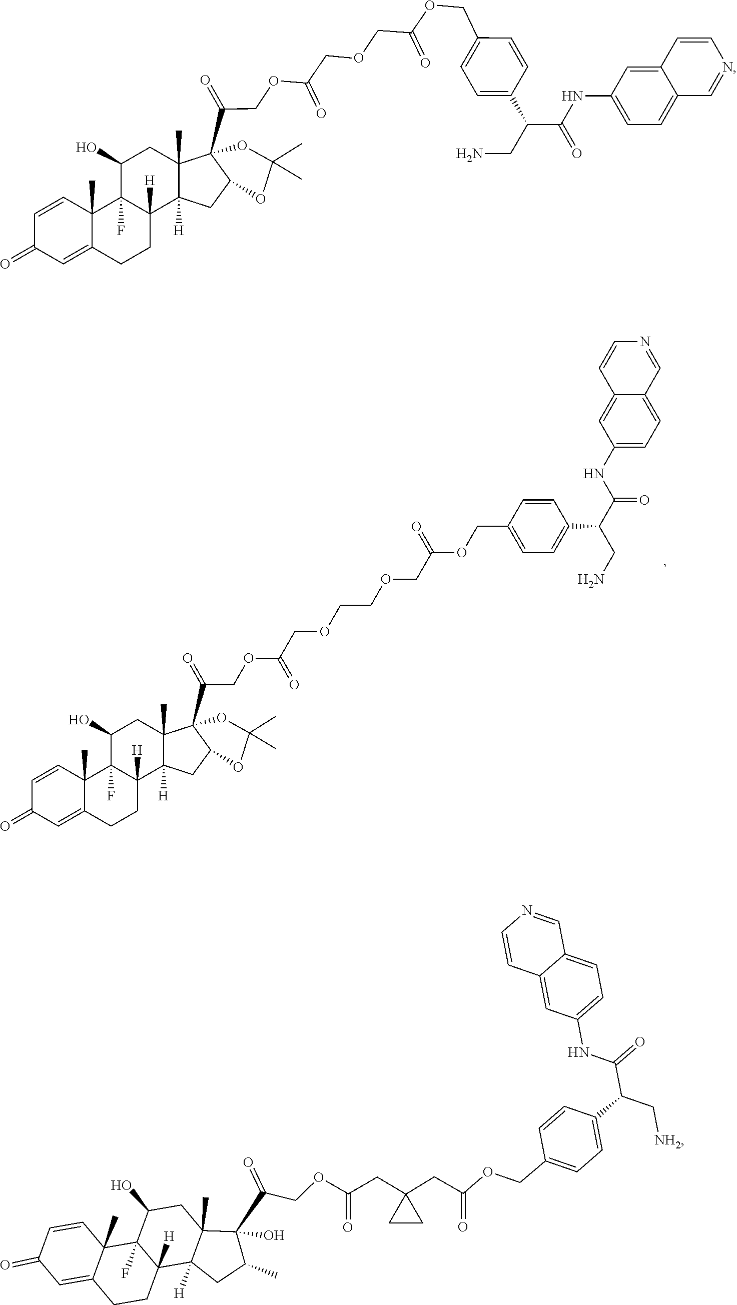 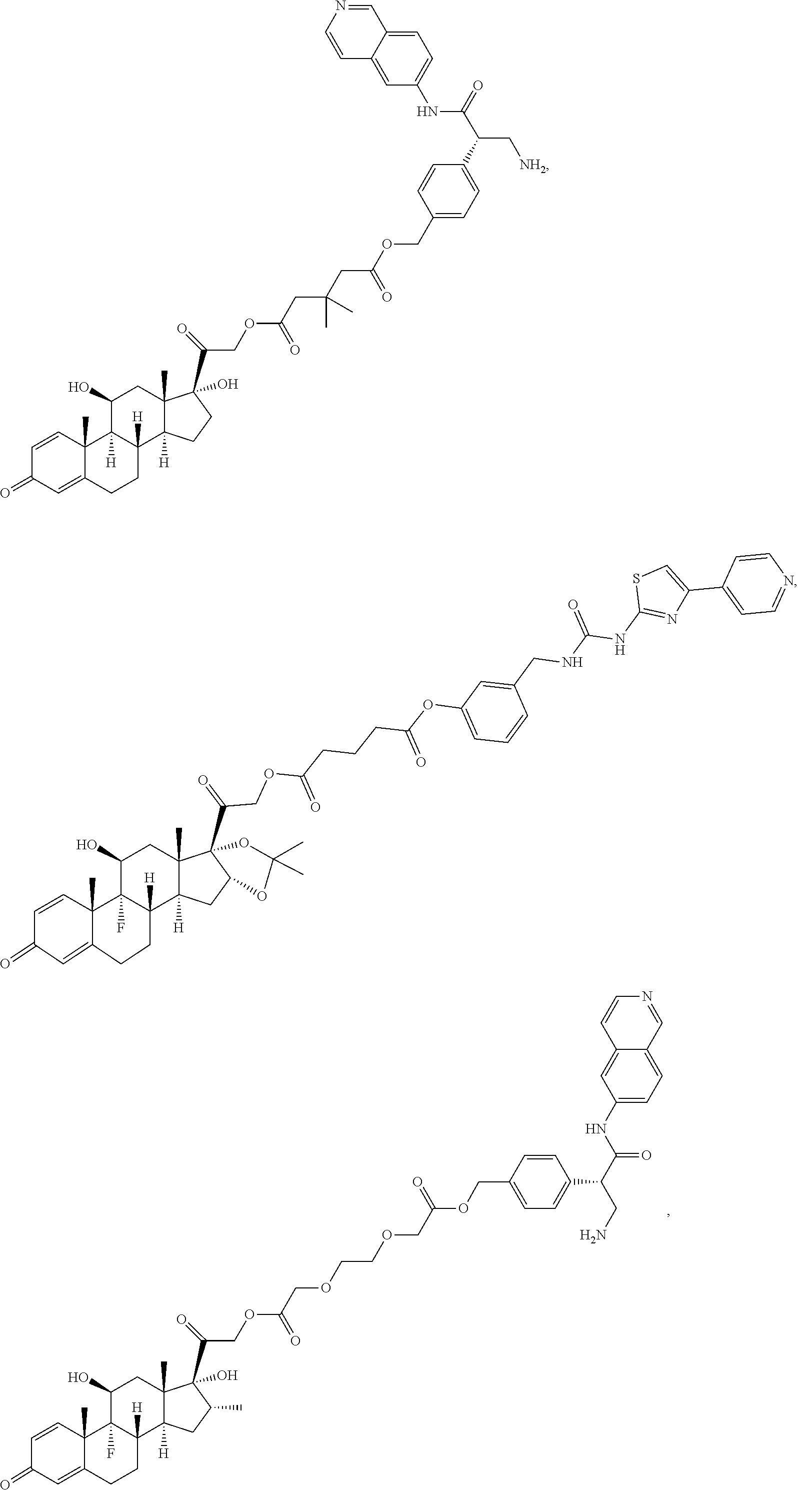 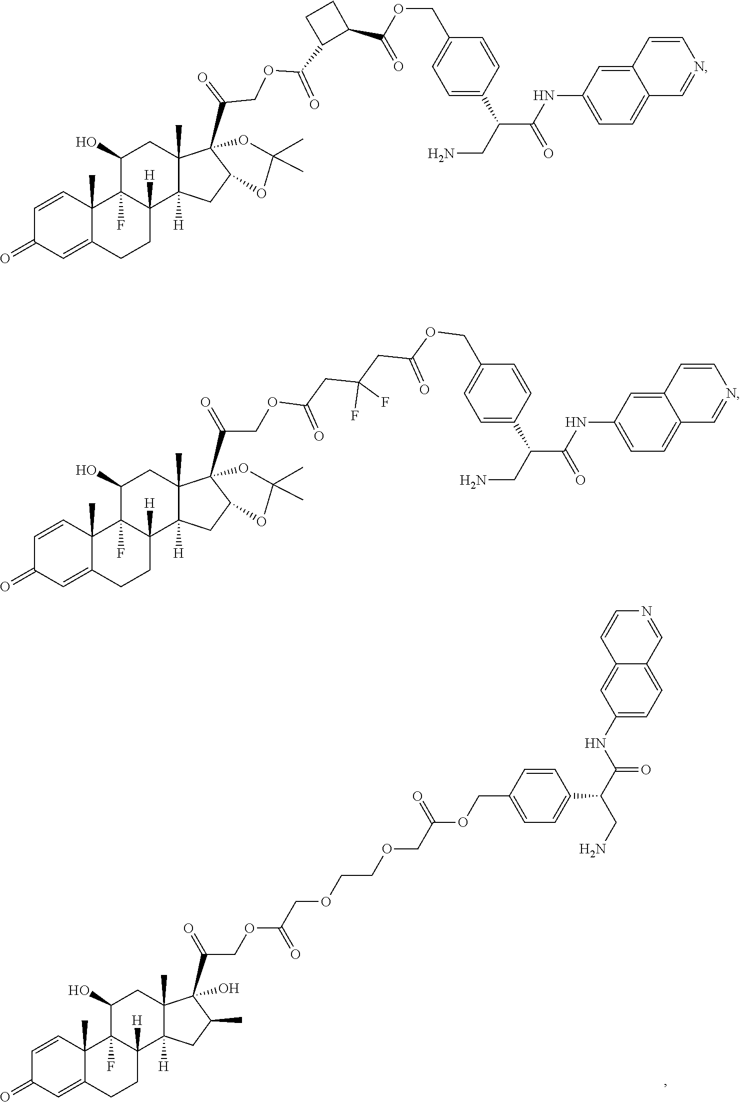 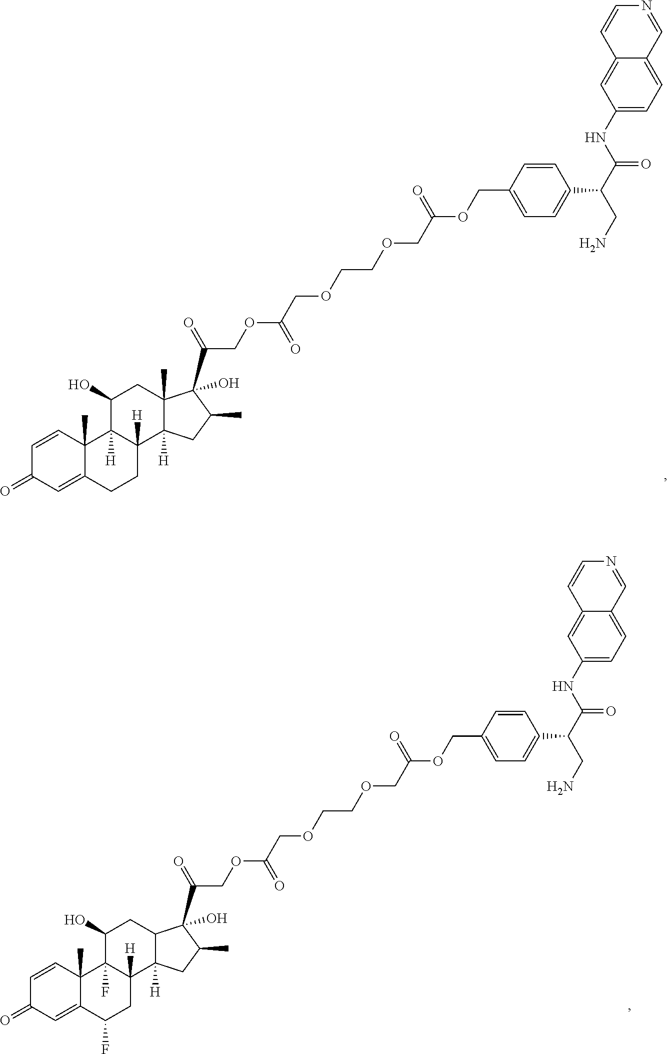 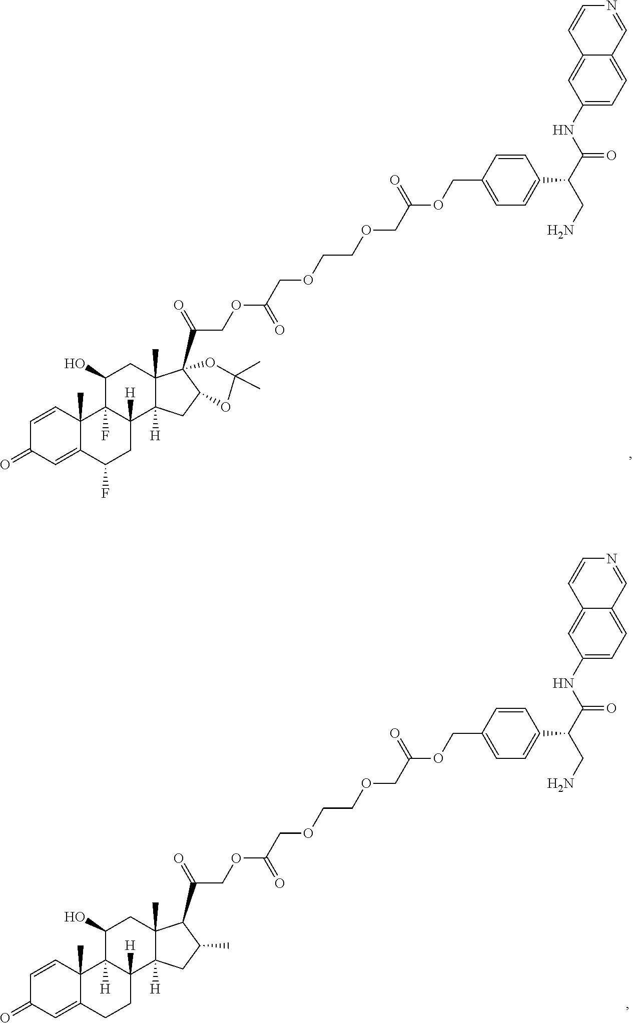 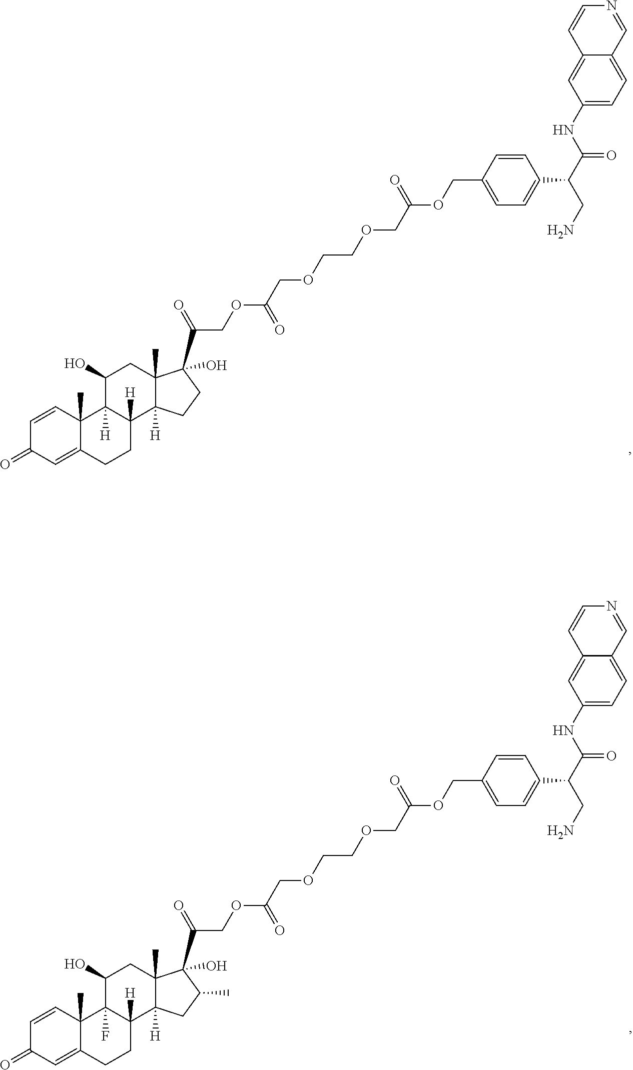 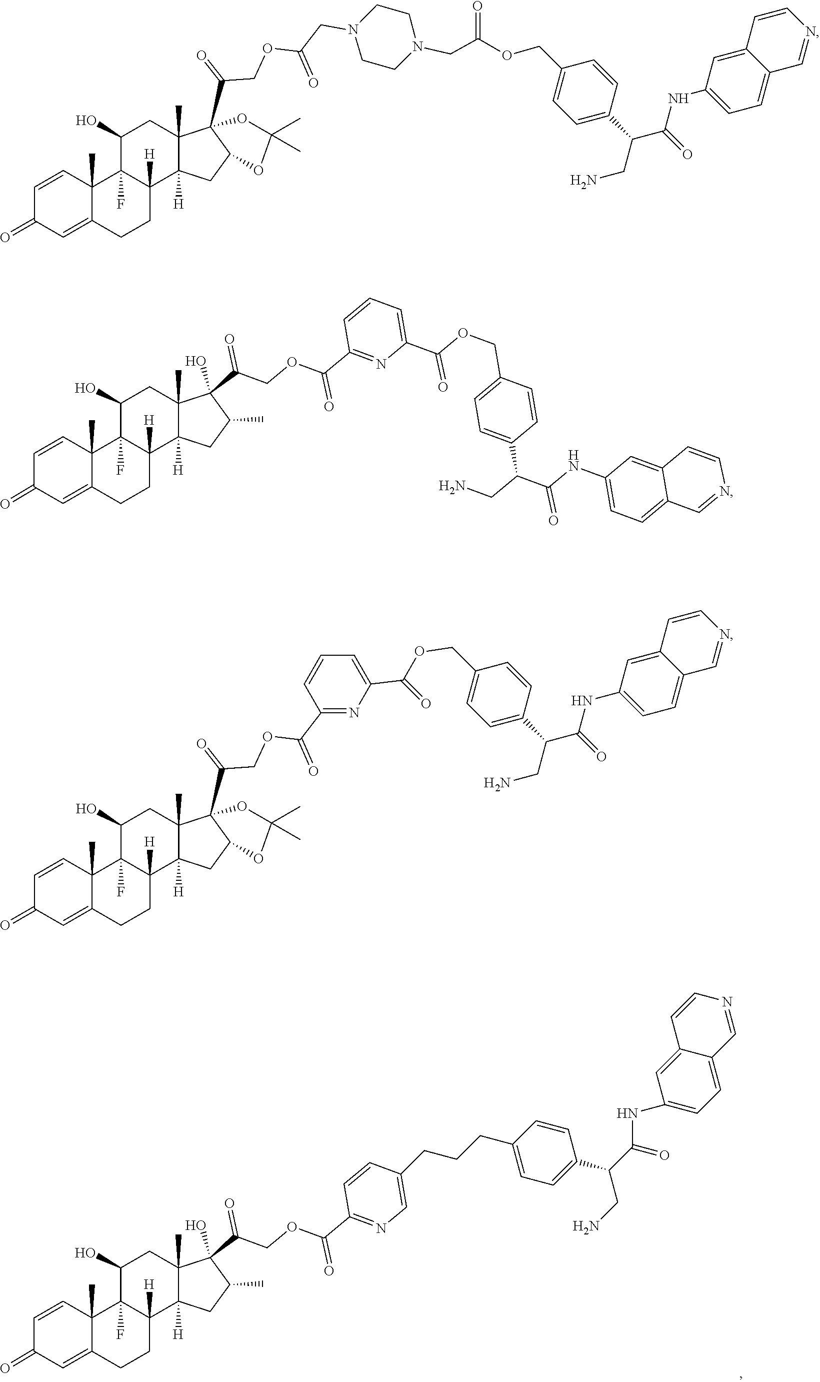 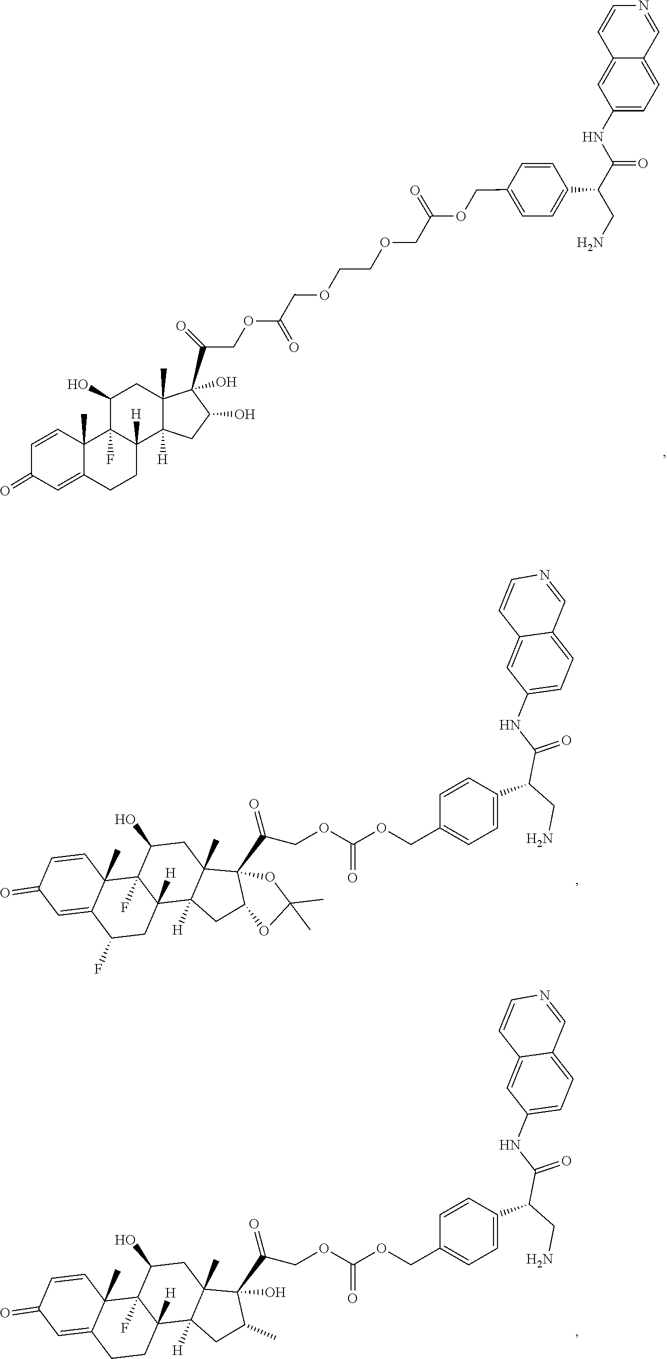 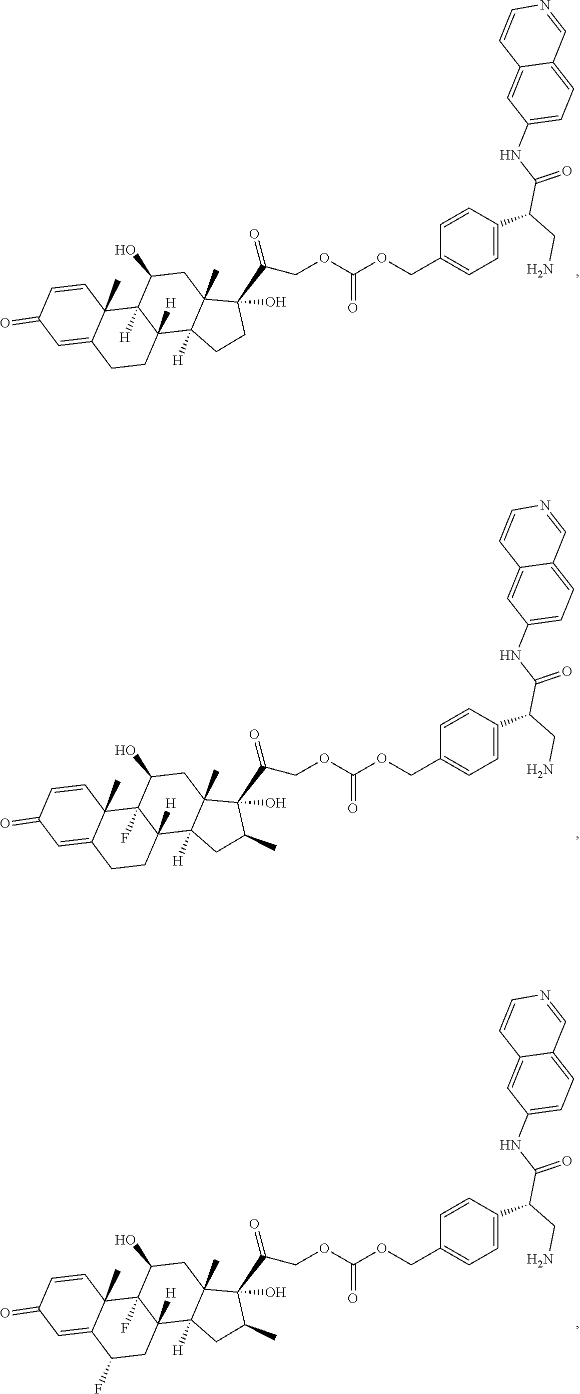 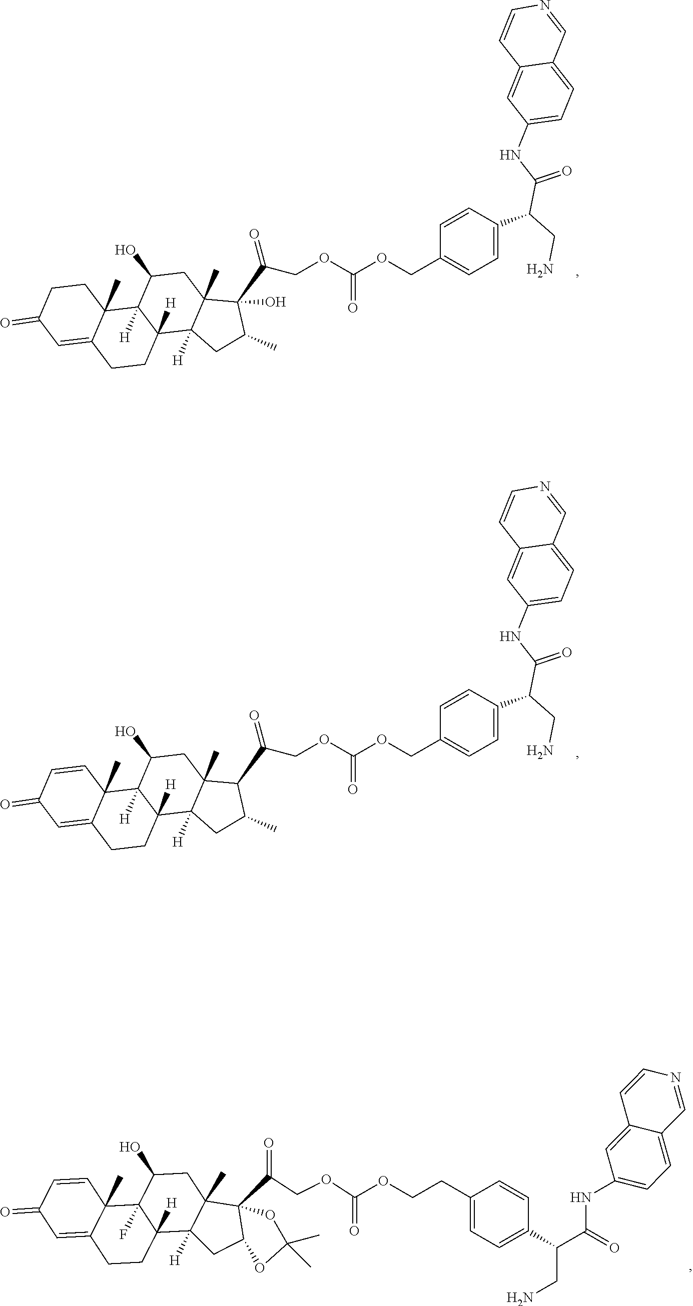 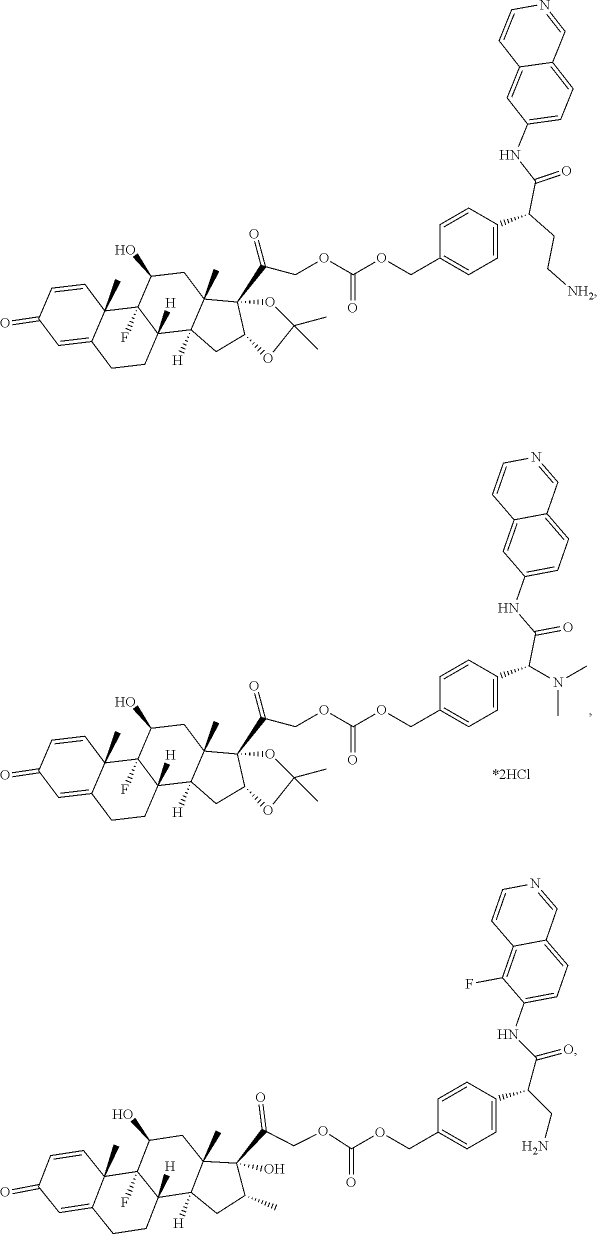 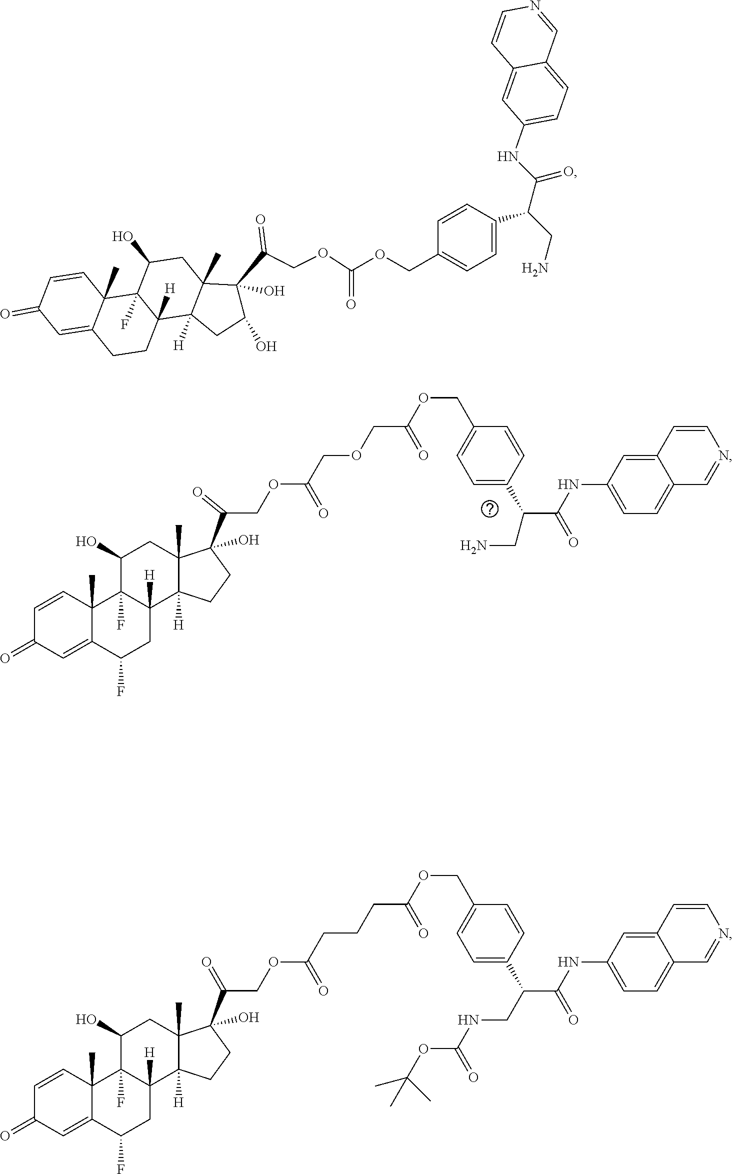 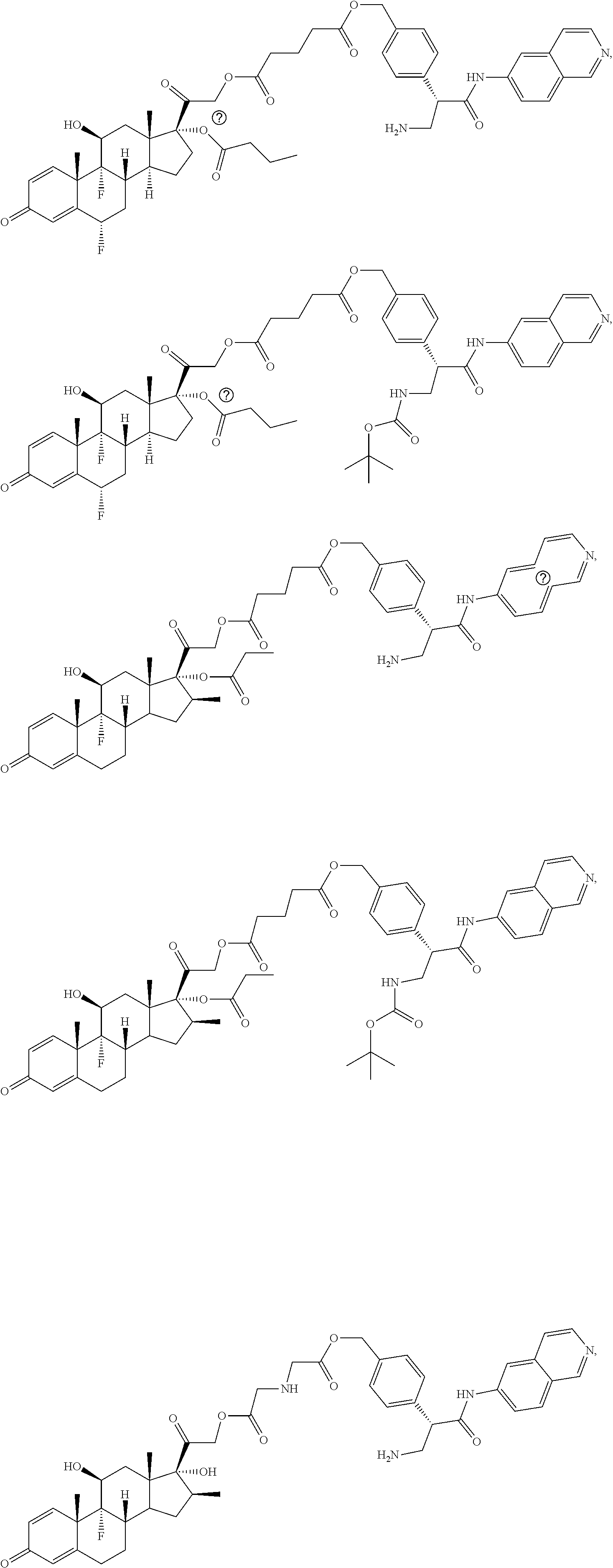 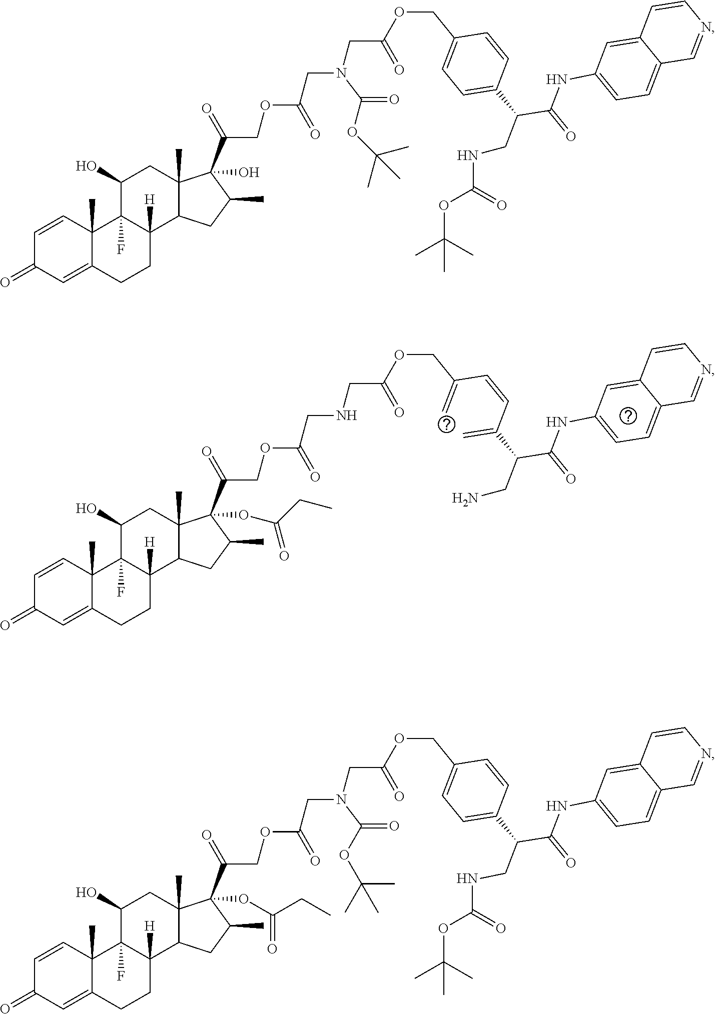 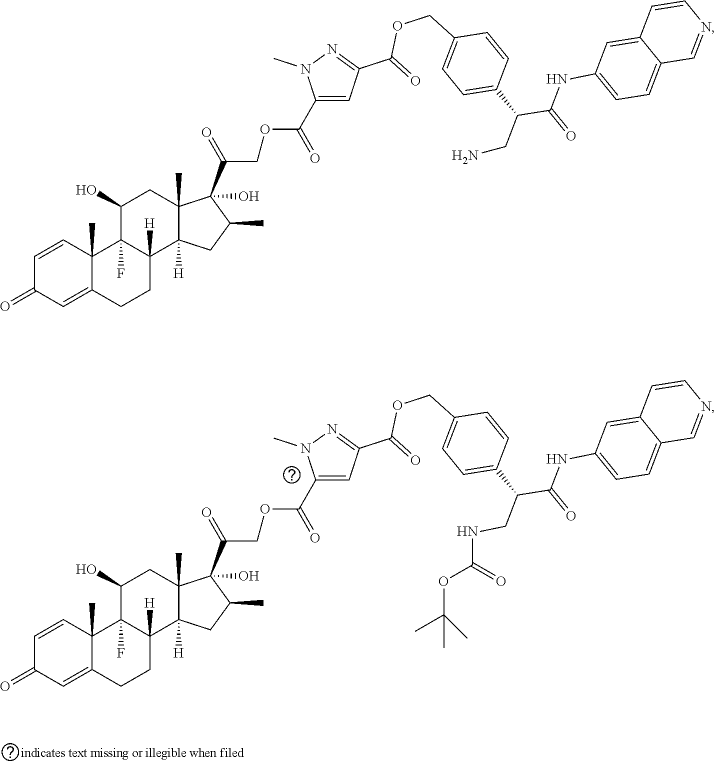
or a pharmaceutically acceptable salt thereof.

9. A compound, having a formula selected from: 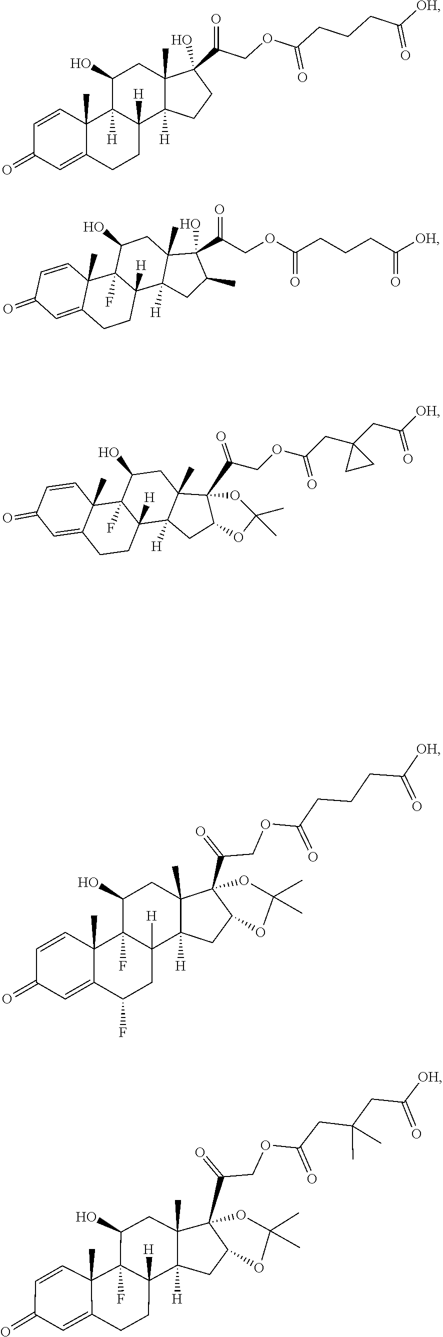 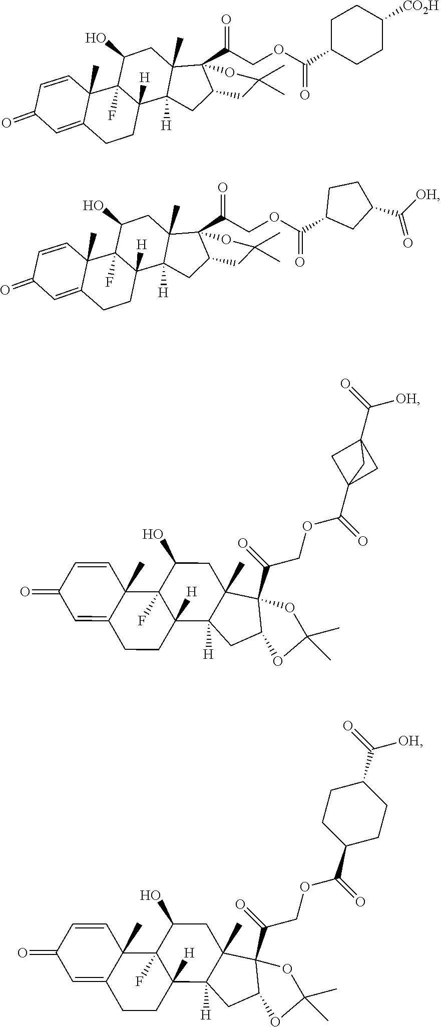 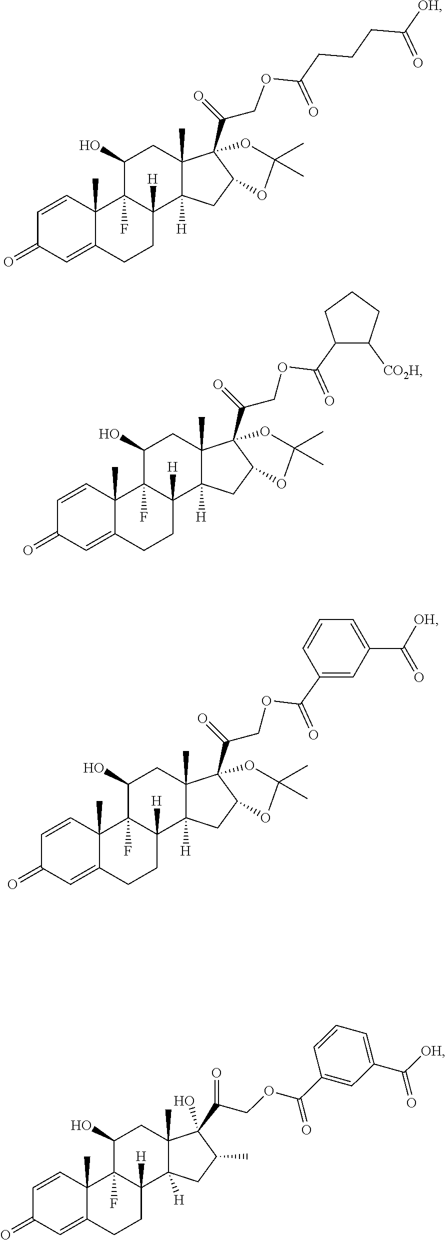 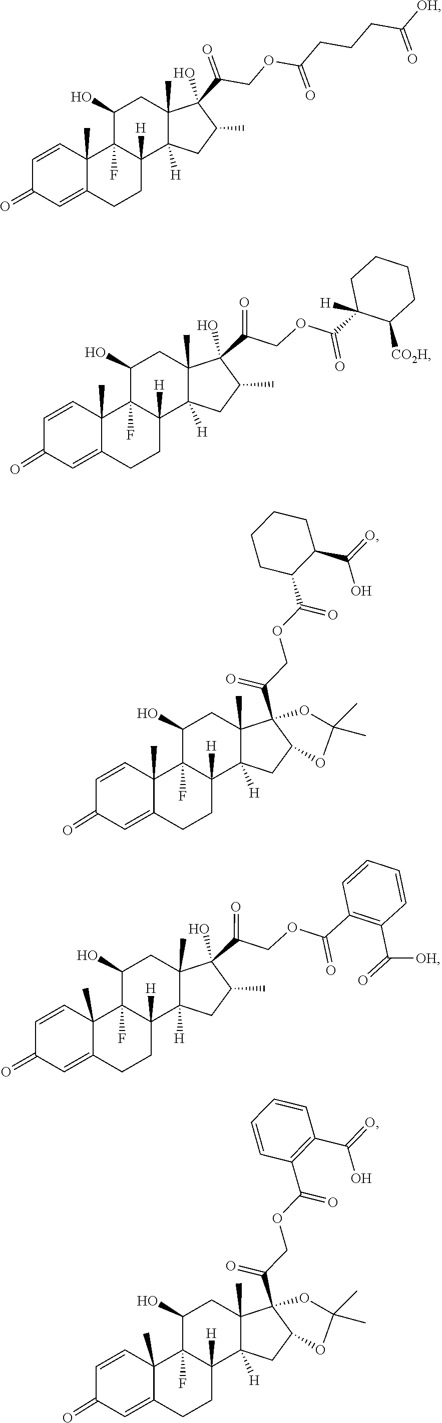 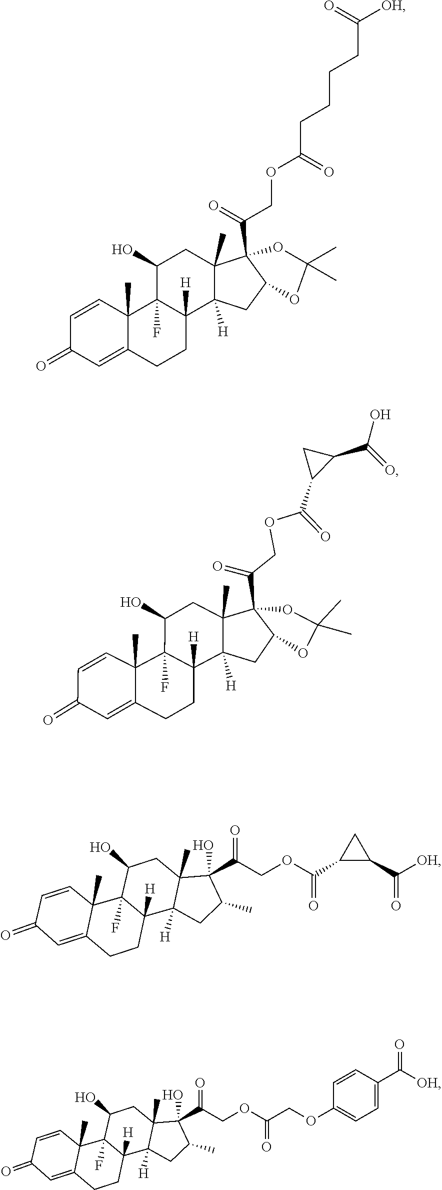 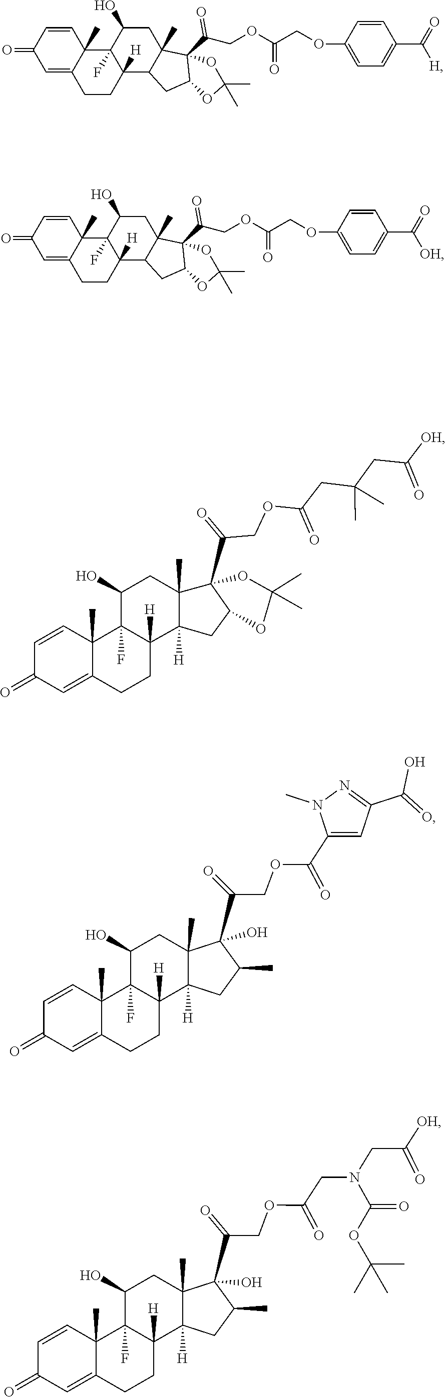 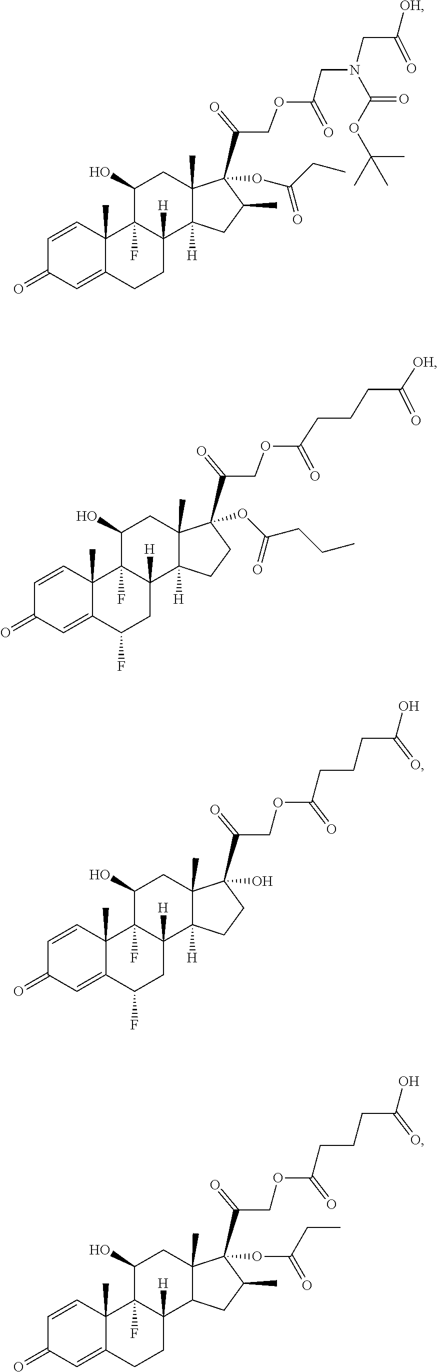
or a salt thereof.

10. A composition, comprising the compound of claim 1.

11. A pharmaceutical composition, comprising the compound of claim 1, further comprising a pharmaceutically acceptable carrier.

12. The compound or claim 1, housed within a container, optionally wherein the container blocks the transmission of visible or ultraviolet light.

13. A method of delivery, comprising administering the compound of claim 1 to a subject.

14. A method of treating an eye disease or disorder in a subject in need thereof, comprising administering to the subject an effective amount of the compound or claim 1.

15. A method of reducing intraocular pressure in an eye of a subject in need thereof, comprising administering to the subject an effective amount of the compound of claim 1.

16. A method of modulating kinase activity in a cell, comprising contacting the cell with an amount effective to modulate kinase activity of the compound of claim 1.

17. A method of treating an ocular inflammatory disease or disorder in a subject in need thereof, comprising administering to the subject an effective amount of the compound of claim 1.

18. A method of reducing the likelihood of contracting elevated intraocular pressure in an eye of a subject in need thereof, comprising administering to the subject an effective amount of the compound of claim 1.

19. A method of preventing an increase in intraocular pressure in an eye of a subject in need thereof, comprising administering to the subject an effective amount of the compound of claim 1.

20. A method of modulating intraocular pressure in an eye of a subject in need thereof, comprising administering to the subject an effective amount of the compound of claim 1, wherein the likelihood of contracting elevated intraocular pressure in the eye of the subject is reduced or not enhanced by administration of the compound.

21. The method of claim 20, wherein the administration is topical administration to an eye of the subject.
